(19) United States
(12) Patent Application Publication
(10) Pub. No.: US 2024/0308976 A1
(43) Pub. Date: Sep. 19, 2024

(54) COMPOUNDS THAT MEDIATE PROTEIN DEGRADATION AND USES THEREOF

(71) Applicant: Monte Rosa Therapeutics, Inc., Boston, MA (US)

(72) Inventors: Bernhard Fasching, Basel (CH); Thomas Ryckmans, Basel (CH); Oliv Eidam, Basel (CH); Alexander Flohr, Basel (CH); Laura Ann McAllister, Basel (CH); Andreas Ritzén, Basel (CH)

(21) Appl. No.: 18/640,889

(22) Filed: Apr. 19, 2024

Related U.S. Application Data
(63) Continuation of application No. PCT/US2022/047421, filed on Oct. 21, 2022.
(60) Provisional application No. 63/262,922, filed on Oct. 22, 2021.
(60) Provisional application No. 63/270,935, filed on Oct. 22, 2021.

(51) Int. Cl. C07D 401/10; A61K 31/4523; A61K 31/4525; A61K 31/453; A61K 31/4535; A61K 31/454; A61K 31/4545; A61K 31/506; A61K 31/5377; A61K 31/5386; C07D 401/14; C07D 405/14; C07D 409/14; C07D 491/107; C07D 498/08

(52) U.S. Cl. CPC C07D 401/10; A61K 31/4523; A61K 31/4525; A61K 31/453; A61K 31/4535; A61K 31/454; A61K 31/4545; A61K 31/506; A61K 31/5377; A61K 31/5386; C07D 401/14; C07D 405/14; C07D 409/14; C07D 491/107; C07D 498/08

(57) ABSTRACT

Described herein, in part, are compounds that bind to and modulate the surface of cereblon and mediate the degradation of GSPT1, and are therefore useful in the treatment of various disorders, such as cancer.

### CROSS-REFERENCE TO RELATED APPLICATIONS

This application is a continuation of International Application Number PCT/US2022/047421, filed on Oct. 21, 2022, which claims the benefit of, and priority to U.S. Provisional Application No. 63/262,922, filed Oct. 22, 2021 and U.S. Provisional Application No. 63/270,935, filed Oct. 22, 2021, the contents of each of which are incorporated herein by reference.

### BACKGROUND

The ubiquitin proteasome system can be manipulated with different small molecules to trigger targeted degradation of specific proteins of interest. Promoting the targeted degradation of pathogenic proteins using small molecule degraders is emerging as a new modality in the treatment of diseases. One such modality relies on redirecting the activity of E3 ligases such as cereblon (a phenomenon known as E3 reprogramming) using low molecular weight compounds, which have been termed molecular glues to promote the poly-ubiquitination and ultimately proteasomal degradation of new protein substrates involved in the development of diseases. The molecular glues bind to both the E3 ligase and the target protein, thereby mediating an alteration of the ligase surface and enabling an interaction with the target protein.

There exists a need for therapeutics that effectively mediate the degradation of certain proteins for the treatment of diseases.

### SUMMARY

Described herein, in part, are compounds contemplated as molecular glues that bind cereblon and mediate the degradation of a protein, and are therefore are useful in the treatment of disorders, such as cancer. For example, it has been found that compounds of the present disclosure bind to and modulate the surface of cereblon to subsequently mediate the targeted degradation of the protein GSPT1.

In one aspect, described herein is a compound of Formula (X):

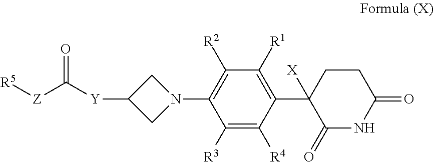

or a pharmaceutically acceptable salt thereof, wherein: X is H or deuterium; Y is O or NH; Z is NR^{A}; each of R¹, R², R³, and R⁴ is independently H, halogen, cyano, or C₁₋₆ alkoxy; R⁵ is C₁₋₆ alkyl, C₂₋₆ alkenyl, C₂₋₆ alkynyl, C₃₋₁₀ cycloalkyl, 3 to 10 membered heterocyclyl, aryl, or heteroaryl, wherein each of C₁₋₆ alkyl, C₂₋₆ alkenyl, C₂₋₆ alkynyl, C₃₋₁₀ cycloalkyl, C₆₋₁₀ aryl, and heteroaryl is optionally substituted with one or more substituents each independently selected from R⁶; or R^{A} and R⁵ are joined together to form a 3 to 10 membered nitrogen-containing heterocyclyl optionally substituted with one or more substituents each independently selected from R⁶; each occurrence of R⁶ is independently halogen, cyano, —S(CH₃), C₁₋₆ alkyl, C₁₋₆ alkoxy, C₁₋₆ thioalkoxy, C₃₋₁₀ cycloalkyl, 3 to 10 membered heterocyclyl, aryl, heteroaryl, —C(O)OR^{B}, or —OC(O)R^{C}, wherein each of C₁₋₆ alkyl, C₁₋₆ alkoxy, C₃₋₁₀ cycloalkyl, 3 to 10 membered heterocyclyl, aryl, and heteroaryl is optionally substituted with one or more substituents each independently selected from R⁷, or two R⁶ are joined to together to form a 3 to 10 membered ring optionally substituted with one or more substituents each independently selected from R⁷; each occurrence of R⁷ is independently halogen, —OH, C₁₋₆ alkyl, 3 to 10 membered heterocyclyl, or aryl, wherein each of C₁₋₆ alkyl, 3 to 10 membered heterocyclyl, and aryl is optionally substituted with R⁸; each occurrence of R⁸ is C₁₋₆ alkoxy; R^{A} is H or C₁₋₃ alkyl (e.g., —CH₃), or R^{A} and R⁵ are joined together to form a 3 to 10 membered nitrogen-containing heterocyclyl optionally substituted with one or more substituents each independently selected from R⁶; and each of R^{B} and R^{C} is independently H or C₁₋₆ alkyl.

In one aspect, described herein is a compound of Formula (I):

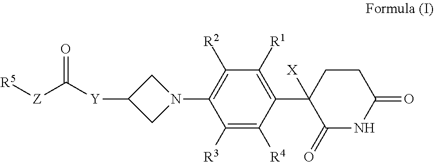

or a pharmaceutically acceptable salt thereof, wherein: X is H or deuterium; Y is O or NH; Z is NR^{A}; each of R¹, R², R³, and R⁴ is independently H, halogen, cyano, or C₁₋₆ alkoxy; R⁵ is C₁₋₆ alkyl, C₂₋₆ alkenyl, C₂₋₆ alkynyl, C₃₋₁₀ cycloalkyl, 3 to 10 membered heterocyclyl, aryl, or heteroaryl, wherein each of C₁₋₆ alkyl, C₂₋₆ alkenyl, C₂₋₆ alkenyl, C₃₋₁₀ cycloalkyl, C₆₋₁₀ aryl, and heteroaryl is optionally substituted with one or more occurrences of R⁶; or R^{A} and R⁵ are joined together to form a 3 to 10 membered nitrogen-containing heterocyclyl optionally substituted with one or more occurrences of R⁶; each occurrence of R⁶ is independently halogen, cyano, C₁₋₆ alkyl, C₁₋₆ alkoxy, C₁₋₆ thioalkoxy, C₃₋₁₀ cycloalkyl, 3 to 10 membered heterocyclyl, aryl, heteroaryl, —C(O)OR^{B}, or —OC(O)R^{C}, wherein each of C₁₋₆ alkyl, C₁₋₆ alkoxy, C₃₋₁₀ cycloalkyl, 3 to 10 membered heterocyclyl, aryl, and heteroaryl is optionally substituted with one or more occurrences of R⁷, or two R⁶ are joined to together to form a 3 to 10 membered ring optionally substituted with one or more occurrences of R⁷; each occurrence of R⁷ is independently halogen, C₁₋₆ alkyl, 3 to 10 membered heterocyclyl, or aryl, wherein each of C₁₋₆ alkyl, 3 to 10 membered heterocyclyl, and aryl is optionally substituted with R⁷; each occurrence of R⁷ is C₁₋₆ alkoxy; R^{A} is H, or R^{A} and R⁵ are joined together to form a 3 to 10 membered nitrogen-containing heterocyclyl optionally substituted with one or more occurrences of R⁶; and each of R^{B} and R^{C} is independently H or C₁₋₆ alkyl.

In another aspect, provided herein is a pharmaceutical composition comprising a compound described herein, or pharmaceutically acceptable salt thereof, and a pharmaceutically acceptable excipient.

In another aspect, described herein is a method of degrading GSPT1 in a subject suffering from cancer, comprising administering to the subject an effective amount of a compound described herein, or pharmaceutically acceptable salt thereof, or a pharmaceutical composition described herein.

In another aspect, described herein is a method of treating cancer in a subject in need thereof, comprising administering to the subject a therapeutically effective amount of a compound described herein, or pharmaceutically acceptable salt thereof, or a pharmaceutical composition described herein.

In another aspect, described herein is a method of treating a solid tumor in a subject in need thereof, comprising administering to the subject a therapeutically effective amount of a compound described herein, or pharmaceutically acceptable salt thereof, or a pharmaceutical composition described herein.

In another aspect, described herein is a method of treating a liquid tumor in a subject in need thereof, comprising administering to the subject a therapeutically effective amount of a compound described herein, or pharmaceutically acceptable salt thereof, or a pharmaceutical composition described herein.

### DETAILED DESCRIPTION

The features and other details of the disclosure will now be more particularly described. Certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### Compounds

In one aspect, described herein is a compound of Formula (X):

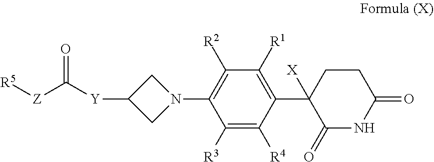

or a pharmaceutically acceptable salt thereof, wherein: X is H or deuterium; Y is O or NH; Z is NR^{A}; each of R¹, R², R³, and R⁴ is independently H, halogen, cyano, or C₁₋₆ alkoxy; R⁵ is C₁₋₆ alkyl, C₂₋₆ alkenyl, C₂₋₆ alkynyl, C³⁻¹⁰ cycloalkyl, 3 to 10 membered heterocyclyl, aryl, or heteroaryl, wherein each of C₁₋₆ alkyl, C₂₋₆ alkenyl, C₂₋₆ alkynyl, C₃₋₁₀ cycloalkyl, C₆₋₁₀ aryl, and heteroaryl is optionally substituted with one or more substituents each independently selected from R⁶; or R^{A} and R⁵ are joined together to form a 3 to 10 membered nitrogen-containing heterocyclyl optionally substituted with one or more substituents each independently selected from R⁶; each occurrence of R⁶ is independently halogen, cyano, —S(CH₃), C₁₋₆ alkyl, C₁₋₆ alkoxy, C₁₋₆ thioalkoxy, C₃₋₁₀ cycloalkyl, 3 to 10 membered heterocyclyl, aryl, heteroaryl, —C(O)OR^{B}, or —OC(O)R^{C}, wherein each of C₁₋₆ alkyl, C₁₋₆ alkoxy, C₃₋₁₀ cycloalkyl, 3 to 10 membered heterocyclyl, aryl, and heteroaryl is optionally substituted with one or more substituents each independently selected from R⁷, or two R⁶ are joined to together to form a 3 to 10 membered ring optionally substituted with one or more substituents each independently selected from R⁷; each occurrence of R⁷ is independently halogen, —OH, C₁₋₆ alkyl, 3 to 10 membered heterocyclyl, or aryl, wherein each of C₁₋₆ alkyl, 3 to 10 membered heterocyclyl, and aryl is optionally substituted with R⁸; each occurrence of R⁸ is C₁₋₆ alkoxy; R^{A} is H or C₁₋₃ alkyl (e.g., —CH₃), or R^{A} and R⁵ are joined together to form a 3 to 10 membered nitrogen-containing heterocyclyl optionally substituted with one or more substituents each independently selected from R⁶; and each of R^{B} and R^{C} is independently H or C₁₋₆ alkyl.

In some embodiments, Y is O. In some embodiments, Z is N(CH₃). In some embodiments, Y is O and Z is N(CH₃).

In one aspect, described herein is a compound of Formula (I):

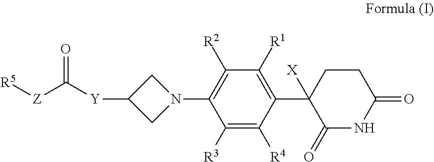

or a pharmaceutically acceptable salt thereof, wherein: X is H or deuterium; Y is O or NH; Z is NR^{A}; each of R¹, R², R³, and R⁴ is independently H, halogen, cyano, or C₁₋₆ alkoxy; R⁵ is C₁₋₆ alkyl, C₂₋₆ alkenyl, C₂₋₆ alkynyl, C₃₋₁₀ cycloalkyl, 3 to 10 membered heterocyclyl, aryl, or heteroaryl, wherein each of C₁₋₆ alkyl, C₂₋₆ alkenyl, C₂₋₆ alkenyl, C₃₋₁₀ cycloalkyl, C⁶⁻¹⁰ aryl, and heteroaryl is optionally substituted with one or more occurrences of R⁶; or R^{A} and R⁵ are joined together to form a 3 to 10 membered nitrogen-containing heterocyclyl optionally substituted with one or more occurrences of R⁶; each occurrence of R⁶ is independently halogen, cyano, C₁₋₆ alkyl, C₁₋₆ alkoxy, C₁₋₆ thioalkoxy, C₃₋₁₀ cycloalkyl, 3 to 10 membered heterocyclyl, aryl, heteroaryl, —C(O)OR^{B}, or —OC(O)R^{C}, wherein each of C₁₋₆ alkyl, C₁₋₆ alkoxy, C₃₋₁₀ cycloalkyl, 3 to 10 membered heterocyclyl, aryl, and heteroaryl is optionally substituted with one or more occurrences of R⁷, or two R⁶ are joined to together to form a 3 to 10 membered ring optionally substituted with one or more occurrences of R⁷; each occurrence of R⁷ is independently halogen, C₁₋₆ alkyl, 3 to 10 membered heterocyclyl, or aryl, wherein each of C₁₋₆ alkyl, 3 to 10 membered heterocyclyl, and aryl is optionally substituted with R⁸; each occurrence of R⁸ is C₁₋₆ alkoxy; R^{A} is H, or R^{A} and R⁵ are joined together to form a 3 to 10 membered nitrogen-containing heterocyclyl optionally substituted with one or more occurrences of R⁶; and each of R^{B} and R^{C} is independently H or C₁₋₆ alkyl.

In some embodiments, the compound is a compound of Formula (I-A):

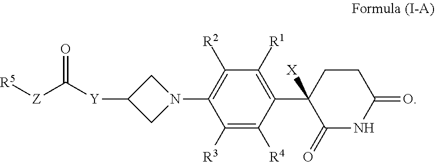

In some embodiments, the compound is a compound of Formula (I-B):

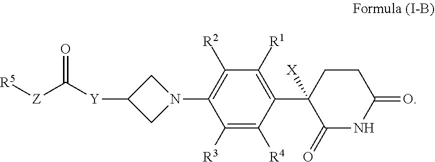

In some embodiments, X is H. In some embodiments, Y is O and Z is NH. In some embodiments, Y and Z are NH. In some embodiments, R^{A} is H. In some embodiments, R¹ and R⁴ are halogen. In some embodiments, R¹ and R⁴ are fluorine. In some embodiments, R² and R³ are H. In some embodiments, R⁵ is C₃₋₁₀ cycloalkyl, C⁶⁻¹⁰ aryl, 5-membered heteroaryl, or 6-membered heteroaryl.

In some embodiments, R⁵ is:

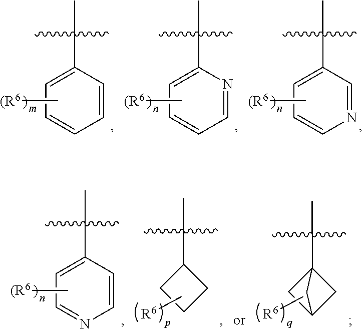

wherein m is an integer from 0 to 5, each occurrence of n is independently an integer from 0 to 4, p is an integer from 0 to 3, and q is an integer from 0 to 1. In some embodiments, R⁶ is halogen, cyano, C₁₋₄ alkyl, or C₁₋₆ alkoxy, wherein each of C₁₋₆ alkyl or C₁₋₆ alkoxy is optionally substituted with one or more occurrences of halogen. In some embodiments, R⁶ is chlorine, cyano, —CH₃, or —OCH₃. In some embodiments, R⁶ is chlorine, cyano, or —CH₃. In some embodiments, R⁷ is halogen or C₁₋₆ alkyl. In some embodiments, R⁷ is chlorine or —CH₃. In some embodiments, m is 1, 2, or 3. In some embodiments, each occurrence of n is independently 1, 2, or 3. In some embodiments, p is 1.

In another aspect, described herein is a compound of Formula (II):

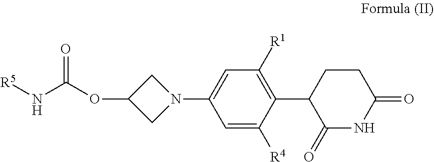

or a pharmaceutically acceptable salt thereof, wherein: R⁵ is heteroaryl, aryl, or C₃₋₁₀ cycloalkyl, wherein each of heteroaryl, aryl, and C₃₋₁₀ cycloalkyl is optionally substituted with one or more occurrences of R⁶; each occurrence of R⁶ is independently halogen, cyano, C₁₋₆ alkyl, C₁₋₆ alkoxy, C₃₋₁₀ cycloalkyl, C₃₋₁₀ heterocyclyl, phenyl, or 5-membered heteroaryl, or 6-membered heteroaryl, wherein each of C₁₋₆ alkyl, C₁₋₆ alkoxy, C₃₋₁₀ cycloalkyl, 3 to 10 membered heterocyclyl, phenyl, 5-membered heteroaryl, and 6-membered heteroaryl is optionally substituted with one or more occurrences of R⁷; and each occurrence of R⁷ is independently halogen or C₁₋₆ alkyl.

In some embodiments, the compound is a compound of Formula (II-A):

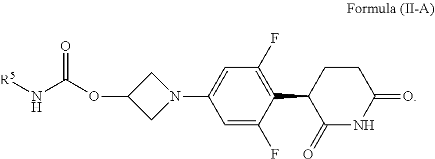

In some embodiments, the compound is a compound of Formula (II-B):

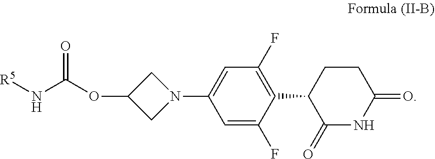

In some embodiments, R⁵ is C₃₋₁₀ cycloalkyl, C₆₋₁₀ aryl, 5-membered heteroaryl, or 6-membered heteroaryl.

In some embodiments, R⁵ is:

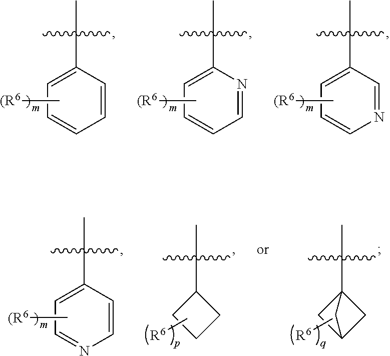

wherein m is an integer from 0 to 5, each occurrence of n is independently an integer from 0 to 4, p is an integer from 0 to 3, and q is an integer from 0 to 1. In some embodiments, R⁶ is halogen, cyano, C₁₋₄ alkyl, or C₁₋₆ alkoxy, wherein each of C₁₋₆ alkyl or C₁₋₆ alkoxy is optionally substituted with one or more occurrences of halogen. In some embodiments, R⁶ is chlorine, cyano, —CH₃, or —OCH₃. In some embodiments, R⁶ is chlorine, cyano, or —CH₃. In some embodiments, R⁷ is halogen or C₁₋₆ alkyl. In some embodiments, R⁷ is chlorine or —CH₃. In some embodiments, m is 1, 2, or 3. In some embodiments, each occurrence of n is independently 1, 2, or 3. In some embodiments, p is 1.

In another aspect, described herein is a compound of Formula (III):

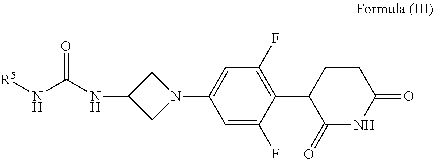

or a pharmaceutically acceptable salt thereof, wherein: R⁵ is heteroaryl, aryl, or C₃₋₁₀ ycloalkyl, wherein each of heteroaryl, aryl, and C₃₋₁₀ cycloalkyl is optionally substituted with one or more substituents each independently selected from R⁶; each occurrence of R⁶ is independently halogen, cyano, C₁₋₆ alkyl, C₁₋₆ alkoxy, C₃₋₁₀ cycloalkyl, 3 to 10 membered heterocyclyl, phenyl, or 5-membered heteroaryl, or 6-membered heteroaryl, wherein each of C₁₋₆ alkyl, C₁₋₆ alkoxy, C₃₋₁₀ cycloalkyl, 3 to 10 membered heterocyclyl, phenyl, 5-membered heteroaryl, and 6-membered heteroaryl is optionally substituted with one or more substituents each independently selected from R⁷; and each occurrence of R⁷ is independently halogen or C₁₋₆ alkyl.

In some embodiments, the compound is a compound of Formula (III-A):

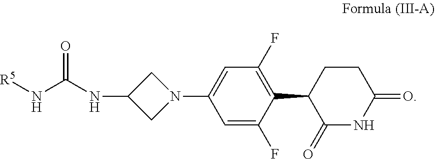

In some embodiments, the compound is a compound of Formula (III-B):

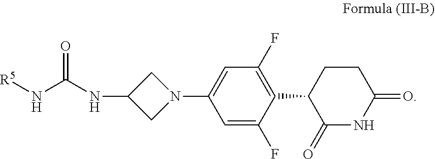

In some embodiments, R⁵ is C₃₋₁₀ cycloalkyl, C₆₋₁₀ aryl, 5-membered heteroaryl, or 6-membered heteroaryl.

In some embodiments, R⁵ is:

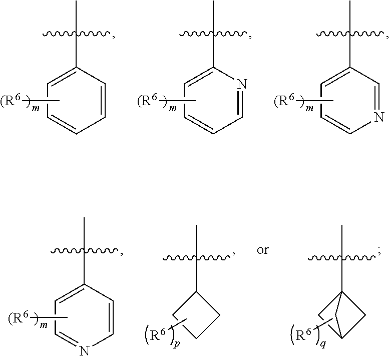

wherein m is an integer from 0 to 5, each occurrence of n is independently an integer from 0 to 4, p is an integer from 0 to 3, and q is an integer from 0 to 1.

In some embodiments, R⁶ is halogen, cyano, C₁₋₆ alkyl, or C₁₋₆ alkoxy, wherein each of C₁₋₆ alkyl or C₁₋₆ alkoxy is optionally substituted with one or more occurrences of halogen. In some embodiments, R⁶ is chlorine, cyano, —CH₃, or —OCH₃. In some embodiments, R⁶ is chlorine, cyano, or —CH₃. In some embodiments, R⁷ is halogen or C₁₋₆ alkyl. In some embodiments, R⁷ is chlorine or —CH₃. In some embodiments, m is 1, 2, or 3. In some embodiments, each occurrence of n is independently 1, 2, or 3. In some embodiments, p is 1.

In some embodiments, described herein is a compound of Formula (XI):

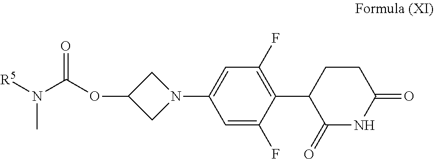

or a pharmaceutically acceptable salt thereof, wherein: R⁵ is C₁₋₆ alkyl, C₂₋₆ alkenyl, C₂₋₆ alkynyl, C₃₋₁₀ cycloalkyl, 3 to 10 membered heterocyclyl, aryl, or heteroaryl, wherein each of C₁₋₆ alkyl, C₂₋₆ alkenyl, C₂₋₆ alkynyl, C₃₋₁₀ cycloalkyl, C⁶⁻¹⁰ aryl, and heteroaryl is optionally substituted with one or more substituents each independently selected from R⁶; each occurrence of R⁶ is independently halogen, cyano, C₁₋₆ alkyl, C₁₋₆ alkoxy, C₃₋₁₀ cycloalkyl, 3 to 10 membered heterocyclyl, phenyl, or 5-membered heteroaryl, or 6-membered heteroaryl, wherein each of C₁₋₆ alkyl, C₁₋₆ alkoxy, C₃₋₁₀ cycloalkyl, 3 to 10 membered heterocyclyl, phenyl, 5-membered heteroaryl, and 6-membered heteroaryl is optionally substituted with one or more substituents each independently selected from R⁷; and each occurrence of R⁷ is independently halogen or C₁₋₆ alkyl.

In some embodiments, the compound of claim 4X, wherein R⁵ is:

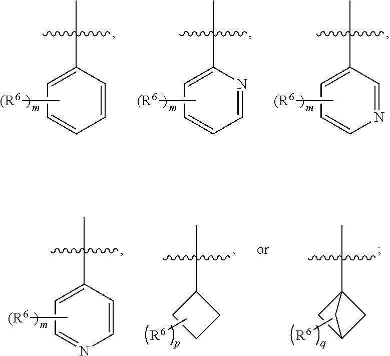

wherein m is an integer from 0 to 5, each occurrence of n is independently an integer from 0 to 4, p is an integer from 0 to 3, and q is an integer from 0 to 1. In some embodiments, R⁶ is halogen, cyano, C₁₋₆ alkyl, or C₁₋₆ alkoxy, wherein each of C₁₋₆ alkyl or C₁₋₆ alkoxy is optionally substituted with one or more occurrences of halogen. In some embodiments, R⁶ is chlorine, cyano, —CH₃, or —OCH₃. In some embodiments, R⁶ is chlorine, cyano, or —CH₃. In some embodiments, R⁷ is halogen or C₁₋₆ alkyl. In some embodiments, R⁷ is chlorine or —CH₃. In some embodiments, m is 1, 2, or 3. In some embodiments, n is independently 1, 2, or 3. In some embodiments, p is 1.

In some embodiments, provided herein is a compound having the structure:

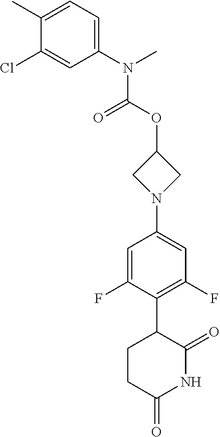 [SMILES: Cc1ccc(N(C)C(=O)OC2CN(c3cc(F)c(C4CCC(=O)NC4=O)c(F)c3)C2)cc1Cl]

or a pharmaceutically acceptable salt thereof.

In some embodiments, the compound is a compound described in Table 1 below. Table 1 also includes the compound number of each compound in accordance with the contents of the present specification.

TABLE 1
Exemplary Compounds

| No. | Compound |
|---|---|
| 1 | 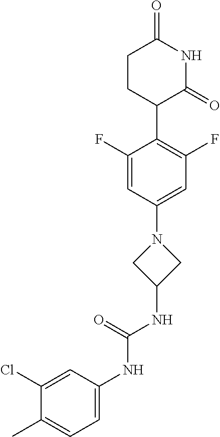 [SMILES: Cc1ccc(NC(=O)NC2CN(c3cc(F)c(C4CCC(=O)NC4=O)c(F)c3)C2)cc1Cl] |
| 2 | 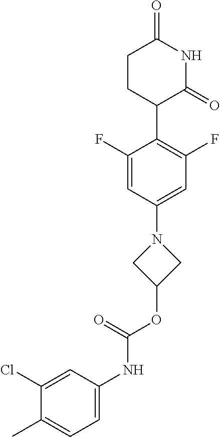 [SMILES: Cc1ccc(NC(=O)OC2CN(c3cc(F)c(C4CCC(=O)NC4=O)c(F)c3)C2)cc1Cl] |
| 3 | 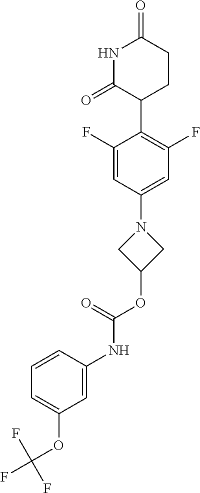 [SMILES: O=C1CCC(c2c(F)cc(N3CC(OC(=O)Nc4cccc(OC(F)(F)F)c4)C3)cc2F)C(=O)N1] |
| 4 | 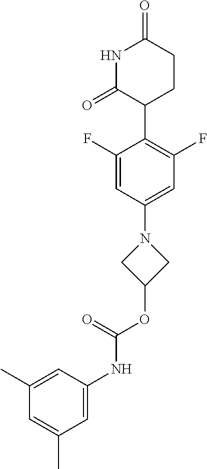 [SMILES: Cc1cc(C)cc(NC(=O)OC2CN(c3cc(F)c(C4CCC(=O)NC4=O)c(F)c3)C2)c1] |
| 5 | 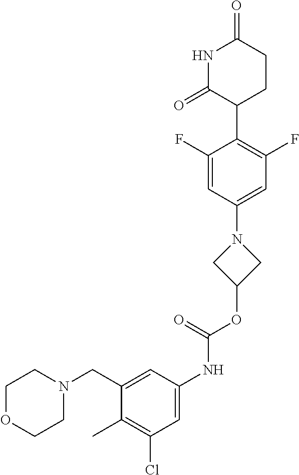 [SMILES: Cc1c(Cl)cc(NC(=O)OC2CN(c3cc(F)c(C4CCC(=O)NC4=O)c(F)c3)C2)cc1CN1CCOCC1] |
| 6 | 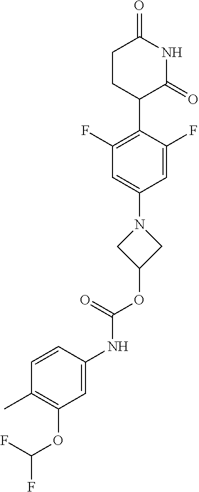 [SMILES: Cc1ccc(NC(=O)OC2CN(c3cc(F)c(C4CCC(=O)NC4=O)c(F)c3)C2)cc1OC(F)F] |
| 7 | 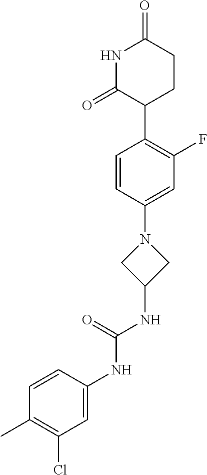 [SMILES: Cc1ccc(NC(=O)NC2CN(c3ccc(C4CCC(=O)NC4=O)c(F)c3)C2)cc1Cl] |
| 8 | 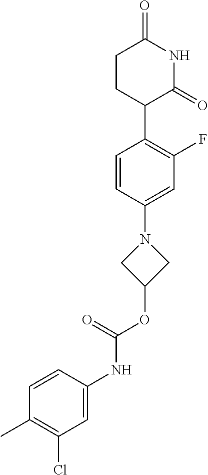 [SMILES: Cc1ccc(NC(=O)OC2CN(c3ccc(C4CCC(=O)NC4=O)c(F)c3)C2)cc1Cl] |
| 9 | 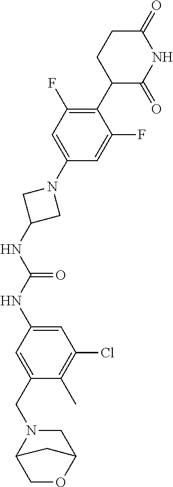 [SMILES: Cc1c(Cl)cc(NC(=O)NC2CN(c3cc(F)c(C4CCC(=O)NC4=O)c(F)c3)C2)cc1CN1CC2CC1CO2] |
| 10 | 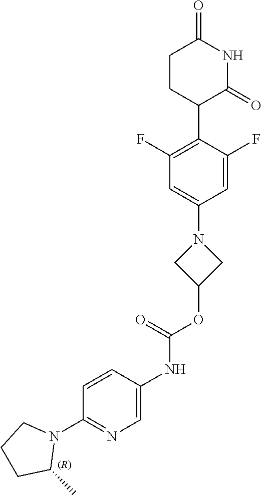 |
| 11 | 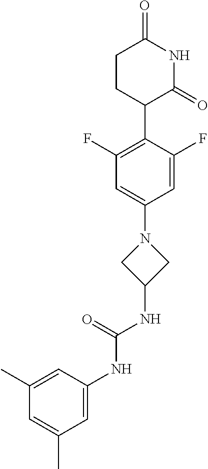 [SMILES: Cc1cc(C)cc(NC(=O)NC2CN(c3cc(F)c(C4CCC(=O)NC4=O)c(F)c3)C2)c1] |
| 12 | 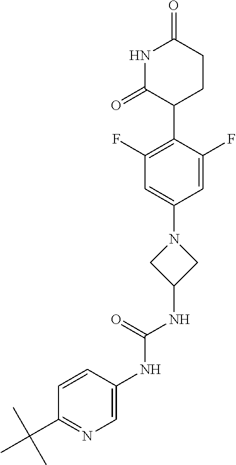 [SMILES: CC(C)(C)c1ccc(NC(=O)NC2CN(c3cc(F)c(C4CCC(=O)NC4=O)c(F)c3)C2)cn1] |
| 13 | 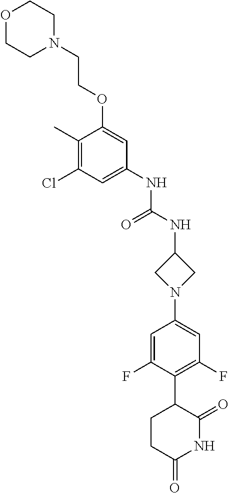 [SMILES: Cc1c(Cl)cc(NC(=O)NC2CN(c3cc(F)c(C4CCC(=O)NC4=O)c(F)c3)C2)cc1OCCN1CCOCC1] |
| 14 | 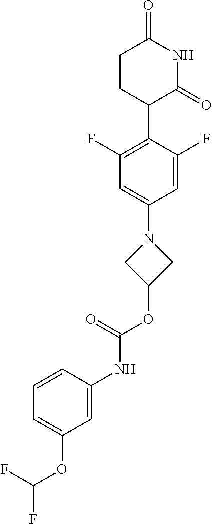 [SMILES: O=C1CCC(c2c(F)cc(N3CC(OC(=O)Nc4cccc(OC(F)F)c4)C3)cc2F)C(=O)N1] |
| 15 | 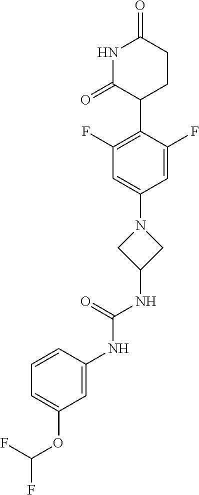 [SMILES: O=C1CCC(c2c(F)cc(N3CC(NC(=O)Nc4cccc(OC(F)F)c4)C3)cc2F)C(=O)N1] |
| 16 | 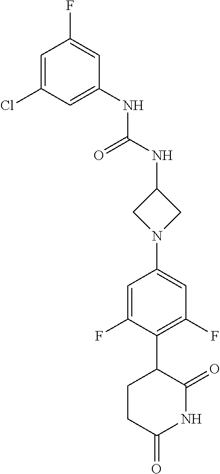 [SMILES: O=C1CCC(c2c(F)cc(N3CC(NC(=O)Nc4cc(F)cc(Cl)c4)C3)cc2F)C(=O)N1] |
| 17 | 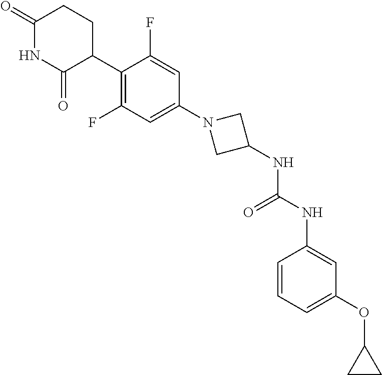 [SMILES: O=C1CCC(c2c(F)cc(N3CC(NC(=O)Nc4cccc(OC5CC5)c4)C3)cc2F)C(=O)N1] |
| 18 | 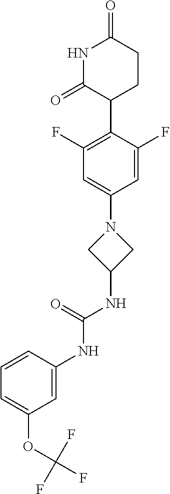 [SMILES: O=C1CCC(c2c(F)cc(N3CC(NC(=O)Nc4cccc(OC(F)(F)F)c4)C3)cc2F)C(=O)N1] |
| 19 | 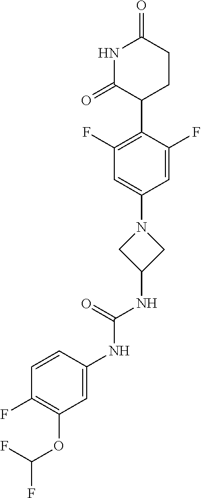 [SMILES: O=C1CCC(c2c(F)cc(N3CC(NC(=O)Nc4ccc(F)c(OC(F)F)c4)C3)cc2F)C(=O)N1] |
| 20 | 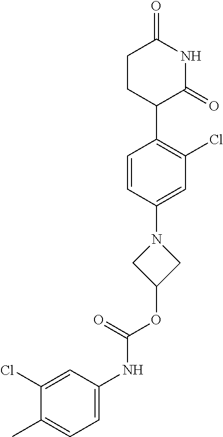 [SMILES: Cc1ccc(NC(=O)OC2CN(c3ccc(C4CCC(=O)NC4=O)c(Cl)c3)C2)cc1Cl] |
| 21 | 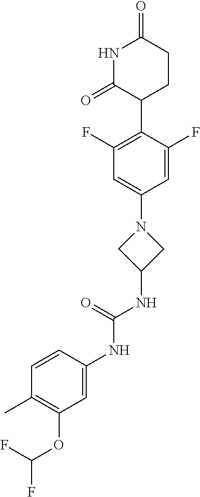 [SMILES: Cc1ccc(NC(=O)NC2CN(c3cc(F)c(C4CCC(=O)NC4=O)c(F)c3)C2)cc1OC(F)F] |
| 22 | 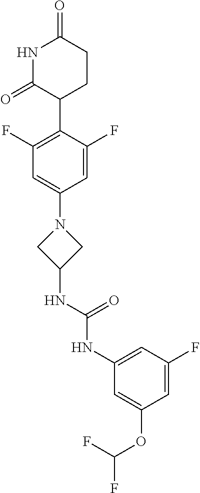 [SMILES: O=C1CCC(c2c(F)cc(N3CC(NC(=O)Nc4cc(F)cc(OC(F)F)c4)C3)cc2F)C(=O)N1] |
| 23 | 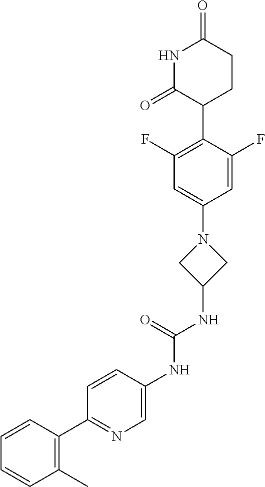 [SMILES: Cc1ccccc1-c1ccc(NC(=O)NC2CN(c3cc(F)c(C4CCC(=O)NC4=O)c(F)c3)C2)cn1] |
| 24 | 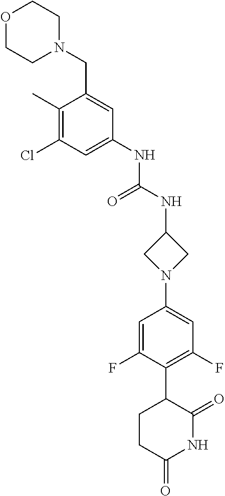 [SMILES: Cc1c(Cl)cc(NC(=O)NC2CN(c3cc(F)c(C4CCC(=O)NC4=O)c(F)c3)C2)cc1CN1CCOCC1] |
| 25 | 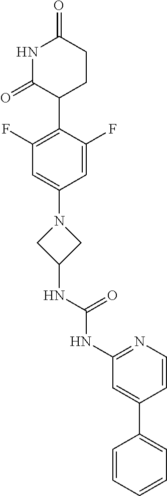 [SMILES: O=C1CCC(c2c(F)cc(N3CC(NC(=O)Nc4cc(-c5ccccc5)ccn4)C3)cc2F)C(=O)N1] |
| 26 | 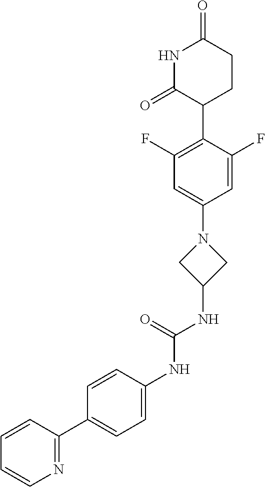 [SMILES: O=C1CCC(c2c(F)cc(N3CC(NC(=O)Nc4ccc(-c5ccccn5)cc4)C3)cc2F)C(=O)N1] |
| 27 | 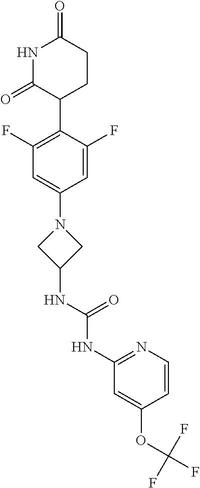 [SMILES: O=C1CCC(c2c(F)cc(N3CC(NC(=O)Nc4cc(OC(F)(F)F)ccn4)C3)cc2F)C(=O)N1] |
| 28 | 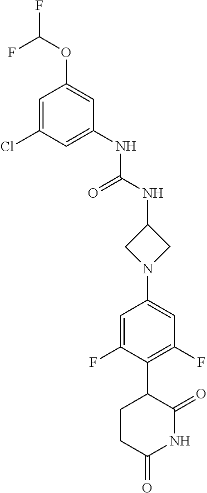 [SMILES: O=C1CCC(c2c(F)cc(N3CC(NC(=O)Nc4cc(Cl)cc(OC(F)F)c4)C3)cc2F)C(=O)N1] |
| 29 | 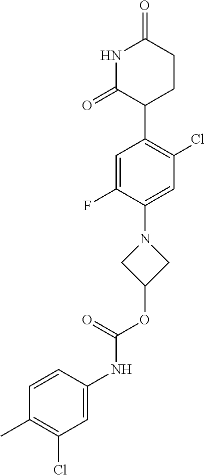 [SMILES: Cc1ccc(NC(=O)OC2CN(c3cc(Cl)c(C4CCC(=O)NC4=O)cc3F)C2)cc1Cl] |
| 30 | 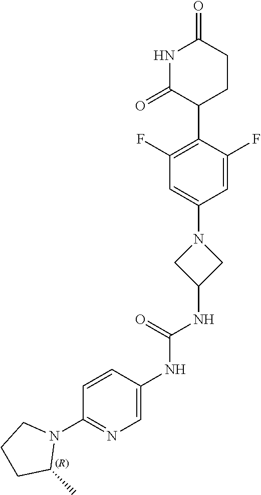 |
| 31 | 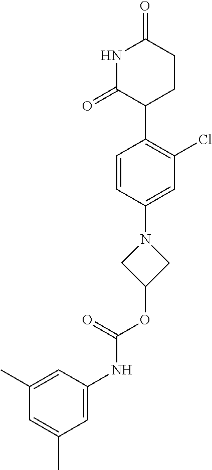 [SMILES: Cc1cc(C)cc(NC(=O)OC2CN(c3ccc(C4CCC(=O)NC4=O)c(Cl)c3)C2)c1] |
| 32 | 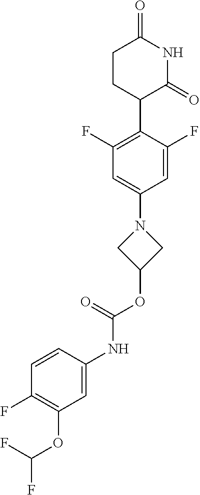 [SMILES: O=C1CCC(c2c(F)cc(N3CC(OC(=O)Nc4ccc(F)c(OC(F)F)c4)C3)cc2F)C(=O)N1] |
| 33 | 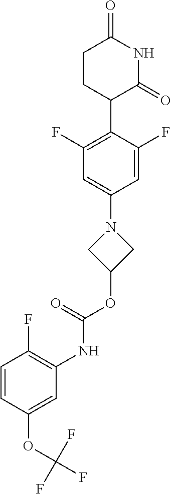 [SMILES: O=C1CCC(c2c(F)cc(N3CC(OC(=O)Nc4cc(OC(F)(F)F)ccc4F)C3)cc2F)C(=O)N1] |
| 34 | 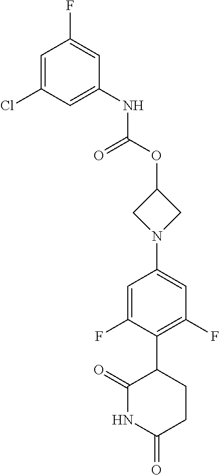 [SMILES: O=C1CCC(c2c(F)cc(N3CC(OC(=O)Nc4cc(F)cc(Cl)c4)C3)cc2F)C(=O)N1] |
| 35 | 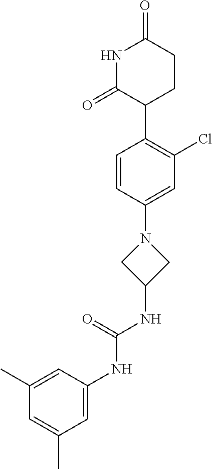 [SMILES: Cc1cc(C)cc(NC(=O)NC2CN(c3ccc(C4CCC(=O)NC4=O)c(Cl)c3)C2)c1] |
| 36 | 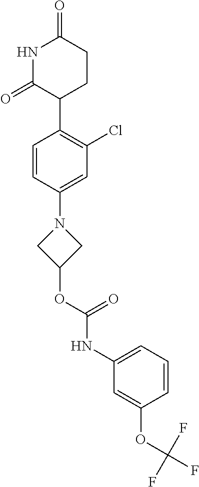 [SMILES: O=C1CCC(c2ccc(N3CC(OC(=O)Nc4cccc(OC(F)(F)F)c4)C3)cc2Cl)C(=O)N1] |
| 37 | 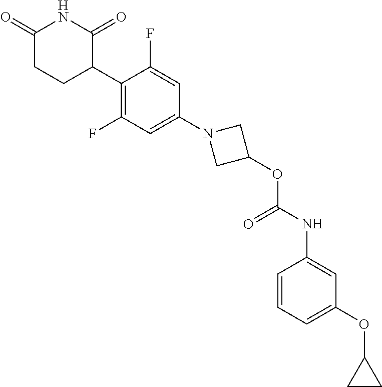 [SMILES: O=C1CCC(c2c(F)cc(N3CC(OC(=O)Nc4cccc(OC5CC5)c4)C3)cc2F)C(=O)N1] |
| 38 | 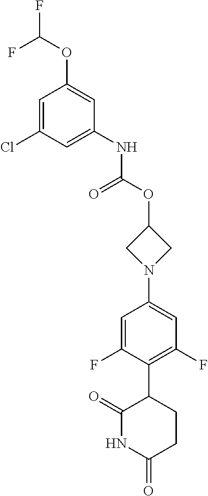 [SMILES: O=C1CCC(c2c(F)cc(N3CC(OC(=O)Nc4cc(Cl)cc(OC(F)F)c4)C3)cc2F)C(=O)N1] |
| 39 | 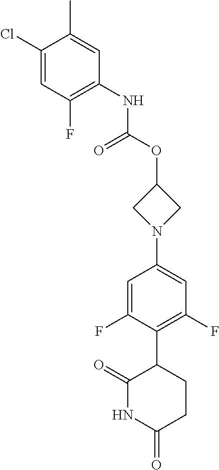 [SMILES: Cc1cc(NC(=O)OC2CN(c3cc(F)c(C4CCC(=O)NC4=O)c(F)c3)C2)c(F)cc1Cl] |
| 40 | 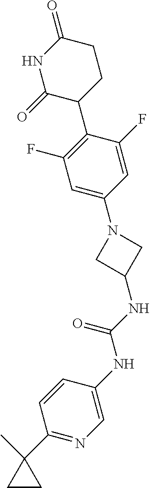 [SMILES: CC1(c2ccc(NC(=O)NC3CN(c4cc(F)c(C5CCC(=O)NC5=O)c(F)c4)C3)cn2)CC1] |
| 41 | 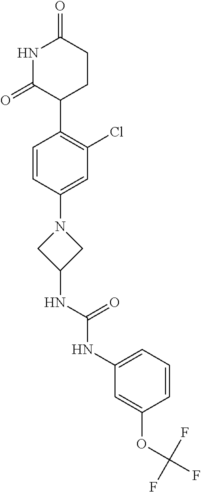 [SMILES: O=C1CCC(c2ccc(N3CC(NC(=O)Nc4cccc(OC(F)(F)F)c4)C3)cc2Cl)C(=O)N1] |
| 42 | 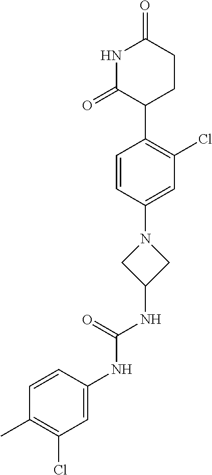 [SMILES: Cc1ccc(NC(=O)NC2CN(c3ccc(C4CCC(=O)NC4=O)c(Cl)c3)C2)cc1Cl] |
| 43 | 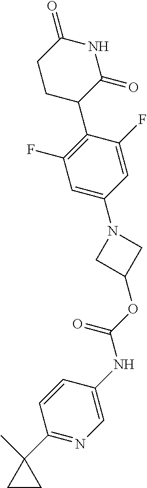 [SMILES: CC1(c2ccc(NC(=O)OC3CN(c4cc(F)c(C5CCC(=O)NC5=O)c(F)c4)C3)cn2)CC1] |
| 44 | 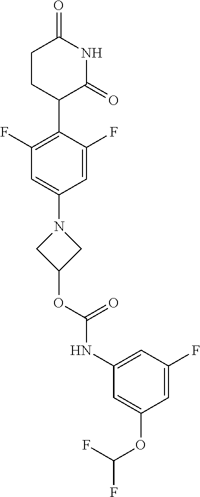 [SMILES: O=C1CCC(c2c(F)cc(N3CC(OC(=O)Nc4cc(F)cc(OC(F)F)c4)C3)cc2F)C(=O)N1] |
| 45 | 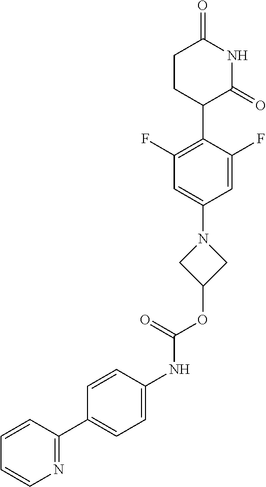 [SMILES: O=C1CCC(c2c(F)cc(N3CC(OC(=O)Nc4ccc(-c5ccccn5)cc4)C3)cc2F)C(=O)N1] |
| 46 | 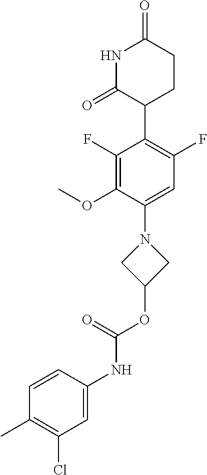 [SMILES: COc1c(N2CC(OC(=O)Nc3ccc(C)c(Cl)c3)C2)cc(F)c(C2CCC(=O)NC2=O)c1F] |
| 47 | 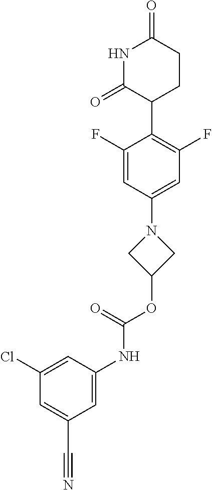 [SMILES: N#Cc1cc(Cl)cc(NC(=O)OC2CN(c3cc(F)c(C4CCC(=O)NC4=O)c(F)c3)C2)c1] |
| 48 | 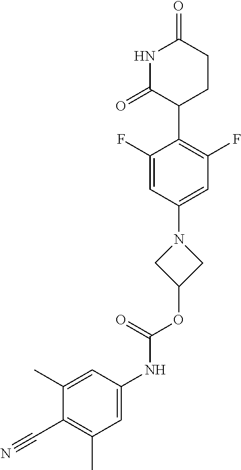 [SMILES: Cc1cc(NC(=O)OC2CN(c3cc(F)c(C4CCC(=O)NC4=O)c(F)c3)C2)cc(C)c1C#N] |
| 49 | 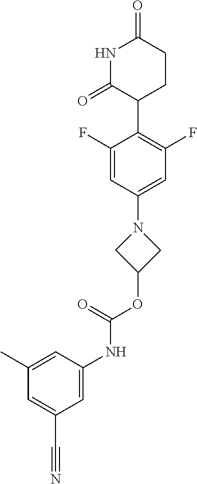 [SMILES: Cc1cc(C#N)cc(NC(=O)OC2CN(c3cc(F)c(C4CCC(=O)NC4=O)c(F)c3)C2)c1] |
| 50 | 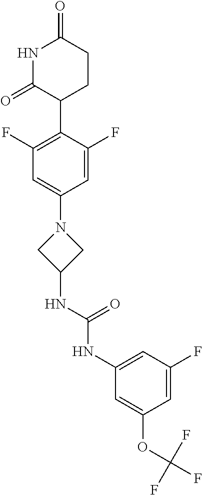 [SMILES: O=C1CCC(c2c(F)cc(N3CC(NC(=O)Nc4cc(F)cc(OC(F)(F)F)c4)C3)cc2F)C(=O)N1] |
| 51 | 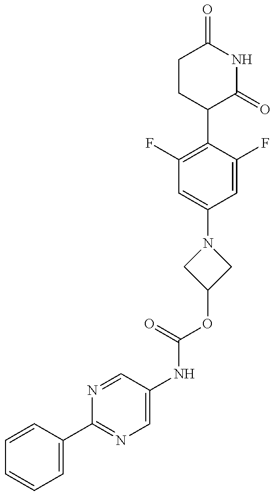 [SMILES: O=C1CCC(c2c(F)cc(N3CC(OC(=O)Nc4cnc(-c5ccccc5)nc4)C3)cc2F)C(=O)N1] |
| 52 | 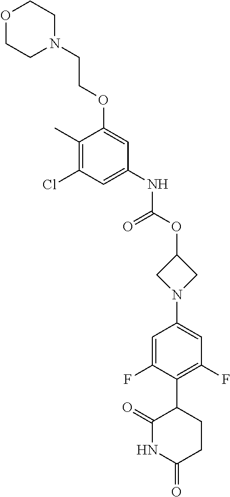 [SMILES: Cc1c(Cl)cc(NC(=O)OC2CN(c3cc(F)c(C4CCC(=O)NC4=O)c(F)c3)C2)cc1OCCN1CCOCC1] |
| 53 | 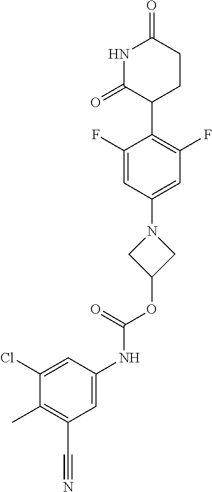 [SMILES: Cc1c(Cl)cc(NC(=O)OC2CN(c3cc(F)c(C4CCC(=O)NC4=O)c(F)c3)C2)cc1C#N] |
| 54 | 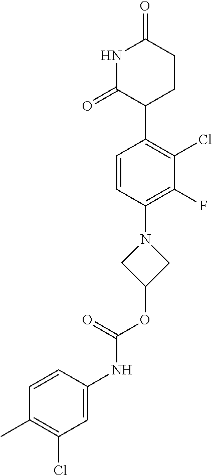 [SMILES: Cc1ccc(NC(=O)OC2CN(c3ccc(C4CCC(=O)NC4=O)c(Cl)c3F)C2)cc1Cl] |
| 55 | 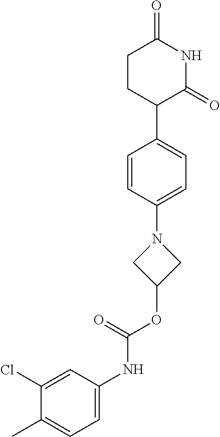 [SMILES: Cc1ccc(NC(=O)OC2CN(c3ccc(C4CCC(=O)NC4=O)cc3)C2)cc1Cl] |
| 56 | 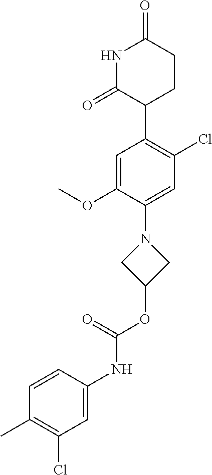 [SMILES: COc1cc(C2CCC(=O)NC2=O)c(Cl)cc1N1CC(OC(=O)Nc2ccc(C)c(Cl)c2)C1] |
| 57 | 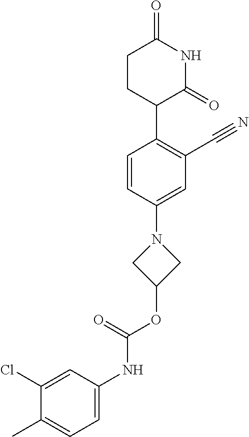 [SMILES: Cc1ccc(NC(=O)OC2CN(c3ccc(C4CCC(=O)NC4=O)c(C#N)c3)C2)cc1Cl] |
| 58 | 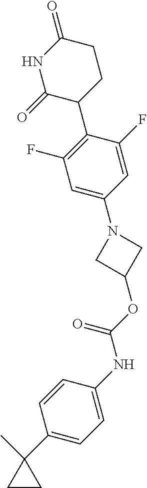 [SMILES: CC1(c2ccc(NC(=O)OC3CN(c4cc(F)c(C5CCC(=O)NC5=O)c(F)c4)C3)cc2)CC1] |
| 59 | 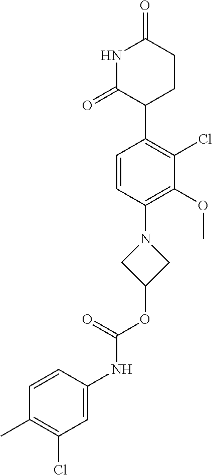 |
| 60 | 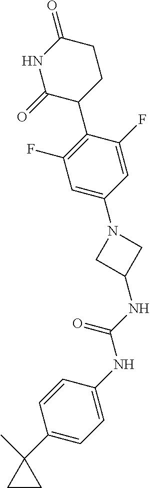 [SMILES: CC1(c2ccc(NC(=O)NC3CN(c4cc(F)c(C5CCC(=O)NC5=O)c(F)c4)C3)cc2)CC1] |
| 61 | 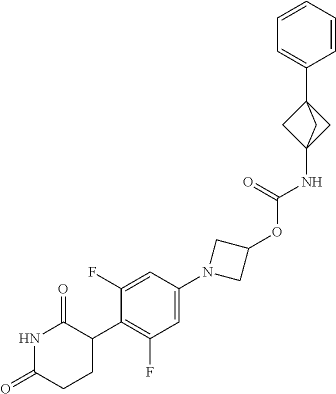 [SMILES: O=C1CCC(c2c(F)cc(N3CC(OC(=O)NC45CC(c6ccccc6)(C4)C5)C3)cc2F)C(=O)N1] |
| 62 | 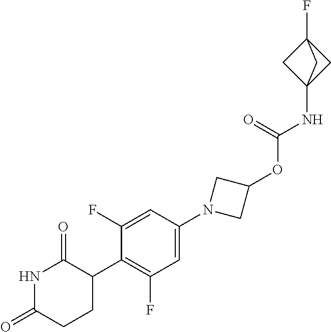 [SMILES: O=C1CCC(c2c(F)cc(N3CC(OC(=O)NC45CC(F)(C4)C5)C3)cc2F)C(=O)N1] |
| 63 | 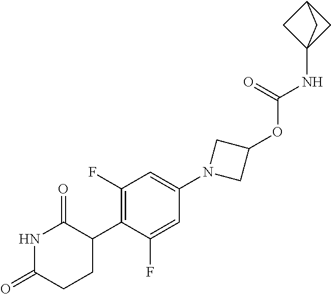 [SMILES: O=C1CCC(c2c(F)cc(N3CC(OC(=O)NC45CC(C4)C5)C3)cc2F)C(=O)N1] |
| 64 | 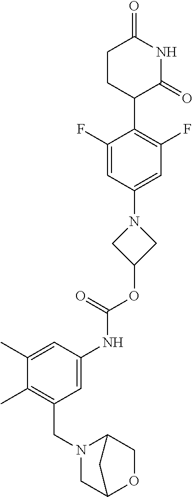 [SMILES: Cc1cc(NC(=O)OC2CN(c3cc(F)c(C4CCC(=O)NC4=O)c(F)c3)C2)cc(CN2CC3CC2CO3)c1C] |
| 65 | 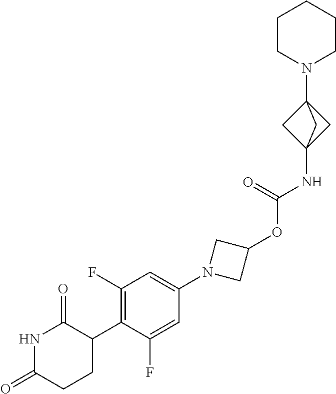 [SMILES: O=C1CCC(c2c(F)cc(N3CC(OC(=O)NC45CC(N6CCCCC6)(C4)C5)C3)cc2F)C(=O)N1] |
| 66 | 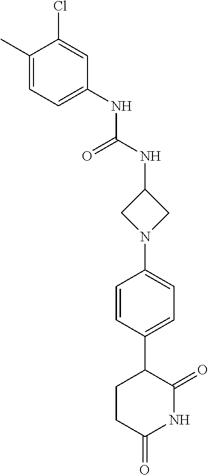 [SMILES: Cc1ccc(NC(=O)NC2CN(c3ccc(C4CCC(=O)NC4=O)cc3)C2)cc1Cl] |
| 67 | 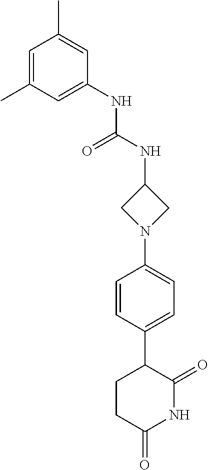 [SMILES: Cc1cc(C)cc(NC(=O)NC2CN(c3ccc(C4CCC(=O)NC4=O)cc3)C2)c1] |
| 68 | 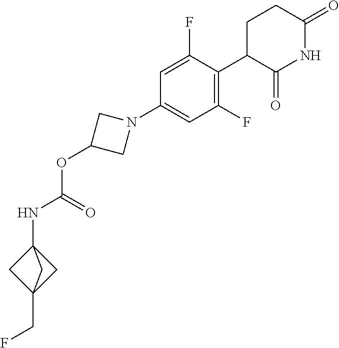 [SMILES: O=C1CCC(c2c(F)cc(N3CC(OC(=O)NC45CC(CF)(C4)C5)C3)cc2F)C(=O)N1] |
| 69 | 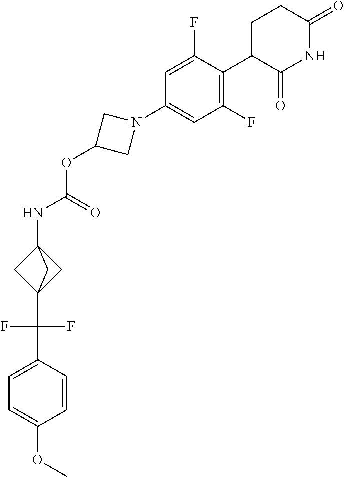 [SMILES: COc1ccc(C(F)(F)C23CC(NC(=O)OC4CN(c5cc(F)c(C6CCC(=O)NC6=O)c(F)c5)C4)(C2)C3)cc1] |
| 70 | 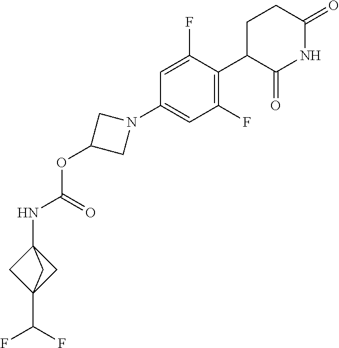 [SMILES: O=C1CCC(c2c(F)cc(N3CC(OC(=O)NC45CC(C(F)F)(C4)C5)C3)cc2F)C(=O)N1] |
| 71 | 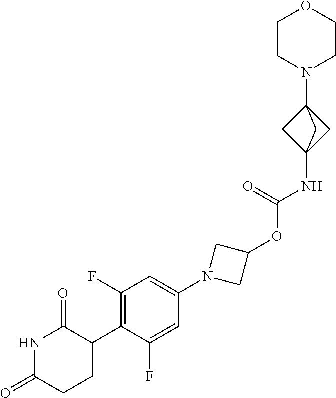 [SMILES: O=C1CCC(c2c(F)cc(N3CC(OC(=O)NC45CC(N6CCOCC6)(C4)C5)C3)cc2F)C(=O)N1] |
| 72 | 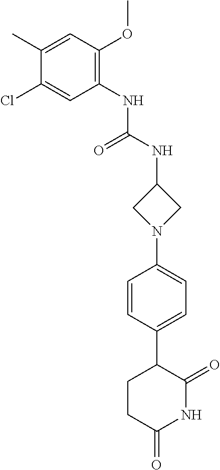 [SMILES: COc1cc(C)c(Cl)cc1NC(=O)NC1CN(c2ccc(C3CCC(=O)NC3=O)cc2)C1] |
| 73 | 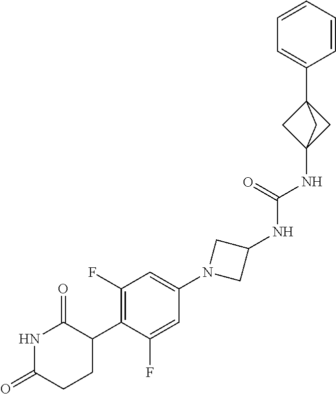 [SMILES: O=C1CCC(c2c(F)cc(N3CC(NC(=O)NC45CC(c6ccccc6)(C4)C5)C3)cc2F)C(=O)N1] |
| 74 | 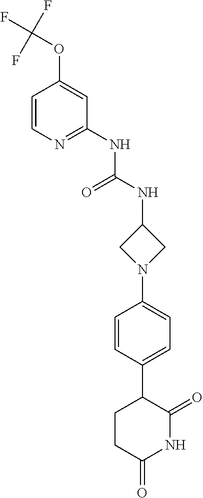 [SMILES: O=C1CCC(c2ccc(N3CC(NC(=O)Nc4cc(OC(F)(F)F)ccn4)C3)cc2)C(=O)N1] |
| 75 | 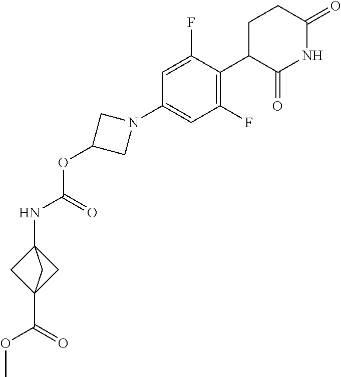 [SMILES: COC(=O)C12CC(NC(=O)OC3CN(c4cc(F)c(C5CCC(=O)NC5=O)c(F)c4)C3)(C1)C2] |
| 76 | 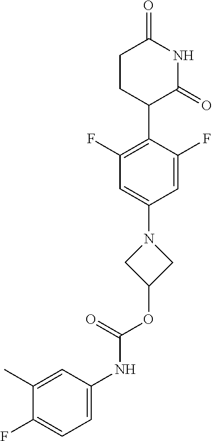 [SMILES: Cc1cc(NC(=O)OC2CN(c3cc(F)c(C4CCC(=O)NC4=O)c(F)c3)C2)ccc1F] |
| 77 | 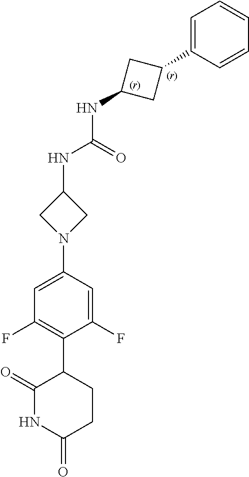 [SMILES: O=C1CCC(c2c(F)cc(N3CC(NC(=O)N[C@H]4C[C@H](c5ccccc5)C4)C3)cc2F)C(=O)N1] |
| 78 | 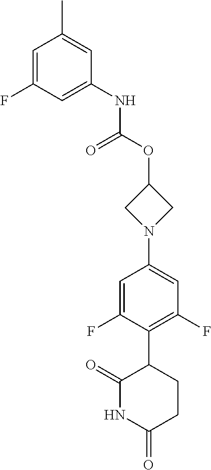 [SMILES: Cc1cc(F)cc(NC(=O)OC2CN(c3cc(F)c(C4CCC(=O)NC4=O)c(F)c3)C2)c1] |
| 79 | 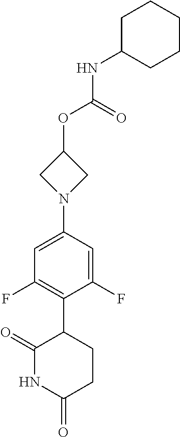 [SMILES: O=C1CCC(c2c(F)cc(N3CC(OC(=O)NC4CCCCC4)C3)cc2F)C(=O)N1] |
| 80 | 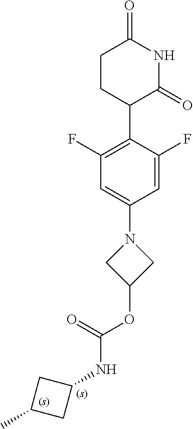 [SMILES: C[C@H]1C[C@@H](NC(=O)OC2CN(c3cc(F)c(C4CCC(=O)NC4=O)c(F)c3)C2)C1] |
| 81 | 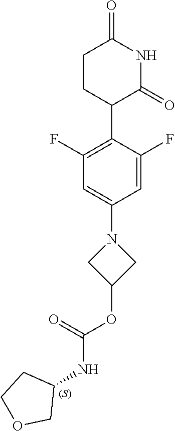 |
| 82 | 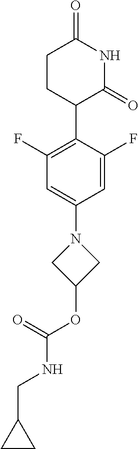 [SMILES: O=C1CCC(c2c(F)cc(N3CC(OC(=O)NCC4CC4)C3)cc2F)C(=O)N1] |
| 83 | 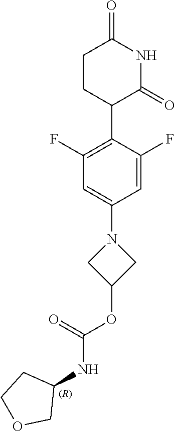 |
| 84 | 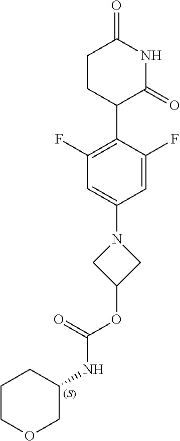 |
| 85 | 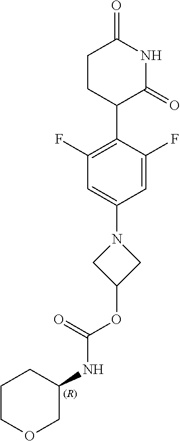 |
| 86 | 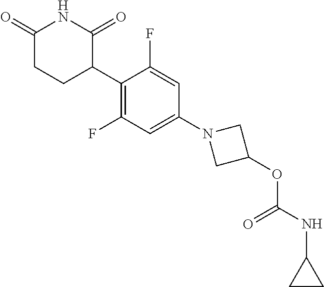 [SMILES: O=C1CCC(c2c(F)cc(N3CC(OC(=O)NC4CC4)C3)cc2F)C(=O)N1] |
| 87 | 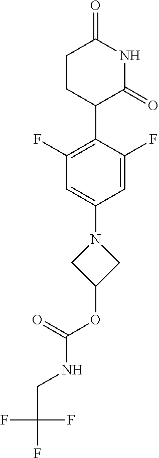 [SMILES: O=C1CCC(c2c(F)cc(N3CC(OC(=O)NCC(F)(F)F)C3)cc2F)C(=O)N1] |
| 88 | 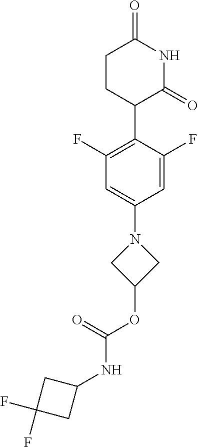 [SMILES: O=C1CCC(c2c(F)cc(N3CC(OC(=O)NC4CC(F)(F)C4)C3)cc2F)C(=O)N1] |
| 89 | 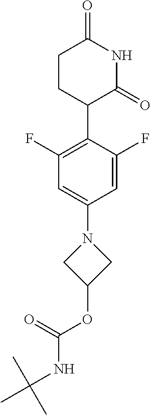 [SMILES: CC(C)(C)NC(=O)OC1CN(c2cc(F)c(C3CCC(=O)NC3=O)c(F)c2)C1] |
| 90 | 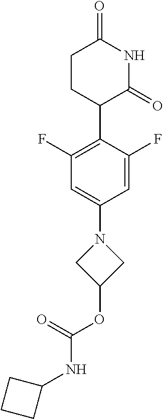 [SMILES: O=C1CCC(c2c(F)cc(N3CC(OC(=O)NC4CCC4)C3)cc2F)C(=O)N1] |
| 91 | 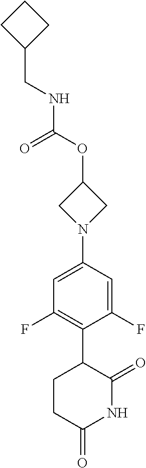 [SMILES: O=C1CCC(c2c(F)cc(N3CC(OC(=O)NCC4CCC4)C3)cc2F)C(=O)N1] |
| 92 | 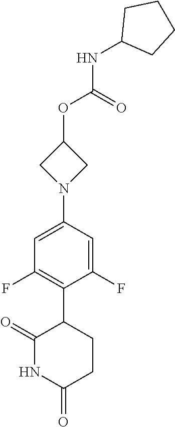 [SMILES: O=C1CCC(c2c(F)cc(N3CC(OC(=O)NC4CCCC4)C3)cc2F)C(=O)N1] |
| 93 | 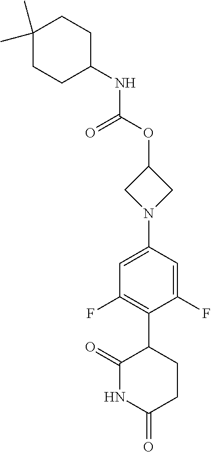 [SMILES: CC1(C)CCC(NC(=O)OC2CN(c3cc(F)c(C4CCC(=O)NC4=O)c(F)c3)C2)CC1] |
| 94 | 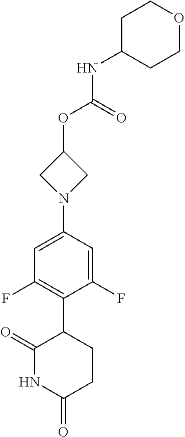 [SMILES: O=C1CCC(c2c(F)cc(N3CC(OC(=O)NC4CCOCC4)C3)cc2F)C(=O)N1] |
| 95 | 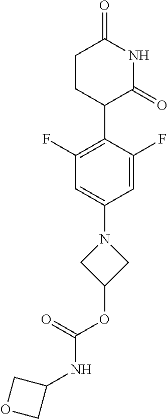 [SMILES: O=C1CCC(c2c(F)cc(N3CC(OC(=O)NC4COC4)C3)cc2F)C(=O)N1] |
| 96 | 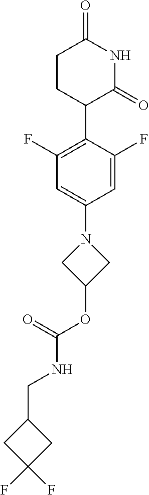 [SMILES: O=C1CCC(c2c(F)cc(N3CC(OC(=O)NCC4CC(F)(F)C4)C3)cc2F)C(=O)N1] |
| 97 | 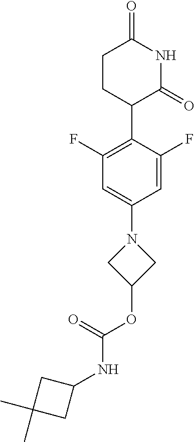 [SMILES: CC1(C)CC(NC(=O)OC2CN(c3cc(F)c(C4CCC(=O)NC4=O)c(F)c3)C2)C1] |
| 98 | 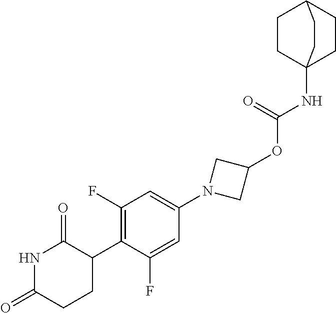 [SMILES: O=C1CCC(c2c(F)cc(N3CC(OC(=O)NC45CCC(CC4)CC5)C3)cc2F)C(=O)N1] |
| 99 | 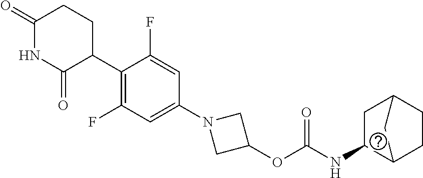 [SMILES: O=C1CCC(c2c(F)cc(N3CC(OC(=O)N[C@H]4CC5CCC4C5)C3)cc2F)C(=O)N1] |
| 100 | 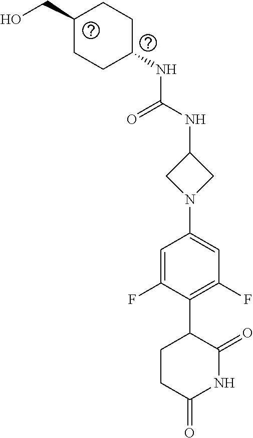 [SMILES: O=C1CCC(c2c(F)cc(N3CC(NC(=O)N[C@H]4CC[C@H](CO)CC4)C3)cc2F)C(=O)N1] |
| 101 | 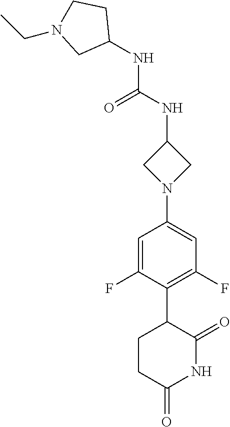 [SMILES: CCN1CCC(NC(=O)NC2CN(c3cc(F)c(C4CCC(=O)NC4=O)c(F)c3)C2)C1] |
| 102 | 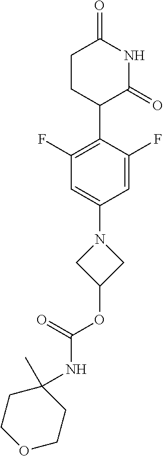 [SMILES: CC1(NC(=O)OC2CN(c3cc(F)c(C4CCC(=O)NC4=O)c(F)c3)C2)CCOCC1] |
| 103 | 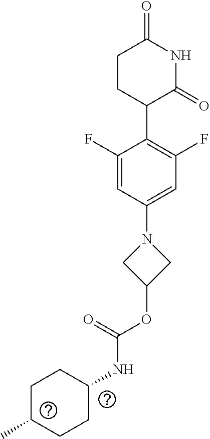 [SMILES: C[C@H]1CC[C@@H](NC(=O)OC2CN(c3cc(F)c(C4CCC(=O)NC4=O)c(F)c3)C2)CC1] |
| 104 | 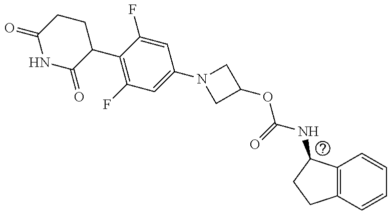 [SMILES: O=C1CCC(c2c(F)cc(N3CC(OC(=O)N[C@@H]4CCc5ccccc54)C3)cc2F)C(=O)N1] |
| 105 | 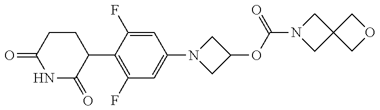 [SMILES: O=C1CCC(c2c(F)cc(N3CC(OC(=O)N4CC5(COC5)C4)C3)cc2F)C(=O)N1] |
| 106 | 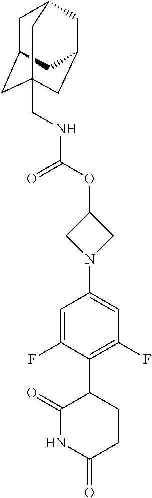 [SMILES: O=C1CCC(c2c(F)cc(N3CC(OC(=O)NCC45C[C@H]6C[C@@H](C4)C[C@@H](C5)C6)C3)cc2F)C(=O)N1] |
| 107 | 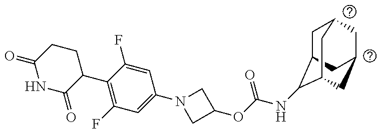 [SMILES: O=C1CCC(c2c(F)cc(N3CC(OC(=O)NC4[C@H]5C[C@@H]6C[C@@H](C[C@H]4C6)C5)C3)cc2F)C(=O)N1] |
| 108 | 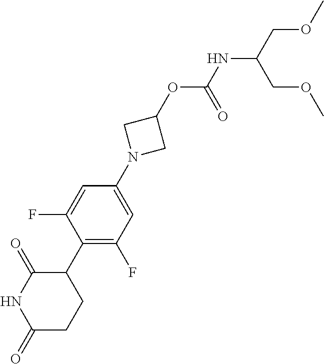 [SMILES: COCC(COC)NC(=O)OC1CN(c2cc(F)c(C3CCC(=O)NC3=O)c(F)c2)C1] |
| 109 | 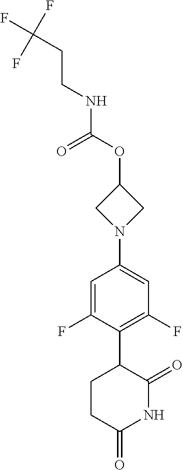 [SMILES: O=C1CCC(c2c(F)cc(N3CC(OC(=O)NCCC(F)(F)F)C3)cc2F)C(=O)N1] |
| 110 | 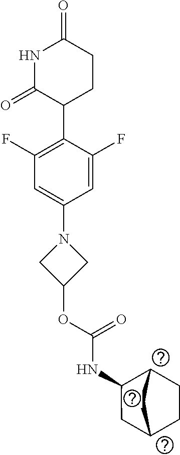 [SMILES: O=C1CCC(c2c(F)cc(N3CC(OC(=O)N[C@@H]4C[C@H]5CC[C@@H]4C5)C3)cc2F)C(=O)N1] |
| 111 | 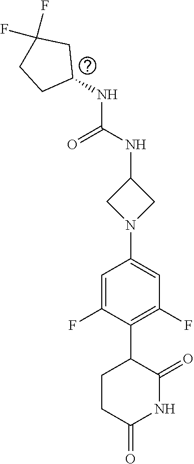 [SMILES: O=C1CCC(c2c(F)cc(N3CC(NC(=O)N[C@@H]4CCC(F)(F)C4)C3)cc2F)C(=O)N1] |
| 112 | 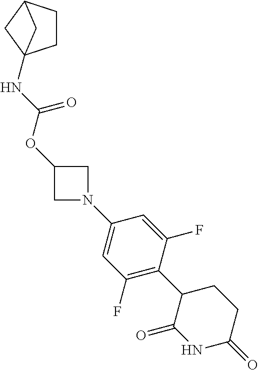 [SMILES: O=C1CCC(c2c(F)cc(N3CC(OC(=O)NC45CCC(C4)C5)C3)cc2F)C(=O)N1] |
| 113 | 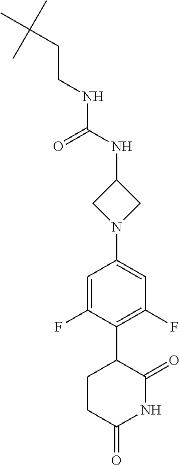 [SMILES: CC(C)(C)CCNC(=O)NC1CN(c2cc(F)c(C3CCC(=O)NC3=O)c(F)c2)C1] |
| 114 | 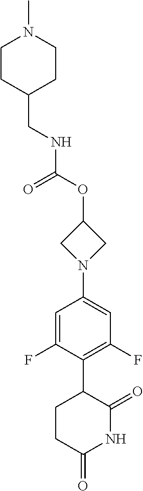 [SMILES: CN1CCC(CNC(=O)OC2CN(c3cc(F)c(C4CCC(=O)NC4=O)c(F)c3)C2)CC1] |
| 115 | 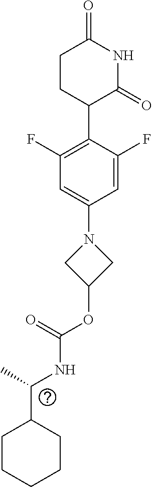 [SMILES: C[C@H](NC(=O)OC1CN(c2cc(F)c(C3CCC(=O)NC3=O)c(F)c2)C1)C1CCCCC1] |
| 116 | 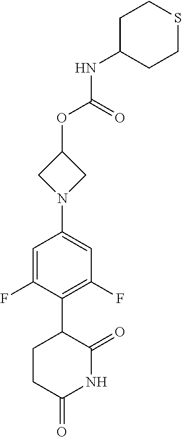 [SMILES: O=C1CCC(c2c(F)cc(N3CC(OC(=O)NC4CCSCC4)C3)cc2F)C(=O)N1] |
| 117 | 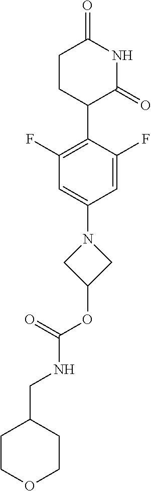 [SMILES: O=C1CCC(c2c(F)cc(N3CC(OC(=O)NCC4CCOCC4)C3)cc2F)C(=O)N1] |
| 118 | 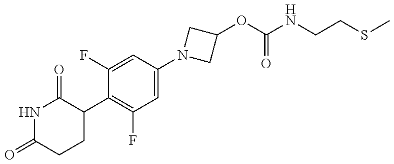 [SMILES: CSCCNC(=O)OC1CN(c2cc(F)c(C3CCC(=O)NC3=O)c(F)c2)C1] |
| 119 | 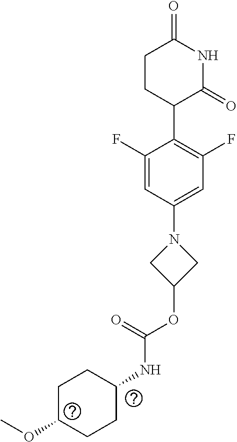 [SMILES: CO[C@H]1CC[C@@H](NC(=O)OC2CN(c3cc(F)c(C4CCC(=O)NC4=O)c(F)c3)C2)CC1] |
| 120 | 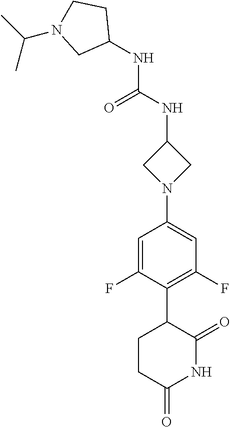 [SMILES: CC(C)N1CCC(NC(=O)NC2CN(c3cc(F)c(C4CCC(=O)NC4=O)c(F)c3)C2)C1] |
| 121 | 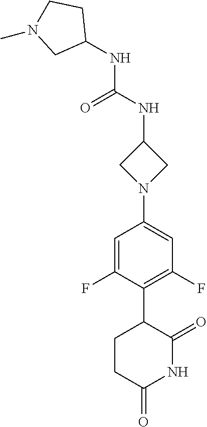 [SMILES: CN1CCC(NC(=O)NC2CN(c3cc(F)c(C4CCC(=O)NC4=O)c(F)c3)C2)C1] |
| 122 | 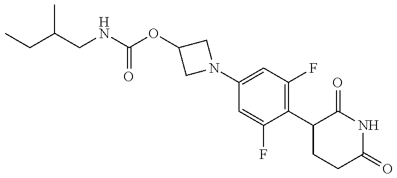 [SMILES: CCC(C)CNC(=O)OC1CN(c2cc(F)c(C3CCC(=O)NC3=O)c(F)c2)C1] |
| 123 | 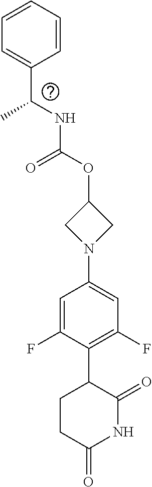 [SMILES: C[C@@H](NC(=O)OC1CN(c2cc(F)c(C3CCC(=O)NC3=O)c(F)c2)C1)c1ccccc1] |
| 124 | 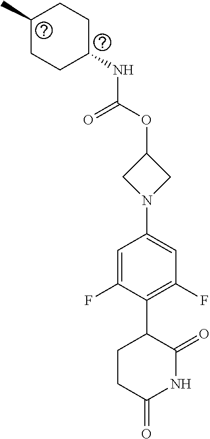 [SMILES: C[C@H]1CC[C@H](NC(=O)OC2CN(c3cc(F)c(C4CCC(=O)NC4=O)c(F)c3)C2)CC1] |
| 125 | 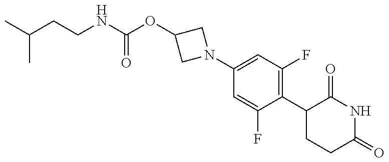 [SMILES: CC(C)CCNC(=O)OC1CN(c2cc(F)c(C3CCC(=O)NC3=O)c(F)c2)C1] |
| 126 | 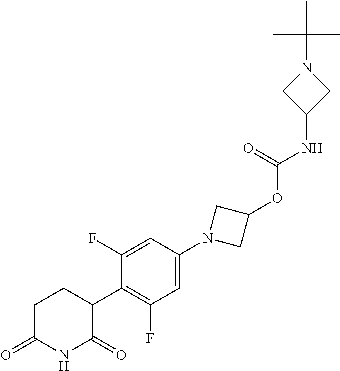 [SMILES: CC(C)(C)N1CC(NC(=O)OC2CN(c3cc(F)c(C4CCC(=O)NC4=O)c(F)c3)C2)C1] |
| 127 | 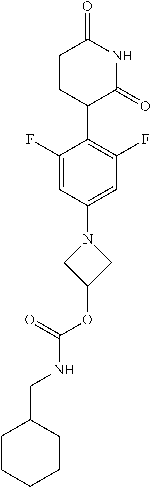 [SMILES: O=C1CCC(c2c(F)cc(N3CC(OC(=O)NCC4CCCCC4)C3)cc2F)C(=O)N1] |
| 128 | 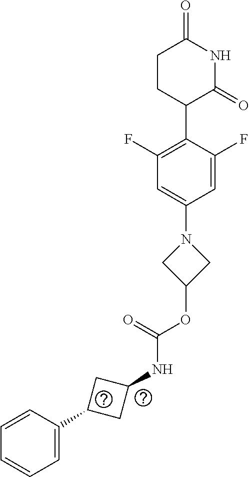 [SMILES: O=C1CCC(c2c(F)cc(N3CC(OC(=O)N[C@H]4C[C@H](c5ccccc5)C4)C3)cc2F)C(=O)N1] |
| 129 | 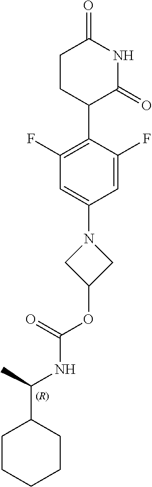 |
| 130 | 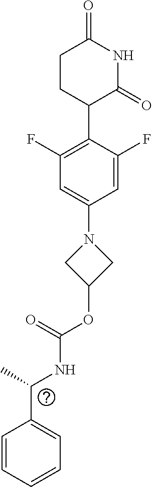 [SMILES: C[C@H](NC(=O)OC1CN(c2cc(F)c(C3CCC(=O)NC3=O)c(F)c2)C1)c1ccccc1] |
| 131 | 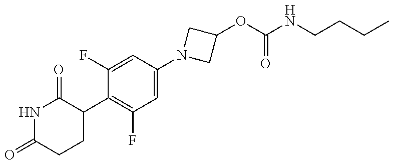 [SMILES: CCCCNC(=O)OC1CN(c2cc(F)c(C3CCC(=O)NC3=O)c(F)c2)C1] |
| 132 | 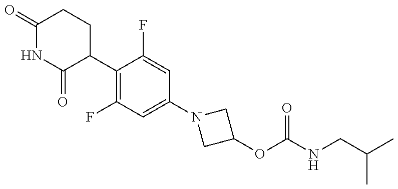 [SMILES: CC(C)CNC(=O)OC1CN(c2cc(F)c(C3CCC(=O)NC3=O)c(F)c2)C1] |
| 133 | 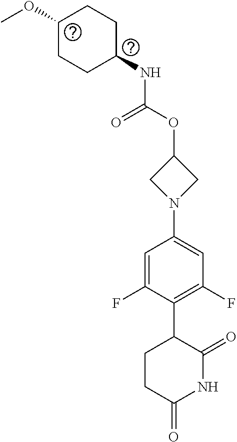 [SMILES: CO[C@H]1CC[C@H](NC(=O)OC2CN(c3cc(F)c(C4CCC(=O)NC4=O)c(F)c3)C2)CC1] |
| 134 | 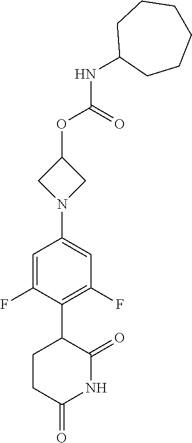 [SMILES: O=C1CCC(c2c(F)cc(N3CC(OC(=O)NC4CCCCCC4)C3)cc2F)C(=O)N1] |
| 135 | 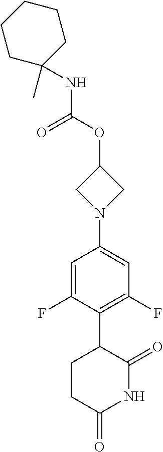 [SMILES: CC1(NC(=O)OC2CN(c3cc(F)c(C4CCC(=O)NC4=O)c(F)c3)C2)CCCCC1] |
| 136 | 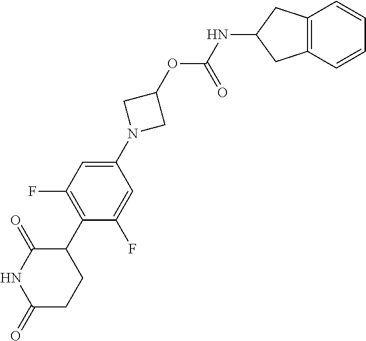 [SMILES: O=C1CCC(c2c(F)cc(N3CC(OC(=O)NC4Cc5ccccc5C4)C3)cc2F)C(=O)N1] |
| 137 | 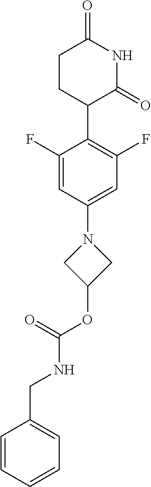 [SMILES: O=C1CCC(c2c(F)cc(N3CC(OC(=O)NCc4ccccc4)C3)cc2F)C(=O)N1] |
| 138 | 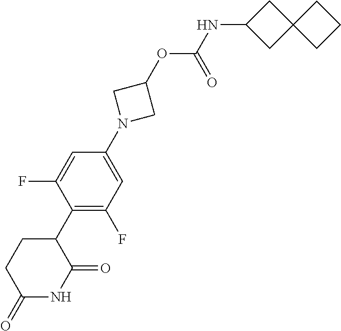 [SMILES: O=C1CCC(c2c(F)cc(N3CC(OC(=O)NC4CC5(CCC5)C4)C3)cc2F)C(=O)N1] |
| 139 | 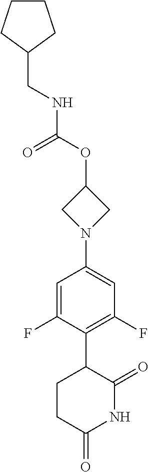 [SMILES: O=C1CCC(c2c(F)cc(N3CC(OC(=O)NCC4CCCC4)C3)cc2F)C(=O)N1] |
| 140 | 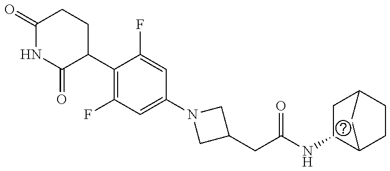 [SMILES: O=C1CCC(c2c(F)cc(N3CC(CC(=O)N[C@@H]4CC5CCC4C5)C3)cc2F)C(=O)N1] |
| 141 | 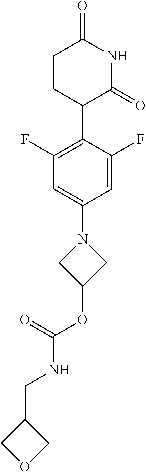 [SMILES: O=C1CCC(c2c(F)cc(N3CC(OC(=O)NCC4COC4)C3)cc2F)C(=O)N1] |
| 142 | 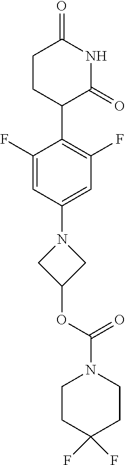 [SMILES: O=C1CCC(c2c(F)cc(N3CC(OC(=O)N4CCC(F)(F)CC4)C3)cc2F)C(=O)N1] |
| 143 | 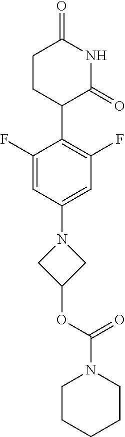 [SMILES: O=C1CCC(c2c(F)cc(N3CC(OC(=O)N4CCCCC4)C3)cc2F)C(=O)N1] |
| 144 | 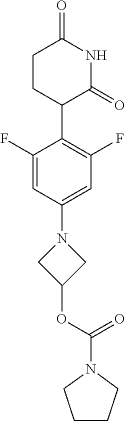 [SMILES: O=C1CCC(c2c(F)cc(N3CC(OC(=O)N4CCCC4)C3)cc2F)C(=O)N1] |
| 145 | 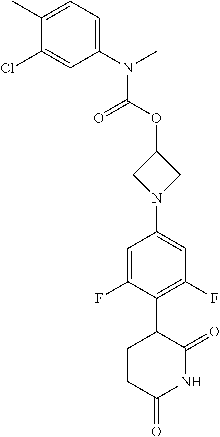 [SMILES: Cc1ccc(N(C)C(=O)OC2CN(c3cc(F)c(C4CCC(=O)NC4=O)c(F)c3)C2)cc1Cl] |

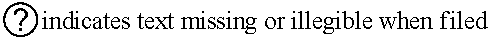

### Pharmaceutical Compositions

In another embodiment, the present disclosure provides a pharmaceutical composition comprising a compound described herein, or pharmaceutically acceptable salt thereof, and a pharmaceutically acceptable excipient. In certain embodiments, the pharmaceutical composition comprises an effective amount of the compound. In certain embodiments, the pharmaceutical composition comprises a therapeutically effective amount of the compound.

The pharmaceutical compositions provided herein can be administered by a variety of routes including, but not limited to, oral (enteral) administration, parenteral (by injection) administration, rectal administration, transdermal administration, intradermal administration, intrathecal administration, subcutaneous (SC) administration, intravenous (IV) administration, intramuscular (IM) administration, and intranasal administration.

Compositions for oral administration can take the form of bulk liquid solutions or suspensions, or bulk powders. In some embodiments, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component with the remainder being various vehicles or excipients and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable excipients known in the art. As before, the active compound in such compositions is typically a minor component with the remainder being the injectable excipient and the like.

Transdermal compositions are typically formulated as a topical ointment or cream containing the active ingredient(s). When formulated as a ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or Formulation. All such known transdermal formulations and ingredients are included within the scope of the disclosure provided herein.

The compounds provided herein can also be administered by a transdermal device. Accordingly, transdermal administration can be accomplished using a patch either of the reservoir or porous membrane type, or of a solid matrix variety.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pennsylvania, which is incorporated herein by reference.

### Methods of Treatment and Uses

Furthermore, the compounds and pharmaceutical compositions described herein are contemplated as useful in the treatment or prevention of disorders in subjects in need thereof. Compounds described herein, in one embodiment, are used to bind to and modulate the surface of cereblon to mediate ubiquitination of GSPT1 and subsequent degradation of said GSPT1 for the treatment of prevention of a disorder.

Accordingly, in one embodiment of the disclosure, a compound, or pharmaceutically acceptable salt thereof, or pharmaceutical composition described herein is administered to a subject to degrade GSPT1 in the subject. GSPT1 is a translational termination factor associated with cancers overexpressing one of the Myc family genes (c-Myc, N-Myc and L-Myc). The Myc transcription factors are some of the most frequently mutated, translocated and overexpressed oncogenes in human cancers. For example, around 10% of non-small cell lung cancer, or NSCLC, overexpress N-Myc and over 50% of small cell lung cancer, or SCLC, overexpress L-Myc. Myc-driven cancer cells are highly addicted to protein translation.

In one aspect of the disclosure, described herein is a method of treating or preventing a disorder in a subject in need thereof, comprising administering to the subject an effective amount of a compound, or pharmaceutically acceptable salt thereof, or pharmaceutical composition described herein.

In another aspect, described herein is a method of degrading GSPT1 in a subject suffering from a disorder, comprising administering to the subject a therapeutically effective amount of a compound described herein, or pharmaceutically acceptable salt thereof, or pharmaceutical composition described herein.

Exemplary disorders that can be treated or prevented by the methods of the present disclosure include but are not limited to, cancer of the bladder, bone, brain, breast, cervix, chest, colon, endometrium, esophagus, eye, head, kidney, liver, lymph nodes, lung, upper aerodigestive tract (including nasal cavity and paranasal sinuses, nasopharynx or cavum, oral cavity, oropharynx, larynx, hypopharynx and salivary glands, neck, ovaries, pancreas, prostate, rectum, skin, stomach, testis, throat, or uterus. Other exemplary disorders include, but are not limited to, amyloidosis, neuroblastoma, meningioma, hemangiopericytoma, multiple brain metastase, glioblastoma multiforms, glioblastoma, brain stem glioma, poor prognosis malignant brain tumor, malignant glioma, recurrent malignant glioma, anaplastic astrocytoma, anaplastic oligodendroglioma, neuroendocrine tumor, e.g., neuroendocrine prostate cancer such as castration-resistant neuroendocrine prostate cancer (NEPC) and lung neuroendocrine tumors (Lu-NETs), rectal adenocarcinoma, colorectal cancer, including stage 3 and stage 4 colorectal cancer, unresectable colorectal carcinoma, metastatic hepatocellular carcinoma, Kaposi's sarcoma, malignant melanoma, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma; and blood bourne (liquid) or hematological cancers, including but not limited to leukemias, lymphomas, and myelomas, such as diffuse large B-cell lymphoma (DLBCL), B-cell immunoblastic lymphoma, small non-cleaved cell lymphoma, human lymphotropic virus-type 1 (HTLV-1) leukemia/lymphoma, adult T-cell lymphoma, peripheral T-cell lymphoma (PTCL), cutaneous T-cell lymphoma (CTCL), mantle cell lymphoma (MCL), Hodgkin's lymphoma (HL), non-Hodgkin's lymphoma (NHL), AIDS-related lymphoma, follicular lymphoma, small lymphocytic lymphoma, T-cell/histiocyte rich large B-cell lymphoma, transformed lymphoma, primary mediastinal (thymic) large B-cell lymphoma, splenic marginal zone lymphoma, Richter's transformation, nodal marginal zone lymphoma, ALK-positive large B-cell lymphoma, indolent lymphoma (for example, DLBCL, follicular lymphoma, or marginal zone lymphoma), acute myelogenous leukemia (AML), acute lymphocytic leukemia (ALL), adult T-cell leukemia, chronic lymphocytic leukemia (CLL), small lymphocytic lymphoma (SLL), hairy cell leukemia, myelodysplasia, myeloproliferative disorders, chronic myelogenous leukemia (CML), acute monocytic leukemia (AMoL), myelodysplastic syndrome (MDS), human lymphotropic virus-type 1 (HTLV-1) leukemia, mastocytosis, B-cell acute lymphoblastic leukemia, Non-Hodgkin's Lymphoma, Hodgkin's Lymphoma, and multiple myeloma (MM).

In another aspect of the disclosure, described herein is a method of treating cancer (e.g., a cancer described herein) in a subject in need thereof, comprising administering to the subject a therapeutically effective amount of a compound described herein, or pharmaceutically acceptable salt thereof, or pharmaceutical composition described herein.

In another aspect, described herein is a method of degrading GSPT1 in a subject suffering from cancer (e.g., a cancer described herein), comprising administering to the subject a therapeutically effective amount of a compound described herein, or pharmaceutically acceptable salt thereof, or pharmaceutical composition described herein.

In some embodiments, a cancer described herein is a Myc-driven cancer. In some embodiments, a cancer described herein is lung cancer, breast cancer, neuroendocrine cancer, or haematological cancer. In some embodiments, the lung cancer is non-small cell lung cancer (NSCLC). In some embodiments, the lung cancer is small-cell lung cancer (SCLC). In some embodiments, the hematological cancer is leukaemia or myeloma. In some embodiments, the myeloma is multiple myeloma.

In another aspect, described herein is a method of treating a solid tumor (e.g., a solid tumor described herein) in a subject in need thereof, comprising administering to the subject a therapeutically effective amount of a compound described herein, or pharmaceutically acceptable salt thereof, or pharmaceutical composition described herein.

In another aspect, described herein is a method of treating a liquid tumor (e.g., a liquid tumor described herein) in a subject in need thereof, comprising administering to the subject a therapeutically effective amount of a compound described herein, or pharmaceutically acceptable salt thereof, or pharmaceutical composition described herein. In some embodiments, the liquid tumor is that of a haematological cancer (e.g., a haematological cancer described herein).

In some embodiments, a method described herein comprises administering to the subject an additional therapeutic agent. In some embodiments, the additional therapeutic agent is an aminoglycoside or pharmaceutically acceptable salt thereof.

In another embodiment, provided herein is a method of preventing or treating a disease or disorder (e.g., a disease or disorder described herein) caused by or associated with one or more premature termination codons in a subject in need thereof, comprising administering to the subject a compound described herein or pharmaceutically acceptable salt thereof, or pharmaceutical composition thereof. In some embodiments, the method further comprises administering to the subject an aminoglycoside or pharmaceutically acceptable salt thereof. In some embodiments, the aminoglycoside is selected from geneticin, rhodostreptomycin, streptomycin, gentamicin, kanamycin A, tobramycin, neomycin B, neomycin C, framycetin, paromomycin, ribostamycin, amikacin, arbekacin, bekanamycin (kanamycin B), dibekacin, spectinomycin, hygromycin B, paromomycin sulfate, netilmicin, sisomicin, isepamicin, verdamicin, astromicin, neamine, ribostamycin, paromomycin, lividomycin, apramycin, and derivatives thereof. In some embodiments, the compound and aminoglycoside are administered in a simultaneous or sequential manner.

In another embodiment, described herein is a compound described herein or pharmaceutically acceptable salt thereof, or a pharmaceutical composition described herein, for use in degrading GSPT1 in a subject suffering from cancer, the use comprising administering a therapeutically effective amount of the compound or pharmaceutically acceptable salt thereof, or the pharmaceutical composition to the subject.

In another embodiment, provided herein is a compound described herein or pharmaceutically acceptable salt thereof, or a pharmaceutical composition described herein, for use in treating cancer in a subject in need thereof, the use comprising administering a therapeutically effective amount of the compound or pharmaceutically acceptable salt thereof, or the pharmaceutical composition to the subject.

In some embodiments, the cancer is lung cancer, breast cancer neuroendocrine cancer, or haematological cancer. In some embodiments, the haematological cancer is leukaemia or myeloma. In some embodiments, the myeloma is multiple myeloma. In some embodiments, the cancer is a Myc-driven cancer.

In another embodiment, provided herein is a compound described herein or pharmaceutically acceptable salt thereof, or a pharmaceutical composition described herein, for use in treating a solid tumor in a subject in need thereof, the use comprising administering a therapeutically effective amount of the compound or pharmaceutically acceptable salt thereof, or the pharmaceutical composition to the subject.

In another embodiment, provided herein is a compound described herein or pharmaceutically acceptable salt thereof, or a pharmaceutical composition described herein, for use in treating a liquid tumor in a subject in need thereof, the use comprising administering a therapeutically effective amount of the compound or pharmaceutically acceptable salt thereof, or the pharmaceutical composition to the subject.

In some embodiments, the use further comprises administering to the subject an additional therapeutic agent.

In another embodiment, provided herein is a compound described herein or pharmaceutically acceptable salt thereof, or a pharmaceutical composition described herein, for use in preventing or treating a disease or disorder (e.g., a disease or disorder described herein) caused by or associated with one or more premature termination codons in a subject in need thereof, the use comprising administering to the subject the compound or pharmaceutically acceptable salt thereof, or pharmaceutical composition thereof. In some embodiments, the method further comprises administering to the subject an aminoglycoside or pharmaceutically acceptable salt thereof. In some embodiments, the aminoglycoside is selected from geneticin, rhodostreptomycin, streptomycin, gentamicin, kanamycin A, tobramycin, neomycin B, neomycin C, framycetin, paromomycin, ribostamycin, amikacin, arbekacin, bekanamycin (kanamycin B), dibekacin, spectinomycin, hygromycin B, paromomycin sulfate, netilmicin, sisomicin, isepamicin, verdamicin, astromicin, neamine, ribostamycin, paromomycin, lividomycin, apramycin, and derivatives thereof. In some embodiments, the compound and aminoglycoside are administered in a simultaneous or sequential manner.

### Myc-Driven Cancers

Described herein, in some embodiments, are cancers exhibiting increased expression of one or more of c-Myc, L-Myc, N-Myc, EIF4EBP1, and EIF4EBP2 as well as ones with increase phosphorylation of one or both of EIF4EBP1 and EIF4EBP2.

Myc-driven cancers refer to cancers where there is abnormal activation of Myc oncogene, either due to transcriptional overexpression (e.g., caused by gene amplification, translocation, alterations in upstream signaling pathways) and/or protein stabilization. A myc-driven cancer cell includes a cancer cell that has an increased expression or overexpression (and/or increased activity) of at least one myc transcription factor such as N-myc and/or L-myc and/or C-myc, or a surrogate marker thereof, relative to a control cell such as a normal (e.g., non-cancerous) cell of the same or corresponding cell type. The term “cancerous” when referring to a sample such as a cell or tissue, generally refers to any sample, such as cells or tissues that exhibit, or are predisposed to exhibiting, unregulated growth, including, for example, a neoplastic cell/tissue such as a premalignant cell/tissue or a cancer cell (e.g., carcinoma cell or sarcoma cell).

In some embodiments the Myc-driven cancer or tumor as defined herein refers to a blood borne tumor cancer, such as a hematological cancer, preferably a cancer of hematopoietic and lymphoid tissues and lymphatic system, such as blood cancer, bone marrow cancer, lymph node cancer, acute lymphoblastic leukemia (ALL), chronic lymphocytic lymphoma (CLL), small lymphocytic lymphoma (SLL), acute myelogenous leukemia (AML), chronic myelogenous leukemia (CML), acute monocytic leukemia (AMoL), Hodgkin's lymphoma, non-Hodgkin's lymphomas and multiple myeloma (MM).

In some embodiments, the Myc-driven cancer or tumour is a solid tumor cancer, such as breast cancer, colorectal cancer, lung cancer, e.g. SCLC, NSCLC, neuroendocrine cancer, e.g., neuroendocrine prostate cancer (for example, NEPC (castration-resistant neuroendocrine prostate cancer)) and lung neuroendocrine tumors (Lu-NETs), liver cancer, stomach cancer, pancreatic cancer, gastric cancer, esophageal cancer, bladder cancer, skin cancer, brain cancer, cervical cancer, ovarian cancer, melanoma and head and neck cancer.

In some embodiments the Myc-driven cancer as used herein refers in particular to breast cancer and SCLC. In some embodiments the myc-driven cancer as used herein refers in particular to NSCLC. In some embodiments, the cancer is solid tumor cancer exhibiting amplification of the N-Myc gene and/or the L-Myc gene. In some embodiments the Myc-driven cancer as used herein refers to neuroendocrine cancer, for example, neuroendocrine prostate cancer (for example, NEPC (castration-resistant neuroendocrine prostate cancer)) and lung neuroendocrine tumors (Lu-NETs), acute myelogenous leukemia (AML), lymphoma, and multiple myeloma (MM).

### Solid and Liquid Cancers

The term “solid cancer” or “solid tumor” refers to disease of tissues or organs, such as to malignant, neoplastic, or cancerous solid tumors, i.e. sarcomas, carcinomas. The tissue structure of solid tumors includes interdependent tissue compartments and usually does not contain cysts or fluid areas. A solid cancer or solid tumor includes cancers of the bladder, bone, brain, breast, cervix, chest, colon, endometrium, esophagus, eye, head, kidney, liver, lymph nodes, lung, upper aerodigestive tract (including nasal cavity and paranasal sinuses, nasopharynx or cavum, oral cavity, oropharynx, larynx, hypopharynx and salivary glands), neck, ovaries, pancreas, prostate, rectum, skin, stomach, testis, throat, and uterus. Specific cancers include, but are not limited to, advanced malignancy, amyloidosis, neuroblastoma, meningioma, hemangiopericytoma, multiple brain metastase, glioblastoma multiforms, glioblastoma, brain stem glioma, poor prognosis malignant brain tumor, malignant glioma, recurrent malignant glioma, anaplastic astrocytoma, anaplastic oligodendroglioma, neuroendocrine tumor, e.g., neuroendocrine prostate cancer (for example, NEPC (castration-resistant neuroendocrine prostate cancer)) and lung neuroendocrine tumors (Lu-NETs), rectal adenocarcinoma, colorectal cancer, including stage 3 and stage 4 colorectal cancer, unresectable colorectal carcinoma, metastatic hepatocellular carcinoma, Kaposi's sarcoma, malignant melanoma, cervical cancer, ovarian cancer, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma. In some embodiments, a solid cancer or solid tumor is a cancer of the breast, lung, stomach, colon, bladder, brain, pancreas, liver, head and neck, prostate, ovaries, upper aerodigestive tract and the like.

The term “blood borne cancer” or “blood borne tumor” (also typically referred to as “hematological cancer”) refers to cancer of the body's blood-forming and immune system—the bone marrow and lymphatic tissue. The tissue structure of blood-borne cancers or tumors includes an abnormal mass of cells that is fluid in nature. Such cancers include leukemias (malignant neoplasms of the blood-forming tissues), lymphomas (Non-Hodgkin's Lymphoma), Hodgkin's disease (Hodgkin's Lymphoma) and myeloma. In one embodiment, the myeloma is multiple myeloma (MM). In some embodiments, the leukemia is, for example, acute myelogenous leukemia (AML), acute lymphocytic leukemia (ALL), adult T-cell leukemia, chronic lymphocytic leukemia (CLL), small lymphocytic lymphoma (SLL), hairy cell leukemia, myelodysplasia, myeloproliferative disorders, chronic myelogenous leukemia (CML), acute monocytic leukemia (AMoL), myelodysplastic syndrome (MDS), human lymphotropic virus-type 1 (HTLV-1) leukemia, mastocytosis, or B-cell acute lymphoblastic leukemia. The leukemia can be relapsed, refractory or resistant to conventional therapy. In some embodiments, the lymphoma is, for example, diffuse large B-cell lymphoma (DLBCL), B-cell immunoblastic lymphoma, small non-cleaved cell lymphoma, human lymphotropic virus-type 1 (HTLV-1) leukemia/lymphoma, adult T-cell lymphoma, peripheral T-cell lymphoma (PTCL), cutaneous T-cell lymphoma (CTCL), mantle cell lymphoma (MCL), Hodgkin's lymphoma (HL), non-Hodgkin's lymphoma (NHL), AIDS-related lymphoma, follicular lymphoma, small lymphocytic lymphoma, T-cell/histiocyte rich large B-cell lymphoma, transformed lymphoma, primary mediastinal (thymic) large B-cell lymphoma, splenic marginal zone lymphoma, Richter's transformation, nodal marginal zone lymphoma, or ALK-positive large B-cell lymphoma. In one embodiment, the hematological cancer is indolent lymphoma including, for example, DLBCL, follicular lymphoma, or marginal zone lymphoma. In some embodiments blood-borne cancers or hematological cancers include acute lymphoblastic leukemia (ALL), chronic lymphocytic lymphoma (CLL), small lymphocytic lymphoma (SLL), acute myelogenous leukemia (AML), chronic myelogenous leukemia (CML), acute monocytic leukemia (AMoL), Hodgkin's lymphoma, non-Hodgkin's lymphomas and multiple myeloma (MM).

In particular embodiments, the compounds of the disclosure or pharmaceutically acceptable salts or stereoisomers thereof or a pharmaceutical composition thereof are used for the treatment of cancer associated with GSPT1, such as solid cancers including but not limited to cancers of the bladder, bone, brain, breast, cervix, chest, colon, endometrium, esophagus, eye, head, kidney, liver, lymph nodes, lung, upper aerodigestive tract (including nasal cavity and paranasal sinuses, nasopharynx or cavum, oral cavity, oropharynx, larynx, hypopharynx and salivary glands), neck, ovaries, pancreas, prostate, rectum, skin, stomach, testis, throat, uterus, amyloidosis, neuroblastoma, meningioma, hemangiopericytoma, multiple brain metastase, glioblastoma multiforms, glioblastoma, brain stem glioma, poor prognosis malignant brain tumor, malignant glioma, recurrent malignant glioma, anaplastic astrocytoma, anaplastic oligodendroglioma, neuroendocrine tumor, e.g., neuroendocrine prostate cancer such as castration-resistant neuroendocrine prostate cancer (NEPC) and lung neuroendocrine tumors (Lu-NETs), rectal adenocarcinoma, colorectal cancer, including stage 3 and stage 4 colorectal cancer, unresectable colorectal carcinoma, metastatic hepatocellular carcinoma, Kaposi's sarcoma, malignant melanoma, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma; and blood bourne (liquid) or hematological cancers, including but not limited to leukemias, lymphomas, and myelomas, such as diffuse large B-cell lymphoma (DLBCL), B-cell immunoblastic lymphoma, small non-cleaved cell lymphoma, human lymphotropic virus-type 1 (HTLV-1) leukemia/lymphoma, adult T-cell lymphoma, peripheral T-cell lymphoma (PTCL), cutaneous T-cell lymphoma (CTCL), mantle cell lymphoma (MCL), Hodgkin's lymphoma (HL), non-Hodgkin's lymphoma (NHL), AIDS-related lymphoma, follicular lymphoma, small lymphocytic lymphoma, T-cell/histiocyte rich large B-cell lymphoma, transformed lymphoma, primary mediastinal (thymic) large B-cell lymphoma, splenic marginal zone lymphoma, Richter's transformation, nodal marginal zone lymphoma, ALK-positive large B-cell lymphoma, indolent lymphoma (for example, DLBCL, follicular lymphoma, or marginal zone lymphoma), acute myelogenous leukemia (AML), acute lymphocytic leukemia (ALL), adult T-cell leukemia, chronic lymphocytic leukemia (CLL), small lymphocytic lymphoma (SLL), hairy cell leukemia, myelodysplasia, myeloproliferative disorders, chronic myelogenous leukemia (CML), acute monocytic leukemia (AMoL), myelodysplastic syndrome (MDS), human lymphotropic virus-type 1 (HTLV-1) leukemia, mastocytosis, B-cell acute lymphoblastic leukemia, Non-Hodgkin's Lymphoma, Hodgkin's Lymphoma, and multiple myeloma (MM).

Such a use (or method of treatment) of a subject comprises administering to a subject in need of such treatment a therapeutically effective amount of a compound of the disclosure or pharmaceutically acceptable salts thereof or a pharmaceutical composition thereof by targeting cereblon.

Disclosed herein, in part, is a method of treating an Myc-driven cancer in a subject in need thereof, comprising administering the subject a therapeutically effective amount of a compound described herein or a composition as described herein.

In some embodiments, the Myc-driven cancer is an Myc-driven lung cancer.

In some embodiments, the Myc-driven cancer is characterized by high driven Myc tumor.

In some embodiments, the Myc-driven cancer is an Myc-driven small cell lung cancer.

In some embodiments, the Myc-driven small cell lung cancer is a high L-Myc small cell lung cancer.

In some embodiments, the Myc-driven cancer is an Myc-driven non-small cell lung cancer.

In some embodiments, the Myc-driven non-small cell lung cancer is a high N-Myc non-small cell lung cancer.

In some embodiments, the compound or the composition is administered to the subject via oral administration.

In another aspect, provided herein is a method of degrading GSPT1 in a subject suffering from cancer, comprising administering to the subject a therapeutically effective amount of a compound described herein or a composition described herein.

In some embodiments, the cancer is a Myc-driven cancer.

In some embodiments, the Myc-driven cancer is an Myc-driven lung cancer.

In some embodiments, the Myc-driven cancer is an Myc-driven small cell lung cancer.

In some embodiments, the Myc-driven small cell lung cancer is a high L-Myc small cell lung cancer.

In some embodiments, the Myc-driven cancer is an Myc-driven non-small cell lung cancer.

In some embodiments, the Myc-driven non-small cell lung cancer is a high N-Myc non-small cell lung cancer.

In some embodiments, the compound or the composition is administered to the subject via oral administration.

In another aspect, the disclosure is directed to a method of reducing the level of GSPT1 in a subject suffering from cancer, comprising administering the subject a therapeutically effective amount of a compound or a composition as described herein.

In some embodiments, the cancer is a Myc-driven cancer.

In some embodiments, the Myc-driven cancer is an Myc-driven lung cancer.

In some embodiments, the Myc-driven cancer is an Myc-driven small cell lung cancer.

In some embodiments, the Myc-driven small cell lung cancer is a high L-Myc small cell lung cancer.

In some embodiments, the Myc-driven cancer is an Myc-driven non-small cell lung cancer.

In some embodiments, the Myc-driven non-small cell lung cancer is a high N-Myc non-small cell lung cancer.

In some embodiments, the compound or the composition is administered to the subject via oral administration.

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “alkyl” as used herein refers to a radical of a straight-chain or branched saturated hydrocarbon group. In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”, also referred to herein as “lower alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Common alkyl abbreviations include Me (—CH₃), Et (—CH₂CH₃), iPr (—CH(CH₃)₂), nPr (—CH₂CH₂CH₃), n-Bu (—CH₂CH₂CH₂CH₃), or i-Bu (—CH₂CH(CH₃)₂).

The term “alkenyl” as used herein refers to a radical of a straight-chain or branched hydrocarbon group having, one or more carbon-carbon double bonds. In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C), and the like. Additional examples of alkenyl include heptenyl (C₇), octenyl (C₈), octatrienyl (C₈), and the like.

The term “alkynyl” as used herein refers to a radical of a straight-chain or branched hydrocarbon group having one or more carbon-carbon triple bonds (e.g., 1, 2, 3, or 4 carbon-carbon triple bonds). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like.

The term “aminoglycoside” or “aminoglycoside of the disclosure” as used herein, refers to any aminoglycoside of the prior art and in particular to any aminoglycoside as defined in the description, as well as pharmaceutically acceptable salts and/or stereoisomers thereof.

The term “cycloalkyl” as used herein refers to a radical of a saturated or partially unsaturated cyclic hydrocarbon group having from 3 to 10 ring carbon atoms (“C₃₋₁₀ cycloalkyl”) and zero heteroatoms in the ring system. In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 3 to 6 ring carbon atoms (“C₃₋₆ cycloalkyl”). In some embodiments, a cycloalkyl group has 5 to 10 ring carbon atoms (“C₅₋₁₀ cycloalkyl”). Exemplary C₃₋₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ cycloalkyl groups include, without limitation, the aforementioned C₃₋₆ cycloalkyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ cycloalkyl groups include, without limitation, the aforementioned C₃₋₈ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”). “Cycloalkyl” also includes ring systems wherein the cycloalkyl ring, as defined above, is fused with one or more aryl or heteroaryl groups wherein the point of attachment is on the cycloalkyl ring or the one or more aryl or heteroaryl groups, and in such instances, the number of carbons continue to designate the number of carbons in the cycloalkyl ring system.

The term “heterocyclyl” as used herein refers to a radical of a saturated or partially unsaturated 3 to 10 membered ring system having ring carbon atoms and 1 to 4 ring heteroatoms, wherein each heteroatom is independently selected from nitrogen, oxygen, sulfur, boron, phosphorus, and silicon (“3 to 10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”). Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring or the one or more aryl or heteroaryl groups, and in such instances, the number of ring members continue to designate the number of ring members in the heterocyclyl ring system.

In some embodiments, a heterocyclyl group is a 5 to 10 membered saturated or partially unsaturated ring system having ring carbon atoms and 1 to 4 ring heteroatoms, wherein each heteroatom is independently selected from nitrogen, oxygen, sulfur, boron, phosphorus, and silicon (“5 to 10 membered heterocyclyl”). In some embodiments, a heterocyclyl group is a 5 to 8 membered saturated or partially unsaturated ring system having ring carbon atoms and 1 to 4 ring heteroatoms, wherein each heteroatom is independently selected from nitrogen, oxygen, and sulfur (“5 to 8 membered heterocyclyl”). In some embodiments, a heterocyclyl group is a 5 to 6 membered saturated or partially unsaturated ring system having ring carbon atoms and 1 to 4 ring heteroatoms, wherein each heteroatom is independently selected from nitrogen, oxygen, and sulfur (“5 to 6 membered heterocyclyl”). In some embodiments, the 5 to 6 membered heterocyclyl has 1 to 3 ring heteroatoms selected from nitrogen, oxygen, and sulfur. In some embodiments, the 5 to 6 membered heterocyclyl has 1 to 2 ring heteroatoms selected from nitrogen, oxygen, and sulfur. In some embodiments, the 5 to 6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

The term “aryl” as used herein refers to a radical of a monocyclic or polycyclic (e.g., bicyclic or tricyclic) 4n+2 aromatic ring system (e.g., having 6, 10, or 14 π electrons shared in a cyclic array) having 6 to 14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particularly aryl groups include phenyl, naphthyl, indenyl, and tetrahydronaphthyl.

The term “heteroaryl” as used herein refers to a radical of a 5 to 10 membered monocyclic or bicyclic 4n+2 aromatic ring system (e.g., having 6 or 10 π electrons shared in a cyclic array) having ring carbon atoms and 1 to 4 ring heteroatoms provided in the aromatic ring system, wherein each heteroatom is independently selected from nitrogen, oxygen and sulfur (“5 to 10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5 to 10 membered aromatic ring system having ring carbon atoms and 1 to 4 ring heteroatoms provided in the aromatic ring system, wherein each heteroatom is independently selected from nitrogen, oxygen, and sulfur (“5 to 10 membered heteroaryl”). In some embodiments, a heteroaryl group is a 5 to 8 membered aromatic ring system having ring carbon atoms and 1 to 4 ring heteroatoms provided in the aromatic ring system, wherein each heteroatom is independently selected from nitrogen, oxygen, and sulfur (“5 to 8 membered heteroaryl”). In some embodiments, a heteroaryl group is a monocyclic 5 to 6 membered aromatic ring system having ring carbon atoms and 1 to 4 ring heteroatoms provided in the aromatic ring system, wherein each heteroatom is independently selected from nitrogen, oxygen, and sulfur (“5 to 6 membered heteroaryl”). In some embodiments, the 5 to 6 membered heteroaryl has 1 to 3 ring heteroatoms selected from nitrogen, oxygen, and sulfur. In some embodiments, the 5 to 6 membered heteroaryl has 1 to 2 ring heteroatoms selected from nitrogen, oxygen, and sulfur. In some embodiments, the 5 to 6 membered heteroaryl has 1 ring heteroatom selected from nitrogen, oxygen, and sulfur. In some embodiments, a heteroaryl group is a monocyclic 5 membered aromatic ring system having ring carbon atoms and 1 to 4 ring heteroatoms provided in the aromatic ring system, wherein each heteroatom is independently selected from nitrogen, oxygen, and sulfur (“5-membered heteroaryl”). In some embodiments, a heteroaryl group is a monocyclic 6 membered aromatic ring system having ring carbon atoms and 1 to 4 ring heteroatoms provided in the aromatic ring system, wherein each heteroatom is independently selected from nitrogen, oxygen, and sulfur (“6-membered heteroaryl”).

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

The term “alkoxy” as used herein refers to the group —OR¹⁰⁰ where R¹⁰⁰ is alkyl, alkenyl, alkynyl, cycloalkyl, heterocyclyl, aryl, or heteroaryl. Exemplary alkoxy groups include methoxy, ethoxy, n-propoxy, isopropoxy, n-butoxy, tert-butoxy, sec-butoxy, n-pentoxy, n-hexoxy, and 1,2-dimethylbutoxy. Other exemplary alkoxy groups are lower alkoxy, i.e. with between 1 and 6 carbon atoms. In other examples, alkoxy groups have between 1 and 4 carbon atoms.

The term “cyano” as used herein refers to the radical—CN.

The term “halogen” as used herein refers to F, Cl, Br, or I.

The term “thioalkoxy” as used herein refers to the group —SR¹⁰¹ where R¹⁰¹ is alkyl, alkenyl, alkynyl, cycloalkyl, heterocyclyl, aryl, or heteroaryl. Exemplary thioalkoxy groups are lower thioalkoxy, i.e. with between 1 and 6 carbon atoms. In other examples, thioalkoxy groups have between 1 and 4 carbon atoms.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of the present disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

A “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” and “subject” are used interchangeably herein.

The terms “disease,” “disorder,” and “condition” are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition. In an alternative embodiment, the present disclosure contemplates administration of the compounds described herein as a prophylactic before a subject begins to suffer from the specified disease, disorder or condition.

In general, the “effective amount” of a compound as used herein refers to an amount sufficient to elicit the desired biological response. As will be appreciated by those of ordinary skill in this art, the effective amount of a compound of the present disclosure may vary depending on such factors as the desired biological endpoint, the pharmacokinetics of the compound, the disease being treated, the mode of administration, and the age, health, and condition of the subject.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.” Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

Isomers, e.g., stereoisomers, can be isolated from mixtures by methods known to those skilled in the art, including chiral high pressure liquid chromatography (HPLC) and the formation of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, IN 1972). The present disclosure additionally encompasses compounds described herein as individual isomers substantially free of other isomers, and alternatively, as mixtures of various isomers.

The compounds provided herein can be administered as the sole active agent, or they can be administered in combination with other active agents. In some embodiments, the present invention provides a combination of a compound of the present invention and another pharmacologically active agent. Administration in combination can proceed by any technique apparent to those of skill in the art including, for example, separate, sequential, concurrent, and alternating administration.

The present disclosure, in an alternative embodiment, also embraces isotopically labeled compounds which are identical to those recited herein, except that one or more atoms are replaced by an atom having an atomic mass or mass number different from the atomic mass or mass number usually found in nature. Examples of isotopes that can be incorporated into compounds described herein include isotopes of hydrogen, carbon, nitrogen, oxygen, phosphorus, sulfur, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. For example, a compound of the disclosure may have one or more H atom replaced with deuterium.

### EXAMPLES

The compounds provided herein can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization.

Abbreviations: Bn: benzyl; Boc: tert-butyloxycarbonyl; CRBN: cereblon; DMF: N,N-dimethylformamide; DMSO: dimethyl sulfoxide; eq: equivalents; EI: electron ionization; ESI: electrospray ionization; h: hours; HPLC: high-performance liquid chromatography; MS: mass spectrometry; MTBE: tert-butyl methyl ether; NMR: nuclear magnetic resonance; Py: pyridine; SEM: trimethylsilylethoxymethyl.

### Example 1: Synthesis of Intermediates, General Procedure A

### Synthesis of Intermediate IX

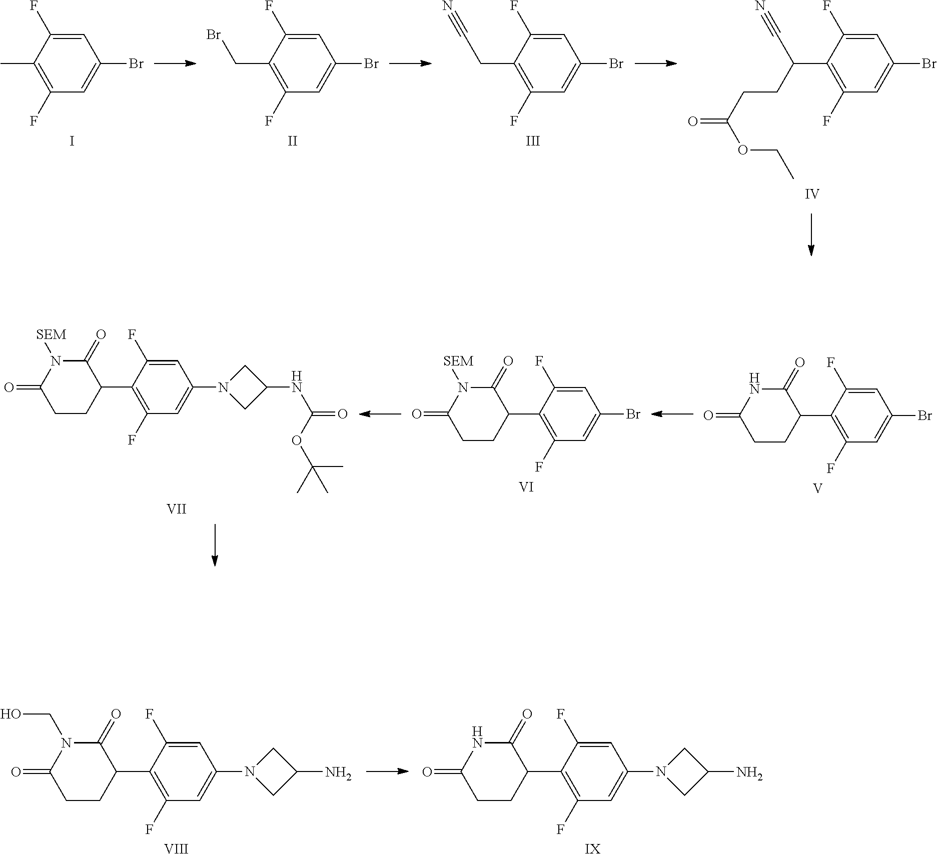

Intermediate II: To a solution of 5-bromo-1,3-difluoro-2-methylbenzene I (19.3 g, 93.1 mmol, 1.00 eq) in tetrachloromethane (190 mL) was added N-bromosuccinimide (16.6 g, 93.1 mmol, 1.00 eq) and (E)-3,3′-(diazene-1,2-diyl)bis(2-methylpropanenitrile) (764 mg, 4.65 mmol, 0.0500 eq). The mixture was stirred at 80° C. for 2 h. The mixture was filtered and the filtrate was concentrated under reduced pressure to give a residue. The residue was purified by column chromatography (SiO₂, petroleum ether/ethyl acetate=1/0 to 20/1) and concentrated under reduced pressure to afford 5-bromo-2-(bromomethyl)-1,3-difluorobenzene II (21.2 g, 74.2 mmol, 80% yield) as a yellow oil. ¹H NMR (400 MHz, DMSO-d₆) δ=7.59-7.56 (m, 1H), 7.56-7.52 (m, 1H), 4.61 (s, 2H).

Intermediate III: To a solution of 5-bromo-2-(bromomethyl)-1,3-difluorobenzene II (21.2 g, 74.2 mmol, 1.00 eq) in ethanol (150 mL) and water (50 mL) was added potassium cyanide (3.81 mL, 89.0 mmol, 1.20 eq). The mixture was stirred at 60° C. for 2 h. The mixture was diluted with water (400 mL) and extracted with ethyl acetate (3×300 mL). The combined organic layers were dried over anhydrous sodium sulfate, filtered and concentrated under reduced pressure to give a residue. The residue was purified by column chromatography (SiO₂, petroleum ether/ethyl acetate=1/0 to 20/1) and concentrated under reduced pressure to afford 2-(4-bromo-2,6-difluorophenyl)acetonitrile III (12.2 g, 52.6 mmol, 71% yield) as a white solid. 1H NMR (400 MHz, DMSO-d6) δ=7.58 (s, 1H), 7.56 (s, 1H), 4.02 (s, 2H).

Intermediate IV: To a solution of 2-(4-bromo-2,6-difluorophenyl)acetonitrile (12.2 g, 52.6 mmol, 1.00 eq), methyl acrylate (4.73 mL, 52.6 mmol, 1.00 eq) in tetrahydrofuran (120 mL) was added sodium methoxide (284.06 mg, 5.26 mmol, 0.100 eq) at 0° C. The mixture was stirred at 20° C. for 1 h. The mixture was quenched with saturated ammonium chloride aqueous solution (50 mL) and extracted with ethyl acetate (3×50 mL). The combined organic layers were washed with brine (2×300 mL), dried over anhydrous sodium sulfate, filtered and concentrated under reduced pressure to give a residue. The residue was purified by column chromatography (SiO₂, petroleum ether/ethyl acetate=1/0 to 10/1) and concentrated under reduced pressure to afford methyl 4-(4-bromo-2,6-difluorophenyl)-4-cyanobutanoate IV (13.5 g, 42.4 mmol, 81% yield) as a yellow oil. ¹H NMR (400 MHz, DMSO-d₆) δ=7.64-7.62 (m, 1H), 7.61-7.58 (m, 1H), 4.52 (t, J=7.8 Hz, 1H), 3.56 (s, 3H), 2.48-2.41 (m, 2H), 2.22 (qd, J=7.2, 14.0 Hz, 1H), 2.13-2.03 (m, 1H).

Intermediate V: To a solution of methyl 4-(4-bromo-2,6-difluorophenyl)-4-cyanobutanoate IV (13.5 g, 42.4 mmol, 1.00 eq) in acetic acid (135 mL) was added sulfuric acid (13.5 mL, 253 mmol, 5.97 eq). The mixture was stirred at 90° C. for 2 h. The mixture was poured into ice water (400 mL) and filtered. The filter cake was lyophilized to afford 3-(4-bromo-2,6-difluorophenyl)piperidine-2,6-dione V (15.0 g, crude) as a white solid. ¹H NMR (400 MHz, DMSO-d₆) δ=11.00 (s, 1H), 7.52 (s, 1H), 7.50 (s, 1H), 4.25 (dd, J=5.1, 12.7 Hz, 1H), 2.87-2.75 (m, 1H), 2.55 (br d, J=3.2 Hz, 1H), 2.12 (dq, J=3.9, 13.0 Hz, 1H), 2.06-1.97 (m, 1H).

Intermediate VI: To a solution of 3-(4-bromo-2,6-difluorophenyl)piperidine-2,6-dione V (5.00 g, 16.4 mmol, 1.00 eq) and 2,3,4,6,7,8,9,10-octahydropyrimido[1,2-a]azepine (DBU) (4.96 mL, 32.9 mmol, 2.00 eq) in tetrahydrofuran (50 mL) was added (2-trimethylethylsilylethoxy)methyl chloride (4.93 g, 29.6 mmol, 5.24 mL, 1.80 eq) at 0° C. The reaction mixture was stirred at 20° C. for 12 h. The mixture was quenched with saturated sodium bicarbonate aqueous solution (80 mL) and extracted with ethyl acetate (3×70 mL). The combined organic layers were dried over anhydrous sodium sulfate, filtered and concentrated under reduced pressure to give a residue. The residue was purified by column chromatography (SiO₂, petroleum ether/ethyl acetate=1/0 to 3/1) and concentrated under reduced pressure to afford 3-(4-bromo-2,6-difluorophenyl)-1-((2-(trimethylsilyl)ethoxy)methyl)piperidine-2,6-dione VI (5.31 g, 12.2 mmol, 74% yield) as a yellow oil. ¹H NMR (400 MHz, DMSO-d₆) δ=7.55-7.52 (m, 1H), 7.52-7.48 (m, 1H), 5.10 (s, 2H), 4.41 (dd, J=5.1, 12.8 Hz, 1H), 3.55-3.49 (m, 2H), 3.04-2.92 (m, 1H), 2.78-2.70 (m, 1H), 2.15 (dq, J=4.0, 13.2 Hz, 1H), 2.08-1.99 (m, 1H), 0.86-0.79 (m, 2H), −0.01-−0.04 (m, 9H).

Intermediate VII: To a solution of 3-(4-bromo-2,6-difluorophenyl)-1-((2-(trimethylsilyl)ethoxy)methyl)piperidine-2,6-dione VI (3.00 g, 6.91 mmol, 1.00 eq), tert-butyl azetidin-3-ylcarbamate (2.88 g, 13.8 mmol, 2.00 eq, hydrochloride) and cesium carbonate (6.75 g, 20.7 mmol, 3.00 eq) in toluene (30 mL) were added 4,5-bis(diphenylphosphino)-9,9-dimethylxanthene (599 mg, 1.04 mmol, 0.150 eq) and tris(dibenzylideneacetone)dipalladium(0) (316 mg, 345 μmol, 0.0500 eq). The mixture was stirred at 110° C. for 12 h under nitrogen atmosphere. The mixture was filtered and concentrated under residue pressure to give a residue. The residue was purified by column chromatography (SiO₂, petroleum ether/ethyl acetate=1/0 to 5/1) to afford tert-butyl (1-(4-(2,6-dioxo-1-((2-(trimethylsilyl) ethoxy)methyl)piperidin-3-yl)-3,5-difluorophenyl)azetidin-3-yl)carbamate VII (2.70 g, 5.14 mmol, 74% yield) as a yellow oil. ¹H NMR (400 MHz, DMSO-d₆) δ=7.55 (br d, J=7.3 Hz, 1H), 6.14 (s, 1H), 6.12 (s, 1H), 5.12-5.05 (m, 2H), 4.45-4.34 (m, 1H), 4.18 (br dd, J=5.0, 12.6 Hz, 1H), 4.10-4.05 (m, 2H), 3.63-3.58 (m, 2H), 3.55-3.47 (m, 2H), 3.00-2.88 (m, 1H), 2.73-2.66 (m, 1H), 2.10 (dq, J=3.2, 12.9 Hz, 1H), 1.98-1.90 (m, 1H), 1.39 (s, 9H), 0.86-0.80 (m, 2H), −0.01-−0.05 (m, 9H).

Intermediate VIII: To a solution of tert-butyl (1-(4-(2,6-dioxo-1-((2-(trimethylsilyl)ethoxy)methyl)piperidin-3-yl)-3,5-difluorophenyl)azetidin-3-yl)carbamate VII (2.70 g, 5.14 mmol, 1.00 eq) in dichloromethane (20 mL) was added trifluoroacetic acid (10.0 mL, 135 mmol, 26.3 eq). The mixture was stirred at 20° C. for 1 h. The mixture was concentrated under reduced pressure to afford 3-(4-(3-aminoazetidin-1-yl)-2,6-difluorophenyl)-1-(hydroxymethyl)piperidine-2,6-dione VIII (3.50 g, crude) as a yellow solid and was used into the next step without further purification. MS (ESI) m/z 326.1 [M+H]⁺.

Intermediate IX: To a solution of 3-(4-(3-aminoazetidin-1-yl)-2,6-difluorophenyl)-1-(hydroxymethyl)piperidine-2,6-dione VIII (3.50 g, 10.8 mmol, 1.00 eq) in acetonitrile (40 mL) was added ammonium hydroxide (2.00 mL, 18.2 mmol, 35% purity, 1.69 eq). The mixture was stirred at 20° C. for 1 h. The mixture was concentrated under reduced pressure to give a residue. The residue was purified by reversed phase column chromatography (C18, 330 g; condition: water/acetonitrile=1/0 to 0/1, 0.1% formic acid) and lyophilized to afford 3-(4-(3-aminoazetidin-1-yl)-2,6-difluorophenyl)piperidine-2,6-dione IX (780 mg, 2.29 mmol, 21% yield, formate) as a white solid and 3-(4-(3-aminoazetidin-1-yl)-2,6-difluorophenyl)piperidine-2,6-dione IX (650 mg, 1.59 mmol, 15% yield, trifluoroacetate) as an off-white solid. 1H NMR (400 MHz, DMSO-d₆) δ=10.87 (s, 1H), 8.33 (br s, 3H), 6.29 (s, 1H), 6.26 (s, 1H), 4.11 (br s, 3H), 4.05 (br dd, J=5.1, 12.6 Hz, 1H), 3.85-3.76 (m, 2H), 2.84-2.72 (m, 1H), 2.52 (br d, J=2.0 Hz, 1H), 2.14-2.02 (m, 1H), 1.98-1.89 (m, 1H).

### Synthesis of Intermediate XII

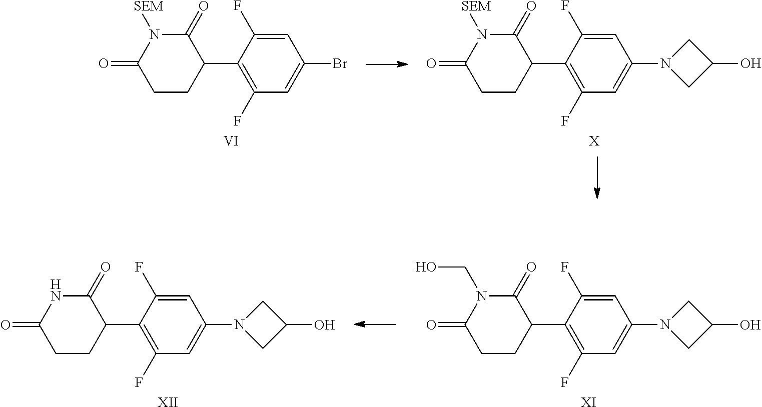

Intermediate X: To a solution of 3-(4-bromo-2,6-difluorophenyl)-1-((2-(trimethylsilyl)ethoxy)methyl)piperidine-2,6-dione VI (1.00 g, 2.30 mmol, 1.00 eq), azetidin-3-ol (378 mg, 3.45 mmol, 1.50 eq, hydrochloride), cesium carbonate (2.25 g, 6.91 mmol, 3.00 eq) in dioxane (10 mL) were added 4,5-bis(diphenylphosphino)-9,9-dimethylxanthene (133 mg, 0.230 mmol, 0.100 eq) and palladium acetate (51.7 mg, 0.230 mmol, 0.100 eq). The mixture was stirred at 100° C. for 12 h under nitrogen atmosphere. The mixture was diluted with water (100 mL) and extracted with ethyl acetate (3×100 mL). The combined organic layers were dried over anhydrous sodium sulfate, filtered and concentrated under reduced pressure to give a residue. The residue was purified by column chromatography (SiO₂, petroleum ether/ethyl acetate=1/0 to 3/1) and concentrated under reduced pressure to afford 3-(2,6-difluoro-4-(3-hydroxyazetidin-1-yl)phenyl)-1-((2-(trimethylsilyl) ethoxy)methyl)piperidine-2,6-dione X (300 mg, 0.703 mmol, 30% yield) as a yellow oil. ¹H NMR (400 MHz, DMSO-d₆) δ=6.12 (s, 1H), 6.10 (s, 1H), 5.66 (d, J=6.5 Hz, 1H), 5.12-5.06 (m, 2H), 4.58-4.52 (m, 1H), 4.18 (br dd, J=5.0, 12.7 Hz, 1H), 4.08-4.04 (m, 2H), 3.56-3.50 (m, 4H), 2.98-2.89 (m, 1H), 2.73-2.66 (m, 1H), 2.15-2.05 (m, 1H), 1.97-1.89 (m, 1H), 0.85-0.81 (m, 2H), −0.01-−0.04 (m, 9H).

Intermediate XI: To a solution of 3-(2,6-difluoro-4-(3-hydroxyazetidin-1-yl)phenyl)-1-((2-(trimethylsilyl)ethoxy)methyl) piperidine-2,6-dione X (250 mg, 586 μmol, 1.00 eq) in dichloromethane (10 mL) was added trifluoroacetic acid (1 mL). The mixture was stirred at 20° C. for 4 h. The mixture was concentrated under reduced pressure to afford 3-(2,6-difluoro-4-(3-hydroxyazetidin-1-yl)phenyl)-1-(hydroxymethyl)piperidine-2,6-dione XI (250 mg, crude) as yellow oil and was used for the next step without further purification.

Intermediate XII: To a solution of 3-(2,6-difluoro-4-(3-hydroxyazetidin-1-yl)phenyl)-1-(hydroxymethyl)piperidine-2,6-dione (250 mg, 766 μmol, 1.00 eq) in acetonitrile (10 mL) was added ammonium hydroxide (0.200 mL, 25% purity). The mixture was stirred at 20° C. for 12 h. The mixture was concentrated under reduced pressure to give a residue. The residue was purified by reverse phase column chromatography (C18, 40 g; condition: water/acetonitrile=1/0 to 0/1, 0.1% formic acid) and lyophilized to afford 3-(2,6-difluoro-4-(3-hydroxyazetidin-1-yl)phenyl)piperidine-2,6-dione XII (140 mg, 472 μmol, 61% yield) as a white solid. ¹H NMR (400 MHz, DMSO-d₆) δ=10.85 (s, 1H), 6.13 (s, 1H), 6.10 (s, 1H), 5.66 (br d, J=6.1 Hz, 1H), 4.58-4.49 (m, 1H), 4.06 (br t, J=7.4 Hz, 2H), 4.01 (br s, 1H), 3.53 (dd, J=4.8, 8.1 Hz, 2H), 2.83-2.72 (m, 1H), 2.47 (br s, 1H), 2.12-2.01 (m, 1H), 1.94 (ttd, J=2.6, 5.3, 10.5 Hz, 1H).

### Synthesis of Intermediate XVIII

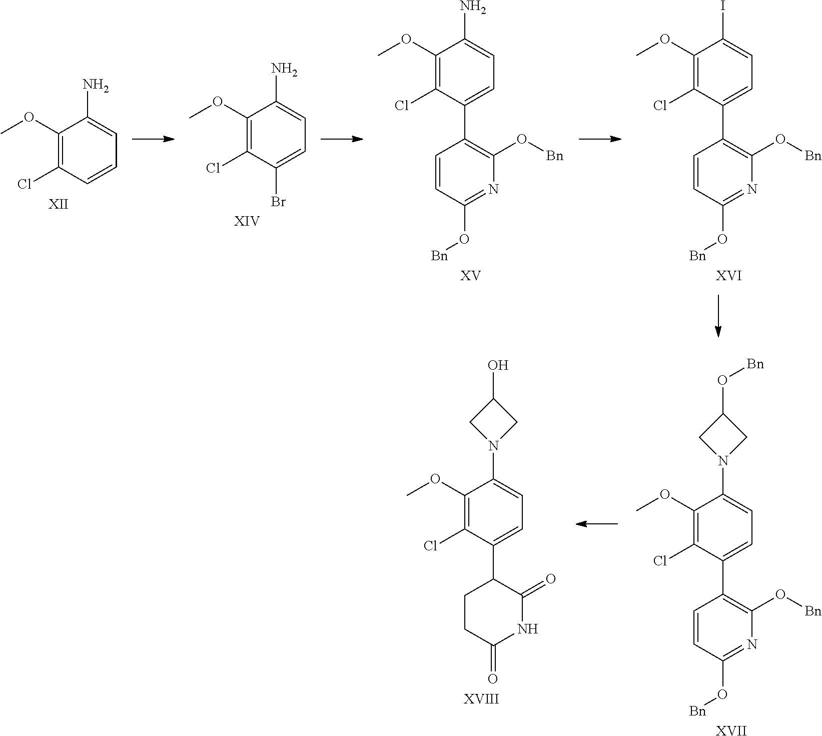

Intermediate XIV: To a stirred solution of 3-chloro-2-methoxy-aniline XII (0.500 g, 3.17 mmol, 1.00 eq) in dimethylformamide (5 mL) was added dropwise of N-bromosuccinimide (0.564 g, 3.17 mmol, 1.00 eq) in dimethylformamide (5 mL), then the resulting mixture was stirred at 20° C. for 2 h. The reaction mixture was quenched by addition water (20 mL), and then extracted with ethyl acetate (3×20 mL). The combined organic layers were washed with brine (3×20 mL), dried over sodium sulfate, filtered and concentrated under reduced pressure to give 4-bromo-3-chloro-2-methoxyaniline XIV (0.680 g, 2.88 mmol, 90% yield) as a brown solid. ¹H NMR (400 MHz, CDCl₃) δ=7.18 (d, J=8.63 Hz, 1H), 6.58 (d, J=8.63 Hz, 1H), 4.0 (s, 2H), 3.86 (s, 3H). MS (ESI) m/z 237.9 [M+H]⁺.

Intermediate XV: To a dried flask were added 4-bromo-3-chloro-2-methoxy-aniline XIV (0.527 g, 2.23 mmol, 1.00 eq), 2,6-bis(benzyloxy)-3-(4,4,5,5-tetramethyl-1,3,2-dioxaborolan-2-yl)pyridine (1.12 g, 2.67 mmol, 1.20 eq), [1,1-bis(diphenylphosphino)ferrocene]dichloropalladium(II) (0.163 g, 0.223 mmol, 0.100 eq), potassium phosphate (1.42 g, 6.68 mmol, 3.00 eq) and dioxane (10 mL), then the resulting solution was stirred at 90° C. for 16 h under nitrogen. The reaction mixture was quenched by addition water (10 mL), and then extracted with ethyl acetate (3×20 mL). The combined organic layers were washed with brine (3×20 mL), dried over sodium sulfate, filtered and concentrated under reduced pressure to give a residue. The residue was purified by flash silica gel chromatography (ISCO®; 20 g SepaFlash® Silica Flash Column, Eluent of 0-20% ethyl acetate/petroleum ether gradient at 25 mL/min) to give 4-(2,6-bis(benzyloxy)pyridin-3-yl)-3-chloro-2-methoxyaniline (0.700 g, 1.57 mmol, 70% yield) as a brown oil. ¹H NMR (400 MHz, CDCl₃) δ=7.36 (d, J=7.75 Hz, 3H), 7.31-7.16 (m, 8H), 6.8 (d, J=8.25 Hz, 1H), 6.62 (d, J=8.25 Hz, 1H), 6.36 (d, J=7.88 Hz, 1H), 5.30 (s, 2H), 5.27 (s, 2H), 3.80 (s, 3H). MS (ESI) m/z 447.1 [M+H]⁺.

Intermediate XVI: To a solution of 4-(2,6-bis(benzyloxy)pyridin-3-yl)-3-chloro-2-methoxyaniline XV (0.600 g, 1.34 mmol, 1.00 eq) in acetone (10 mL) and water (12 mL) was added concentrated hydrochloric acid (12 M, 6.00 mL, 60.0 eq), then the reaction mixture was cooled to 0° C., a solution of sodium nitrite (0.112 g, 1.61 mmol, 1.20 eq) in water (3 mL) was added, then the mixture was stirred at 0° C. for 1 h. A solution of potassium iodide (0.334 g, 2.01 mmol, 1.50 eq) in water (3 mL) was added dropwise, and the mixture was warm to 10° C. with stirring for 2 h. The reaction mixture was quenched by addition of water (10 mL), and then extracted with ethyl acetate (3×20.0 mL). The combined organic layers were washed with brine (3×20 mL), dried over sodium sulfate, filtered and concentrated under reduced pressure to give a residue. The residue was purified by flash silica gel chromatography (ISCO®; 20 g SepaFlash® Silica Flash Column, Eluent of 0-20% ethyl acetate/petroleum ether gradient at 25 mL/min) to give 2,6-bis(benzyloxy)-3-(2-chloro-4-iodo-3-methoxyphenyl)pyridine XVI (0.400 g, 0.72 mmol, 53% yield) as a brown oil. ¹H NMR (400 MHz, CDCl₃) δ=7.6 (d, J=8.13 Hz, 1H), 7.38-7.35 (m, 3H), 7.32-7.18 (m, 8H), 6.74 (d, J=8.13 Hz, 1H), 6.39 (d, J=8.00 Hz, 1H), 5.29 (d, J=1.13 Hz, 4H), 3.82 (s, 3H). MS (ESI) m/z 558.1 [M+H]⁺.

Intermediate XVII: To a stirred solution of 3-(benzyloxy)azetidine (0.716 g, 3.59 mmol, 2.00 eq, hydride acid) and 2,6-bis(benzyloxy)-3-(2-chloro-4-iodo-3-methoxyphenyl)pyridine XVI (1.00 g, 1.79 mmol, 1.00 eq) in dioxane (10 mL) were added cesium carbonate (1.75 g, 5.38 mmol, 3.00 eq), 4,5-bis(diphenylphosphino)-9,9-dimethylxanthene (0.156 g, 0.269 mmol, 0.150 eq), tris(dibenzylideneacetone)dipalladium(0) (82.1 mg, 0.0896 mmol, 0.0500 eq), then the reaction mixture was stirred at 100° C. under nitrogen for 16 h. The reaction mixture was quenched by addition water (20 mL), and then extracted with ethyl acetate (3×30 mL). The combined organic layers were washed with brine (3×30 mL), dried over sodium sulfate, filtered and concentrated under reduced pressure to give a residue. The residue was purified by flash silica gel chromatography (ISCO®; 20 g SepaFlash® Silica Flash Column, Eluent of 0-20% ethyl acetate/petroleum ether gradient at 25 mL/min) to give 2,6-bis(benzyloxy)-3-(4-(3-(benzyloxy)azetidin-1-yl)-2-chloro-3-methoxyphenyl)pyridine XVII (0.800 g, 1.35 mmol, 75% yield) was obtained as a brown oil. ¹H NMR (400 MHz, CDCl₃) δ=7.46-7.44 (m, 3H), 7.42-7.28 (m, 13H), 6.95 (d, J=8.31 Hz, 1H), 6.47-6.42 (m, 2H), 5.38 (d, J=11.98 Hz, 4H), 4.55-4.50 (m, 3H), 4.25-4.21 (m, 2H), 3.90-3.86 (m, 2H), 3.77 (s, 3H). MS (ESI) m/z 593.3 [M+H]⁺.

Intermediate XVIII: To a stirred solution of 2,6-bis(benzyloxy)-3-(4-(3-(benzyloxy)azetidin-1-yl)-2-chloro-3-methoxyphenyl) pyridine XVII (40.0 mg, 67.4 μmol, 1.00 eq) in dioxane (3 mL) were added palladium on carbon (50.0 mg, 10% purity) and lithium chloride (8.58 mg, 202 μmol, 3.00 eq), then the resulting solution was degassed three times with nitrogen and degassed three times with hydrogen. The reaction mixture was stirred at 15° C. for 16 h and was filtrated through diatomite. The filter cake was washed with ethyl alcohol, the filtrate was concentrated under reduced pressure to give 3-(2-chloro-4-(3-hydroxyazetidin-1-yl)-3-methoxyphenyl)piperidine-2,6-dione XVIII (25.0 mg, crude) as a colorless oil and was used for the next step without further purification. MS (ESI) m/z 325.1 [M+H]⁺.

### Synthesis of Intermediate XXII

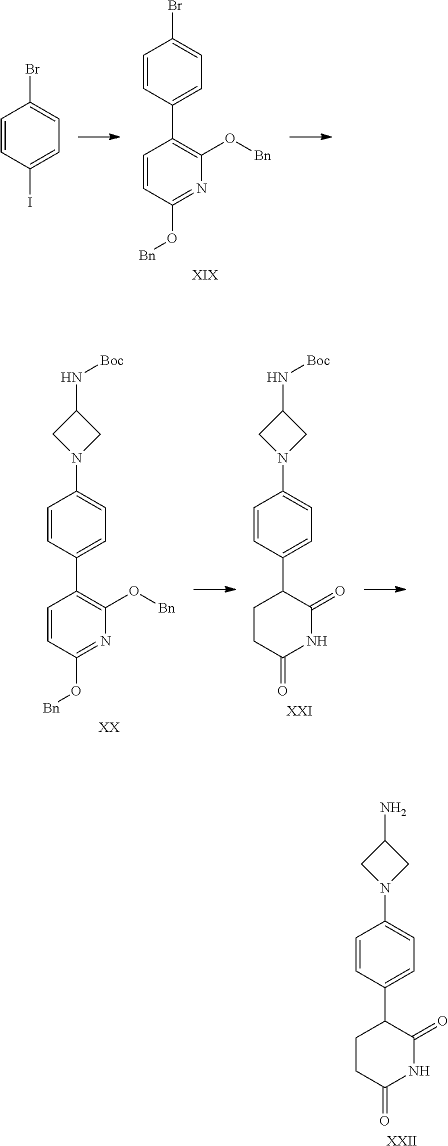

Intermediate XIX: To a solution of 1-bromo-4-iodobenzene (5.00 g, 17.7 mmol, 1.00 eq) in dioxane (50 mL) was added 2,6-bis(benzyloxy)-3-(4,4,5,5-tetramethyl-1,3,2-dioxaborolan-2-yl)pyridine (7.38 g, 17.7 mmol, 1.00 eq), [1,1′-bis(diphenylphosphino)ferrocene]dichloropalladium(II) (12.9 g, 17.7 mmol, 1.00 eq) and potassium phosphate (9.38 g, 44.2 mmol, 2.50 eq) under nitrogen atmosphere. The mixture was stirred at 80° C. for 12 h. The residue was diluted with water (400 mL) and extracted with ethyl acetate (3×80.0 mL). Then the organic phase was combined and concentrated under reduced pressure to afford a residue. The residue was purified by column chromatography (SiO₂, petroleum ether/ethyl acetate=1/0 to 10/1) to afford 2,6-bis(benzyloxy)-3-(4-bromophenyl)pyridine XIX (10.5 g, 23.3 mmol, 66% yield) as a yellow solid. ¹H NMR (400 MHz, DMSO-d₆) δ=7.75 (d, J=8.4 Hz, 1H), 7.61-7.55 (m, 2H), 7.53-7.48 (m, 2H), 7.46-7.41 (m, 2H), 7.41-7.25 (m, 8H), 6.56 (d, J=8.4 Hz, 1H), 5.41 (s, 2H), 5.37 (s, 2H). MS (ESI) m/z 446.1 [M+H]⁺.

Intermediate XX: To a solution of 2,6-bis(benzyloxy)-3-(4-bromophenyl)pyridine (2.35 g, 5.27 mmol, 1.00 eq) in dioxane (30 mL) was added tert-butyl azetidin-3-ylcarbamate (2.20 g, 10.5 mmol, 2.00 eq), cesium carbonate (5.15 g, 15.8 mmol, 3.00 eq) and methanesulfonato[9,9-dimethyl-4,5-bis(diphenylphosphino)xanthene] [2-amino-1,1-biphenyl]palladium(II) dichloromethane adduct (400 mg, 0.422 mmol, 0.0800 eq) under nitrogen atmosphere. The mixture was stirred at 110° C. for 12 h. The residue was diluted with water (200 mL) and extracted with ethyl acetate (3×40 mL). Then the organic phase was combined and concentrated under reduced pressure to afford a residue. The residue was purified by column chromatography on silica gel (petroleum ether/ethyl acetate=1/0 to 10/1) to afford tert-butyl (1-(4-(2,6-bis(benzyloxy)pyridin-3-yl) phenyl)azetidin-3-yl)carbamate XX (1.10 g, 2.05 mmol, 39% yield) as a yellow solid. ¹H NMR (400 MHz, DMSO-d₆) δ=7.65 (d, J=8.0 Hz, 1H), 7.52 (br d, J=7.6 Hz, 1H), 7.45-7.26 (m, 13H), 6.50 (d, J=8.0 Hz, 1H), 6.44 (d, J=8.4 Hz, 2H), 5.38 (s, 2H), 5.35 (s, 2H), 4.42 (qd, J=6.8, 13.2 Hz, 1H), 4.07 (t, J=7.2 Hz, 2H), 3.56 (t, J=6.8 Hz, 2H), 1.39 (s, 9H).

Intermediate XXI: To a solution of tert-butyl (1-(4-(2,6-bis(benzyloxy)pyridin-3-yl)phenyl)azetidin-3-yl)carbamate XX (1.10 g, 2.05 mmol, 1.00 eq) in dioxane (20 mL) was added palladium on carbon (0.600 g, 10% purity) and palladium hydroxide (0.600 g, 4.27 mmol, 2.09 eq). The mixture was stirred at 25° C. for 12 h under hydrogen atmosphere. The mixture was concentrated under reduced pressure to afford tert-butyl (1-(4-(2,6-dioxopiperidin-3-yl)phenyl)azetidin-3-yl)carbamate XXI (0.600 g, 1.67 mmol, 81% yield) as a green solid. ¹H NMR (400 MHz, DMSO-d₆) δ=10.76 (s, 1H), 7.51 (br d, J=7.6 Hz, 1H), 7.00 (d, J=8.4 Hz, 2H), 6.39 (d, J=8.4 Hz, 2H), 4.48-4.32 (m, 1H), 4.04 (t, J=7.2 Hz, 2H), 3.69 (dd, J=5.2, 11.0 Hz, 1H), 3.52 (t, J=6.8 Hz, 2H), 2.62 (ddd, J=5.3, 11.4, 17.2 Hz, 1H), 2.47 (br t, J=4.4 Hz, 1H), 2.16-2.05 (m, 1H), 2.04-1.94 (m, 1H), 1.39 (s, 9H).

Intermediate XXII: To a solution of tert-butyl (1-(4-(2,6-dioxopiperidin-3-yl)phenyl)azetidin-3-yl)carbamate XXI (0.300 g, 0.835 mmol, 1.00 eq) in dichloromethane (6 mL) was added trifluoroacetic acid (1.20 mL, 16.2 mmol, 19.4 eq). The mixture was stirred at 25° C. for 1 h. The reaction mixture was concentrated under reduced pressure to afford 3-(4-(3-aminoazetidin-1-yl)phenyl)piperidine-2,6-dione XXII (0.216 g, 0.833 mmol, 99.8% yield) as green oil that was used without further purification.

### Synthesis of Intermediate XXVI

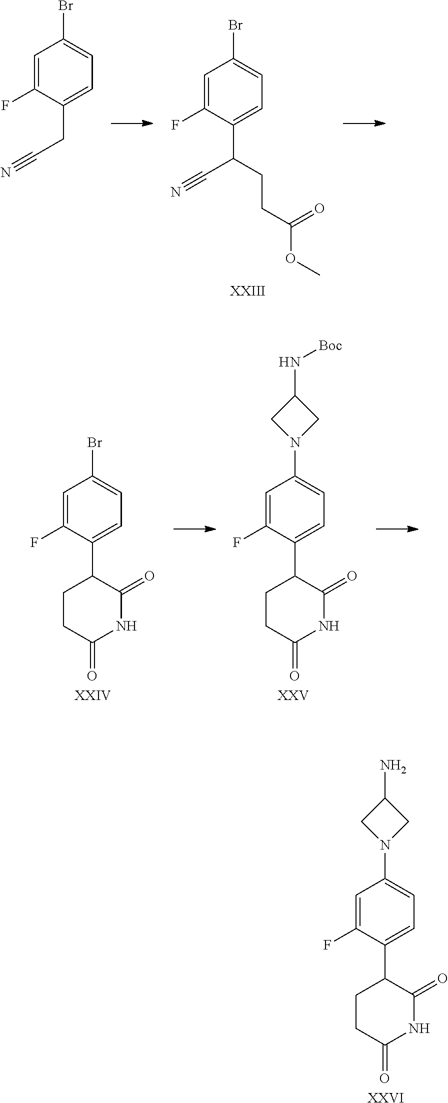

Intermediate XXIII: To a mixture of 2-(4-bromo-2-fluorophenyl)acetonitrile (25.0 g, 116 mmol, 1.00 eq) and methyl acrylate (11.5 mL, 128 mmol, 1.10 eq) in tetrahydrofuran (300 mL) was added sodium methylate (0.631 g, 11.6 mmol, 0.100 eq) at 0° C. The reaction mixture was stirred at 20° C. for 2 h. The mixture was quenched with saturated ammonium chloride solution (100 mL) and extracted with ethyl acetate (3×50 mL). The combined organic phase was washed with brine (50 mL), dried with anhydrous sodium sulfate, filtered and concentrated in vacuum to give a residue. The residue was purified by silica gel chromatography (petroleum ether/ethyl acetate=10/1 to 5/1) to give methyl 4-(4-bromo-2-fluorophenyl)-4-cyanobutanoate XXIII (22.0 g, 65.9 mmol, 56% yield, 90% purity) as a yellow solid. 1H NMR (400 MHz, DMSO-d₆) δ=7.67 (dd, J=1.6, 10.0 Hz, 1H), 7.54-7.49 (m, 1H), 7.49-7.43 (m, 1H), 4.46 (t, J=7.6 Hz, 1H), 3.58 (s, 3H), 2.48-2.39 (m, 2H), 2.25-2.16 (m, 1H), 2.15-2.07 (m, 1H).

Intermediate XXIV: A mixture of methyl 4-(4-bromo-2-fluorophenyl)-4-cyanobutanoate XXIII (22.0 g, 73.3 mmol, 1.00 eq) and sulfuric acid (20 mL) in acetic acid (200 mL) was stirred at 90° C. for 2 h. The reaction mixture was poured into ice water (1.00 L) and filtered to give 3-(4-bromo-2-fluorophenyl)piperidine-2,6-dione XXIV (18.0 g, 62.9 mmol, 85% yield) as a white solid. 1H NMR (400 MHz, DMSO-d₆) δ=10.91 (s, 1H), 7.54 (dd, J=2.0, 10.0 Hz, 1H), 7.41 (dd, J=1.6, 8.0 Hz, 1H), 7.33-7.27 (m, 1H), 4.07 (dd, J=4.8, 12.8 Hz, 1H), 2.80-2.68 (m, 1H), 2.57-2.51 (m, 1H), 2.21 (dq, J=4.0, 13.2 Hz, 1H), 2.03-1.95 (m, 1H).

Intermediate XXV: To a mixture of 3-(4-bromo-2-fluorophenyl)piperidine-2,6-dione XXIV (100 mg, 0.349 mmol, 1.00 eq), tert-butyl azetidin-3-ylcarbamate (60.2 mg, 0.349 mmol, 1.00 eq), cesium carbonate (341 mg, 1.05 mmol, 3.00 eq) in dioxane (3 mL) was added Pd-PEPPSI-IHepCl (17.0 mg, 0.0174 mmol, 0.050 eq) under nitrogen atmosphere. The reaction mixture was stirred at 100° C. for 12 h. The reaction mixture was concentrated to give a residue. The residue was purified by silica gel chromatography (petroleum ether/ethyl acetate=10/1 to 0/1) to give tert-butyl (1-(4-(2,6-dioxopiperidin-3-yl)-3-fluorophenyl)azetidin-3-yl)carbamate XXV (90.0 mg, 0.238 mmol, 68% yield) as a yellow solid. ¹H NMR (400 MHz, DMSO-d₆) δ=10.79 (s, 1H), 7.54 (br d, J=7.2 Hz, 1H), 7.05 (t, J=8.8 Hz, 1H), 6.27-6.19 (m, 2H), 4.48-4.33 (m, 1H), 4.06 (t, J=7.6 Hz, 2H), 3.87 (dd, J=5.2, 12.4 Hz, 1H), 3.57 (t, J=6.8 Hz, 2H), 2.77-2.66 (m, 1H), 2.48 (br s, 1H), 2.13 (dq, J=4.4, 12.8 Hz, 1H), 1.99-1.90 (m, 1H), 1.40 (s, 9H). MS (ESI) m/z 378.2 [M+H]⁺.

Intermediate XXVI: A mixture of tert-butyl (1-(4-(2,6-dioxopiperidin-3-yl)-3-fluorophenyl)azetidin-3-yl)carbamate (85.0 mg, 225 μmol, 1.00 eq) in dichloromethane (5 mL) was added trifluoroacetic acid (1 mL). The reaction mixture was stirred at 20° C. for 2 h. The reaction mixture was concentrated to give 3-(4-(3-aminoazetidin-1-yl)-2-fluorophenyl)piperidine-2,6-dione (60 mg, crude) as a yellow solid and was used without further purification MS (ESI) m/z 278.1 [M+H]⁺.

### Synthesis of Intermediate XXVII

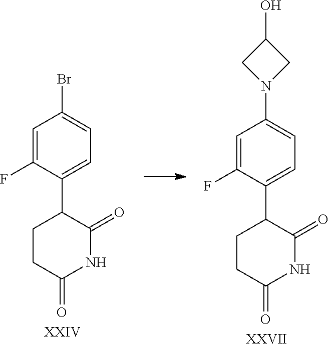

Intermediate XXVII: To a mixture of 3-(4-bromo-2-fluorophenyl)piperidine-2,6-dione XXIV (1.00 g, 3.50 mmol, 1.00 eq), azetidin-3-ol (765 mg, 6.99 mmol, 2.00 eq, hydrochloride), cesium carbonate (3.42 g, 10.4 mmol, 3.00 eq) and 2-di-tert-butylphosphino-2′,4′,6′-triisopropylbiphenyl (0.0833 mg, 0.174 mmol, 0.050 eq) in dioxane (10 mL) was added tris(dibenzylideneacetone)dipalladium(0) (0.160 g, 0.174 mmol, 0.050 eq) under nitrogen atmosphere. The reaction mixture was stirred at 100° C. for 12 h. The mixture was concentrated in vacuum to give a residue, which was purified by reversed-phase HPLC (0.1% formic acid condition) to give 3-(2-fluoro-4-(3-hydroxyazetidin-1-yl)phenyl)piperidine-2,6-dione XXVII (0.300 g, 1.08 mmol, 30% yield) as a yellow solid. MS (ESI) m/z 279.1 [M+H]⁺.

### Synthesis of Intermediate XXXII

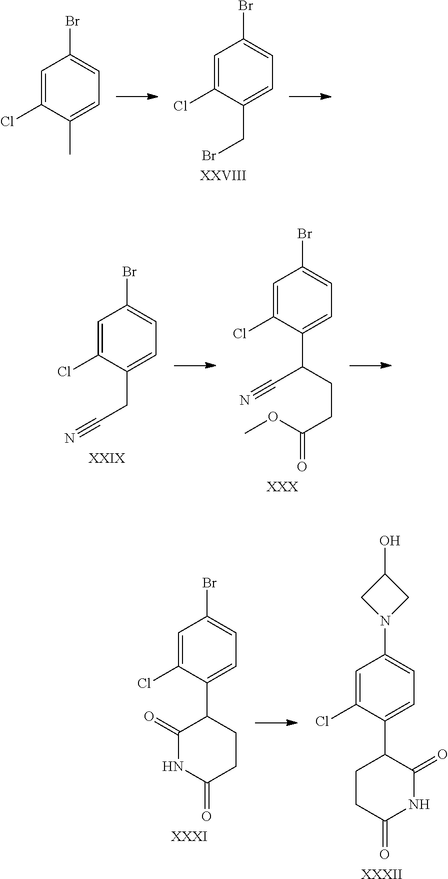

Intermediate XXVIII: To a solution of 4-bromo-2-chloro-1-methylbenzene (80.0 g, 389 mmol, 1.00 eq) in trichloromethane (600 mL) was added N-bromosuccinimide (76.4 g, 428 mmol, 1.10 eq) and benzoyl peroxide (9.44 g, 38.9 mmol, 0.100 eq). The mixture was stirred at 80° C. for 12 h under nitrogen. The mixture was extracted with water/ethyl acetate (800 mL/800 mL). The organic layer was collected and concentrated to give a residue. The residue was purified by column chromatography on silica gel (petroleum ether/ethyl acetate=1/0 to 1/1). The desired fraction was collected and concentrated to give 4-bromo-1-(bromomethyl)-2-chlorobenzene XXVIII (120 g, 359 mmol, 92% yield, 85% purity) as a yellow oil. ¹H NMR (400 MHz, DMSO-d₆) δ=7.81-7.70 (m, 1H), 7.56 (br s, 2H), 4.70 (s, 2H).

Intermediate XXIX: To a solution of 4-bromo-1-(bromomethyl)-2-chlorobenzene (10.0 g, 35.2 mmol, 1.00 eq) in acetonitrile (50 mL) was added trimethylsilyl cyanide (13.2 mL, 105 mmol, 3.00 eq) and tetrabutylammonium fluoride (1 M tetrahydrofuran) (1 M, 105 mL, 3.00 eq). The mixture was stirred at 25° C. for 1 h. The reaction mixture was quenched by addition water (100 mL), and then extracted with ethyl acetate (3×100 mL). The combined organic layers were dried over anhydrous sodium sulfate, filtered and concentrated under reduced pressure to give a residue. The residue was purified by column chromatography on silica gel (petroleum ether/ethyl acetate=1/0 to 50/1) to give 2-(4-bromo-2-chlorophenyl)acetonitrile XXIX (6.16 g, 26.7 mmol, 76% yield) as a yellow oil. ¹H NMR (400 MHz, CDCl₃) δ=7.61 (d, J=2.0 Hz, 1H), 7.49-7.45 (m, 1H), 7.42-7.38 (m, 1H), 3.80 (s, 2H).

Intermediate XXX: To a solution of 2-(4-bromo-2-chlorophenyl)acetonitrile XXIX (6.16 g, 26.7 mmol, 1.00 eq) and sodium methoxide (144 mg, 2.67 mmol, 0.100 eq) in tetrahydrofuran (50 mL) was added methyl acrylate (2.41 mL, 26.7 mmol, 1.00 eq) at 0° C. The mixture was stirred at 20° C. for 2 h. The reaction mixture was quenched by addition water (100 mL) at 0° C., and then extracted with ethyl acetate (3×100 mL). The combined organic layers were dried over anhydrous sodium sulfate, filtered and concentrated under reduced pressure to give a residue. The residue was purified by flash silica gel chromatography (ISCO®; 80 g SepaFlash® Silica Flash Column, eluent of 0-100% ethyl acetate/petroleum ether gradient at 100 mL/min). Methyl 4-(4-bromo-2-chlorophenyl)-4-cyanobutanoate XXX (6.16 g, 19.5 mmol, 73% yield) was obtained as a yellow solid. ¹H NMR (400 MHz, DMSO-d₆) δ=7.84 (d, J=2.1 Hz, 1H), 7.67 (dd, J=2.0, 8.4 Hz, 1H), 7.53 (d, J=8.4 Hz, 1H), 4.54 (dd, J=6.5, 8.4 Hz, 1H), 3.58 (s, 3H), 2.48-2.44 (m, 2H), 2.21-2.12 (m, 2H).

Intermediate XXXI: A mixture of methyl 4-(4-bromo-2-chlorophenyl)-4-cyanobutanoate XXX (6.15 g, 19.4 mmol, 1.00 eq), sulfuric acid (2.00 mL, 37.5 mmol, 1.93 eq) in acetic acid (20.0 mL, 349 mmol, 18.0 eq) was stirred at 90° C. for 2 h. The reaction mixture was quenched by addition saturated aqueous saturated sodium bicarbonate (150 mL), and extracted with ethyl acetate/dioxane (150 mL/100 mL). The combined organic layers were dried over anhydrous sodium sulfate, filtered and concentrated under reduced pressure to give 3-(4-bromo-2-chlorophenyl)piperidine-2,6-dione XXXI (4.2 g, 13.8 mmol, 71% yield) as a yellow solid. ¹H NMR (400 MHz, DMSO-d₆) δ=10.93 (s, 1H), 7.74 (d, J=2.1 Hz, 1H), 7.55 (dd, J=2.1, 8.3 Hz, 1H), 7.32 (d, J=8.4 Hz, 1H), 4.21 (dd, J=5.0, 12.5 Hz, 1H), 2.84-2.71 (m, 1H), 2.59-2.52 (m, 1H), 2.30 (dq, J=4.3, 12.9 Hz, 1H), 1.97 (dtd, J=2.8, 5.2, 13.1 Hz, 1H).

Intermediate XXXII: To a solution of 3-(4-bromo-2-chlorophenyl)piperidine-2,6-dione XXXI (1.00 g, 3.31 mmol, 1.00 eq) and 3-hydroxyazetidine hydrochloride (0.725 g, 6.62 mmol, 2.00 eq) in dioxane (20 mL) was added cesium carbonate (2.16 g, 6.62 mmol, 2.00 eq), tris(dibenzylideneacetone)dipalladium(0) (0.303 g, 0.331 mmol, 0.100 eq) and 2-dicyclohexylphosphino-2,4,6-triisopropylbiphenyl (0.316 g, 0.662 mmol, 0.200 eq) in portions under nitrogen atmosphere. The reaction mixture was stirred at 100° C. for 12 h under nitrogen atmosphere. The reaction mixture was quenched by addition of water (50 mL), and then extracted with ethyl acetate (3×50 mL). The combined organic layers were dried over anhydrous sodium sulfate, filtered and concentrated under reduced pressure to give a residue. The residue was purified by column chromatography (SiO₂, petroleum ether/ethyl acetate=1/0 to 0/1) to give 3-(2-chloro-4-(3-hydroxyazetidin-1-yl)phenyl)piperidine-2,6-dione XXXII (0.130 g, 0.428 mmol, 13% yield, 97% purity) as a yellow solid. ¹H NMR (400 MHz, DMSO-d₆) δ=10.80 (s, 1H), 7.07 (d, J=8.5 Hz, 1H), 6.45 (d, J=2.4 Hz, 1H), 6.37 (dd, J=2.4, 8.4 Hz, 1H), 5.62 (d, J=6.5 Hz, 1H), 4.59-4.50 (m, 1H), 4.08-4.04 (m, 2H), 4.04-4.02 (m, 1H), 3.50 (dd, J=5.0, 7.9 Hz, 2H), 2.77-2.68 (m, 1H), 2.47 (br s, 1H), 2.20 (dd, J=4.2, 12.7 Hz, 1H), 1.95-1.87 (m, 1H). MS (ESI) m/z 295.2 [M+H]⁺.

### Synthesis of Intermediate XL

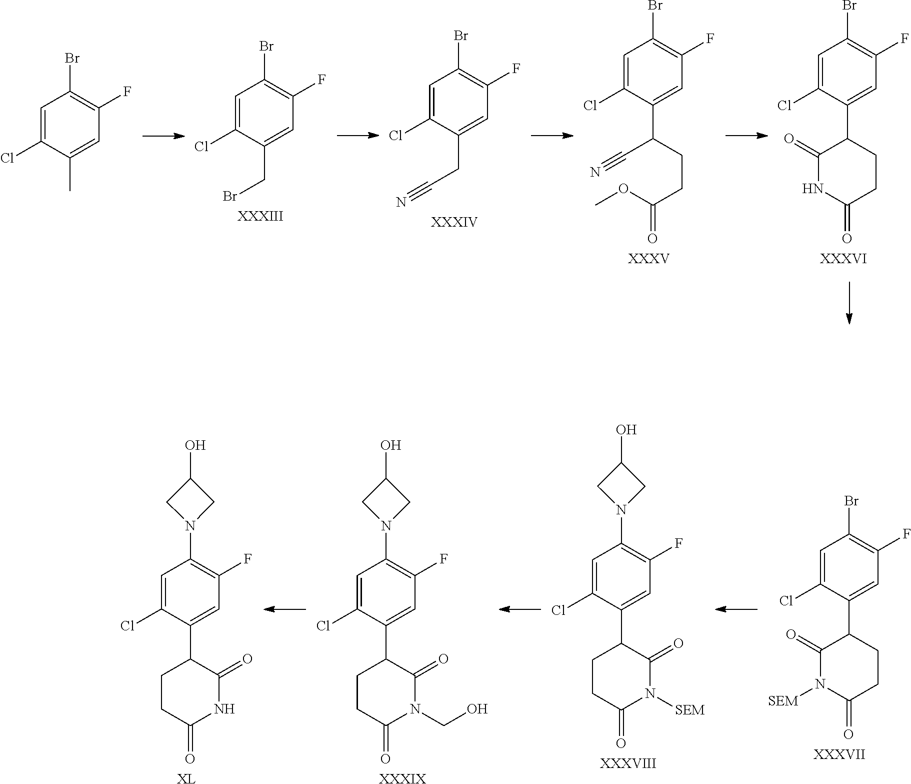

Intermediate XXXIII: To a solution of 1-bromo-5-chloro-2-fluoro-4-methylbenzene XXXII (5.00 g, 22.4 mmol, 1.00 eq) in trichloromethane (50 mL) was added N-Bromosuccinimide (3.98 g, 22.4 mmol, 1.00 eq) and benzoyl peroxide (1.08 g, 4.47 mmol, 0.200 eq). The mixture was stirred at 80° C. for 12 h. The mixture was filtered and the filtrate was concentrated under reduced pressure to give a residue. The residue was purified by column chromatography (SiO₂, petroleum ether/ethyl acetate=0/1 to 5/1) to afford 1-bromo-4-(bromomethyl)-5-chloro-2-fluorobenzene XXXIII (4.50 g, 14.9 mmol, 67% yield) as yellow oil. ¹H NMR (400 MHz, DMSO-d₆) δ=7.94 (br d, J=6.4 Hz, 1H), 7.72 (br d, J=9.2 Hz, 1H), 4.68 (s, 2H).

Intermediate XXXIV: To a solution of 1-bromo-4-(bromomethyl)-5-chloro-2-fluorobenzene XXXIII (4.00 g, 13.2 mmol, 1.00 eq) in acetonitrile (10 mL) was added trimethylsilylcyanide (4.96 mL, 39.7 mmol, 3.00 eq) and tetrabutylammonium; fluoride; trihydrate (1 M, 39.7 mL, 3.00 eq). The mixture was stirred at 25° C. for 1 h. The mixture was diluted with water (30 mL) and extracted with ethyl acetate (3×20. mL). The combined organic layers were dried over anhydrous sodium sulfate, filtered and concentrated under reduced pressure to give a residue. The residue was purified by column chromatography (SiO₂, petroleum ether/ethyl acetate=1/0 to 1/2) and concentrated under reduced pressure to afford 2-(4-bromo-2-chloro-5-fluorophenyl)acetonitrile XXXIV (2.90 g, 11.7 mmol, 88% yield) as a white solid. ¹H NMR (400 MHz, DMSO-d₆) δ=8.04-7.94 (m, 1H), 7.64-7.56 (m, 1H), 4.12-4.08 (m, 2H).

Intermediate XXXV: To a solution of 2-(4-bromo-2-chloro-5-fluorophenyl)acetonitrile XXXIV (2.90 g, 11.7 mmol, 1.00 eq) and methyl acrylate (1.16 mL, 12.8 mmol, 1.10 eq) in tetrahydrofuran (30 mL) was added sodium methoxide (63.0 mg, 1.17 mmol, 0.100 eq) at 0° C. The mixture was stirred at 20° C. for 1 h. The mixture was quenched with saturated ammonium chloride aqueous solution (50 mL) and extracted with ethyl acetate (3×50 mL). The combined organic layers were washed with brine (2×50 mL), dried over anhydrous sodium sulfate, filtered and concentrated under reduced pressure to afford methyl 4-(4-bromo-2-chloro-5-fluorophenyl)-4-cyanobutanoate XXXV (3.60 g, 10.8 mmol, 92% yield) as yellow oil. ¹H NMR (400 MHz, DMSO-d6) δ=8.02-7.98 (m, 1H), 7.68 (d, J=9.2 Hz, 1H), 4.58 (dd, J=6.8, 8.4 Hz, 1H), 3.64 (s, 3H), 2.56-2.50 (m, 2H), 2.34-2.26 (m, 1H), 2.24-2.18 (m, 1H).

Intermediate XXXVI: To a solution of methyl 4-(4-bromo-2-chloro-5-fluorophenyl)-4-cyanobutanoate XXXV (3.60 g, 10.8 mmol, 1.00 eq) in acetic acid (30 mL) was added sulfuric acid (3 mL). The mixture was stirred at 90° C. for 2 h. The mixture was poured into ice water (20 mL) and filtered. The filter cake was dried under reduced pressure to afford 3-(4-bromo-2-chloro-5-fluorophenyl)piperidine-2,6-dione XXXVI (2.90 g, 9.05 mmol, 84% yield) as a white solid. ¹H NMR (400 MHz, DMSO-d₆) δ=10.95 (s, 1H), 7.92 (td, J=1.6, 6.4 Hz, 1H), 7.50 (d, J=9.8 Hz, 1H), 4.24 (dd, J=4.8, 12.8 Hz, 1H), 2.86-2.74 (m, 1H), 2.60-2.54 (m, 1H), 2.36 (dq, J=4.2, 13.2 Hz, 1H), 1.98 (dtd, J=2.4, 5.2, 12.8 Hz, 1H).

Intermediate XXXVII: To a solution of 3-(4-bromo-2-chloro-5-fluorophenyl)piperidine-2,6-dione XXXVI (2.90 g, 9.05 mmol, 1.00 eq) in tetrahydrofuran (30 mL) was added 1,8-diazabicyclo[5.4.0]undec-7-ene (5.45 mL, 36.2 mmol, 4.00 eq) and (2-(chloromethoxy)ethyl)trimethylsilane (4.80 mL, 27.1 mmol, 3.00 eq) at 0° C. The mixture was stirred at 20° C. for 2 h. The mixture was quenched with saturated sodium bicarbonate aqueous solution (50 mL) and extracted with ethyl acetate (3×50 mL). The combined organic layers were dried over anhydrous sodium sulfate, filtered and concentrated under reduced pressure to give a residue. The residue was purified by column chromatography on silica gel (petroleum ether/ethyl acetate=1/0 to 1/1) and concentrated under reduced pressure to afford 3-(4-bromo-2-chloro-5-fluorophenyl)-1-((2-(trimethylsilyl) ethoxy)methyl)piperidine-2,6-dione XXXVII (2.90 g, 6.43 mmol, 71% yield) as a yellow oil. ¹H NMR (400 MHz, DMSO-d₆) δ=7.94 (dd, J=2.4, 6.4 Hz, 1H), 7.47 (dd, J=2.2, 9.6 Hz, 1H), 5.09 (br d, J=2.0 Hz, 2H), 4.43-4.36 (m, 1H), 3.56-3.50 (m, 2H), 3.01-2.92 (m, 1H), 2.79-2.72 (m, 1H), 2.41-2.32 (m, 1H), 2.04-1.99 (m, 1H), 0.87-0.79 (m, 2H), 0.00 (s, 9H).

Intermediate XXXVIII: To a solution of 3-(4-bromo-2-chloro-5-fluorophenyl)-1-((2-(trimethylsilyl)ethoxy)methyl)piperidine-2,6-dione XXXVII (1.40 g, 3.11 mmol, 1.00 eq), azetidin-3-ol (341 mg, 4.66 mmol, 1.50 eq, hydrochloric acid) and cesium carbonate (3.04 g, 9.32 mmol, 3.00 eq) in dioxane (20 mL) was added palladium acetate (0.0697 g, 0.311 mmol, 0.100 eq) and 4,5-bis(diphenylphosphino)-9,9-dimethylxanthene (0.180 g, 0.311 mmol, 0.100 eq). The mixture was stirred at 100° C. for 12 h under nitrogen atmosphere. The mixture was filtered. The filtrate was diluted with water (30 mL) and extracted with ethyl acetate (3×30 mL). The combined organic layers were dried over anhydrous sodium sulfate, filtered and concentrated under reduced pressure to give a residue. The residue was purified by column chromatography (SiO₂, petroleum ether/ethyl acetate=1/0 to 1/1) to afford 3-(2-chloro-5-fluoro-4-(3-hydroxyazetidin-1-yl)phenyl)-1-((2-(trimethylsilyl) ethoxy)methyl)piperidine-2,6-dione XXXVIII (300 mg, 0.677 mmol, 22% yield) as a white solid. ¹H NMR (400 MHz, DMSO-d₆) δ=7.06 (d, J=13.2 Hz, 1H), 6.62 (d, J=8.6 Hz, 1H), 5.10 (s, 2H), 4.56 (q, J=5.8 Hz, 1H), 4.20-4.18 (m, 2H), 3.68-3.64 (m, 2H), 3.58-3.54 (m, 2H), 2.98-2.88 (m, 1H), 2.76-2.68 (m, 1H), 2.34-2.24 (m, 1H), 2.00-1.92 (m, 1H), 1.20 (t, J=7.1 Hz, 2H), 0.88-0.84 (m, 2H), 0.00 (s, 9H).

Intermediate XXXIX: To a solution of 3-(2-chloro-5-fluoro-4-(3-hydroxyazetidin-1-yl)phenyl)-1-((2-(trimethylsilyl)ethoxy)methyl) piperidine-2,6-dione XXXVIII (250 mg, 564 μmol, 1.00 eq) in dichloromethane (2 mL) was added trifluoroacetic acid (0.200 mL). The mixture was stirred at 20° C. for 4 h. The mixture was concentrated under reduced pressure to afford 3-[2-Chloro-5-fluoro-4-(3-hydroxy-1-azetidinyl)phenyl]-1-(hydroxymethyl)-2,6-piperidinedione XXXIX (190 mg, 554 μmol, 98% yield) as a yellow oil. MS (ESI) m/z 343.1 [M+H]⁺.

Intermediate XL: To a solution of 3-[2-Chloro-5-fluoro-4-(3-hydroxy-1-azetidinyl)phenyl]-1-(hydroxymethyl)-2,6-piperidinedione XXXIX (190 mg, 554 μmol, 1.00 eq) in acetonitrile (5 mL) was added ammonium hydroxide (0.100 mL, 25% purity). The mixture was stirred at 20° C. for 12 h. The mixture was concentrated under reduced pressure to give a residue. The residue was purified by reverse phase column chromatography (C18, 40 g; condition: water/acetonitrile=1/0 to 0/1, 0.1% formic acid) and lyophilized to afford 3-(2-chloro-5-fluoro-4-(3-hydroxyazetidin-1-yl)phenyl)piperidine-2,6-dione XL (100 mg, 320 μmol, 58% yield) as a white solid. MS (ESI) m/z 313.0 [M+H]⁺.

### Synthesis of Intermediate XLIII

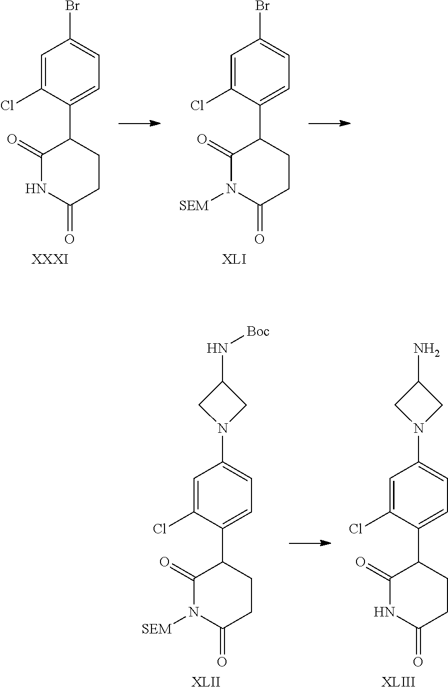

Intermediate XLI: To a solution of 3-(4-bromo-2-chlorophenyl)piperidine-2,6-dione XXXI (2.50 g, 8.26 mmol, 1.00 eq) in tetrahydrofuran (30 mL) and dimethyl formamide (3 mL) was added 1,8-diazabicyclo[5.4.0]-7-undecene (2.49 mL, 16.53 mmol, 2.00 eq) and (2-trimethylethylsilylethoxy)methyl chloride (2.63 mL, 14.9 mmol, 1.80 eq) at 0° C. The mixture was stirred at 25° C. for 1 h. The reaction mixture was quenched with water (50 mL), and then extracted with ethyl acetate (3×50 mL). The combined organic layers were washed with water (2×100 mL), dried over anhydrous sodium sulfate, filtered and concentrated under reduced pressure to give a residue. The residue was purified by column chromatography (SiO₂, petroleum ether/ethyl acetate=1/0 to 8/1) to give 3-(4-bromo-2-chlorophenyl)-1-((2-(trimethylsilyl)ethoxy) methyl)piperidine-2,6-dione XLI (5.73 g, 13.3 mmol, 80% yield) as a yellow oil. 1H NMR (400 MHz, DMSO-d₆) δ=7.76 (d, J=2.0 Hz, 1H), 7.56 (dd, J=2.1, 8.3 Hz, 1H), 7.31 (d, J=8.4 Hz, 1H), 5.08 (s, 2H), 4.36 (dd, J=4.9, 12.7 Hz, 1H), 3.56-3.50 (m, 2H), 3.00-2.87 (m, 1H), 2.78-2.69 (m, 1H), 2.38-2.27 (m, 1H), 2.04-1.94 (m, 1H), 0.85 (t, J=1.8 Hz, 1H), 0.83 (d, J=1.4 Hz, 1H), −0.03 (s, 9H).

Intermediate XLII: A mixture of 3-(4-bromo-2-chlorophenyl)-1-((2-(trimethylsilyl)ethoxy)methyl)piperidine-2,6-dione XLI (5.70 g, 13.2 mmol, 1.00 eq), tert-butyl azetidin-3-ylcarbamate (5.50 g, 26.3 mmol, 2.00 eq, hydrochloric acid), cesium carbonate (12.9 g, 39.5 mmol, 3.00 eq) in dioxane (100 mL) was added tris(dibenzylideneacetone) dipalladium(0) (603 mg, 658 μmol, 0.05 eq) and 4,5-bis(diphenylphosphino)-9,9-dimethylxanthene (1.14 g, 1.98 mmol, 0.150 eq). The mixture was stirred at 90° C. for 36 h under nitrogen atmosphere. The reaction mixture was diluted with water (100 mL) and extracted with ethyl acetate (3×100 mL). The combined organic layers were dried over anhydrous sodium sulfate, filtered and concentrated under reduced pressure to give a residue. The residue was purified by column chromatography (SiO₂, petroleum ether/ethyl acetate=0/1 to 5/1) to give tert-butyl (1-(3-chloro-4-(2,6-dioxo-1-((2-(trimethylsilyl)ethoxy)methyl) piperidin-3-yl)phenyl)azetidin-3-yl)carbamate XLII (4.80 g, 8.97 mmol, 68% yield, 98% purity) as a yellow oil. ¹H NMR (400 MHz, DMSO-d₆) δ=7.53 (br d, J=7.5 Hz, 1H), 7.06 (d, J=8.4 Hz, 1H), 6.47 (d, J=2.2 Hz, 1H), 6.37 (dd, J=2.3, 8.4 Hz, 1H), 5.08 (s, 2H), 4.45-4.35 (m, 1H), 4.16 (dd, J=5.0, 12.2 Hz, 1H), 4.10-4.05 (m, 2H), 3.60-3.50 (m, 4H), 2.89 (ddd, J=5.3, 12.6, 17.5 Hz, 1H), 2.73-2.65 (m, 1H), 2.23 (dq, J=4.2, 12.8 Hz, 1H), 1.96-1.90 (m, 1H), 1.39 (s, 9H), 0.85-0.81 (m, 2H), 0.01-−0.05 (m, 9H). MS (ESI) m/z 524.1 [M+H]⁺.

Intermediate XLIII: To a solution of tert-butyl (1-(3-chloro-4-(2,6-dioxo-1-((2-(trimethylsilyl)ethoxy)methyl)piperidin-3-yl) phenyl)azetidin-3-yl)carbamate XLII (4.80 g, 9.16 mmol, 1.00 eq) in dichloromethane (50 mL) was added trifluoroacetic acid (15.4 g, 135 mmol, 10 mL, 14.7 eq). The mixture was stirred at 25° C. for 1 h. The reaction mixture was concentrated under reduced pressure to give 3-(4-(3-aminoazetidin-1-yl)-2-chlorophenyl)-1-(hydroxymethyl)piperidine-2,6-dione (3.80 g, crude) as yellow oil. The crude product was dissolved in acetonitrile (50 mL)/ammonium hydroxide (5.24 mL). The mixture was stirred at 25° C. for 10 min. The reaction mixture was concentrated under reduced pressure to give a residue. The residue was purified by reverse phase HPLC (column: spherical C18, 20-45 μm, 100 Å, SW 120, mobile phase: [water (0.1% formic acid)-acetonitrile) to give 3-(4-(3-aminoazetidin-1-yl)-2-chlorophenyl)piperidine-2,6-dione XLIII (1.02 g, 3.16 mmol, 27% yield, 91% purity) as an off-white solid. MS (ESI) m/z 294.0 [M+H]⁺.

### Synthesis of Intermediate XLVII

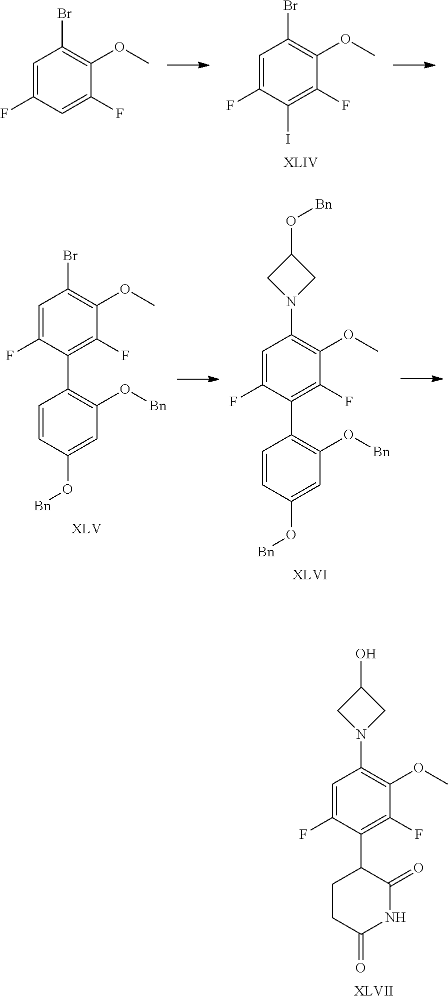 [SMILES: COc1c(F)cc(F)cc1Br ; COc1c(Br)cc(F)c(I)c1F ; COc1c(Br)cc(F)c(-c2ccc(OCc3ccccc3)cc2OCc2ccccc2)c1F ; COc1c(N2CC(OCc3ccccc3)C2)cc(F)c(-c2ccc(OCc3ccccc3)cc2OCc2ccccc2)c1F ; COc1c(N2CC(O)C2)cc(F)c(C2CCC(=O)NC2=O)c1F]

Intermediate XLIV: To a solution of 1-bromo-3,5-difluoro-2-methoxybenzene XLIII (1.00 g, 4.48 mmol, 1.00 eq) in tetrahydrofuran (2 mL) was added lithium diisopropylamide (2 M, 2.35 mL, 4.70 mmol, 1.05 eq) in tetrahydrofuran (10 mL) at −78° C. The mixture was stirred at −78° C. for 1 h. Then iodine (0.994 mL, 4.93 mmol, 1.10 eq) was added into the solution and the mixture was stirred at 20° C. for 2 h. The mixture was quenched by ice water (60 mL), extracted with ethyl acetate (3×100 mL). The organic layers were washed with brine (80 mL), dried over anhydrous sodium sulfate, filtered and concentrated to afford a crude product. The crude product was purified by silica gel chromatography (petroleum ether/ethyl acetate=1/0 to 100/1) and concentrated to afford 1-bromo-3,5-difluoro-4-iodo-2-methoxybenzene XLIV (1.10 g, 3.15 mmol, 70% yield) as a white solid ¹H NMR (400 MHz, DMSO-d₆) δ=7.60-7.55 (m, 1H), 3.83 (s, 3H). Intermediate XLV: To a solution of 1-bromo-3,5-difluoro-4-iodo-2-methoxybenzene XLIV (0.900 g, 2.58 mmol, 1.00 eq), 2,6-bis(benzyloxy)-3-(4,4,5,5-tetramethyl-1,3,2-dioxaborolan-2-yl)pyridine (1.08 g, 2.58 mmol, 1.00 eq) and potassium phosphate (1.10 g, 5.16 mmol, 2.00 eq) in dioxane (10 mL) was added [1,1′-bis(diphenylphosphino)ferrocene]dichloropalladium(II) (0.189 g, 0.258 mmol, 0.100 eq). The mixture was stirred at 80° C. for 12 h. The mixture was filtered and concentrated under reduced pressure to give a residue. The residue was purified by silica gel chromatography (petroleum ether/ethyl acetate=1/0 to 50/1) to afford 2,6-bis(benzyloxy)-3-(4-bromo-2,6-difluoro-3-methoxyphenyl)pyridine XLV (0.800 g, 1.56 mmol, 61% yield) as a white solid. ¹H NMR (400 MHz, DMSO-d₆) δ=7.73 (d, J=8.1 Hz, 1H), 7.59 (dd, J=2.1, 8.8 Hz, 1H), 7.46-7.37 (m, 5H), 7.36-7.32 (m, 5H), 6.59 (d, J=8.1 Hz, 1H), 5.39-5.36 (m, 4H), 3.81 (s, 3H).

Intermediate XLVI: To a solution of 2,6-bis(benzyloxy)-3-(4-bromo-2,6-difluoro-3-methoxyphenyl)pyridine XLV (400 mg, 0.781 mmol, 1.00 eq), 3-(benzyloxy)azetidine (312 mg, 1.56 mmol, 2.00 eq, hydride acid), cesium carbonate (763 mg, 2.34 mmol, 3.00 eq) and tris(dibenzylideneacetone)dipalladium(0) (35.8 mg, 0.039 mmol, 0.0500 eq) in dioxane (5 mL) was added 4,5-bis(diphenylphosphino)-9,9-dimethylxanthene (0.0226 mg, 0.039 mmol, 0.0500 eq). The mixture was stirred at 110° C. for 12 h. The mixture was filtered and concentrated to give a residue. The residue was added water (80 mL), extracted with ethyl acetate (3×80 mL). The organic layers were washed with brine (60 mL), dried over anhydrous sodium sulfate, filtered and concentrated to afford a crude product. The crude product was purified by silica gel chromatography (petroleum ether/ethyl acetate=100/1 to 10/1) to afford 2,6-bis(benzyloxy)-3-(4-(3-(benzyloxy)azetidin-1-yl)-2,6-difluoro-3-methoxyphenyl)pyridine XLVI (310 mg, 0.521 mmol, 67% yield) as a yellow solid. ¹H NMR (400 MHz, DMSO-d₆) δ=7.59 (d, J=8.0 Hz, 1H), 7.29 (br d, J=3.0 Hz, 15H), 6.53 (d, J=8.0 Hz, 1H), 6.21 (dd, J=1.6, 11.4 Hz, 1H), 5.39-5.30 (m, 4H), 4.49 (s, 2H), 4.48-4.44 (m, 1H), 4.24-4.14 (m, 2H), 3.78 (dd, J=4.4, 8.6 Hz, 2H), 3.65 (s, 3H).

Intermediate XLVII: To a solution of 2,6-bis(benzyloxy)-3-(4-(3-(benzyloxy)azetidin-1-yl)-2,6-difluoro-3-methoxyphenyl) pyridine XLVI (100 mg, 168 μmol, 1.00 eq) in dioxane (2 mL) was added palladium on carbon (107 mg, 101 μmol, 10% purity, 0.600 eq) under nitrogen. The suspension was degassed under vacuum and purged several times. The mixture was stirred under hydrogen (15 psi) at 35° C. for 12 h. The mixture was filtered and concentrated to afford 3-(2,6-difluoro-4-(3-hydroxyazetidin-1-yl)-3-methoxyphenyl) piperidine-2,6-dione XLVII (50.0 mg, 153 μmol, 91% yield) as a white solid. ¹H NMR (400 MHz, DMSO-d₆) δ=10.88 (s, 1H), 6.16 (br d, J=12.1 Hz, 1H), 5.60 (d, J=6.2 Hz, 1H), 4.53 (qd, J=5.6, 11.2 Hz, 1H), 4.16 (t, J=7.3 Hz, 2H), 4.05 (br dd, J=5.4, 12.3 Hz, 1H), 3.66 (s, 3H), 3.63-3.60 (m, 2H), 2.85-2.74 (m, 1H), 2.53-2.53 (m, 1H), 2.15-2.03 (m, 1H), 2.02-1.93 (m, 1H).

### Synthesis of Intermediate LII

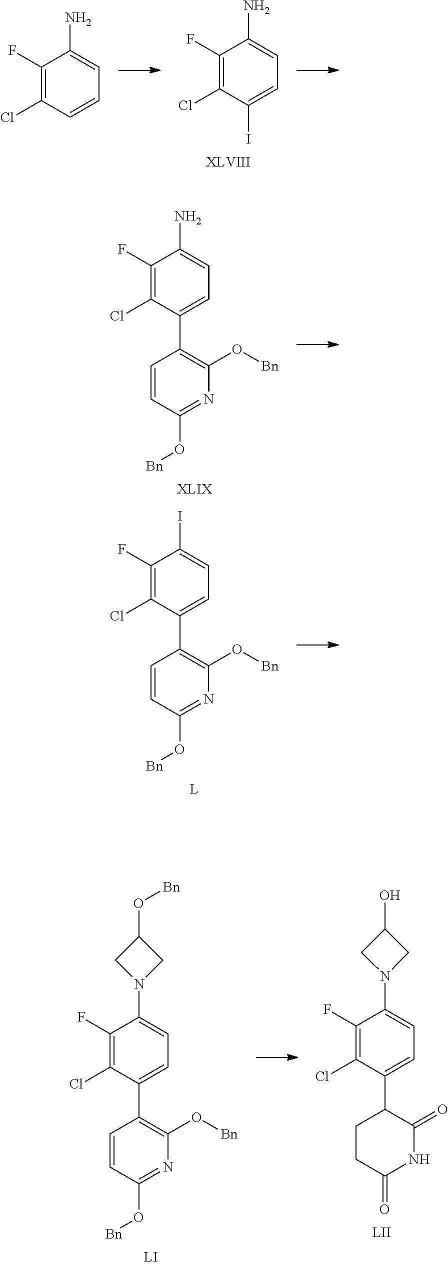 [SMILES: Nc1cccc(Cl)c1F ; Nc1ccc(I)c(Cl)c1F ; Nc1ccc(-c2ccc(OCc3ccccc3)nc2OCc2ccccc2)c(Cl)c1F ; Fc1c(I)ccc(-c2ccc(OCc3ccccc3)nc2OCc2ccccc2)c1Cl ; Fc1c(N2CC(OCc3ccccc3)C2)ccc(-c2ccc(OCc3ccccc3)nc2OCc2ccccc2)c1Cl ; O=C1CCC(c2ccc(N3CC(O)C3)c(F)c2Cl)C(=O)N1]

Intermediate XLVIII: To a solution of 3-chloro-2-fluoro-aniline (5.00 g, 34.3 mmol, 1.00 eq) in dimethyl formamide (40 mL) was added a solution of 1-bromopyrrolidine-2,5-dione (6.11 g, 34.3 mmol, 1.00 eq) in dimethyl formamide (10 mL) dropwise at 15° C. The mixture was stirred at 15° C. for 2 h. The reaction mixture was quenched with water (50 mL) and diluted with ethyl acetate (50 mL). The layers were separated and the aqueous phase was extracted with ethyl acetate (50 mL). The combined organic layers were washed with water (80 mL) and brine (80 mL), dried over sodium sulfate, filtered and concentrated under reduced pressure to give 4-bromo-3-chloro-2-fluoro-aniline XLVIII (7.50 g, 32.7 mmol, 95% yield) as a brown solid. ¹H NMR (400 MHz, CHCl₃) δ=7.18 (dd, J=2.0, 8.8 Hz, 1H), 6.59 (t, J=8.8 Hz, 1H), 3.84 (br s, 2H).

Intermediate XLIX: To a solution of 4-bromo-3-chloro-2-fluoro-aniline XLVIII (0.968 g, 4.31 mmol, 1.20 eq) and 2,6-dibenzyloxy-3-(4,4,5,5-tetramethyl-1,3,2-dioxaborolan-2-yl)pyridine (1.50 g, 3.59 mmol, 1.00 eq) in dioxane (20 mL) was added potassium phosphate (1.53 g, 7.19 mmol, 2.00 eq) and [1,1-bis(diphenylphosphino)ferrocene]dichloropalladium(II) (0.300 g, 0.367 mmol, 0.10 eq) under nitrogen atmosphere. The mixture was stirred at 90° C. for 12 h. After being cooled to room temperature, the mixture was filtered through a pad of Celite. The filtrate was concentrated under reduced pressure to give a residue. The residue was purified by flash silica gel chromatography (ISCO®; 20 g SepaFlash® Silica Flash Column, eluent of 0-36% ethyl acetate/petroleum ether gradient at 40 mL/min) to give 4-(2,6-bis(benzyloxy)pyridin-3-yl)-3-chloro-2-fluoroaniline XLIX (0.650 g, 1.46 mmol, 41% yield) as a brown oil. ¹H NMR (400 MHz, CHCl₃) δ=7.44 (d, J=8.0 Hz, 3H), 7.42-7.30 (m, 7H), 7.30-7.27 (m, 1H), 6.92 (dd, J=1.6, 8.3 Hz, 1H), 6.79-6.72 (m, 1H), 6.45 (d, J=8.0 Hz, 1H), 5.37 (d, J=12.3 Hz, 4H). MS (ESI) m/z 435.2 [M+H]⁺.

Intermediate L: To a solution of 4-(2,6-bis(benzyloxy)pyridin-3-yl)-3-chloro-2-fluoroaniline XLIX (650 mg, 1.49 mmol, 1.00 eq) in hydrochloric acid (10 mL, 6 M in water) and acetonitrile (5 mL) was added a solution of sodium nitrite (130 mg, 1.88 mmol, 1.26 eq) in water (2 mL) dropwise at 0° C. The he mixture was stirred at 0° C. for 30 min and a solution of potassium iodide (750 mg, 4.52 mmol, 3.02 eq) in water (2 mL) was added. The ice bath was removed and the mixture was stirred at 20° C. for 1.5 h. The reaction mixture was quenched with water (20 mL) and diluted with ethyl acetate (30 mL). The layers were separated and the aqueous phase was extracted with ethyl acetate (30 mL). The combined organic layers were washed with brine (50 mL), dried over sodium sulfate, filtered and concentrated under reduced pressure to give a residue. The residue was purified by flash silica gel chromatography (ISCO®; 12 g SepaFlash® Silica Flash Column, eluent of 0-12% ethyl acetate/petroleum ether gradient at 20 mL/min) to give 6-bis(benzyloxy)-3-(2-chloro-3-fluoro-4-iodophenyl)pyridine L (400 mg, 0.732 mmol, 49% yield) as brown oil. MS (ESI) m/z 546.0 [M+H]⁺.

Intermediate LI: To a solution of 3-benzyloxyazetidine hydrochloride L (320 mg, 1.60 mmol, 2.00 eq) and 6-bis(benzyloxy)-3-(2-chloro-3-fluoro-4-iodophenyl)pyridine (0.60 g, 0.802 mmol, 73% purity, 1.00 eq) in dioxane (15 mL) were added cesium carbonate (780 mg, 2.39 mmol, 2.98 eq) and [4,5-Bis(diphenylphosphino)-9,9-dimethylxanthene][2′-amino-2-biphenylyl][(methylsulfonyl)oxy]palladium(II) (80.0 mg, 0.0844 mmol, 0.100 eq) under nitrogen atmosphere. The mixture was stirred at 90° C. for 12 h. After being cooled to room temperature, the mixture was filtered through a pad of Celite. The filtrate was concentrated under reduced pressure to give a residue. The residue was purified by flash silica gel chromatography ((ISCO®; 12 g SepaFlash® Silica Flash Column, eluent of 0-12% ethyl acetate/petroleum ether gradient at 30 mL/min) to give 2,6-dibenzyloxy-3-[4-(3-benzyloxyazetidin-1-yl)-2-chloro-3-fluoro-phenyl]pyridine LI (360 mg, 0.613 mmol, 76% yield) as a brown oil. ¹H NMR (400 MHz, DMSO-d₆) δ=7.45-7.41 (m, 2H), 7.41-7.24 (m, 14H), 6.99 (dd, J=1.2, 8.4 Hz, 1H), 6.59-6.48 (m, 2H), 5.34 (s, 4H), 4.49 (s, 3H), 4.22-4.14 (m, 2H), 3.80 (dd, J=4.8, 7.6 Hz, 2H), 3.32 (s, 3H). MS (ESI) m/z 581.1 [M+H]⁺.

Intermediate LII: To a solution of 2,6-dibenzyloxy-3-[4-(3-benzyloxyazetidin-1-yl)-2-chloro-3-fluoro-phenyl]pyridine (100 mg, 172 μmol, 1.00 eq) and lithium chloride (25.0 mg, 589 μmol, 3.40 eq) in dioxane (2 mL) was added palladium on active carbon (100 mg, 10% purity) under nitrogen atmosphere. Then the mixture was stirred at 35° C. for 22 h under hydrogen (15 psi). After being cooled to room temperature, the mixture was filtered through a pad of Celite. The filtrate was concentrated under reduced pressure to give a residue. The residue was purified by prep-TLC (SiO₂, petroleum ether/ethyl acetate=1/2) to give 3-(2-chloro-3-fluoro-4-(3-hydroxyazetidin-1-yl)phenyl)piperidine-2,6-dione LII (30.0 mg, 70.0 μmol, 40% yield, 73% purity) as a white solid. MS (ESI) m/z 313.1/315.0 [M+H]⁺.

### Synthesis of Intermediate LVI

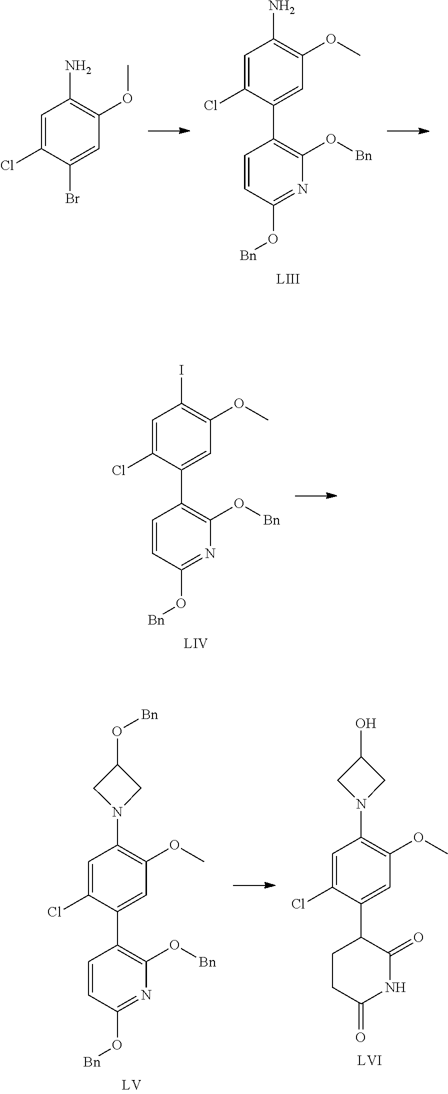 [SMILES: COc1cc(Br)c(Cl)cc1N ; COc1cc(-c2ccc(OCc3ccccc3)nc2OCc2ccccc2)c(Cl)cc1N ; COc1cc(-c2ccc(OCc3ccccc3)nc2OCc2ccccc2)c(Cl)cc1I ; COc1cc(-c2ccc(OCc3ccccc3)nc2OCc2ccccc2)c(Cl)cc1N1CC(OCc2ccccc2)C1 ; COc1cc(C2CCC(=O)NC2=O)c(Cl)cc1N1CC(O)C1]

Intermediate LIII: To a solution of 4-bromo-5-chloro-2-methoxyaniline (1.50 g, 6.34 mmol, 1.00 eq) and 2,6-bis(benzyloxy)-3-(4,4,5,5-tetramethyl-1,3,2-dioxaborolan-2-yl)pyridine (2.91 g, 6.98 mmol, 1.10 eq) in dioxane (20 mL) was added potassium phosphate (2.69 g, 12.7 mmol, 2.00 eq) and [1,1′-bis(diphenylphosphino)ferrocene]dichloropalladium(II) (0.464 mg, 0.634 mmol, 0.100 eq) under nitrogen atmosphere. The mixture was stirred at 80° C. for 12 h. The mixture was filtered, and the filtrate was concentrated under reduced pressure to afford a residue. The residue was purified by column chromatography on silica gel (petroleum ether/ethyl acetate=1/0 to =8/1) to afford 4-(2,6-bis(benzyloxy)pyridin-3-yl)-5-chloro-2-methoxyaniline LIII (2.00 g, 4.39 mmol, 69% yield) as a white solid. MS (ESI) m/z 447.1 [M+H]⁺.

Intermediate LIV: To a solution of 4-(2,6-bis(benzyloxy)pyridin-3-yl)-5-chloro-2-methoxyaniline LIII (2.00 g, 4.48 mmol, 1.00 eq) and p-toluenesulfonic acid (2.31 g, 13.4 mmol, 3.00 eq) in acetonitrile (25 mL) were added the mixture of sodium nitrite (0.618 g, 8.95 mmol, 2.00 eq) and potassium iodide (3.71 g, 22.4 mmol, 5.00 eq) in water (8 mL) at 0° C. The mixture was stirred at 20° C. for 2 h. The mixture was diluted with water (400 mL) and extracted with ethyl acetate (3×100 mL). The combined organic layers were washed with brine (50 mL) and dried over anhydrous sodium sulfate, filtered and concentrated to give crude product. The crude product was purified by silica gel chromatography (petroleum ether/ethyl acetate=1/0 to 4/1) to afford 2,6-bis(benzyloxy)-3-(2-chloro-4-iodo-5-methoxyphenyl)pyridine LIV (1.40 g, 2.43 mmol, 54% yield) as a white solid. ¹H NMR (400 MHz, DMSO-d₆) δ=7.87 (s, 1H), 7.62 (d, J=8.4 Hz, 1H), 7.49-7.42 (m, 2H), 7.42-7.25 (m, 8H), 6.96 (s, 1H), 6.56 (d, J=8.0 Hz, 1H), 5.37 (d, J=12.8 Hz, 4H), 3.78 (s, 3H). MS (ESI) m/z 557.9 [M+H]⁺.

Intermediate LV: To a mixture of 2,6-bis(benzyloxy)-3-(2-chloro-4-iodo-5-methoxyphenyl)pyridine LIV (1.00 g, 1.79 mmol, 1.00 eq), 3-(benzyloxy)azetidine (0.716 g, 3.59 mmol, 2.00 eq, hydrochloric acid), cesium carbonate (1.75 g, 5.38 mmol, 3.00 eq) in dioxane (20 mL) was added methanesulfonato[9,9-dimethyl-4,5-bis(diphenylphosphino) xanthene] [2-amino-1,1-biphenyl]palladium(II)dichloromethane adduct (170 mg, 0.179 mmol, 0.100 eq). The mixture was diluted with water (200 mL) and extracted by ethyl acetate (3×30 mL). Then the combined organic phases were dried over anhydrous sodium sulfate, filtered and concentrated under reduced pressure to give a residue. The residue was purified by column chromatography on silica gel (petroleum ether/ethyl acetate=5/1 to 0/1) to give a mixture and then the mixture was purified by reverse-phase HPLC (column: spherical C18, 20-45 μm, 100 Å, SW 120, mobile phase: [water (0.1% ammonium hydroxide)-acetonitrile]) and the desired eluent was lyophilized to afford 2,6-bis(benzyloxy)-3-(4-(3-(benzyloxy)azetidin-1-yl)-2-chloro-5-methoxyphenyl)pyridine LV (1.00 g, 1.67 mmol, 93% yield) as a yellow solid. MS (ESI) m/z 593.4 [M+H]⁺.

Intermediate LVI: To a solution of 2,6-bis(benzyloxy)-3-(4-(3-(benzyloxy)azetidin-1-yl)-2-chloro-5-methoxyphenyl)pyridine LV (200 mg, 337 μmol, 1.00 eq) in dioxane (20 mL) was added palladium on carbon (100 mg, 10% purity, 1.00 eq) and lithium chloride (42.9 mg, 1.01 mmol, 20.7 μL, 3.00 eq) under hydrogen atmosphere. The mixture was stirred at 25° C. for 12 h. The residue was diluted with water (300 mL) and extracted with ethyl acetate (3×40 mL). Then the combined organic layers were dried over anhydrous sodium sulfate, filtered and concentrated under reduced pressure to give a residue. The residue was purified by column chromatography on silica gel (petroleum ether/ethyl acetate=1/0 to 1/3) to afford 3-(2-chloro-4-(3-hydroxyazetidin-1-yl)-5-methoxyphenyl)piperidine-2,6-dione LVI (70.0 mg, 216 μmol, 32% yield) as a white solid. ¹H NMR (400 MHz, DMSO-d₆) δ=10.76 (s, 1H), 6.67 (d, J=1.6 Hz, 1H), 6.59 (dd, J=1.5, 8.0 Hz, 1H), 5.52 (d, J=6.4 Hz, 1H), 4.50-4.40 (m, 1H), 4.09 (dd, J=6.6, 8.0 Hz, 2H), 4.04-3.98 (m, 1H), 3.76 (s, 3H), 3.53-3.45 (m, 2H), 3.09-3.01 (m, 1H), 2.46-2.40 (m, 1H), 2.22-2.09 (m, 1H), 1.96-1.85 (m, 1H).

### Synthesis of Intermediate LIX

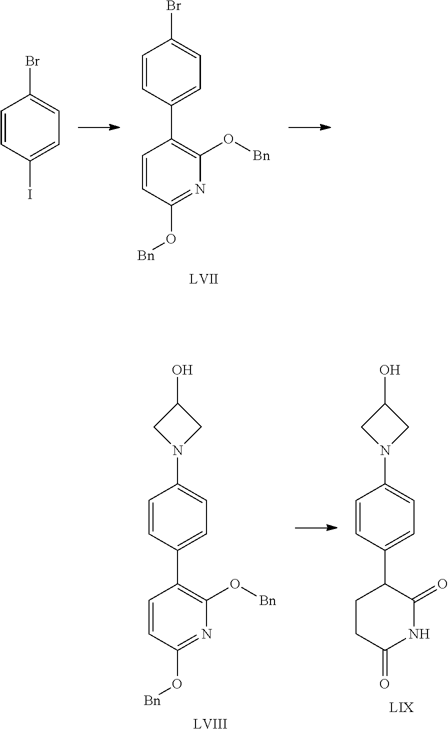 [SMILES: Brc1ccc(I)cc1 ; Brc1ccc(-c2ccc(OCc3ccccc3)nc2OCc2ccccc2)cc1 ; OC1CN(c2ccc(-c3ccc(OCc4ccccc4)nc3OCc3ccccc3)cc2)C1 ; O=C1CCC(c2ccc(N3CC(O)C3)cc2)C(=O)N1]

Intermediate LVII: To a solution of 1-bromo-4-iodobenzene (1.00 g, 3.53 mmol, 1.00 eq) and 2,6-bis(benzyloxy)-3-(4,4,5,5-tetramethyl-1,3,2-dioxaborolan-2-yl)pyridine (1.48 g, 3.53 mmol, 1.00 eq) in dioxane (15 mL) was added potassium phosphate (1.50 g, 7.07 mmol, 2.00 eq) and [1,1′-bis(diphenylphosphino)ferrocene] dichloropalladium(II) (0.258 g, 0.353 mmol, 0.100 eq) under nitrogen atmosphere. The mixture was stirred at 80° C. for 12 h. The mixture was diluted with water (300 mL) and extracted with ethyl acetate (3×80 mL). The combined organic layer was washed with brine (50 mL), dried over anhydrous sodium sulfate, filtered and concentrated to give a crude product. The crude product was purified by column chromatography on silica gel (petroleum ether/ethyl acetate=1/0 to 10/1) to afford 2,6-bis(benzyloxy)-3-(4-bromophenyl)pyridine LVII (0.700 g, 1.43 mmol, 40% yield, 91% purity) as a yellow solid. 1H NMR (400 MHz, DMSO-d₆) δ=7.75 (d, J=8.2 Hz, 1H), 7.60-7.55 (m, 2H), 7.54-7.48 (m, 2H), 7.46-7.28 (m, 10H), 6.56 (d, J=8.2 Hz, 1H), 5.41 (s, 2H), 5.38 (s, 2H). MS (ESI) m/z 447.8 [M+Br+H]⁺.

Intermediate LVIII: To a mixture of 2,6-bis(benzyloxy)-3-(4-bromophenyl)pyridine LVII (1.00 g, 2.24 mmol, 1.00 eq), azetidin-3-ol (0.540 g, 4.93 mmol, 2.20 eq, HCl), cesium carbonate (2.41 g, 7.40 mmol, 3.30 eq) in dioxane (15 mL) was added 4,5-bis(diphenylphosphino)-9,9-dimethylxanthene (0.214 mg, 0.370 mmol, 0.165 eq) and tris(dibenzylideneacetone)dipalladium(0) (0.113 g, 0.123 mmol, 0.0550 eq) under nitrogen atmosphere, and then the mixture was stirred at 110° C. for 12 h under nitrogen atmosphere. The residue was diluted with water (200 mL) and extracted with ethyl acetate (3×40 mL). Then the combined organic phase was concentrated under reduced pressure to afford a residue. The residue was purified by column chromatography on silica gel (petroleum ether/ethyl acetate=5/1 to 3/1) to afford 1-(4-(2,6-bis(benzyloxy)pyridin-3-yl)phenyl)azetidin-3-ol LVIII (0.300 mg, 0.643 mmol, 29% yield, 94% purity) as a yellow solid. MS (ESI) m/z 439.2 [M+H]⁺.

Intermediate LIX: To a solution of 1-(4-(2,6-bis(benzyloxy)pyridin-3-yl)phenyl)azetidin-3-ol (270 mg, 0.616 mmol, 1.00 eq) in dioxane (5 mL) was added palladium on carbon (150 mg, 10% purity) and palladium hydroxide (150 mg, 1.07 mmol, 1.73 eq). The mixture was stirred at 25° C. for 12 h under hydrogen atmosphere. The mixture was filtered and the filtrate was concentrated under reduced pressure to afford 3-(4-(3-hydroxyazetidin-1-yl) phenyl)piperidine-2,6-dione LIX (220 mg, crude) as a white solid. 1H NMR (400 MHz, DMSO-d₆) δ=10.75 (s, 1H), 6.99 (d, J=8.4 Hz, 2H), 6.39 (d, J=8.4 Hz, 2H), 5.57 (d, J=6.4 Hz, 1H), 4.60-4.49 (m, 1H), 4.04 (t, J=7.2 Hz, 2H), 3.69 (dd, J=5.0, 11.2 Hz, 1H), 3.45 (dd, J=5.2, 7.8 Hz, 2H), 2.69-2.57 (m, 1H), 2.47 (br t, J=4.8 Hz, 1H), 2.16-2.04 (m, 1H), 2.03-1.95 (m, 1H).

### Synthesis of Intermediate LXII

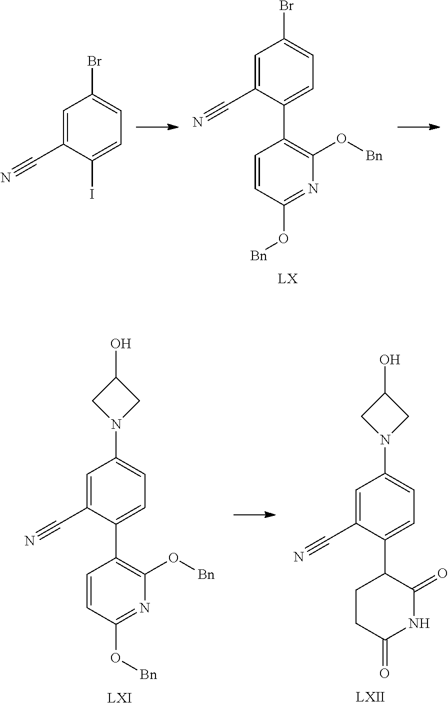 [SMILES: N#Cc1cc(Br)ccc1I ; N#Cc1cc(Br)ccc1-c1ccc(OCc2ccccc2)nc1OCc1ccccc1 ; N#Cc1cc(N2CC(O)C2)ccc1-c1ccc(OCc2ccccc2)nc1OCc1ccccc1 ; N#Cc1cc(N2CC(O)C2)ccc1C1CCC(=O)NC1=O]

Intermediate LX: To a solution of 5-bromo-2-iodobenzonitrile (1.33 g, 4.31 mmol, 1.20 eq), 2,6-bis(benzyloxy)-3-(4,4,5,5-tetramethyl-1,3,2-dioxaborolan-2-yl)pyridine (1.50 g, 3.59 mmol, 1.00 eq) and potassium carbonate (1.99 g, 14.4 mmol, 4.00 eq) in dimethylformamide (20 mL) was added tetrakis[triphenylphosphine] palladium(0) (249 mg, 0.216 nmol, 0.0600 eq). The mixture was stirred at 100° C. for 12 h under nitrogen atmosphere. The reaction mixture was diluted with water (20 mL) and extracted with ethyl acetate (3×20 mL). The combined organic layers were dried over anhydrous sodium sulfate, filtered and concentrated under reduced pressure to give a residue. The residue was purified by column chromatography (SiO₂, petroleum ether/ethyl acetate=1/0 to 20/1) to afford 2-(2,6-bis(benzyloxy)pyridin-3-yl)-5-bromobenzonitrile LX (1.30 g, 2.76 mmol, 77% yield) as a yellow solid. ¹H NMR (400 MHz, DMSO-d6) δ=8.20-8.18 (m, 1H), 7.96-7.92 (m, 1H), 7.76 (dd, J=1.2, 8.1 Hz, 1H), 7.52-7.48 (m, 3H), 7.42-7.32 (m, 7H), 6.64 (dd, J=1.0, 8.1 Hz, 1H), 5.42 (s, 4H).

Intermediate LXI: To a solution of 2-(2,6-bis(benzyloxy)pyridin-3-yl)-5-bromobenzonitrile LX (1.20 g, 2.55 mmol, 1.00 eq), azetidin-3-ol (0.558 g, 5.09 mmol, 2.00 eq, HCl) and cesium carbonate (2.49 g, 7.64 mmol, 3.00 eq) in dioxane (10 mL) was added tris(dibenzylideneacetone)dipalladium(0) (0.117 mg, 0.127 mmol, 0.0500 eq) and 4,5-bis(diphenylphosphino)-9,9-dimethylxanthene (0.221 g, 0.382 mmol, 0.150 eq). The mixture was stirred at 110° C. for 12 h under nitrogen atmosphere. The reaction mixture was diluted with water (20 mL) and exacted with ethyl acetate (3×20 mL). The organic phase was separated, washed with brine (2×10 mL), dried over sodium sulfate, filtered and concentrated under reduced pressure to give a residue. The residue was purified by silica gel chromatography (column height: 250 mm, diameter: 100 mm, 100-200 mesh silica gel, petroleum ether/ethyl acetate=0/1 to 1/1) to afford 2-(2,6-bis(benzyloxy)pyridin-3-yl)-5-(3-hydroxyazetidin-1-yl)benzonitrile LXI (0.480 g, 1.09 mmol, 49% yield) as a yellow solid. ¹H NMR (400 MHz, DMSO-d₆) δ=7.64 (d, J=8.1 Hz, 1H), 7.62 (s, 1H), 7.46-7.26 (m, 10H), 6.86 (d, J=2.6 Hz, 1H), 6.74 (dd, J=2.5, 8.5 Hz, 1H), 6.56 (d, J=8.1 Hz, 1H), 5.66 (d, J=6.6 Hz, 1H), 5.38 (d, J=4.4 Hz, 4H), 4.64-4.54 (m, 1H), 4.14 (t, J=7.3 Hz, 2H), 3.58 (dd, J=4.8, 8.2 Hz, 2H).

Intermediate LXII: To a mixture of 2-(2,6-bis(benzyloxy)pyridin-3-yl)-5-(3-hydroxyazetidin-1-yl)benzonitrile (400 mg, 863 μmol, 1.00 eq) in dioxane (20 mL) was added palladium hydroxide (40 mg, 863 μmol, 1.00 eq) at 25° C. under hydrogen atmosphere. The mixture was stirred at 25° C. for 12 h. The mixture was filtered to give filter liquor, the filtrate was concentrated under reduced pressure to give a residue. The residue was purified by Prep-HPLC (column: Phenomenex Gemini-NX C18 75*30 mm*3 μm; mobile phase: [water(0.225% formic acid)-acetonitrile]; B %: 8%-38%, 7 min) to afford 2-(2,6-dioxopiperidin-3-yl)-5-(3-hydroxyazetidin-1-yl)benzonitrile LXII (25.0 mg, 87.7 μmol, 10% yield) as a yellow solid. MS (ESI) m/z 286.0 [M+H]⁺.

### Synthesis of Exemplary Compounds

### Example 2. Synthesis of Compound 1

To a solution of 3-(4-(3-aminoazetidin-1-yl)-2,6-difluorophenyl)piperidine-2,6-dione IX (100 mg, 0.338 mmol, 1.00 eq) and triethylamine (141 μL, 1.02 mmol, 3.00 eq) in dimethylformamide (2 mL) was added phenyl (3-chloro-4-methylphenyl)carbamate (106 mg, 0.406 mmol, 1.20 eq). The mixture was stirred at 20° C. for 2 h. The mixture was filtered. The filtrate was purified by Prep-HPLC (column: Phenomenex Synergi C18 150*25 mm*10 μm; mobile phase: [water (0.225% formic acid)-acetonitrile]; B %: 38%-68%, 10 min) and lyophilized to afford 1-(3-chloro-4-methylphenyl)-3-(1-(4-(2,6-dioxopiperidin-3-yl)-3,5-difluorophenyl)azetidin-3-yl)urea #1 (26.04 mg, 0.0507 mmol, 14% yield, 99% purity, formate) as a white solid. ¹H NMR (400 MHz, DMSO-d₆) δ=10.86 (s, 1H), 8.85 (s, 1H), 8.46 (s, 1H), 7.63 (d, J=1.2 Hz, 1H), 7.19-7.16 (m, 1H), 7.16-7.13 (m, 1H), 7.11 (br d, J=7.2 Hz, 1H), 6.18 (s, 1H), 6.16 (s, 1H), 4.58-4.51 (m, 1H), 4.12 (t, J=7.6 Hz, 2H), 4.04 (br dd, J=4.8, 12.4 Hz, 1H), 3.65 (br t, J=6.4 Hz, 2H), 2.83-2.73 (m, 1H), 2.53-2.52 (m, 1H), 2.23 (s, 3H), 2.11-2.05 (m, 1H), 1.98-1.91 (m, 1H). ¹H NMR (400 MHz, CD₃OD) δ=8.52 (s, 1H), 7.52 (d, J=2.1 Hz, 1H), 7.17-7.14 (m, 1H), 7.13-7.09 (m, 1H), 6.11 (s, 1H), 6.08 (s, 1H), 4.71-4.61 (m, 1H), 4.21 (t, J=7.6 Hz, 2H), 4.07 (dd, J=4.8, 12.4 Hz, 1H), 3.68 (dd, J=5.6, 7.6 Hz, 2H), 2.84-2.73 (m, 1H), 2.70-2.64 (m, 1H), 2.29 (s, 3H), 2.23 (m, 1H), 2.10-2.03 (m, 1H). MS (ESI) m/z 463.2 [M+H]⁺.

### Example 3. Synthesis of Compound 2

To a solution of 3-(2,6-difluoro-4-(3-hydroxyazetidin-1-yl)phenyl)piperidine-2,6-dione XII (140 mg, 0.472 mmol, 1.00 eq) in dimethylformamide (2 mL) was added sodium hydride (37.8 mg, 0.945 mmol, 60% purity, 2.00 eq) at 0° C. Then to the mixture was added phenyl (3-chloro-4-methylphenyl)carbamate (148 mg, 567 μmol, 1.20 eq). The mixture was stirred at 20° C. for 1 h. The mixture was quenched with hydrochloric acid (1 M) until pH=3-4, diluted with water (20 mL) and extracted with ethyl acetate (3×20 mL). The combined organic layers were washed with brine (40 mL), dried over anhydrous sodium sulfate, filtered and concentrated under reduced pressure to give a residue. The residue was purified by Prep-HPLC (column: Phenomenex Synergi C18 150*25 mm*10 μm; mobile phase: [water (0.225% formic acid)-acetonitrile]; B %: 45%-81%, 12 min) and lyophilized to afford 1-(4-(2,6-dioxopiperidin-3-yl)-3,5-difluorophenyl)azetidin-3-yl(3-chloro-4-methylphenyl) carbamate #2 (91.15 mg, 0.196 mmol, 41% yield, 99% purity) as a white solid. ¹H NMR (400 MHz, DMSO-d₆) δ=10.86 (s, 1H), 10.01 (br s, 1H), 7.58 (s, 1H), 7.31-7.27 (m, 1H), 7.27-7.24 (m, 1H), 6.23 (s, 1H), 6.20 (s, 1H), 5.31-5.25 (m, 1H), 4.22 (br dd, J=7.2, 8.0 Hz, 2H), 4.04 (br dd, J=5.2, 12.8 Hz, 1H), 3.83 (br dd, J=3.6, 9.2 Hz, 2H), 2.83-2.73 (m, 1H), 2.52 (br s, 1H), 2.25 (s, 3H), 2.13-2.03 (m, 1H), 1.98-1.91 (m, 1H). ¹H NMR (400 MHz, CD₃OD) δ=7.55 (s, 1H), 7.21 (br d, J=2.0 Hz, 1H), 7.20-7.16 (m, 1H), 6.12 (s, 1H), 6.10 (s, 1H), 5.34 (m, 1H), 4.29-4.23 (m, 2H), 4.08 (dd, J=5.1, 12.6 Hz, 1H), 3.87 (dd, J=4.0, 9.0 Hz, 2H), 2.83-2.72 (m, 1H), 2.70-2.62 (m, 1H), 2.30 (s, 3H), 2.27-2.19 (m, 1H), 2.11-2.04 (m, 1H). MS (ESI) m/z 464.2 [M+H]⁺.

### Example 4. Synthesis of Compound 3

To a solution of 3-(2,6-difluoro-4-(3-hydroxyazetidin-1-yl)phenyl)piperidine-2,6-dione XII (60.0 mg, 203 μmol, 1.00 eq) in dimethyl formamide (0.500 mL) was added phenyl (3-(trifluoromethoxy)phenyl)carbamate (60.0 mg, 213 μmol, 1.05 eq) and sodium hydride (16.2 mg, 405 μmol, 60% purity, 2.00 eq). The mixture was stirred at 25° C. for 1 h. The pH of the mixture was adjusted to 7 with hydrochloric acid (1 M) and diluted with dimethyl formamide (1 mL). The solution was purified by Prep-HPLC (column: Phenomenex luna C18 150*25 mm*10 μm; mobile phase: [water (0.225% formic acid)-acetonitrile]; B %: 47%-77%, 10 min) to afford 1-(4-(2,6-dioxopiperidin-3-yl)-3,5-difluorophenyl)azetidin-3-yl(3-(trifluoromethoxy)phenyl) carbamate #3 (87.3 mg, 173 μmol, 86% yield, 99% purity) as a white solid. ¹H NMR (400 MHz, DMSO-d₆) δ=10.87 (s, 1H), 10.24 (br s, 1H), 7.58 (br s, 1H), 7.49-7.33 (m, 2H), 7.00 (br d, J=6.8 Hz, 1H), 6.22 (d, J=10.8 Hz, 2H), 5.36-5.22 (m, 1H), 4.32-4.16 (m, 2H), 4.05 (br dd, J=4.8, 12.7 Hz, 1H), 3.85 (br dd, J=3.4, 8.8 Hz, 2H), 2.87-2.71 (m, 1H), 2.49-2.49 (m, 1H), 2.17-2.00 (m, 1H), 1.99-1.89 (m, 1H). MS (ESI) m/z 500.0 [M+H]⁺.

### Example 5. Synthesis of Compound 4

To a solution of 3-(2,6-difluoro-4-(3-hydroxyazetidin-1-yl)phenyl)piperidine-2,6-dione XII (90.0 mg, 304 μmol, 1.00 eq) in dimethyl formamide (0.500 mL) was added phenyl (3,5-dimethylphenyl)carbamate (80.0 mg, 332 μmol, 1.09 eq) and sodium hydride (24.3 mg, 607 μmol, 60% purity, 2.00 eq). The mixture was stirred at 25° C. for 1 h. The pH of the mixture was adjusted to 7 with hydrochloric acid (1 M), then the mixture was diluted with dimethyl formamide (1 mL). The mixture was purified by Prep-HPLC (column: Phenomenex luna C18 150*25 mm*10 μm; mobile phase: [water(0.225% formic acid)-acetonitrile]; B %: 44%-74%, 10 min) to afford 1-(4-(2,6-dioxopiperidin-3-yl)-3,5-difluorophenyl)azetidin-3-yl (3,5-dimethyl phenyl)carbamate #4 (54.9 mg, 123 μmol, 40% yield, 99% purity) as a white solid. ¹H NMR (400 MHz, DMSO-d₆) δ=10.86 (br s, 1H), 9.74 (br s, 1H), 7.08 (s, 2H), 6.65 (s, 1H), 6.22 (d, J=10.8 Hz, 2H), 5.25 (td, J=3.2, 6.8 Hz, 1H), 4.21 (br t, J=7.6 Hz, 2H), 4.04 (br dd, J=4.4, 12.4 Hz, 1H), 3.82 (br dd, J=2.8, 8.8 Hz, 2H), 2.83-2.73 (m, 1H), 2.54-2.52 (m, 1H), 2.21 (s, 6H), 2.14-2.02 (m, 1H), 1.98-1.90 (m, 1H). MS (ESI) m/z 444.2 [M+H]⁺.

### Example 6. Synthesis of Compound 5

To a solution of 3-(2,6-difluoro-4-(3-hydroxyazetidin-1-yl)phenyl)piperidine-2,6-dione XII (80.0 mg, 270 μmol, 1.00 eq) in dimethyl formamide (0.500 mL) was added phenyl (3-chloro-4-methyl-5-(morpholinomethyl)phenyl)carbamate (87.7 mg, 243 μmol, 0.900 eq) and sodium hydride (21.6 mg, 540 umol, 60% purity, 2.00 eq). The mixture was stirred at 25° C. for 1 h. The pH of the mixture was adjusted to 7 with formic acid and diluted with dimethyl formamide (1 mL). The solution was purified by Prep-HPLC (column: Phenomenex Synergi C18 150*25 mm*10 μm; mobile phase: [water (0.225% formic acid)-acetonitrile]; B %: 13%-43%, 10 min) to afford 1-(4-(2,6-dioxopiperidin-3-yl)-3,5-difluorophenyl) azetidin-3-yl(3-chloro-4-methyl-5-(morpholinomethyl)phenyl)carbamate #5 (37.6 mg, 65.0 μmol, 24% yield, 97% purity) as a white solid. ¹H NMR (400 MHz, DMSO-d₆) δ=10.87 (s, 1H), 9.98 (br s, 1H), 7.51 (s, 1H), 7.34 (d, J=1.6 Hz, 1H), 6.22 (d, J=11.2 Hz, 2H), 5.34-5.23 (m, 1H), 4.22 (br t, J=7.6 Hz, 2H), 4.04 (br dd, J=4.8, 12.8 Hz, 1H), 3.83 (br dd, J=4.0, 8.8 Hz, 2H), 3.56 (br t, J=4.4 Hz, 4H), 3.40 (s, 2H), 2.85-2.71 (m, 1H), 2.59-2.54 (m, 1H), 2.38-2.32 (m, 4H), 2.28 (s, 3H), 2.14-2.02 (m, 1H), 1.99-1.90 (m, 1H). MS (ESI) m/z 563.2 [M+H]⁺.

### Example 7. Synthesis of Compound 6

To a solution of 3-(2,6-difluoro-4-(3-hydroxyazetidin-1-yl)phenyl)piperidine-2,6-dione XII (80.0 mg, 270 μmol, 1.00 eq) in dimethyl formamide (0.500 mL) was added phenyl (3-(difluoromethoxy)-4-methylphenyl) carbamate (71.3 mg, 243 μmol, 0.900 eq) and sodium hydride (21.6 mg, 540 μmol, 60% purity, 2.00 eq). The mixture was stirred at 25° C. for 1 h. The pH of the mixture was adjusted to 7 with formic acid and diluted with dimethyl formamide (1 mL). The solution was purified by Prep-HPLC (column: Phenomenex Synergi C18 150*25 mm*10 μm; mobile phase: [water (0.225% formic acid)-acetonitrile]; B %: 44%-74%, 10 min) and the desired eluent was lyophilized. Then the desired eluent was lyophilized to afford 1-(4-(2,6-dioxopiperidin-3-yl)-3,5-difluoropheny) azetidin-3-yl(3-(difluoromethoxy)-4-methylphenyl) carbamate #6 (42.4 mg, 77.6 μmol, 29% yield, 99% purity) as a white solid. ¹H NMR (400 MHz, DMSO-d₆) δ=10.87 (br s, 1H), 10.01 (br s, 1H), 7.42 (br s, 1H), 7.23-7.15 (m, 2H), 7.09 (t, J=74.0 Hz, 1H), 6.22 (d, J=11.2 Hz, 2H), 5.33-5.23 (m, 1H), 4.22 (br t, J=7.6 Hz, 2H), 4.05 (br dd, J=4.4, 12.4 Hz, 1H), 3.83 (br dd, J=3.6, 9.0 Hz, 2H), 2.85-2.71 (m, 1H), 2.61-2.56 (m, 1H), 2.15 (s, 3H), 2.11-2.01 (m, 1H), 1.99-1.89 (m, 1H). MS (ESI) m/z 496.2 [M+H]⁺.

### Example 8. Synthesis of Compound 7

To a mixture of 3-[4-(3-aminoazetidin-1-yl)-2-fluoro-phenyl]piperidine-2,6-dione XXVI: (60.0 mg, 216 μmol, 1.00 eq) and triethylamine (90.3 μL, 649 μmol, 3.00 eq) in dimethyl formamide (2 mL) was added phenyl (3-chloro-4-methylphenyl)carbamate (67.9 mg, 259 μmol, 1.20 eq). The reaction mixture was stirred at 20° C. for 12 h. The reaction mixture was concentrated to give a residue. The residue was purified by Prep-HPLC (column: Phenomenex Luna C18 150*25 mm*10 μm; mobile phase: [water (0.225% formic acid)-acetonitrile]; B %: 51%-81%, 10 min) followed by a subsequent Prep-HPLC (column: Phenomenex Luna C18 150*25 mm*10 μm; mobile phase: [water (0.225% formic acid)-acetonitrile]; B %: 45%-75%, 10 min) and lyophilized to give 1-(3-chloro-4-methylphenyl)-3-(1-(4-(2,6-dioxopiperidin-3-yl)-3-fluorophenyl)azetidin-3-yl)urea #7 (25.2 mg, 50.8 μmol, 23% yield, 99% purity, formic acid) as an off-white solid. ¹H NMR (400 MHz, DMSO-d₆) δ=10.80 (s, 1H), 9.11 (br s, 1H), 7.65 (s, 1H), 7.48-7.30 (m, 1H), 7.17 (s, 2H), 7.06 (t, J=8.8 Hz, 1H), 6.32-6.22 (m, 2H), 4.56 (qd, J=6.8, 13.2 Hz, 1H), 4.11 (t, J=7.6 Hz, 2H), 3.87 (dd, J=4.8, 12.4 Hz, 1H), 3.62 (t, J=6.8 Hz, 2H), 2.77-2.68 (m, 1H), 2.47-2.36 (m, 1H), 2.23 (s, 3H), 2.20-2.09 (m, 1H), 2.00-1.90 (m, 1H). MS (ESI) m/z 445.0 [M+H]⁺.

### Example 9. Synthesis of Compound 8

A mixture of 3-(2-fluoro-4-(3-hydroxyazetidin-1-yl)phenyl)piperidine-2,6-dione XXVII (220 mg, 0.790 mmol, 1.00 eq) and phenyl (3-chloro-4-methylphenyl)carbamate (248 mg, 0.948 mmol, 1.20 eq) in dimethylformamide (3 mL) was added sodium hydride (63.2 mg, 1.58 mmol, 60% purity, 2.00 eq) at 0° C. The reaction mixture was stirred at 20° C. for 2 h. The reaction mixture was quenched by addition formic acid (2 mL) and filtered. The filtrate was purified by Prep-HPLC (column: Phenomenex Synergi C18 150*25 mm*10 μm; mobile phase: [water (0.225% formic acid)-acetonitrile]; B %: 45%-75%, 10 min) and lyophilized to give 1-(4-(2,6-dioxopiperidin-3-yl)-3-fluorophenyl) azetidin-3-yl(3-chloro-4-methylphenyl)carbamate #8 (32.7 mg, 0.0718 mmol, 9% yield, 98% purity) as an off-white solid. ¹H NMR (400 MHz, DMSO-d₆) δ=10.80 (s, 1H), 10.01 (br s, 1H), 7.59 (s, 1H), 7.32-7.24 (m, 2H), 7.08 (t, J=8.4 Hz, 1H), 6.35-6.26 (m, 2H), 5.35-5.24 (m, 1H), 4.21 (dd, J=6.8, 8.4 Hz, 2H), 3.88 (dd, J=4.8, 12.4 Hz, 1H), 3.81 (dd, J=4.0, 8.8 Hz, 2H), 2.77-2.68 (m, 1H), 2.54-2.53 (m, 1H), 2.26 (s, 3H), 2.14 (dq, J=4.4, 12.8 Hz, 1H), 2.00-1.91 (m, 1H). MS (ESI) m/z 446.2 [M+H]⁺.

### Example 10. Synthesis of Compound 9

Step 1. To a mixture of 2-methyl-5-nitrobenzoic acid (10.0 g, 55.2 mmol, 1.00 eq) and 1,3-dichloro-5,5-dimethylimidazolidine-2,4-dione (13.1 g, 66.3 mmol, 1.20 eq) in sulfuric acid (30 mL) was stirred at 80° C. for 12 h. The reaction mixture was poured into ice water (about 300 mL) and after stirring, the precipitated solid was collected by filtration and washed with water to afford 3-chloro-2-methyl-5-nitrobenzoic acid (12.5 g, crude) as a white solid. ¹H NMR (400 MHz, CDCl₃) δ=8.79 (d, J=2.4 Hz, 1H), 8.47 (d, J=2.2 Hz, 1H), 2.83 (s, 3H).

Step 2. To a solution of 3-chloro-2-methyl-5-nitro-benzoic acid (12.5 g, 57.9 mmol, 1.00 eq) in tetrahydrofuran (100 mL) was added borane dimethyl sulfide complex (10.0 M, 11.6 mL, 2.00 eq) at 0° C. Then the mixture was stirred at 25° C. for 12 h. The reaction mixture was quenched by addition water (50.0 mL), and then extracted with ethyl acetate (3×200 mL). The combined organic layers were washed with brine (100 mL), dried over sodium sulfate, filtered and concentrated under reduced pressure to afford (3-chloro-2-methyl-5-nitro-phenyl)methanol (10.8 g, crude) as a white solid. ¹H NMR (400 MHz, CDCl₃) δ=8.26 (d, J=2.0 Hz, 1H), 8.19 (d, J=2.4 Hz, 1H), 4.82 (s, 2H), 2.45 (s, 3H).

Step 3. A mixture of (3-chloro-2-methyl-5-nitrophenyl)methanol (1.00 g, 4.96 mmol, 1.00 eq) and thionyl chloride (1.80 mL, 24.8 mmol, 5.00 eq) in dichloromethane (5 mL) was stirred at 25° C. for 12 h. The reaction mixture was concentrated under reduced pressure to remove dichloromethane. The residue was diluted with water (30 mL) and extracted with ethyl acetate (3×30 mL). The combined organic layers were washed with brine (3×30 mL), dried over magnesium sulfate, filtered and concentrated under reduced pressure to afford 1-chloro-3-(chloromethyl)-2-methyl-5-nitrobenzene (1.00 g, 4.54 mmol, 92% yield) as yellow oil. ¹H NMR (400 MHz, CDCl₃) δ=8.26 (d, J=2.2 Hz, 1H), 8.16 (d, J=2.2 Hz, 1H), 4.68 (s, 2H), 2.58 (s, 3H).

Step 4. To a solution of 1-chloro-3-(chloromethyl)-2-methyl-5-nitrobenzene (1.00 g, 4.54 mmol, 1.00 eq) and 2-oxa-5-azabicyclo[2.2.1]heptane (770 mg, 5.68 mmol, 1.25 eq, HCl) in dichloromethane (10 mL) was added triethylamine (1.90 mL, 13.6 mmol, 3.00 eq) at 0° C. Then the mixture was stirred at 25° C. for 12 h. The reaction mixture was concentrated under reduced pressure to give a residue. The residue was purified by column chromatography (SiO₂, petroleum ether/ethyl acetate=1/0 to 3/1) to afford 5-(3-chloro-2-methyl-5-nitrobenzyl)-2-oxa-5-azabicyclo[2.2.1]heptane (1.00 g, 3.54 mmol, 77.8% yield) as a light yellow solid. ¹H NMR (400 MHz, CDCl₃) δ=8.17 (s, 2H), 4.48 (s, 1H), 4.14 (d, J=7.2 Hz, 2H), 3.83 (d, J=4.4 Hz, 2H), 3.49 (s, 1H), 2.94 (dd, J=1.2, 9.8 Hz, 1H), 2.59 (d, J=10.0 Hz, 1H), 2.49 (s, 3H), 1.96 (br d, J=9.8 Hz, 1H), 1.81 (td, J=1.2, 9.8 Hz, 1H).

Step 5. A mixture of 5-(3-chloro-2-methyl-5-nitrobenzyl)-2-oxa-5-azabicyclo[2.2.1]heptane (1.00 g, 3.54 mmol, 1.00 eq), ammonium chloride (284 mg, 5.31 mmol, 1.50 eq) and ferrous powder (1.58 g, 28.3 mmol, 8.00 eq) in ethanol (10 mL) and water (5 mL) was stirred at 80° C. for 10 h. The reaction mixture was filtered. The residue was purified by prep-HPLC (basic condition) to afford 3-(2-oxa-5-azabicyclo[2.2.1]heptan-5-ylmethyl)-5-chloro-4-methylaniline (0.550 g, 2.18 mmol, 62% yield) as a white solid. ¹H NMR (400 MHz, CDCl₃) δ=6.68-6.63 (m, 2H), 4.44 (s, 1H), 4.11 (d, J=7.8 Hz, 1H), 3.69-3.65 (m, 3H), 3.63-3.56 (m, 2H), 3.46 (s, 1H), 2.89 (dd, J=1.6, 10.2 Hz, 1H), 2.60 (d, J=10.2 Hz, 1H), 2.28 (s, 3H), 1.90 (dd, J=1.8, 9.7 Hz, 1H), 1.75 (td, J=1.2, 9.6 Hz, 1H).

Step 6. To a solution of 3-(2-oxa-5-azabicyclo[2.2.1]heptan-5-ylmethyl)-5-chloro-4-methylaniline (0.550 g, 2.18 mmol, 1.00 eq) and potassium carbonate (0.601 g, 4.35 mmol, 2.00 eq) in acetone (5 mL) was added phenyl carbonochloridate (327 μL, 2.61 mmol, 1.20 eq) at 0° C. Then the mixture was stirred at 25° C. for 12 h. The reaction mixture was concentrated under reduced pressure to remove acetone (5 mL). The residue was diluted with water (50 mL) and extracted with ethyl acetate (3×50 mL). The combined organic layers were washed with brine (2×50 mL), dried over sodium sulfate, filtered and concentrated under reduced pressure to afford phenyl(3-(2-oxa-5-azabicyclo[2.2.1]heptan-5-ylmethyl)-5-chloro-4-methylphenyl)carbamate (0.85 g, crude) as a yellow solid and was used for the next step without purification.

Step 7. To a solution of 3-(4-(3-aminoazetidin-1-yl)-2,6-difluorophenyl)piperidine-2,6-dione IX (70.0 mg, 237 μmol, 1.00 eq) and phenyl (3-(2-oxa-5-azabicyclo[2.2.1]heptan-5-ylmethyl)-5-chloro-4-methylphenyl)carbamate (106 mg, 285 μmol, 1.20 eq) in N,N-dimethylformamide (2 mL) was added triethylamine (65.9 μL, 474 μmol, 2.00 eq). Then the mixture was stirred at 25° C. for 12 h. The reaction mixture was diluted with water (50 mL) and extracted with ethyl acetate (3×50 mL). The combined organic layers were washed with brine (2×50 mL), dried over sodium sulfate, filtered and concentrated under reduced pressure to give a residue. The residue was purified by prep-HPLC (column: Welch Ultimate XB-CN 250*50 mm*10 μm; mobile phase: [hexane-ethyl alcohol]; B %: 20%-60%, 12 min). The residue was further purified by prep-HPLC (column: Phenomenex Gemini-NX C18 75*30 mm*3 μm; mobile phase: [water (10 mM ammonium bicarbonate)-acetonitrile]; B %: 26%-56%, 8 min) to afford 1-(3-(2-oxa-5-azabicyclo[2.2.1]heptan-5-ylmethyl)-5-chloro-4-methylphenyl)-3-(1-(4-(2,6-dioxopiperidin-3-yl)-3,5-difluorophenyl)azetidin-3-yl)urea #9 (22.1 mg, 38.6 μmol, 16% yield) as a white solid. ¹H NMR (400 MHz, DMSO-d₆) δ=10.87 (s, 1H), 8.73-8.66 (m, 1H), 7.59 (d, J=2.0 Hz, 1H), 7.19 (s, 1H), 6.91-6.84 (m, 1H), 6.18 (d, J=11.0 Hz, 2H), 4.60-4.51 (m, 1H), 4.37 (s, 1H), 4.13 (t, J=7.6 Hz, 2H), 4.05 (br dd, J=5.2, 12.6 Hz, 1H), 3.91 (d, J=7.6 Hz, 1H), 3.66 (br s, 2H), 3.54 (br d, J=6.2 Hz, 1H), 3.44 (br s, 1H), 3.31 (br s, 1H), 2.80-2.67 (m, 4H), 2.45 (br d, J=10.0 Hz, 1H), 2.25 (s, 3H), 2.12-2.03 (m, 1H), 1.99-1.91 (m, 1H), 1.80 (br d, J=9.6 Hz, 1H), 1.60 (br d, J=9.2 Hz, 1H). MS (ESI) m/z 574.0 [M+H]⁺.

### Example 11. Synthesis of Compound 10

To a solution of (R)-phenyl (6-(2-methylpyrrolidin-1-yl)pyridin-3-yl)carbamate (88.7 mg, 298 μmol, 1.30 eq) in dimethyl formamide (1 mL) was added 3-(2,6-difluoro-4-(3-hydroxyazetidin-1-yl)phenyl)piperidine-2,6-dione XII (68.0 mg, 230 μmol, 1.00 eq) and sodium hydride (18.4 mg, 459 μmol, 60% purity, 2.00 eq) at 0° C. The mixture was stirred at 25° C. for 1 h. The pH of the mixture was adjusted to 7 with hydrochloric acid (1 M), then the mixture was diluted with dimethyl formamide (1.00 mL). The mixture was purified by Prep-HPLC (column: Phenomenex Luna C18 150*25 mm*10 μm; mobile phase: [water (0.225% formic acid)-acetonitrile]; B %: 12%-42%, 10 min) to afford 1-(4-(2,6-dioxopiperidin-3-yl)-3,5-difluorophenyl)azetidin-3-yl (6-((R)-2-methylpyrrolidin-1-yl)pyridin-3-yl)carbamate #10 (57.7 mg, 115 μmol, 50% yield) as a white solid. ¹H NMR (400 MHz, DMSO-d₆) δ=10.98 (s, 1H), 9.60 (br s, 1H), 8.20 (br s, 1H), 7.66 (br d, J=8.0 Hz, 1H), 6.52 (d, J=8.8 Hz, 1H), 6.34 (d, J=11.2 Hz, 2H), 5.42-5.32 (m, 1H), 4.32 (br t, J=7.6 Hz, 2H), 4.22-4.12 (m, 2H), 3.92 (br d, J=6.6 Hz, 2H), 3.54 (ddd, J=2.4, 7.2, 9.6 Hz, 2H), 3.36-3.28 (m, 1H), 2.96-2.84 (m, 1H), 2.26-2.16 (m, 1H), 2.14-2.08 (m, 2H), 2.08-1.98 (m, 2H), 1.82-1.70 (m, 1H), 1.24 (d, J=6.4 Hz, 3H). MS (ESI) m/z 500.3 [M+H]⁺.

### Example 12. Synthesis of Compound 11

Step 1. To a solution of 3-(4-bromo-2,6-difluorophenyl)piperidine-2,6-dione V (1.00 g, 3.29 mmol, 1.00 eq), tert-butyl azetidin-3-ylcarbamate (892 mg, 4.28 mmol, 1.30 eq, hydrochloride) and cesium carbonate (3.21 g, 9.87 mmol, 3.00 eq) in dioxane (10 mL) were added Pd-PEPPSI-IHeptCl 3-chloropyridine precatalyst (100 mg). The reaction mixture was stirred at 100° C. for 12 h under nitrogen atmosphere. The mixture was concentrated under residue pressure to give a residue. The residue was dissolved in dimethylformamide (10 mL) and filtered. The filtrate was purified by reversed phase column chromatography (C18, 330 g; condition: water/acetonitrile=1/0 to 0/1, 0.1% formic acid) and lyophilized to afford tert-butyl (1-(4-(2,6-dioxopiperidin-3-yl)-3,5-difluorophenyl)azetidin-3-yl)carbamate (0.570 g, 1.44 mmol, 44% yield) as a brown solid. ¹H NMR (400 MHz, DMSO-d₆) δ=10.85 (s, 1H), 7.61-7.44 (m, 1H), 6.14 (s, 1H), 6.12 (s, 1H), 4.45-4.32 (m, 1H), 4.07 (t, J=7.4 Hz, 2H), 4.04-3.99 (m, 1H), 3.60 (t, J=6.8 Hz, 2H), 2.83-2.72 (m, 1H), 2.47 (br s, 1H), 2.09-2.05 (m, 1H), 1.98-1.90 (m, 1H), 1.39 (s, 9H).

Step 2. A solution of tert-butyl (1-(4-(2,6-dioxopiperidin-3-yl)-3,5-difluorophenyl)azetidin-3-yl)carbamate (100 mg, 253 μmol, 1.00 eq) in 1,1,1,3,3,3-hexafluoropropan-2-ol (1 mL) was stirred at 100° C. for 2 h under microwave. Then the reaction mixture was stirred at 150° C. for 3 h under microwave. The reaction mixture was concentrated under reduced pressure to afford 3-(4-(3-aminoazetidin-1-yl)-2,6-difluorophenyl) piperidine-2,6-dione (80.0 mg, crude) as a brown oil. ¹H NMR (400 MHz, DMSO-d₆) δ=10.85 (br s, 1H), 8.27-7.82 (m, 1H), 6.11 (s, 1H), 6.08 (s, 1H), 5.15 (td, J=6.8, 13.6 Hz, 1H), 4.04-4.00 (m, 2H), 3.77 (quin, J=6.4 Hz, 1H), 3.39-3.36 (m, 2H), 2.83-2.72 (m, 1H), 2.48 (br d, J=3.1 Hz, 1H), 2.07 (br dd, J=3.7, 13.0 Hz, 1H), 1.93 (tdd, J=2.6, 5.2, 13.1 Hz, 1H).

Step 3. To a solution of 3-(4-(3-aminoazetidin-1-yl)-2,6-difluorophenyl)piperidine-2,6-dione IX (72.0 mg, 244 μmol, 1.00 eq) in dimethylformamide (1 mL) were added triethylamine (67.9 μL, 488 μmol, 2.00 eq) and phenyl (3,5-dimethylphenyl)carbamate (58.8 mg, 244 μmol, 1.00 eq). The reaction mixture was stirred at 30° C. for 1 h. The reaction mixture was filtered. The filtrate was purified by Prep-HPLC (column: Phenomenex Synergi C18 150*25 mm*10 μm; mobile phase: [water (0.225% formic acid)-acetonitrile]; B %: 39%-69%, 10 min) and lyophilized to afford 1-(3,5-dimethylphenyl)-3-(1-(4-(2,6-dioxopiperidin-3-yl)-3,5-difluorophenyl)azetidin-3-yl)urea #11 (50.54 mg, 113 μmol, 46% yield, 99% purity) as a white solid. ¹H NMR (400 MHz, DMSO-d₆) δ=10.86 (s, 1H), 8.41 (s, 1H), 7.01 (s, 2H), 6.85 (d, J=7.2 Hz, 1H), 6.55 (s, 1H), 6.19 (s, 1H), 6.16 (s, 1H), 4.58-4.48 (m, 1H), 4.12 (t, J=7.6 Hz, 2H), 4.04 (dd, J=5.0, 12.7 Hz, 1H), 3.64 (br t, J=6.1 Hz, 2H), 2.84-2.72 (m, 1H), 2.53-2.52 (m, 1H), 2.19 (s, 6H), 2.14-2.02 (m, 1H), 1.99-1.89 (m, 1H). MS (ESI) m/z 443.0 [M+H]⁺.

### Example 13. Synthesis of Compound 12

To a solution of 3-(4-(3-aminoazetidin-1-yl)-2,6-difluorophenyl)piperidine-2,6-dione IX (100 mg, 244 μmol, 1.00 eq, trifluoroacetic acid) and triethylamine (102 μL, 733 μmol, 3.00 eq) in dimethyl formamide (1 mL) was added phenyl (6-(tert-butyl)pyridin-3-yl)carbamate (79.3 mg, 293 μmol, 1.20 eq) at 0° C. The mixture was stirred at 30° C. for 1 h and was quenched by acetic acid (1 mL) to give a solution. The solution was purified by prep-HPLC (column: Phenomenex luna C18 150*25 mm*10 μm; mobile phase: [water (0.225% formic acid)-acetonitrile]; B %: 13%-43%, 10 min) and lyophilized to give 1-(6-(tert-butyl)pyridin-3-yl)-3-(1-(4-(2,6-dioxopiperidin-3-yl)-3,5-difluorophenyl) azetidin-3-yl)urea #12 (34.53 mg, 66.1 μmol, 27% yield, 99% purity, formic acid) as a white solid. ¹H NMR (400 MHz, DMSO-d₆) δ=10.86 (s, 1H), 8.75 (s, 1H), 8.44 (d, J=2.6 Hz, 1H), 7.80 (dd, J=2.7, 8.7 Hz, 1H), 7.28 (d, J=8.7 Hz, 1H), 7.10-7.04 (m, 1H), 6.19 (s, 1H), 6.16 (s, 1H), 4.57 (qd, J=6.6, 13.1 Hz, 1H), 4.13 (t, J=7.6 Hz, 2H), 4.04 (dd, J=5.0, 12.6 Hz, 1H), 3.66 (br t, J=6.1 Hz, 2H), 2.83-2.72 (m, 1H), 2.52-2.52 (m, 1H), 2.14-2.02 (m, 1H), 1.99-1.91 (m, 1H), 1.27 (s, 9H). MS (ESI) m/z 472.3 [M+H]⁺.

### Example 14. Synthesis of Compound 13

Step 1. To a mixture of 2-chloro-1-methyl-4-nitrobenzene (6.02 mL, 29.1 mmol, 1.00 eq) in N-iodo-succinimide (7.21 g, 32.1 mmol, 1.10 eq) was added sulfuric acid (50 mL). The mixture was stirred at 60° C. for 2 h. The reaction mixture was diluted with water (20 mL) and extracted with ethyl acetate (3×30 mL). The combined organic layers were washed with brine (2×20 mL), dried over magnesium sulfate, filtered and concentrated under reduced pressure to afford 1-chloro-3-iodo-2-methyl-5-nitrobenzene (10.2 g, crude) as an orange solid. ¹H NMR (400 MHz, CDCl₃) δ=8.59 (d, J=2.2 Hz, 1H), 8.25 (d, J=2.2 Hz, 1H), 2.70 (s, 3H).

Step 2. A mixture of 1-chloro-3-iodo-2-methyl-5-nitrobenzene (5.00 g, 16.8 mmol, 1.00 eq), potassium hydroxide (2.83 g, 50.4 mmol, 3.00 eq), tris(dibenzylideneacetone)dipalladium (1.54 g, 1.68 mmol, 0.10 eq), t-Bu XPhos (0.714 g, 1.68 mmol, 0.10 eq) and water (6 mL) in dioxane (30 mL) was degassed and purged 3 times with nitrogen, and then the mixture was stirred at 80° C. for 12 h under nitrogen atmosphere. The reaction mixture was diluted with water (20 mL) and extracted with ethyl acetate (3×30 mL). The combined organic layers were washed with brine (2×20 mL), dried over magnesium sulfate, filtered and concentrated under reduced pressure to give a residue. The residue was purified by column chromatography (SiO₂, petroleum ether/ethyl acetate=1/0 to 0/1) to afford 3-chloro-2-methyl-5-nitrophenol (1.20 g, 6.40 mmol, 38% yield) as a yellow solid. 1H NMR (400 MHz, CDCl₃) δ=7.82 (d, J=2.2 Hz, 1H), 7.64 (d, J=2.2 Hz, 1H), 7.09 (s, 1H), 2.37 (s, 3H).

Step 3. A mixture of 3-chloro-2-methyl-5-nitrophenol (0.500 g, 2.67 mmol, 1.00 eq), 4-(2-chloroethyl)morpholine (0.598 mg, 4.00 mmol, 1.50 eq), potassium carbonate (1.11 g, 8.00 mmol, 3.00 eq) and potassium iodide (0.664 mg, 4.00 mmol, 1.50 eq) in dimethylformamide (5 mL) was stirred at 80° C. for 1 h. The reaction mixture was filtered. The crude product was purified by reversed-phase HPLC (0.1% FA condition) to afford 4-(2-(3-chloro-2-methyl-5-nitrophenoxy)ethyl)morpholine (100 mg, 332 μmol, 12% yield) as a yellow solid. ¹H NMR (400 MHz, CDCl₃) δ=7.83 (d, J=2.2 Hz, 1H), 7.54 (d, J=2.0 Hz, 1H), 4.14 (t, J=5.6 Hz, 2H), 3.69-3.65 (m, 4H), 2.81 (t, J=5.6 Hz, 2H), 2.58-2.51 (m, 4H), 2.28 (s, 3H).

Step 4. A mixture of 4-(2-(3-chloro-2-methyl-5-nitrophenoxy)ethyl)morpholine (100 mg, 0.333 mmol, 1.00 eq), ferrous powder (92.9 mg, 1.66 mmol, 5.00 eq) and ammonium chloride (17.8 mg, 0.333 mmol, 1.00 eq) in methanol (2 mL) and water (1 mL) was stirred at 80° C. for 12 h. The reaction mixture was concentrated under reduced pressure to remove methanol. The residue was diluted with water (30 mL) and extracted with ethyl acetate (3×30 mL). The combined organic layers were washed with brine (3×30 mL), dried over sodium sulfate, filtered and concentrated under reduced pressure to give a residue to afford 3-chloro-4-methyl-5-(2-morpholinoethoxy)aniline (80 mg, crude) as a white solid and was used for the next step without purification

Step 5. A mixture of 3-chloro-4-methyl-5-(2-morpholinoethoxy)aniline (80.0 mg, 296 μmol, 1.00 eq), phenyl carbonochloridate (44.4 μL, 355 μmol, 1.20 eq) and pyridine (35.8 μL, 443 μmol, 1.50 eq) in dimethylformamide (2 mL) was stirred at 25° C. for 12 h. The mixture was diluted with water (30 mL) and extracted with ethyl acetate (3×30 mL). The combined organic layers were washed with brine (3×30 mL), dried over sodium sulfate, filtered and concentrated under reduced pressure to afford phenyl (3-chloro-4-methyl-5-(2-morpholinoethoxy)phenyl)carbamate (130 mg, crude) as a yellow oil and was used for the next step without purification.

Step 6. To a mixture of phenyl (3-chloro-4-methyl-5-(2-morpholinoethoxy)phenyl)carbamate (130 mg, 333 μmol, 1.00 eq) and 3-(4-(3-aminoazetidin-1-yl)-2,6-difluorophenyl)piperidine-2,6-dione IX (98.2 mg, 333 μmol, 1.00 eq) in N,N-dimethylformamide (2 mL) was added triethylamine (92.6 μL, 665 μmol, 2.00 eq). Then the mixture was stirred at 30° C. for 10 h. The reaction mixture filtered. The residue was purified by prep-HPLC (column: Waters Xbridge 150*25 mm*5 μm; mobile phase: [water (10 mM ammonium bicarbonate)-acetonitrile]; B %: 38%-68%, 10 min) to afford 1-(3-chloro-4-methyl-5-(2-morpholinoethoxy) phenyl)-3-(1-(4-(2,6-dioxopiperidin-3-yl)-3,5-difluorophenyl)azetidin-3-yl)urea #13 (50.7 mg, 83.0 μmol, 25% yield, 97% purity) as a white solid. ¹H NMR (400 MHz, DMSO-d₆) δ=10.87 (s, 1H), 8.74-8.68 (m, 1H), 7.14 (d, J=1.6 Hz, 1H), 7.05 (s, 1H), 6.96 (br d, J=6.6 Hz, 1H), 6.18 (d, J=11.0 Hz, 2H), 4.58-4.52 (m, 1H), 4.13 (br t, J=7.6 Hz, 2H), 4.04 (br t, J=5.6 Hz, 4H), 3.66 (br t, J=6.2 Hz, 2H), 3.60-3.55 (m, 5H), 2.84-2.71 (m, 4H), 2.12-2.04 (m, 5H), 2.03-1.89 (m, 2H). MS (ESI) m/z 592.3 [M+H]⁺.

### Example 15. Synthesis of Compound 14

To a solution of phenyl (3-(difluoromethoxy)phenyl)carbamate (98.0 mg, 351 μmol, 1.30 eq) in dimethyl formamide (1 mL) was added 3-(2,6-difluoro-4-(3-hydroxyazetidin-1-yl)phenyl)piperidine-2,6-dione XII (80.0 mg, 270 μmol, 1.00 eq) and sodium hydride (21.6 mg, 540 μmol, 60% purity, 2.00 eq) at 0° C. The mixture was stirred at 20° C. for 1 h. The pH of the mixture was adjusted to 7 with hydrochloric acid (1 M), then the mixture was diluted with dimethyl formamide (1 mL). The mixture was purified by Prep-HPLC (column: Phenomenex Luna C18 150*25 mm*10 μm; mobile phase: [water (0.225% formic acid)-acetonitrile]; B %: 43%-73%, 10 min) to afford 1-(4-(2,6-dioxopiperidin-3-yl)-3,5-difluorophenyl)azetidin-3-yl (3-(difluoromethoxy) phenyl)carbamate #14 (60.6 mg, 126 μmol, 47% yield) as a white solid. ¹H NMR (400 MHz, DMSO-d₆) δ=10.98-10.82 (m, 1H), 10.24-10.08 (m, 1H), 7.40 (br s, 1H), 7.39-7.01 (t, J=74 Hz, 1H), 7.37-7.26 (m, 2H), 6.90-6.75 (m, 1H), 6.24 (br dd, J=3.6, 11.0 Hz, 2H), 5.38-5.23 (m, 1H), 4.25 (br d, J=5.7 Hz, 2H), 4.08-4.02 (m, 1H), 3.89-3.83 (m, 2H), 2.85-2.76 (m, 2H), 2.16-2.04 (m, 1H), 2.01-1.88 (m, 1H). MS (ESI) m/z 482.1 [M+H]⁺.

### Example 16. Synthesis of Compound 15

To a solution of 3-(4-(3-aminoazetidin-1-yl)-2,6-difluorophenyl)piperidine-2,6-dione IX (100 mg, 293 μmol, 1.00 eq, formic acid) and triethylamine (122 μL, 879 μmol, 3.00 eq) in dimethyl formamide (1 mL) was added phenyl (3-(difluoromethoxy)phenyl)carbamate (90.0 mg, 322 μmol, 1.10 eq) at 0° C. The mixture was stirred at 30° C. for 1 h. The mixture was quenched by acetic acid (1 mL) to give a solution. The solution was purified by prep-HPLC (column: Phenomenex luna C18 150*25 mm*10 μm; mobile phase: [water (0.225% formic acid)-acetonitrile]; B %: 35%-65%, 10 min) and lyophilized to give 1-(3-(difluoromethoxy)phenyl)-3-(1-(4-(2,6-dioxopiperidin-3-yl)-3,5-difluorophenyl)azetidin-3-yl)urea #15 (13.03 mg, 26.9 μmol, 9% yield, 99% purity) as a white solid. ¹H NMR (400 MHz, DMSO-d₆) δ=10.86 (s, 1H), 8.98-8.82 (m, 1H), 7.41 (t, J=2.1 Hz, 1H), 7.29-7.22 (m, 1H), 7.16-7.15 (s, 1H), 7.13 (t, J=64 Hz, 1H), 7.04-6.98 (m, 1H), 6.70 (dd, J=2.1, 8.0 Hz, 1H), 6.19 (s, 1H), 6.16 (s, 1H), 4.59-4.50 (m, 1H), 4.13 (t, J=7.6 Hz, 2H), 4.04 (br dd, J=5.0, 12.5 Hz, 1H), 3.66 (br t, J=6.1 Hz, 2H), 2.83-2.73 (m, 1H), 2.42 (br d, J=2.9 Hz, 1H), 2.13-2.02 (m, 1H), 1.98-1.91 (m, 1H). MS (ESI) m/z 508.0 [M+H]⁺.

### Example 17. Synthesis of Compound 16

To a solution of 3-(4-(3-aminoazetidin-1-yl)-2,6-difluorophenyl)piperidine-2,6-dione IX (100 mg, 293 μmol, 1.00 eq, formate) and triethylamine (81.6 μL, 586 μmol, 2.00 eq) in dimethylformamide (1 mL) was added phenyl (3-chloro-5-fluorophenyl)carbamate (85.6 mg, 322 μmol, 1.10 eq). The mixture was stirred at 30° C. for 12 h. The mixture was filtered. The filtrate was purified by Prep-HPLC (column: Phenomenex luna C18 150*25 mm*10 μm; mobile phase: [water (0.225% formic acid)-acetonitrile]; B %: 43%-73%, 10 min) and lyophilized to afford 1-(3-chloro-5-fluorophenyl)-3-(1-(4-(2,6-dioxopiperidin-3-yl)-3,5-difluorophenyl)azetidin-3-yl)urea #16 (56.99 mg, 120 μmol, 41% yield, 99% purity) as a white solid. ¹H NMR (400 MHz, DMSO-d₆) δ=10.86 (s, 1H), 9.16-9.03 (m, 1H), 7.34 (s, 1H), 7.30 (dd, J=1.8, 11.6 Hz, 1H), 7.25-7.15 (m, 1H), 6.91 (td, J=2.1, 8.6 Hz, 1H), 6.19 (s, 1H), 6.16 (s, 1H), 4.60-4.50 (m, 1H), 4.13 (t, J=7.6 Hz, 2H), 4.04 (br dd, J=5.0, 12.7 Hz, 1H), 3.73-3.63 (m, 2H), 2.84-2.72 (m, 1H), 2.53-2.52 (m, 1H), 2.11-2.03 (m, 1H), 1.98-1.90 (m, 1H). MS (ESI) m/z 466.9 [M+H]⁺.

### Example 18. Synthesis of Compound 17

Step 1. To a solution of 3-cyclopropoxyaniline (300 mg, 2.01 mmol, 1.00 eq) and pyridine (487 μL, 6.03 mmol, 3.00 eq) in acetonitrile (3 mL) was added phenyl carbonochloridate (277 μL, 2.21 mmol, 1.10 eq) at 0° C. The reaction mixture was stirred at 20° C. for 1 h. The reaction mixture was concentrated under reduced pressure to give a residue. The residue was purified by column chromatography (SiO₂, petroleum ether/ethyl acetate=1/0 to 3/1) and concentrated under reduced pressure to afford phenyl (3-cyclopropoxyphenyl)carbamate (500 mg, 1.86 mmol, 92% yield) as red oil ¹H NMR (400 MHz, CDCl₃) δ=7.34-7.29 (m, 2H), 7.27 (br s, 1H), 7.18-7.14 (m, 2H), 7.13-7.09 (m, 2H), 6.89-6.80 (m, 2H), 6.75-6.68 (m, 1H), 3.68-3.61 (m, 1H), 0.71-0.66 (m, 4H).

Step 2. To a solution of 3-(4-(3-aminoazetidin-1-yl)-2,6-difluorophenyl)piperidine-2,6-dione IX (100 mg, 293 μmol, 1.00 eq, formate) and triethylamine (81.6 μL, 586 μmol, 2.00 eq) in dimethylformamide (1 mL) was added phenyl (3-cyclopropoxyphenyl)carbamate (86.8 mg, 322 μmol, 1.10 eq). The mixture was stirred at 30° C. for 12 h. The mixture was filtered and the filtrate was purified by Prep-HPLC (column: Phenomenex luna C18 150*25 mm*10 μm; mobile phase: [water (0.225% formic acid)-acetonitrile]; B %: 40%-70%, 10 min) and lyophilized to afford 1-(3-cyclopropoxyphenyl)-3-(1-(4-(2,6-dioxopiperidin-3-yl)-3,5-difluorophenyl)azetidin-3-yl)urea #17 (60.81 mg, 127 μmol, 44% yield, 99% purity) as a white solid. ¹H NMR (400 MHz, DMSO-d₆) δ=10.86 (s, 1H), 8.60 (br d, J=14.9 Hz, 1H), 7.25 (s, 1H), 7.11 (t, J=8.1 Hz, 1H), 6.97-6.82 (m, 2H), 6.59 (dd, J=2.0, 8.1 Hz, 1H), 6.19 (s, 1H), 6.16 (s, 1H), 4.60-4.49 (m, 1H), 4.13 (t, J=7.7 Hz, 2H), 4.04 (br dd, J=5.0, 12.5 Hz, 1H), 3.75 (tt, J=3.0, 5.9 Hz, 1H), 3.64 (br t, J=6.2 Hz, 2H), 2.84-2.72 (m, 1H), 2.52 (br s, 1H), 2.13-2.02 (m, 1H), 1.99-1.88 (m, 1H), 0.78-0.69 (m, 2H), 0.69-0.59 (m, 2H). MS (ESI) m/z 471.0 [M+H]⁺.

### Example 19. Synthesis of Compound 18

To a solution of phenyl (3-(trifluoromethoxy)phenyl)carbamate (72.6 mg, 244 μmol, 1.00 eq) in dimethylformamide (1 mL) was added 3-(4-(3-aminoazetidin-1-yl)-2,6-difluorophenyl) piperidine-2,6-dione (100 mg, 244 μmol, 1.00 eq, trifluoroacetic acid) and triethylamine (136.2 μL, 977 μmol, 4.00 eq). The reaction mixture was stirred at 30° C. for 1 h. The reaction was filtered and the filtrate was purified by Prep-HPLC (column: Unisil 3-100 C18 Ultra 150*5 0 mm*3 μm; mobile phase: [water (0.225% formic acid)-acetonitrile]; B %: 40%-70%, 10 min) and lyophilized to afford 1-(1-(4-(2,6-dioxopiperidin-3-yl)-3,5-difluorophenyl)azetidin-3-yl)-3-(3-(trifluoromethoxy)phenyl)urea #18 (49.48 mg, 99.2 μmol, 41% yield) as a white solid. ¹H NMR (400 MHz, DMSO-d₆) δ=10.8 (s, 1H), 8.90 (s, 1H), 7.65 (s, 1H), 7.37-7.30 (m, 1H), 7.28-7.20 (m, 1H), 6.96 (d, J=7.2 Hz, 1H), 6.87 (br d, J=8.3 Hz, 1H), 6.18 (d, J=11.1 Hz, 2H), 4.63-4.49 (m, 1H), 4.13 (t, J=7.7 Hz, 2H), 4.04 (dd, J=5.1, 12.3 Hz, 1H), 3.67 (br t, J=6.4 Hz, 2H), 2.84-2.72 (m, 1H), 2.64-2.58 (m, 1H), 2.16-2.01 (m, 1H), 1.99-1.87 (m, 1H). MS (ESI) m/z 499.2 [M+H]⁺.

### Example 20. Synthesis of Compound 19

To a solution of To a solution of phenyl (3-(difluoromethoxy)-4-fluorophenyl)carbamate (72.6 mg, 244 μmol, 1.0 eq) in dimethyformamide (1 mL) was added 3-(4-(3-aminoazetidin-1-yl)-2,6-difluorophenyl)piperidine-2,6-dione IX (100 mg, 244. μmol, 1.00 eq, trifluoroacetic acid) and triethylamine (98.9 mg, 977 μmol, 4.00 eq). The reaction mixture was stirred at 30° C. for 1 h. The reaction mixture was filtered. The filtrate was purified by Prep-HPLC (column: Unisil 3-100 C18 Ultra 150*50 mm*3 μm; mobile phase: [water (0.225% formic acid)-acetonitrile]; B %: 35%-65%, 10 min) and lyophilized to afford 1-(3-(difluoromethoxy)-4-fluorophenyl)-3-(1-(4-(2,6-dioxopiperidin-3-yl)-3,5-difluorophenyl) azetidin-3-yl)urea #19 (47.3 mg, 94.9 μmol, 39% yield) as a white solid. ¹H NMR (400 MHz, DMSO-d₆) δ=10.86 (s, 1H), 8.82 (s, 1H), 7.60 (dd, J=2.4, 7.3 Hz, 1H), 7.36-7.00 (t, J=76 Hz, 1H), 7.28-7.22 (m, 1H), 7.20-7.13 (m, 1H), 7.03-6.91 (m, 1H), 6.17 (d, J=11.0 Hz, 2H), 4.61-4.49 (m, 1H), 4.13 (t, J=7.6 Hz, 2H), 4.04 (dd, J=5.0, 12.7 Hz, 1H), 3.66 (br t, J=6.1 Hz, 2H), 2.84-2.72 (m, 1H), 2.59 (br d, J=1.6 Hz, 1H), 2.16-2.01 (m, 1H), 1.99-1.87 (m, 1H). MS (ESI) m/z 499.3 [M+H]⁺.

### Example 21. Synthesis of Compound 20

To a solution of 3-(2-chloro-4-(3-hydroxyazetidin-1-yl)phenyl)piperidine-2,6-dione XXXII (90.0 mg, 328 μmol, 1.00 eq) in dimethyl formamide (2 mL) was added phenyl (3-chloro-4-methylphenyl)carbamate (105 mg, 394 μmol, 1.20 eq) and sodium hydride (26.2 mg, 656 μmol, 60% purity, 2.00 eq) at 10° C. The mixture was stirred at 10° C. for 1 h. The mixture was adjusted pH=6 with formic acid (0.100 mL). The mixture was filtered to give filtrate. The filtrate was purified by prep-HPLC (column: Phenomenex Synergi C18 150*25 mm*10 μm; mobile phase: [water (0.225% formic acid)-acetonitrile]; B %: 30%-60%, 10 min). The desired fraction was collected and lyophilized to give (2-(2,6-dioxopiperidin-3-yl)-3-oxoisoindolin-5-yl)methyl (4-cyano-3,5-dimethylphenyl)carbamate #20 (42.91 mg, 95.1 μmol, 29% yield, 99% purity) as a white solid. ¹H NMR (400 MHz, DMSO-d₆) δ=10.82 (s, 1H), 10.01 (br s, 1H), 7.58 (s, 1H), 7.34-7.21 (m, 2H), 7.10 (d, J=8.4 Hz, 1H), 6.54 (d, J=2.4 Hz, 1H), 6.44 (dd, J=2.3, 8.4 Hz, 1H), 5.38-5.21 (m, 1H), 4.21 (dd, J=6.6, 8.6 Hz, 2H), 4.04 (dd, J=5.0, 12.2 Hz, 1H), 3.81 (dd, J=3.9, 8.9 Hz, 2H), 2.74 (ddd, J=5.3, 12.6, 17.5 Hz, 1H), 2.47 (br s, 1H), 2.29-2.16 (m, 4H), 1.97-1.88 (m, 1H). MS (ESI) m/z 461.9 [M+H]⁺.

### Example 22. Synthesis of Compound 21

Step 1. To a solution of 3-(difluoromethoxy)-4-methylaniline (200 mg, 1.16 mmol, 1.00 eq) in acetonitrile (2 mL) were added pyridine (279 μL, 3.47 mmol, 3.00 eq) and phenyl carbonochloridate (173 μL, 1.39 mmol, 1.20 eq) at 0° C. Then the mixture was stirred at 20° C. for 2 h. The mixture was concentrated under reduced pressure to give a residue. The residue was diluted with water (10 mL), extracted with ethyl acetate (3×10 mL). The organic layer was dried over anhydrous sodium sulfate, filtered and concentrated under reduced pressure to give a crude product. The crude product was purified by reverse phase column chromatography (column: spherical C18, 20-45 μm, 100 Å, SW 120, mobile phase: [water (0.1% formic acid)-acetonitrile) and lyophilized to afford phenyl (3-(difluoromethoxy)-4-methylphenyl) carbamate (180 mg, 552 μmol, 47% yield, 90% purity) as an orange oil. ¹H NMR (400 MHz, DMSO-d₆) δ=10.33 (br s, 1H), 7.48-7.43 (m, 3H), 7.41-7.39 (m, 1H), 7.25-7.23 (m, 3H), 7.21 (s, 1H), 7.10 (t, J=74 Hz, 1H), 2.17 (s, 3H).

Step 2. To a solution of 3-(4-(3-aminoazetidin-1-yl)-2,6-difluorophenyl)piperidine-2,6-dione IX (100 mg, 292 μmol, 1.00 eq, formic acid) in dimethylformamide (1 mL) were added triethylamine (81.5 μL, 585 μmol, 2.00 eq) and phenyl (3-(difluoromethoxy)-4-methylphenyl)carbamate (94.5 mg, 322 μmol, 1.10 eq). Then the mixture was stirred at 30° C. for 12 h. The mixture was filtered. The filtrate was purified by Prep-HPLC (column: Phenomenex Synergi C18 150*25 mm*10 μm; mobile phase: [water (0.225% formic acid)-acetonitrile]; B %: 36%-66%, 10 min) and lyophilized to afford 1-(3-(difluoromethoxy)-4-methylphenyl)-3-(1-(4-(2,6-dioxopiperidin-3-yl)-3,5-difluorophenyl)azetidin-3-yl)urea #21 (37.4 mg, 67.8 μmol, 23% yield, 98% purity, formic acid) as a white solid. ¹H NMR (400 MHz, DMSO-d6) δ=10.86 (s, 1H), 8.78 (s, 1H), 7.41 (s, 1H), 7.15-7.11 (m, 1H), 7.07 (s, 1H), 7.05 (t, J=81.2 Hz, 1H), 6.97 (d, J=7.2 Hz, 1H), 6.19 (s, 1H), 6.16 (s, 1H), 4.59-4.51 (m, 1H), 4.12 (t, J=7.5 Hz, 2H), 4.04 (br dd, J=4.8, 12.5 Hz, 1H), 3.65 (br t, J=6.2 Hz, 2H), 2.83-2.73 (m, 1H), 2.56-2.52 (m, 1H), 2.13 (s, 3H), 2.08 (br dd, J=3.5, 13.0 Hz, 1H), 1.97-1.91 (m, 1H). MS (ESI) m/z 495.2 [M+H]⁺.

### Example 23. Synthesis of Compound 22

To a solution of 3-(4-(3-aminoazetidin-1-yl)-2,6-difluorophenyl)piperidine-2,6-dione IX (100 mg, 244 μmol, 1.00 eq, trifluoroacetic acid) in dimethylformamide (1 mL) was added triethylamine (102 μL, 732 μmol, 3.00 eq) and phenyl (3-(difluoromethoxy)-5-fluorophenyl)carbamate (79.8 mg, 268 μmol, 1.10 eq). Then the mixture was stirred at 30° C. for 2 h. The mixture was filtered. The filtrate was purified by Prep-HPLC (column: Phenomenex Synergi C18 150*25 mm*10 μm; mobile phase: [water (0.225% formic acid)-acetonitrile]; B %: 36%-66%, 10 min) and lyophilized to afford 1-(3-(difluoromethoxy)-5-fluorophenyl)-3-(1-(4-(2,6-dioxopiperidin-3-yl)-3,5-difluorophenyl)azetidin-3-yl)urea #22 (24.96 mg, 45.3 μmol, 18% yield, 99% purity, formic acid) as a white solid. ¹H NMR (400 MHz, DMSO-d6) δ=10.86 (s, 1H), 9.18 (s, 1H), 7.25-7.21 (m, 2H), 7.19 (t, J=88 Hz, 1H), 7.10 (s, 1H), 6.63 (br d, J=9.8 Hz, 1H), 6.19 (s, 1H), 6.16 (s, 1H), 4.59-4.51 (m, 1H), 4.13 (t, J=7.7 Hz, 2H), 4.04 (br dd, J=5.0, 12.5 Hz, 1H), 3.67 (br t, J=6.4 Hz, 2H), 2.83-2.73 (m, 1H), 2.53-2.52 (m, 1H), 2.13-2.03 (m, 1H), 1.97-1.91 (m, 1H). MS (ESI) m/z 499.2 [M+H]⁺.

### Example 24. Synthesis of Compound 23

To a mixture of 3-(4-(3-aminoazetidin-1-yl)-2,6-difluorophenyl)piperidine-2,6-dione IX (100 mg, 293 μmol, 1.00 eq, formic acid) and phenyl (6-(o-tolyl)pyridin-3-yl)carbamate (98.2 mg, 322 μmol, 1.10 eq) in dimethylformamide (2 mL) was added triethylamine (81.6 μL, 586 μmol, 2.00 eq). Then the mixture was stirred at 30° C. for 2 h. The reaction mixture was filtered. The residue was purified by Prep-HPLC (neutral condition; column: Waters Xbridge 150*25 mm*5 μm; mobile phase: [water (10 mM ammonium bicarbonate)-acetonitrile]; B %: 31%-61%, 9 min) to afford 1-(1-(4-(2,6-dioxopiperidin-3-yl)-3,5-difluorophenyl) azetidin-3-yl)-3-(6-(o-tolyl)pyridin-3-yl)urea #23 (68.2 mg, 135 μmol, 46% yield, 100% purity) as a white solid. ¹H NMR (400 MHz, DMSO-d₆) δ=11.05-10.69 (m, 1H), 8.99-8.83 (m, 1H), 8.63 (d, J=2.6 Hz, 1H), 7.96 (dd, J=2.8, 8.6 Hz, 1H), 7.40 (d, J=8.6 Hz, 1H), 7.38-7.33 (m, 1H), 7.29-7.23 (m, 3H), 7.13 (br t, J=6.2 Hz, 1H), 6.19 (d, J=11.0 Hz, 2H), 4.64-4.54 (m, 1H), 4.15 (t, J=7.6 Hz, 2H), 4.05 (dd, J=5.0, 12.6 Hz, 1H), 3.69 (br t, J=6.2 Hz, 2H), 2.86-2.72 (m, 1H), 2.49-2.46 (m, 1H), 2.32 (s, 3H), 2.16-2.02 (m, 1H), 2.00-1.90 (m, 1H). MS (ESI) m/z 506.1 [M+H]⁺.

### Example 25. Synthesis of Compound 24

To a mixture of 3-(4-(3-aminoazetidin-1-yl)-2,6-difluorophenyl)piperidine-2,6-dione IX (100 mg, 293 μmol, 1.00 eq, formic acid) and phenyl (3-chloro-4-methyl-5-(morpholinomethyl)phenyl)carbamate (116 mg, 322 μmol, 1.10 eq) in dimethylformamide (2 mL) was added triethylamine (81.6 μL, 586 μmol, 81.6 μL, 2.00 eq). Then the mixture was stirred at 30° C. for 2 h. The reaction mixture was filtered. The residue was purified by Prep-HPLC (neutral condition; column: Waters Xbridge 150*25 mm*5 μm; mobile phase: [water (10 mM ammonium bicarbonate)-acetonitrile]; B %: 40%-70%, 10 min) to afford 1-(3-chloro-4-methyl-5-(morpholinomethyl)phenyl)-3-(1-(4-(2,6-dioxopiperidin-3-yl)-3,5-difluorophenyl) azetidin-3-yl) urea #24 (42.9 mg, 74.7 μmol, 25% yield, 98% purity) as a white solid. ¹H NMR (400 MHz, DMSO-d₆) δ=10.87 (s, 1H), 8.74 (s, 1H), 7.59 (d, J=2.2 Hz, 1H), 7.15 (d, J=1.8 Hz, 1H), 6.93 (d, J=7.4 Hz, 1H), 6.18 (d, J=11.0 Hz, 2H), 4.61-4.50 (m, 1H), 4.13 (t, J=7.6 Hz, 2H), 4.05 (br dd, J=5.0, 12.3 Hz, 1H), 3.66 (br t, J=6.4 Hz, 2H), 3.56 (br t, J=4.2 Hz, 4H), 3.39 (s, 2H), 3.31 (br s, 1H), 2.84-2.73 (m, 1H), 2.36 (br s, 4H), 2.27 (s, 3H), 2.09 (br dd, J=3.8, 12.9 Hz, 1H), 1.99-1.92 (m, 1H). MS (ESI) m/z 562.2 [M+H]⁺.

### Example 26. Synthesis of Compound 25

To a solution of 3-(4-(3-aminoazetidin-1-yl)-2,6-difluorophenyl)piperidine-2,6-dione IX (100 mg, 292 μmol, 1.00 eq, formic acid) in dimethylformamide (2 mL) were added triethylamine (122 μL, 878 μmol, 3.00 eq) and phenyl (4-phenylpyridin-2-yl)carbamate (93.5 mg, 322 μmol, 1.10 eq). Then the mixture was stirred at 30° C. for 2 h. The mixture was filtered. The filtrate was purified by Prep-HPLC (column: Phenomenex Gemini-NX C18 75*30 mm*3 μm; mobile phase: [water-acetonitrile]; B %: 30%-60%, 8 min) and lyophilized to afford 1-(1-(4-(2,6-dioxopiperidin-3-yl)-3,5-difluorophenyl) azetidin-3-yl)-3-(4-phenylpyridin-2-yl) urea #25 (49.73 mg, 91.5 μmol, 31% yield, 99% purity) as a white solid. ¹H NMR (400 MHz, DMSO-d₆) δ=10.87 (s, 1H), 9.38 (s, 1H), 8.70 (br d, J=7.0 Hz, 1H), 8.27 (d, J=5.4 Hz, 1H), 7.70 (s, 2H), 7.68 (s, 1H), 7.55-7.48 (m, 3H), 7.27 (dd, J=1.6, 5.4 Hz, 1H), 6.21 (s, 1H), 6.19 (s, 1H), 4.68-4.61 (m, 1H), 4.18 (t, J=7.6 Hz, 2H), 4.05 (br dd, J=5.1, 12.3 Hz, 1H), 3.74-3.70 (m, 2H), 2.81-2.75 (m, 1H), 2.52 (br s, 1H), 2.14-2.07 (m, 1H), 1.95 (ddd, J=2.4, 5.4, 7.7 Hz, 1H). MS (ESI) m/z 492.2 [M+H]⁺.

### Example 27. Synthesis of Compound 26

To a solution of phenyl (4-(pyridin-2-yl)phenyl)carbamate (80.0 mg, 275 μmol, 1.00 eq) in dimethylformamide (2 mL) were added triethylamine (115 μL, 826 μmol, 3.00 eq) and 3-(4-(3-aminoazetidin-1-yl)-2,6-difluorophenyl)piperidine-2,6-dione IX (90.2 mg, 220 μmol, 0.800 eq, trifluoroacetic acid). Then the mixture was stirred at 30° C. for 4 h. The mixture was filtered. The filtrate was purified by Prep-HPLC (column: Waters Xbridge 150*25 mm*5 μm; mobile phase: [water (10 mM ammonium bicarbonate)-acetonitrile]; B %: 33%-63%, 9 min) and lyophilized to give a residue. The residue was purified by Prep-HPLC (column: Phenomenex Synergi C18 150*25 mm*10 μm; mobile phase: [water (0.225% formic acid)-acetonitrile]; B %: 19%-39%, 9 min) and lyophilized to afford 1-(1-(4-(2,6-dioxopiperidin-3-yl)-3,5-difluorophenyl)azetidin-3-yl)-3-(4-(pyridin-2-yl)phenyl)urea #26 (15.92 mg, 29.3 μmol, 10% yield, 99% purity, formic acid) as a white solid. ¹H NMR (400 MHz, DMSO-d6) δ=10.85 (s, 1H), 8.82 (s, 1H), 8.60 (d, J=4.2 Hz, 1H), 7.98 (d, J=8.8 Hz, 2H), 7.89-7.86 (m, 1H), 7.84-7.79 (m, 1H), 7.52 (d, J=8.7 Hz, 2H), 7.26 (dd, J=5.4, 6.6 Hz, 1H), 7.02 (d, J=7.2 Hz, 1H), 6.19 (s, 1H), 6.17 (s, 1H), 4.61-4.55 (m, 1H), 4.15 (t, J=7.6 Hz, 2H), 4.04 (br dd, J=5.2, 12.5 Hz, 1H), 3.68 (br t, J=6.4 Hz, 2H), 2.83-2.74 (m, 1H), 2.52 (br s, 1H), 2.15-2.07 (m, 1H), 1.98-1.92 (m, 1H). MS (ESI) m/z 492.3 [M+H]⁺.

### Example 28. Synthesis of Compound 27

Step 1. To a solution of 4-(trifluoromethoxy)pyridin-2-amine (100 mg, 561 μmol, 1.00 eq) in acetonitrile (4 mL) were added pyridine (135 μL, 1.68 mmol, 3.00 eq) and phenyl carbonochloridate (77.3 μL, 617 μmol, 1.10 eq). Then the mixture was stirred at 20° C. for 30 min. The reaction mixture was concentrated under reduced pressure to give a residue. The residue was triturated with water (10 mL) and filtered. The filter cake was washed with acetonitrile (1 mL) and concentrated under reduced pressure to afford phenyl (4-(trifluoromethoxy)pyridin-2-yl)carbamate (100 mg, 335 μmol, 59% yield) as a white solid. ¹H NMR (400 MHz, DMSO-d₆) δ=11.18 (s, 1H), 8.44 (d, J=5.7 Hz, 1H), 7.79 (s, 1H), 7.46-7.42 (m, 2H), 7.31-7.28 (m, 1H), 7.27-7.23 (m, 2H), 7.15-7.12 (m, 1H).

Step 2. To a solution of 3-(4-(3-aminoazetidin-1-yl)-2,6-difluorophenyl)piperidine-2,6-dione IX (100 mg, 244 μmol, 1.00 eq, trifluoroacetic acid) in dimethylformamide (2 mL) was added triethylamine (102 μL, 732 μmol, 3.00 eq) and phenyl (4-(trifluoromethoxy)pyridin-2-yl)carbamate (80.1 mg, 268 μmol, 1.10 eq). Then the mixture was stirred at 30° C. for 2 h. Then phenyl (4-(trifluoromethoxy)pyridin-2-yl)carbamate (18.2 mg, 61.0 μmol, 0.250 eq) was added into the mixture and the mixture was stirred at 30° C. for 2 h. The mixture was filtered. The filtrate was purified by Prep-HPLC (column: Phenomenex luna C18 150*25 mm*10 μm; mobile phase: [water (0.2% formic acid)-acetonitrile]; B %: 34%-64%, 12 min) and lyophilized to afford 1-(1-(4-(2,6-dioxopiperidin-3-yl)-3,5-difluorophenyl)azetidin-3-yl)-3-(4-(trifluoromethoxy)pyridin-2-yl)urea #27 (41.36 mg, 72.8 μmol, 29% yield, 96% purity, formic acid) as a white solid. ¹H NMR (400 MHz, DMSO-d₆) δ=10.86 (s, 1H), 9.53 (s, 1H), 8.31 (d, J=5.7 Hz, 1H), 8.13 (br d, J=7.1 Hz, 1H), 7.58 (s, 1H), 6.96 (dd, J=1.1, 5.7 Hz, 1H), 6.20 (s, 1H), 6.17 (s, 1H), 4.62-4.57 (m, 1H), 4.16 (t, J=7.8 Hz, 2H), 4.04 (br dd, J=5.3, 12.8 Hz, 1H), 3.69 (br t, J=6.0 Hz, 2H), 2.82-2.73 (m, 1H), 2.52 (br s, 1H), 2.13-2.07 (m, 1H), 1.97-1.92 (m, 1H). MS (ESI) m/z 500.2 [M+H]⁺.

### Example 29. Synthesis of Compound 28

To a mixture of 3-(4-(3-aminoazetidin-1-yl)-2,6-difluorophenyl)piperidine-2,6-dione IX (100 mg, 293 μmol, 1.00 eq, formic acid) and phenyl (3-chloro-5-(difluoromethoxy)phenyl)carbamate (101 mg, 322 μmol, 1.10 eq) in dimethylformamide (2.50 mL) was added triethylamine (81.6 μL, 586 μmol, 2.00 eq). Then the mixture was stirred at 30° C. 2 h. The reaction mixture was filtered. The residue was purified by Prep-HPLC (neutral condition; column: Waters Xbridge 150*25 mm*5 μm; mobile phase: [water (10 mM ammonium bicarbonate)-acetonitrile]; B %: 42%-72%, 10 min) and lyophilized to afford a crude product. The product was further purified by Prep-HPLC (basic condition; column: Phenomenex Synergi C18 150*25 mm*10 μm; mobile phase: [water (0.225% formic acid)-acetonitrile]; B %: 37%-70%, 11 min) to afford 1-(3-chloro-5-(difluoromethoxy) phenyl)-3-(1-(4-(2,6-dioxopiperidin-3-yl)-3,5-difluorophenyl)azetidin-3-yl)urea #28 (64.9 mg, 126 μmol, 43% yield, 99% purity) as a white solid. ¹H NMR (400 MHz, DMSO-d₆) δ=10.87 (br s, 1H), 10.11 (br s, 1H), 8.43 (br s, 1H), 8.18 (br s, 1H), 7.42 (s, 1H), 7.32 (s, 1H), 7.23 (t, J=74 Hz, 1H), 6.79 (s, 1H), 6.17 (br d, J=11.2 Hz, 2H), 4.60-4.50 (m, 1H), 4.13 (br t, J=7.6 Hz, 2H), 4.05 (br dd, J=4.8, 12.5 Hz, 1H), 3.66 (br t, J=6.2 Hz, 2H), 2.85-2.73 (m, 1H), 2.15-2.02 (m, 1H), 2.01-1.89 (m, 2H). MS (ESI) m/z 562.2 [M+H]⁺.

### Example 30. Synthesis of Compound 29

To a solution of 3-(2-chloro-5-fluoro-4-(3-hydroxyazetidin-1-yl)phenyl)piperidine-2,6-dione XL (80.0 mg, 256 μmol, 1.00 eq) and sodium hydride (20.5 mg, 512 μmol, 60% purity, 2.00 eq) in dimethylformamide (1 mL) was added phenyl (3-chloro-4-methylphenyl)carbamate (80.3 mg, 307 μmol, 1.20 eq). The mixture was stirred at 25° C. for 1 h. The mixture was filtered. The filtrate was purified by Prep-HPLC (column: Phenomenex luna C18 150*25 mm*10 μm; mobile phase: [water (0.2% formic acid)-acetonitrile]; B %: 45%-75%, 12 min) and lyophilized to afford 3-(2-chloro-5-fluoro-4-(3-hydroxyazetidin-1-yl)phenyl)-1-((2-(trimethylsilyl)ethoxy)methyl)piperidine-2,6-dione #29 (41.9 mg, 87.4 μmol, 34% yield) as a white solid. ¹H NMR (400 MHz, DMSO-d₆) δ=10.84 (br s, 1H), 10.02 (br s, 1H), 7.58 (s, 1H), 7.32-7.28 (m, 1H), 7.28-7.26 (m, 1H), 7.12 (d, J=13.2 Hz, 1H), 6.68 (d, J=8.4 Hz, 1H), 5.28 (tt, J=4.1, 6.2 Hz, 1H), 4.32 (br t, J=7.1 Hz, 2H), 4.08 (dd, J=5.0, 12.6 Hz, 1H), 3.94 (br dd, J=3.8, 8.7 Hz, 2H), 2.82-2.72 (m, 1H), 2.54 (br dd, J=2.6, 4.2 Hz, 1H), 2.34-2.28 (m, 1H), 2.26 (s, 3H), 1.96-1.88 (m, 1H). MS (ESI) m/z 480.1 [M+H]⁺.

### Example 31. Synthesis of Compound 30

To a solution of (R)-phenyl (6-(2-methylpyrrolidin-1-yl)pyridin-3-yl)carbamate (95.8 mg, 0.322 mmol, 1.10 eq) in dimethyformamide (1 mL) was added 3-(4-(3-aminoazetidin-1-yl)-2,6-difluorophenyl)piperidine-2,6-dione (100 mg, 0.293 mmol, 1.00 eq, formic acid) and triethylamine (164 μL, 1.17 mmol, 4.00 eq). The reaction was stirred at 30° C. for 30 min. The reaction mixture was filtered. The filtrate was purified by Prep-HPLC (column: Phenomenex Synergi C18 150*25 mm*10 μm; mobile phase: [water (0.225% formic acid)-acetonitrile]; B %: 5%-35%, 10 min) and lyophilized to afford a residue. The residue was purified by Prep-HPLC (column: Phenomenex Gemini-NX C18 75*30 mm*3 μm; mobile phase: [water-acetonitrile]; B %: 20%-50%, 8 min) and lyophilized to afford 1-(1-(4-(2,6-dioxopiperidin-3-yl)-3,5-difluorophenyl)azetidin-3-yl)-3-(6-((R)-2-methylpyrrolidin-1-yl)pyridin-3-yl)urea #30 (51.74 mg, 0.101 mmol, 35% yield, 98% purity) as a white solid. ¹H NMR (400 MHz, DMSO-d₆) δ=10.86 (s, 1H), 8.09 (br s, 1H), 7.99 (d, J=2.2 Hz, 1H), 7.53 (dd, J=2.2, 8.9 Hz, 1H), 6.74 (br d, J=6.5 Hz, 1H), 6.38 (br d, J=8.8 Hz, 1H), 6.17 (d, J=11.0 Hz, 2H), 4.61-4.49 (m, 1H), 4.12 (br t, J=7.8 Hz, 2H), 4.08-3.99 (m, 2H), 3.64 (br t, J=6.4 Hz, 2H), 3.45-3.36 (m, 1H), 3.25-3.16 (m, 1H), 2.86-2.72 (m, 1H), 2.16-1.83 (m, 6H), 1.71-1.57 (m, 1H), 1.13 (d, J=6.2 Hz, 3H). MS (ESI) m/z 499.3 [M+H]⁺.

### Example 32. Synthesis of Compound 31

To a solution of 3-(2-chloro-4-(3-hydroxyazetidin-1-yl)phenyl)piperidine-2,6-dione XXXII (90.0 mg, 305 μmol, 1.00 eq) in dimethyl formamide (2 mL) was added phenyl (3,5-dimethylphenyl)carbamate (88.4 mg, 366 μmol, 1.20 eq) and sodium hydride (24.4 mg, 610 μmol, 60% purity, 2.00 eq) at 0° C. The mixture was stirred at 0° C. for 1 h. The mixture was adjusted pH=5 with formic acid (0.300 mL). The mixture was filtered to give filtrate which was purified by prep-HPLC (column: Phenomenex Synergi C18 150*25 mm*10 μm; mobile phase: [water (0.225% formic acid)-acetonitrile]; B %: 46%-76%, 10 min). The desired fraction was collected and lyophilized to give 1-(3-chloro-4-(2,6-dioxopiperidin-3-yl)phenyl)azetidin-3-yl (3,5-dimethylphenyl)carbamate #31 (83.73 mg, 187 μmol, 61% yield, 99% purity) as a white solid. ¹H NMR (400 MHz, DMSO-d₆) δ=10.82 (s, 1H), 9.73 (s, 1H), 7.14-7.06 (m, 3H), 6.65 (s, 1H), 6.54 (d, J=2.4 Hz, 1H), 6.44 (dd, J=2.4, 8.4 Hz, 1H), 5.26 (ddd, J=2.3, 3.9, 6.3 Hz, 1H), 4.24-4.18 (m, 2H), 4.04 (dd, J=5.0, 12.2 Hz, 1H), 3.80 (dd, J=4.0, 8.8 Hz, 2H), 2.77-2.68 (m, 1H), 2.29-2.12 (m, 8H), 1.97-1.89 (m, 1H). MS (ESI) m/z 442.1 [M+H]⁺.

### Example 33. Synthesis of Compound 32

To a solution of 3-(2,6-difluoro-4-(3-hydroxyazetidin-1-yl)phenyl)piperidine-2,6-dione XII (80.0 mg, 270 μmol, 1.00 eq) in dimethylformamide (1 mL) was added phenyl (3-(difluoromethoxy)-4-fluorophenyl) carbamate (104 mg, 351 μmol, 1.30 eq) and sodium hydride (21.6 mg, 540 μmol, 60% purity, 2.00 eq) at 0° C. The mixture was stirred at 20° C. for 1 h. The pH of the mixture was adjusted to 7 with hydrochloric acid (1 M) and diluted with dimethyl formamide (1 mL). The solution was purified by Prep-HPLC (column: Phenomenex luna C18 150*25 mm*10 μm; mobile phase: [water (0.2% formic acid)-acetonitrile]; B %: 38%-68%, 12 min) to afford 1-(4-(2,6-dioxopiperidin-3-yl)-3,5-difluorophenyl)azetidin-3-yl (3-(difluoromethoxy)-4-fluorophenyl)carbamate #32 (32.85 mg, 65.8 μmol, 24% yield) as a white solid. ¹H NMR (400 MHz, DMSO-d₆) δ=10.88 (br s, 1H), 10.14 (s, 1H), 7.58 (br d, J=6.0 Hz, 1H), 7.20 (t, J=72 Hz, 1H), 7.38-7.32 (m, 1H), 7.32-7.26 (m, 1H), 6.24 (s, 1H), 6.22 (s, 1H), 5.34-5.28 (m, 1H), 4.24 (br t, J=7.8 Hz, 2H), 4.06 (dd, J=5.2, 12.6 Hz, 1H), 3.84 (br dd, J=3.4, 9.2 Hz, 2H), 2.84-2.74 (m, 1H), 2.56-2.54 (m, 1H), 2.14-2.02 (m, 1H), 1.98-1.92 (m, 1H). MS (ESI) m/z 500.0 [M+H]⁺.

### Example 34. Synthesis of Compound 33

To a solution of 3-(2,6-difluoro-4-(3-hydroxyazetidin-1-yl)phenyl)piperidine-2,6-dione XII (100 mg, 338 μmol, 1.10 eq) in dimethyl formamide (0.5 mL) were added sodium hydride (24.6 mg, 614 μmol, 60% purity, 2.00 eq) and phenyl (2-fluoro-5-(trifluoromethoxy)phenyl)carbamate (96.7 mg, 307 μmol, 1.00 eq). The mixture was stirred at 25° C. for 1 h. The pH of the mixture was adjusted to 7 with formic acid and diluted with dimethyl formamide (1 mL). The reaction mixture was purified by Prep-HPLC (column: Phenomenex Synergi C18 150*25 mm*10 μm; mobile phase: [water (0.225% formic acid)-acetonitrile]; B %: 46%-76%, 10 min) and the desired eluent was lyophilized to afford 1-(4-(2,6-dioxopiperidin-3-yl)-3,5-difluorophenyl)azetidin-3-yl (2-fluoro-5-(trifluoromethoxy)phenyl)carbamate #33 (42.3 mg, 80.9 μmol, 26% yield, 99% purity) as a white solid. ¹H NMR (400 MHz, DMSO-d₆) δ=10.87 (s, 1H), 10.12-9.83 (m, 1H), 7.80 (br d, J=2.8 Hz, 1H), 7.39 (dd, J=9.0, 10.4 Hz, 1H), 7.19-7.12 (m, 1H), 6.23 (d, J=11.2 Hz, 2H), 5.37-5.23 (m, 1H), 4.28-4.16 (m, 2H), 4.05 (br dd, J=4.9, 12.4 Hz, 1H), 3.85 (br dd, J=3.3, 8.8 Hz, 2H), 2.88-2.69 (m, 1H), 2.52 (br d, J=2.0 Hz, 1H), 2.14-2.01 (m, 1H), 2.00-1.89 (m, 1H). MS (ESI) m/z 517.9 [M+H]⁺.

### Example 35. Synthesis of Compound 34

To a solution of 3-(2,6-difluoro-4-(3-hydroxyazetidin-1-yl)phenyl)piperidine-2,6-dione XII (100 mg, 338 μmol, 1.00 eq) in dimethylformamide (0.500 mL) was added sodium hydride (27.0 mg, 675 μmol, 60% purity, 2.00 eq) and phenyl (3-chloro-5-fluorophenyl)carbamate (98.6 mg, 371 μmol, 1.10 eq) at 0° C. The mixture was stirred at 25° C. for 1 h. The pH of the mixture was adjusted to 7 with formic acid and diluted with dimethylformamide (1 mL). The reaction mixture was purified by Prep-HPLC (column: Phenomenex Synergi C18 150*25 mm*10 μm; mobile phase: [water (0.225% formic acid)-acetonitrile]; B %: 46%-76%, 10 min) and lyophilized to afford 1-(4-(2,6-dioxopiperidin-3-yl)-3,5-difluorophenyl)azetidin-3-yl(3-chloro-5-fluorophenyl)carbamate #34 (51.2 mg, 108 μmol, 32% yield, 99% purity) as a white solid. ¹H NMR (400 MHz, DMSO-d₆) δ=10.87 (br s, 1H), 10.36 (br s, 1H), 7.37 (s, 1H), 7.31 (td, J=2.0, 11.0 Hz, 1H), 7.07 (td, J=2.0, 8.5 Hz, 1H), 6.22 (d, J=11.0 Hz, 2H), 5.30 (td, J=3.1, 6.8 Hz, 1H), 4.22 (dd, J=7.3, 8.3 Hz, 2H), 4.05 (br dd, J=5.3, 12.8 Hz, 1H), 3.85 (br dd, J=3.5, 9.0 Hz, 2H), 2.84-2.72 (m, 1H), 2.52 (br d, J=1.5 Hz, 1H), 2.16-2.01 (m, 1H), 1.99-1.89 (m, 1H). MS (ESI) m/z 468.2 [M+H]⁺.

### Example 36. Synthesis of Compound 35

To a solution of 3-(4-(3-aminoazetidin-1-yl)-2-chlorophenyl)piperidine-2,6-dione XLIII (100 mg, 0.340 mmol, 1.00 eq) and phenyl (3,5-dimethylphenyl)carbamate (90.4 mg, 0.374 mmol, 1.10 eq) in dimethyl formamide (1.50 mL) was added triethylamine (142 μL, 1.02 mmol, 3.00 eq). The mixture was stirred at 30° C. for 12 h. The reaction mixture was concentrated under reduced pressure to give a residue. The residue was purified by prep-HPLC (column: Phenomenex Synergi C18 150*25 mm*10 μm; mobile phase: [water (0.225% formic acid)-acetonitrile]; B %: 33%-66%, 11 min) to afford 1-(1-(3-chloro-4-(2,6-dioxopiperidin-3-yl)phenyl) azetidin-3-yl)-3-(3,5-dimethylphenyl)urea #35 (31.8 mg, 68.5 μmol, 20% yield, 95% purity) as a white solid. ¹H NMR (400 MHz, DMSO-d₆) δ=10.82 (s, 1H), 8.35 (s, 1H), 7.09 (d, J=8.4 Hz, 1H), 7.01 (s, 2H), 6.78 (br d, J=7.5 Hz, 1H), 6.55 (s, 1H), 6.50 (d, J=2.3 Hz, 1H), 6.41 (dd, J=2.4, 8.4 Hz, 1H), 4.59-4.49 (m, 1H), 4.12 (t, J=7.5 Hz, 2H), 4.04 (dd, J=5.0, 12.2 Hz, 1H), 3.64-3.59 (m, 2H), 2.79-2.66 (m, 2H), 2.24 (br s, 1H), 2.19 (s, 6H), 1.96-1.89 (m, 1H). MS (ESI) m/z 441.3 [M+H]⁺.

### Example 37. Synthesis of Compound 36

To a solution of 3-(2-chloro-4-(3-hydroxyazetidin-1-yl)phenyl)piperidine-2,6-dione XXXII (90.0 mg, 305 μmol, 1.00 eq) in dimethyl formamide (1 mL) was added phenyl (3-(trifluoromethoxy)phenyl)carbamate (109 mg, 367 μmol, 1.20 eq) and sodium hydride (24.4 mg, 610 μmol, 60% purity, 2.00 eq) at 0° C. The mixture was stirred at 0° C. for 1 h. The mixture was adjusted pH=6 with formic acid (0.500 mL). The mixture was filtered to give filtrate. The filtrate was purified by prep-HPLC (column: Phenomenex Synergi C18 150*25 mm*10 μm; mobile phase: [water (0.225% formic acid)-acetonitrile]; B %: 49%-79%, 10 min). The desired fraction was collected and lyophilized to give 1-(3-chloro-4-(2,6-dioxopiperidin-3-yl)phenyl)azetidin-3-yl (3-(trifluoromethoxy)phenyl)carbamate #36 (67.2 mg, 133 μmol, 44% yield, 99% purity) as a white solid. ¹H NMR (400 MHz, DMSO-d₆) δ=10.85 (s, 1H), 10.26 (br s, 1H), 7.60 (br s, 1H), 7.46-7.40 (m, 2H), 7.12 (d, J=8.4 Hz, 1H), 7.04-6.97 (m, 1H), 6.56 (d, J=2.4 Hz, 1H), 6.45 (dd, J=2.3, 8.4 Hz, 1H), 5.34-5.28 (m, 1H), 4.23 (dd, J=6.6, 8.7 Hz, 2H), 4.05 (dd, J=5.0, 12.2 Hz, 1H), 3.84 (dd, J=3.9, 8.9 Hz, 2H), 2.78-2.70 (m, 1H), 2.49-2.47 (m, 1H), 2.23 (dq, J=4.1, 12.8 Hz, 1H), 1.97-1.89 (m, 1H). MS (ESI) m/z 498.1 [M+H]⁺.

### Example 38. Synthesis of Compound 37

To a solution of 3-(2,6-difluoro-4-(3-hydroxyazetidin-1-yl)phenyl)piperidine-2,6-dione XII (100 mg, 337 μmol, 1.10 eq) in dimethyl formamide (0.500 mL) were added phenyl (3-cyclopropoxyphenyl)carbamate (82.6 mg, 307 μmol, 1.00 eq) and sodium hydride (24.6 mg, 614 μmol, 60% purity, 2.00 eq). The mixture was stirred at 25° C. for 1 h. The pH of the mixture was adjusted to 7 with formic acid and diluted with dimethyl formamide (1 mL). The residue was purified by Prep-HPLC (column: Phenomenex Synergi C18 150*25 mm*10 μm; mobile phase: [water (0.225% formic acid)-acetonitrile]; B %: 43%-73%, 10 min) followed by Prep-NPLC (column: Welch Ultimate XB-SiOH 250*50*10 μm; mobile phase: [hexane-isopropyl alcohol]; B %: 15%-15%, 15 min) to afford 1-(4-(2,6-dioxopiperidin-3-yl)-3,5-difluorophenyl)azetidin-3-yl (3-cyclopropoxyphenyl)carbamate #37 (29.12 mg, 61.1 μmol, 20% yield, 99% purity) as a white solid. ¹H NMR (400 MHz, DMSO-d₆) δ=10.88 (br s, 1H), 9.91 (br s, 1H), 7.27 (br s, 1H), 7.22-7.14 (m, 1H), 7.03 (br d, J=7.9 Hz, 1H), 6.70 (dd, J=2.0, 8.2 Hz, 1H), 6.22 (d, J=11.0 Hz, 2H), 5.31-5.23 (m, 1H), 4.22 (br t, J=7.8 Hz, 2H), 4.05 (br dd, J=5.0, 12.5 Hz, 1H), 3.83 (br dd, J=3.4, 9.1 Hz, 2H), 3.76 (tt, J=2.9, 5.9 Hz, 1H), 2.86-2.72 (m, 1H), 2.57 (br d, J=2.1 Hz, 1H), 2.16-2.01 (m, 1H), 1.99-1.89 (m, 1H), 0.81-0.71 (m, 2H), 0.68-0.59 (m, 2H). MS (ESI) m/z 472.2 [M+H]⁺.

### Example 39. Synthesis of Compound 38

To a mixture of phenyl (3-chloro-5-(difluoromethoxy)phenyl)carbamate (138 mg, 439 μmol, 1.30 eq) and 3-(2,6-difluoro-4-(3-hydroxyazetidin-1-yl)phenyl)piperidine-2,6-dione XII (100 mg, 338 μmol, 1.00 eq) in dimethylformamide (1 mL) was added sodium hydride (27.0 mg, 675 μmol, 60% purity, 2.00 eq) at 0° C. The mixture was stirred at 20° C. for 1 h. The mixture was quenched with 1 M hydrochloric and filtered. The filtrate was purified by prep-HPLC (column: Phenomenex luna C18 150*25 mm*10 μm; mobile phase: [water (0.225% formic acid)-acetonitrile]; B %: 49%-79%, 10 min) and lyophilized to afford 1-(4-(2,6-dioxopiperidin-3-yl)-3,5-difluorophenyl)azetidin-3-yl(3-chloro-5-(difluoromethoxy)phenyl) carbamate #38 (33.51 mg, 65.0 μmol, 19% yield, 99% purity) as a white solid. ¹H NMR (400 MHz, DMSO-d₆) δ=10.88 (s, 1H), 10.36 (br s, 1H), 7.46 (t, J=73.6 Hz, 1H), 7.42 (s, 1H), 7.32 (s, 1H), 7.08 (s, 1H), 6.24 (d, J=11.0 Hz, 2H), 5.38-5.24 (m, 1H), 4.24 (br t, J=7.7 Hz, 2H), 4.06 (br dd, J=4.9, 12.5 Hz, 1H), 3.86 (br dd, J=3.2, 9.0 Hz, 2H), 2.86-2.74 (m, 1H), 2.54-2.52 (m, 1H), 2.14-2.04 (m, 1H), 1.98-1.92 (m, 1H). MS (ESI) m/z 516.1 [M+H]⁺.

### Example 40. Synthesis of Compound 39

A mixture of 3-(2,6-difluoro-4-(3-hydroxyazetidin-1-yl)phenyl)piperidine-2,6-dione XII (150 mg, 0.506 mmol, 1.00 eq) and phenyl (4-chloro-2-fluoro-5-methylphenyl) carbamate (169 mg, 0.607 mmol, 1.20 eq) in dimethyl formamide was added sodium hydride (40.5 mg, 1.01 mmol, 60% purity, 2.00 eq) at 0° C. The reaction mixture was stirred at 20° C. for 1 h. The reaction mixture was added formic acid (2.00 mL) and filtered to give a filtrate, which was purified by Prep-HPLC (column: Phenomenex luna C18 150*25 mm*10 μm; mobile phase: [water (0.225% formic acid)-acetonitrile]; B %: 53%-83%, 10 min) and lyophilized to give 1-(4-(2,6-dioxopiperidin-3-yl)-3,5-difluorophenyl)azetidin-3-yl(4-chloro-2-fluoro-5-methylphenyl)carbamate #39 (95.88 mg, 0.196 mmol, 38% yield, 99% purity) as an off-white solid. ¹H NMR (400 MHz, DMSO-d₆) δ=10.89 (s, 1H), 9.70 (br s, 1H), 7.61 (br d, J=8.4 Hz, 1H), 7.44 (d, J=10.4 Hz, 1H), 6.24 (s, 1H), 6.22 (s, 1H), 5.34-5.20 (m, 1H), 4.21 (br t, J=7.6 Hz, 2H), 4.05 (br dd, J=5.2, 12.5 Hz, 1H), 3.83 (br dd, J=2.8, 8.8 Hz, 2H), 2.83-2.74 (m, 1H), 2.53-2.52 (m, 1H), 2.28 (s, 3H), 2.14-2.03 (m, 1H), 2.00-1.90 (m, 1H). MS (ESI) m/z 482.0 [M+H]⁺.

### Example 41. Synthesis of Compound 40

To a solution of 3-(4-(3-aminoazetidin-1-yl)-2,6-difluorophenyl)piperidine-2,6-dione IX (100 mg, 293 μmol, 1.00 eq, formate) and triethylamine (122 μL, 879 μmol, 3.00 eq) in dimethylformamide (1 mL) was added phenyl (6-(1-methylcyclopropyl)pyridin-3-yl)carbamate (86.5 mg, 322 μmol, 1.10 eq). The mixture was stirred at 30° C. for 12 h. The mixture was filtered. The filtrate was purified by Prep-HPLC (column: Waters Xbridge 150*25 mm*5 μm; mobile phase: [water (10 mM ammonium bicarbonate)-acetonitrile]; B %: 34%-64%, 8 min) and lyophilized to afford 1-(1-(4-(2,6-dioxopiperidin-3-yl)-3,5-difluorophenyl)azetidin-3-yl)-3-(6-(1-methylcyclopropyl)pyridin-3-yl)urea #40 (68.68 mg, 145 μmol, 49% yield, 99% purity) as a white solid. ¹H NMR (400 MHz, DMSO-d₆) δ=10.86 (s, 1H), 8.60 (s, 1H), 8.37 (d, J=2.6 Hz, 1H), 7.78 (dd, J=2.6, 8.6 Hz, 1H), 7.21 (d, J=8.7 Hz, 1H), 6.93 (d, J=7.3 Hz, 1H), 6.19 (s, 1H), 6.16 (s, 1H), 4.61-4.51 (m, 1H), 4.13 (t, J=7.6 Hz, 2H), 4.04 (dd, J=5.0, 12.7 Hz, 1H), 3.65 (br t, J=6.2 Hz, 2H), 2.84-2.72 (m, 1H), 2.52-2.52 (m, 1H), 2.15-2.02 (m, 1H), 1.99-1.90 (m, 1H), 1.43 (s, 3H), 1.09-1.03 (m, 2H), 0.74-0.68 (m, 2H). MS (ESI) m/z 470.3 [M+H]⁺.

### Example 42. Synthesis of Compound 41

To a solution of 3-(4-(3-aminoazetidin-1-yl)-2-chlorophenyl)piperidine-2,6-dione XLIII (100 mg, 0.340 mmol, 1.00 eq) and phenyl (3-(trifluoromethoxy)phenyl)carbamate (111 mg, 0.374 mmol, 1.10 eq) in dimethyl formamide (1.50 mL) was added triethylamine (142 μL, 1.02 mmol, 3.00 eq). The mixture was stirred at 30° C. for 12 h. The reaction mixture was concentrated under reduced pressure to give a residue. The residue was purified by prep-HPLC (column: Phenomenex Synergi C18 150*25 mm*10 μm; mobile phase: [water (0.225% formic acid)-acetonitrile]; B %: 37%-70%, 11 min) to afford 1-(1-(3-chloro-4-(2,6-dioxopiperidin-3-yl)phenyl) azetidin-3-yl)-3-(3-(trifluoromethoxy)phenyl)urea #41 (58.8 mg, 0.116 mmol, 34% yield, 98% purity) as a white solid. ¹H NMR (400 MHz, DMSO-d₆) δ=10.82 (s, 1H), 9.01 (s, 1H), 7.65 (s, 1H), 7.36-7.30 (m, 1H), 7.27-7.22 (m, 1H), 7.13-7.04 (m, 2H), 6.87 (br d, J=8.1 Hz, 1H), 6.51 (d, J=2.1 Hz, 1H), 6.41 (dd, J=2.1, 8.3 Hz, 1H), 4.61-4.52 (m, 1H), 4.13 (t, J=7.4 Hz, 2H), 4.04 (dd, J=4.9, 12.1 Hz, 1H), 3.64 (t, J=6.7 Hz, 2H), 2.77-2.67 (m, 1H), 2.48 (br s, 1H), 2.22 (dq, J=4.5, 12.7 Hz, 1H), 1.97-1.88 (m, 1H). MS (ESI) m/z 497.2 [M+H]⁺.

### Example 43. Synthesis of Compound 42

To a solution of 3-(4-(3-aminoazetidin-1-yl)-2-chlorophenyl)piperidine-2,6-dione XLIII (100 mg, 0.340 mmol, 1.00 eq) and phenyl (3-chloro-4-methylphenyl)carbamate (107 mg, 0.408 mmol, 1.20 eq) in dimethyl formamide (1.50 mL) was added triethylamine (142 μL, 1.02 mmol, 3.00 eq) at 30° C. The mixture was stirred at 30° C. for 12 h. The reaction mixture was quenched by addition water (50 mL), and extracted with ethyl acetate (3×50 mL). The combined organic layers were washed with water (3×50 mL), dried over anhydrous sodium sulfate, filtered and concentrated under reduced pressure to give a residue. The residue was purified by column chromatography (SiO₂, petroleum ether/ethyl acetate=1/0 to 1/5) to give a white solid. The white solid was diluted with water (3 mL) and lyophilized to afford 1-(1-(3-chloro-4-(2,6-dioxopiperidin-3-yl)phenyl)azetidin-3-yl)-3-(3-chloro-4-methylphenyl)urea #42 (10.3 mg, 0.0215 mmol, 6% yield, 96% purity) as a white solid. ¹H NMR (400 MHz, DMSO-d₆) δ=10.82 (s, 1H), 8.65 (s, 1H), 7.63 (d, J=1.8 Hz, 1H), 7.20-7.12 (m, 2H), 7.09 (d, J=8.4 Hz, 1H), 6.89 (d, J=7.4 Hz, 1H), 6.50 (d, J=2.1 Hz, 1H), 6.41 (dd, J=2.3, 8.4 Hz, 1H), 4.60-4.51 (m, 1H), 4.13 (t, J=7.5 Hz, 2H), 4.04 (dd, J=5.0, 12.2 Hz, 1H), 3.63 (br t, J=6.7 Hz, 2H), 2.78-2.69 (m, 1H), 2.48-2.45 (m, 1H), 2.27-2.18 (m, 4H), 1.96-1.88 (m, 1H).

### Example 44. Synthesis of Compound 43

A mixture of 3-(2,6-difluoro-4-(3-hydroxyazetidin-1-yl)phenyl)piperidine-2,6-dione XII (100 mg, 337 μmol, 1.00 eq) in dimethyl formamide (2 mL) was added sodium hydride (27.0 mg, 675 μmol, 60% purity, 2.00 eq) and phenyl (6-(1-methylcyclopropyl)pyridin-3-yl)carbamate (108 mg, 405 μmol, 1.20 eq) at 0° C. The reaction mixture was stirred at 20° C. for 1 h. The reaction mixture was added formic acid (2.00 mL) and filtered to give a filtrate, which was purified by Prep-HPLC (column: Phenomenex luna C18 150*25 mm*10 μm; mobile phase: [water (0.225% formic acid)-acetonitrile]; B %: 24%-54%, 10 min) and lyophilized to afford 1-(4-(2,6-dioxopiperidin-3-yl)-3,5-difluorophenyl)azetidin-3-yl(6-(1-methylcyclopropyl)pyridin-3-yl) carbamate #43 (88.22 mg, 169 μmol, 50% yield, 99% purity, formic acid) as a white solid. ¹H NMR (400 MHz, DMSO-d₆) δ=10.88 (s, 1H), 10.00 (br s, 1H), 8.48 (s, 1H), 7.78 (br d, J=7.2 Hz, 1H), 7.29 (d, J=8.8 Hz, 1H), 6.24 (s, 1H), 6.22 (s, 1H), 5.34-5.26 (m, 1H), 4.23 (br t, J=7.6 Hz, 2H), 4.05 (br dd, J=4.8, 12.8 Hz, 1H), 3.84 (br dd, J=3.2, 8.8 Hz, 2H), 2.85-2.73 (m, 1H), 2.43 (br s, 1H), 2.08 (dq, J=3.2, 13.2 Hz, 1H), 2.00-1.90 (m, 1H), 1.44 (s, 3H), 1.12-1.04 (m, 2H), 0.78-0.70 (m, 2H). MS (ESI) m/z 471.3 [M+H]⁺.

### Example 45. Synthesis of Compound 44

To a solution of 3-(2,6-difluoro-4-(3-hydroxyazetidin-1-yl)phenyl)piperidine-2,6-dione XII (100 mg, 338 μmol, 1.00 eq) in dimethylformamide (0.500 mL) were added sodium hydride (27.0 mg, 675 μmol, 60% purity, 2.00 eq) and phenyl (3-(difluoromethoxy)-5-fluorophenyl)carbamate (110 mg, 371 μmol, 1.10 eq) at 0° C. The mixture was stirred at 25° C. for 1 h. The pH of the mixture was adjusted to 7 with formic acid and diluted with dimethylformamide (1 mL). The residue was purified by Prep-HPLC (column: Phenomenex Synergi C18 150*25 mm*10 μm; mobile phase: [water (0.225% formic acid)-acetonitrile]; B %: 41%-74%, 11 min) followed by Prep-NPLC (column: Welch Ultimate XB-SiOH 250*50*10 μm; mobile phase: [Hexane-ethyl alcohol]; B %: 10%-10%, 15 min) to give a crude product. Then the crude product was diluted with ethyl acetate (1 mL) and hexane (10 mL) to afford a white solid. The white solid was diluted with water (10 mL) and lyophilized to afford 1-(4-(2,6-dioxopiperidin-3-yl)-3,5-difluorophenyl)azetidin-3-yl (3-(difluoromethoxy)-5-fluorophenyl)carbamate #44 (25.44 mg, 48.9 μmol, 14% yield, 96% purity) as a white solid. ¹H NMR (400 MHz, DMSO-d₆) δ=10.86 (s, 1H), 10.33 (s, 1H), 7.24 (t, J=74 Hz 1H), 7.22-7.14 (m, 2H), 6.81-6.73 (m, 1H), 6.22 (d, J=10.8 Hz, 2H), 5.34-5.24 (m, 1H), 4.27-4.18 (m, 2H), 4.04 (dd, J=4.9, 12.8 Hz, 1H), 3.84 (br dd, J=2.9, 9.2 Hz, 2H), 2.78 (ddd, J=5.1, 12.6, 17.6 Hz, 1H), 2.62-2.54 (m, 1H), 2.15-2.00 (m, 1H), 1.99-1.89 (m, 1H). MS (ESI) m/z 500.2 [M+H]⁺.

### Example 46. Synthesis of Compound 45

To a mixture of phenyl (4-(pyridin-2-yl)phenyl)carbamate (128 mg, 439 μmol, 1.30 eq) and 3-(2,6-difluoro-4-(3-hydroxyazetidin-1-yl)phenyl)piperidine-2,6-dione XII (100 mg, 338 μmol, 1.00 eq) in dimethylformamide (1 mL) was added sodium hydride (27.0 mg, 675 μmol, 60% purity, 2.00 eq) at 0° C. The mixture was stirred at 25° C. for 1 h. The reaction mixture was added hydrochloric acid (1 M, 2 mL) and filtered to give a filtrate. The filtrate was purified by Prep-HPLC (column: Phenomenex luna C18 150*25 mm*10 μm; mobile phase: [water (0.225% formic acid)-acetonitrile]; B %: 22%-52%, 10 min) to afford 1-(4-(2,6-dioxopiperidin-3-yl)-3,5-difluorophenyl)azetidin-3-yl (4-(pyridin-2-yl)phenyl)carbamate #45 (59.21 mg, 120 μmol, 36% yield, 99% purity) as a white solid. ¹H NMR (400 MHz, DMSO-d₆) δ=10.88 (s, 1H), 10.12 (br s, 1H), 8.64 (br d, J=4.2 Hz, 1H), 8.06 (br d, J=8.6 Hz, 2H), 7.94-7.88 (m, 1H), 7.88-7.84 (m, 1H), 7.58 (br d, J=8.4 Hz, 2H), 7.32-7.28 (m, 1H), 6.24 (br d, J=11.0 Hz, 2H), 5.32 (br d, J=2.8 Hz, 1H), 4.26 (br t, J=7.6 Hz, 2H), 4.06 (br dd, J=4.5, 12.5 Hz, 1H), 3.88 (br d, J=6.0 Hz, 2H), 2.86-2.72 (m, 1H), 2.64 (br dd, J=2.4, 7.8 Hz, 1H), 2.16-2.02 (m, 1H), 2.02-1.90 (m, 1H). MS (ESI) m/z 493.1 [M+H]⁺.

### Example 47. Synthesis of Compound 46

To a solution of 3-(2,6-difluoro-4-(3-hydroxyazetidin-1-yl)-3-methoxyphenyl) piperidine-2,6-dione XLVII (50.0 mg, 153 μmol, 1.00 eq) and phenyl (3-chloro-4-methylphenyl)carbamate (48.1 mg, 184 μmol, 1.20 eq) in N,N-dimethylformamide (1 mL) was added sodium hydride (9.19 mg, 230 μmol, 60% purity, 1.50 eq) at 0° C. The mixture was stirred at 20° C. for 1 h. The mixture was quenched by acetic acid (1 mL) and filtered. The filtrate was purified by Prep-HPLC (column: Phenomenex Synergi C18 150*25 mm*10 μm; mobile phase: [water (0.225% formic acid)-acetonitrile]; B %: 52%-72%, 10 min) and lyophilized to afford 1-(4-(2,6-dioxopiperidin-3-yl)-3,5-difluoro-2-methoxyphenyl)azetidin-3-yl (3-chloro-4-methylphenyl) carbamate #46 (40.63 mg, 74.5 μmol, 49% yield, 99% purity, formic acid) as an off-white solid. ¹H NMR (400 MHz, DMSO-d₆) δ=10.90 (s, 1H), 10.04 (br s, 1H), 8.49 (s, 1H), 7.59 (s, 1H), 7.31-7.24 (m, 2H), 6.29-6.23 (m, 1H), 5.29-5.22 (m, 1H), 4.31 (dd, J=6.5, 9.1 Hz, 2H), 4.07 (dd, J=5.1, 12.5 Hz, 1H), 3.90 (dd, J=3.9, 9.2 Hz, 2H), 3.68 (s, 3H), 2.84-2.73 (m, 1H), 2.52 (br s, 1H), 2.25 (s, 3H), 2.08 (dq, J=3.6, 12.9 Hz, 1H), 2.01-1.92 (m, 1H). MS (ESI) m/z 494.2 [M+H]⁺.

### Example 48. Synthesis of Compound 47

Step 1. To a solution of 3-amino-5-chlorobenzonitrile (500 mg, 3.28 mmol, 1.00 eq) and pyridine (264 μL, 3.28 mmol, 1.00 eq) in acetonitrile (5 mL) was added phenyl carbonochloridate (410 μL, 3.28 mmol, 1.00 eq). The reaction mixture was stirred at 20° C. for 12 h. The reaction mixture was concentrated to give a residue, which was poured into water (100 mL) and extracted with ethyl acetate (3×50 mL). The combined organic phase was separated, washed with brine (20 mL), dried over anhydrous sodium sulfate, filtered and concentrated to afford phenyl (3-chloro-5-cyanophenyl)carbamate (800 mg, 2.93 mmol, 89% yield) as a white solid. ¹H NMR (400 MHz, DMSO-d₆) δ=10.81 (br s, 1H), 7.89 (t, J=2.0 Hz, 1H), 7.83 (t, J=1.6 Hz, 1H), 7.73 (t, J=1.6 Hz, 1H), 7.49-7.43 (m, 2H), 7.33-7.28 (m, 1H), 7.28-7.27 (m, 1H), 7.25 (d, J=0.8 Hz, 1H). MS (ESI) m/z 272.9 [M+H]⁺.

Step 2. A mixture of 3-(2,6-difluoro-4-(3-hydroxyazetidin-1-yl)phenyl)piperidine-2,6-dione XII (100 mg, 337 μmol, 1.00 eq) in dimethyl formamide (2 mL) was added sodium hydride (27.0 mg, 675 μmol, 60% purity, 2.00 eq) and phenyl (3-cyano-5-methylphenyl) carbamate (110 mg, 405 μmol, 1.20 eq) at 0° C. The reaction mixture was stirred at 20° C. for 1 h. The reaction mixture was added formic acid (2 mL) and filtered to give a filtrate, which was purified by Prep-HPLC (column: Phenomenex luna C18 150*25 mm*10 μm; mobile phase: [water (0.225% formic acid)-acetonitrile]; B %: 45%-75%, 10 min) and lyophilized to afford 1-(4-(2,6-dioxopiperidin-3-yl)-3,5-difluorophenyl)azetidin-3-yl(3-chloro-5-cyanophenyl)carbamate #47 (59.51 mg, 124 μmol, 36% yield, 99% purity) as a white solid. ¹H NMR (400 MHz, DMSO-d₆) δ=10.86 (s, 1H), 10.50 (br s, 1H), 7.85 (d, J=2.0 Hz, 1H), 7.79 (d, J=1.6 Hz, 1H), 7.68 (s, 1H), 6.25 (s, 1H), 6.22 (s, 1H), 5.36-5.28 (m, 1H), 4.24 (dd, J=7.2, 8.4 Hz, 2H), 4.05 (dd, J=5.2, 12.4 Hz, 1H), 3.87 (br dd, J=3.2, 9.2 Hz, 2H), 2.85-2.73 (m, 1H), 2.53 (br s, 1H), 2.16-2.02 (m, 1H), 2.00-1.90 (m, 1H). MS (ESI) m/z 474.9 [M+H]⁺.

### Example 49. Synthesis of Compound 48

To a solution of 3-(2,6-difluoro-4-(3-hydroxyazetidin-1-yl)phenyl)piperidine-2,6-dione XII (100 mg, 337 μmol, 1.00 eq) in dimethyl formamide (1 mL) were added phenyl (4-cyano-3,5-dimethylphenyl)carbamate (107 mg, 405 μmol, 1.20 eq) and sodium hydride (20.2 mg, 506 μmol, 60% purity, 1.50 eq) at 0° C., the reaction was stirred at 20° C. for 2 h. The pH of the mixture was adjusted to 7 with formic acid and diluted with dimethylformamide (1 mL). The mixture was purified by Prep-HPLC (column: Phenomenex Luna C18 150*25 mm*10 μm; mobile phase: [water (0.225% formic acid)-acetonitrile]; B %: 41%-71%, 10 min) followed by NPLC (column: Welch Ultimate XB-CN 250*70 mm*10 μm; mobile phase: [hexane-ethanol]; B %: 32%-72%, 12 min) to afford 1-(4-(2,6-dioxopiperidin-3-yl)-3,5-difluorophenyl)azetidin-3-yl (4-cyano-3,5-dimethylphenyl) carbamate #48 (47.4 mg, 100 μmol, 29% yield, 99% purity) as a white solid. ¹H NMR (400 MHz, DMSO-d₆) δ=10.88 (s, 1H), 10.29 (s, 1H), 7.35 (s, 2H), 6.22 (d, J=10.8 Hz, 2H), 5.36-5.26 (m, 1H), 4.22 (br t, J=7.8 Hz, 2H), 4.05 (br dd, J=4.8, 12.5 Hz, 1H), 3.85 (br dd, J=3.2, 9.0 Hz, 2H), 2.85-2.72 (m, 1H), 2.53-2.52 (m, 1H), 2.41 (s, 6H), 2.08 (do, J=3.6, 13.0 Hz, 1H), 1.99-1.88 (m, 1H). MS (ESI) m/z 469.2 [M+H]⁺.

### Example 50. Synthesis of Compound 49

Step 1. To a mixture of 3-methyl-5-nitrobenzonitrile (1.00 g, 6.17 mmol, 1.00 eq) and iron powder (1.72 mg, 30.8 mmol, 5.00 eq) and ammonium chloride (1.65 g, 30.8 mmol, 5.00 eq) in methanol (5 mL) was added water (5). The reaction mixture was stirred at 80° C. for 2 h. The reaction mixture was concentrated to give a residue, which was poured into saturated sodium bicarbonate solution (50 mL) and extracted with ethyl acetate (3×20 mL). The combined organic layers were washed with brine (20 mL), dried over anhydrous sodium sulfate, filtered and concentrated under reduced pressure to give 3-amino-5-methylbenzonitrile (800 mg, crude) as a brown solid. ¹H NMR (400 MHz, DMSO-d₆) δ=6.85-6.56 (m, 3H), 6.12-4.66 (m, 2H), 2.18 (s, 3H). MS (ESI) m/z 133.1 [M+H]⁺.

Step 2. To a solution of 6-(1-methylcyclopropyl)pyridin-3-amine (400 mg, 3.03 mmol, 1.00 eq) and pyridine (732 μL, 9.08 mmol, 3.00 eq) in acetonitrile (5 mL) was added phenyl carbonochloridate (454 μL, 3.63 mmol, 1.20 eq). The reaction mixture was stirred at 20° C. for 1 h. The reaction mixture was concentrated to give a residue, which was poured into water (100 mL) and extracted with ethyl acetate (3×50 mL). The combined organic phase was separated, washed with brine (20. mL), dried over anhydrous sodium sulfate, filtered and concentrated to give a residue. The residue was purified by silica gel chromatography (petroleum ether/ethyl acetate=1/0 to 2/1) to give phenyl (3-cyano-5-methylphenyl) carbamate (300 mg, 1.19 mmol, 39% yield) as a white solid. ¹H NMR (400 MHz, DMSO-d₆) δ=10.55 (br s, 1H), 7.72 (s, 1H), 7.61 (s, 1H), 7.49-7.41 (m, 2H), 7.37 (s, 1H), 7.31-7.22 (m, 3H), 2.34 (s, 3H). MS (ESI) m/z 253.2 [M+H]⁺.

Step 3. A mixture of 3-(2,6-difluoro-4-(3-hydroxyazetidin-1-yl)phenyl)piperidine-2,6-dione XII (100 mg, 337 μmol, 1.00 eq) in dimethyl formamide (2 mL) were added sodium hydride (27.0 mg, 675 μmol, 60% purity, 2.00 eq) and phenyl (3-cyano-5-methylphenyl) carbamate (102 mg, 405 μmol, 1.20 eq) at 0° C. The reaction mixture was stirred at 20° C. for 1 h. The reaction mixture was added formic acid (2.00 mL) and filtered to give a filtrate, which was purified by Prep-HPLC (column: Phenomenex luna C18 150*25 mm*10 μm; mobile phase: [water (0.225% formic acid)-acetonitrile]; B %: 41%-71%, 10 min) and lyophilized to afford 1-(4-(2,6-dioxopiperidin-3-yl)-3,5-difluorophenyl)azetidin-3-yl (3-cyano-5-methylphenyl) carbamate #49 (78.66 mg, 115 μmol, 46% yield, 99% purity, formic acid) as a white solid. ¹H NMR (400 MHz, DMSO-d₆) δ=10.88 (br s, 1H), 10.27 (br s, 1H), 7.69 (s, 1H), 7.57 (s, 1H), 7.33 (s, 1H), 6.25 (s, 1H), 6.22 (s, 1H), 5.34-5.27 (m, 1H), 4.23 (br t, J=8.0 Hz, 2H), 4.05 (br dd, J=5.2, 12.4 Hz, 1H), 3.85 (br dd, J=3.2, 8.8 Hz, 2H), 2.85-2.73 (m, 1H), 2.53-2.52 (m, 1H), 2.32 (s, 3H), 2.15-2.03 (m, 1H), 2.00-1.91 (m, 1H). MS (ESI) m/z 455.0 [M+H]⁺.

### Example 51. Synthesis of Compound 50

Step 1. To a mixture of 3-fluoro-5-(trifluoromethoxy)aniline (200 mg, 1.03 mmol, 1.00 eq) in dimethylformamide (2.00 mL) was added phenyl carbonochloridate (193 μL, 1.54 mmol, 1.50 eq) and pyridine (99.3 μL, 1.23 mmol, 1.20 eq) at 0° C. Then the mixture was stirred at 25° C. for 12 h. The reaction mixture was filtered and concentrated under reduced pressure to give a residue. The residue was purified by reversed phase column chromatography (C18, 40 g; condition: water/acetonitrile=1/0 to 0/1, 0.1% formic acid) to afford phenyl (3-fluoro-5-(trifluoromethoxy)phenyl)carbamate (130 mg, 412 μmol, 40% yield) as a white solid. ¹H NMR (400 MHz, CDCl₃) δ=7.38-7.29 (m, 2H), 7.22-7.08 (m, 5H), 6.99 (br s, 1H), 6.64 (br dd, J=0.9, 8.9 Hz, 1H).

Step 2. To a mixture of phenyl (3-fluoro-5-(trifluoromethoxy)phenyl)carbamate (73.4 mg, 203 μmol, 1.00 eq, formic acid) and 3-(4-(3-aminoazetidin-1-yl)-2,6-difluorophenyl)piperidine-2,6-dione IX (60.0 mg, 203 μmol, 1.00 eq) in dimethylformamide (2 mL) was added triethylamine (56.6 μL, 406 μmol, 2.00 eq). Then the mixture was stirred at 30° C. for 2 h. The reaction mixture was filtered. The filtrate was purified by Prep-HPLC (column: Phenomenex luna C18 150×25 mm×10 um; mobile phase: [water (0.225% formic acid)-acetonitrile]; B %: 43%-73%, 10 min) and lyophilized to afford a residue. The residue was purified by Prep-HPLC (column: Unisil 3-100 C18 Ultra 150*50 mm*3 μm; mobile phase: [water (0.225% formic acid)-acetonitrile]; B %: 42%-72%, 10 min) and lyophilized to afford a crude product. The crude product was purified by Prep-HPLC (column: Unisil 3-100 C18 Ultra 150*50 mm*3 μm; mobile phase: [water (0.225% formic acid)-acetonitrile]; B %: 42%-72%, 10 min) to afford 1-(1-(4-(2,6-dioxopiperidin-3-yl)-3,5-difluorophenyl)azetidin-3-yl)-3-(3-fluoro-5-(trifluoromethoxy)phenyl)urea #50 (36.2 mg, 68.8 μmol, 33% yield, 98% purity) as a white solid. ¹H NMR (400 MHz, DMSO-d₆) δ=10.91-10.75 (m, 1H), 9.96 (br s, 1H), 7.97 (br d, J=6.6 Hz, 1H), 7.43-7.29 (m, 2H), 6.98-6.64 (m, 1H), 6.17 (d, J=11.0 Hz, 2H), 4.55 (br s, 1H), 4.13 (t, J=7.6 Hz, 2H), 4.08-4.00 (m, 1H), 3.67 (br t, J=6.0 Hz, 2H), 2.84-2.73 (m, 1H), 2.47-2.42 (m, 1H), 2.14-2.02 (m, 1H), 2.00-1.87 (m, 1H). MS (ESI) m/z 517.0 [M+H]⁺.

### Example 52. Synthesis of Compound 51

Step 1. To a solution of 2-chloropyrimidin-5-amine (100 mg, 0.772 mmol, 1.00 eq) in dioxane (1 mL) and water (0.100 mL) were added potassium carbonate (213 mg, 1.54 mmol, 2.00 eq), phenylboronic acid (113 mg, 0.926 mmol, 1.20 eq) and [1,1-bis(diphenylphosphino)ferrocene]dichloropalladium(II) (56.5 mg, 77.2 umol, 0.100 eq). The reaction mixture was stirred at 90° C. for 12 h under nitrogen atmosphere. The reaction mixture was filtered and concentrated under residue pressure to give a residue. The residue was purified by reversed phase column chromatography (C18, 40 g; condition: water/acetonitrile=1/0 to 0/1, 0.1% formic acid) and lyophilized to afford 2-phenylpyrimidin-5-amine (90.0 mg, 0.526 mmol, 68% yield) as a yellow solid. ¹H NMR (400 MHz, DMSO-d₆) δ=8.26-8.11 (m, 4H), 7.45-7.39 (m, 2H), 7.38-7.33 (m, 1H), 5.69 (s, 2H).

Step 2. To a solution of 2-phenylpyrimidin-5-amine (90.0 mg, 526 μmol, 1.00 eq) in acetonitrile (3.00 mL) were added pyridine (127 μL, 1.58 mmol, 3.00 eq) and phenyl carbonochloridate (72.4 μL, 578 μmol, 1.10 eq) at 0° C. The reaction mixture was stirred at 20° C. for 1 h. The reaction mixture was concentrated under reduced pressure to give a residue. The residue was purified by reversed phase column chromatography (C18, 40 g; condition: water/acetonitrile=1/0 to 0/1, 0.1% formic acid) and lyophilized to afford phenyl (2-phenylpyrimidin-5-yl)carbamate (130 mg, 446 μmol, 85% yield) as a yellow solid. ¹H NMR (400 MHz, DMSO-d₆) δ=10.74 (br s, 1H), 9.02 (s, 2H), 8.37-8.33 (m, 2H), 7.52-7.49 (m, 3H), 7.46 (t, J=7.9 Hz, 2H), 7.31-7.25 (m, 3H).

Step 3. To a solution of 3-(2,6-difluoro-4-(3-hydroxyazetidin-1-yl)phenyl)piperidine-2,6-dione XII (80.0 mg, 270 μmol, 1.00 eq), phenyl (2-phenylpyrimidin-5-yl)carbamate (86.5 mg, 297 μmol, 1.10 eq) in dimethylformamide (1 mL) was added sodium hydride (21.6 mg, 540 μmol, 60% purity, 2.00 eq) at 0° C. The reaction mixture was stirred at 20° C. for 1 h. The reaction mixture was quenched with formic acid (0.500 mL), then diluted with dimethyl formamide (1 mL) to give a residue. The residue was purified by Prep-HPLC (column: Unisil 3-100 C18 Ultra 150*50 mm*3 μm; mobile phase: [water (0.225% formic acid)-acetonitrile]; B %: 40%-70%, 10 min) and lyophilized to afford 1-(4-(2,6-dioxopiperidin-3-yl)-3,5-difluorophenyl)azetidin-3-yl(2-phenylpyrimidin-5-yl)carbamate #51 (85.65 mg, 172 μmol, 64% yield, 99% purity) as a white solid. ¹H NMR (400 MHz, DMSO-d₆) δ=10.87 (s, 1H), 10.54-10.33 (m, 1H), 8.97 (s, 2H), 8.33 (dd, J=2.0, 7.1 Hz, 2H), 7.55-7.45 (m, 3H), 6.25 (s, 1H), 6.22 (s, 1H), 5.38-5.31 (m, 1H), 4.25 (br t, J=7.7 Hz, 2H), 4.05 (br dd, J=4.7, 12.7 Hz, 1H), 3.93-3.85 (m, 2H), 2.83-2.75 (m, 1H), 2.53-2.52 (m, 1H), 2.08 (dq, J=3.5, 12.9 Hz, 1H), 2.00-1.90 (m, 1H). MS (ESI) m/z 494.1 [M+H]⁺.

### Example 53. Synthesis of Compound 52

A mixture of 3-(2,6-difluoro-4-(3-hydroxyazetidin-1-yl)phenyl)piperidine-2,6-dione XII (100 mg, 337 μmol, 1.00 eq) and phenyl (3-chloro-4-methyl-5-(2-morpholinoethoxy)phenyl)carbamate (see compound 13) (158 mg, 405 μmol, 1.20 eq) in dimethyl formamide was added sodium hydride (27.0 mg, 675 μmol, 60% purity, 2.00 eq) at 0° C. The reaction mixture was stirred at 20° C. for 1 h. The reaction mixture was added formic acid (2 mL) and filtered to give a filtrate. The filtrate was purified by Prep-HPLC (column: Phenomenex luna C18 150*25 mm*10 μm; mobile phase: [water (0.225% formic acid)-acetonitrile]; B %: 19%-49%, 10 min) and lyophilized to give a residue. The residue was triturated with ethyl acetate/n-hexane (1:2) to afford 1-(4-(2,6-dioxopiperidin-3-yl)-3,5-difluorophenyl)azetidin-3-yl(3-chloro-4-methyl-5-(2-morpholinoethoxy)phenyl)carbamate #52 (21.47 mg, 33.2 μmol, 9% yield, 99% purity, formic acid) as a white solid. ¹H NMR (400 MHz, DMSO-d₆) δ=10.88 (s, 1H), 10.00 (br s, 1H), 7.15 (br d, J=8.8 Hz, 2H), 6.23 (d, J=11.2 Hz, 2H), 5.32-5.25 (m, 1H), 4.23 (br t, J=7.6 Hz, 2H), 4.08-4.02 (m, 3H), 3.84 (br dd, J=2.4, 9.2 Hz, 2H), 3.60-3.55 (m, 4H), 2.84-2.76 (m, 1H), 2.73 (t, J=5.6 Hz, 2H), 2.48-2.46 (m, 4H), 2.40-2.35 (m, 1H), 2.13 (s, 3H), 2.11-2.03 (m, 1H), 1.99-1.91 (m, 1H). MS (ESI) m/z 593.1 [M+H]⁺.

### Example 54. Synthesis of Compound 53

Step 1. A mixture of 2-chloro-1-methyl-4-nitrobenzene (6.02 mL, 29.1 mmol, 1.00 eq) in sulfuric acid (50 mL) was added N-iodosuccinimide (7.21 g, 32.0 mmol, 1.10 eq). The reaction mixture was stirred at 60° C. for 12 h. The reaction mixture was poured into ice water (200 mL) and extracted with ethyl acetate (3×50 mL). The combined organic phase was separated, washed with brine (50 mL), dried over anhydrous sodium sulfate, filtered and concentrated to give a residue. The residue was purified by silica gel chromatography (petroleum ether/ethyl acetate=1/0 to 20/1) to afford 1-chloro-3-iodo-2-methyl-5-nitrobenzene (8.00 g, 26.8 mmol, 92% yield) as a brown solid. ¹H NMR (400 MHz, DMSO-d₆) δ=8.52 (d, J=2.4 Hz, 1H), 8.28 (d, J=2.4 Hz, 1H), 2.60 (s, 3H).

Step 2. To a mixture of 1-chloro-3-iodo-2-methyl-5-nitrobenzene (1.00 g, 3.36 mmol, 1.00 eq) in dimethylformamide (20 mL) were added zinc cyanide (128 μL, 2.02 mmol, 0.600 eq), tris(dibenzylideneacetone)dipalladium(0) (153 mg, 0.168 mmol, 0.0500 eq) and 1,1′-bis(diphenylphosphino) ferrocene (93.1 mg, 0.168 mmol, 0.0500 eq) under nitrogen atmosphere. The reaction mixture was stirred at 80° C. for 12 h. The reaction mixture was filtered to give filtrate. The filtrate was poured into water (100 mL) and extracted with ethyl acetate (3×20 mL). The combined organic layers were washed with brine (50 mL), dried over anhydrous sodium sulfate, filtered and the filtrate was concentrated to afford a residue. The residue was purified by silica gel chromatography (petroleum ether/ethyl acetate=1/0) to afford 3-chloro-2-methyl-5-nitrobenzonitrile (450 mg, 2.29 mmol, 68% yield) as a yellow solid. ¹H NMR (400 MHz, DMSO-d₆) δ=8.73 (d, J=2.4 Hz, 1H), 8.61 (d, J=2.4 Hz, 1H), 2.65 (s, 3H).

Step 3. To a mixture of 3-chloro-2-methyl-5-nitrobenzonitrile (450 mg, 2.29 mmol, 1.00 eq) and iron powder (639 mg, 11.4 mmol, 5.00 eq) and ammonium chloride (612 mg, 11.4 mmol, 5.00 eq) in methanol (5 mL) was added water (5 mL). The reaction mixture was stirred at 80° C. for 2 h. The reaction mixture was concentrated to give a residue, which was poured into saturated sodium bicarbonate solution (50 mL) and extracted with ethyl acetate (3×20 mL). The combined organic layers were washed with brine (20.0 mL), dried over anhydrous sodium sulfate, filtered and concentrated under reduced pressure to afford 5-amino-3-chloro-2-methylbenzonitrile (340 mg, 2.04 mmol, 89% yield) as a white solid. ¹H NMR (400 MHz, DMSO-d₆) δ=6.94 (d, J=2.4 Hz, 1H), 6.84 (d, J=2.4 Hz, 1H), 5.68 (br s, 2H), 2.31 (s, 3H). MS (ESI) m/z 167.0 [M+H]⁺.

Step 4. To a solution of 5-amino-3-chloro-2-methylbenzonitrile (340 mg, 2.04 mmol, 1.00 eq) and pyridine (494 μL, 6.12 mmol, 3.00 eq) in acetonitrile (10 mL) was added phenyl carbonochloridate (306 uL, 2.45 mmol, 1.20 eq). The reaction mixture was stirred at 20° C. for 1 h. The reaction mixture was concentrated to give a residue, which was poured into water (100 mL) and extracted with ethyl acetate (3×50 mL). The combined organic phase was separated, washed with brine (20 mL), dried over anhydrous sodium sulfate, filtered and concentrated to give a residue. The residue was purified by silica gel chromatography (petroleum ether/ethyl acetate=20/1 to 5/1) to afford phenyl (3-chloro-5-cyano-4-methylphenyl)carbamate (500 mg, 1.74 mmol, 85% yield) as a white solid. ¹H NMR (400 MHz, DMSO-d₆) δ=10.68 (br s, 1H), 7.88 (d, J=2.0 Hz, 1H), 7.80 (d, J=2.0 Hz, 1H), 7.47-7.42 (m, 2H), 7.32-7.23 (m, 3H), 2.47 (s, 3H). MS (ESI) m/z 287.0 [M+H]⁺.

Step 5. A mixture of 3-(2,6-difluoro-4-(3-hydroxyazetidin-1-yl)phenyl)piperidine-2,6-dione XII (100 mg, 337 μmol, 1.00 eq) in dimethyl formamide (2 mL) were added sodium hydride (27.0 mg, 675 μmol, 60% purity, 2.00 eq) and phenyl (3-chloro-5-cyano-4-methylphenyl)carbamate (116 mg, 405 μmol, 1.20 eq) at 0° C. The reaction mixture was stirred at 20° C. for 1 h. The reaction mixture was added formic acid (2.00 mL) and filtered to give a filtrate, which was purified by Prep-HPLC (column: Phenomenex luna C18 150*25 mm*10 μm; mobile phase: [water (0.225% formic acid)-acetonitrile]; B %: 48%-78%, 10 min) and lyophilized to afford 1-(4-(2,6-dioxopiperidin-3-yl)-3,5-difluorophenyl)azetidin-3-yl(3-chloro-5-cyano-4-methylphenyl) carbamate #53 (91.27 mg, 184 μmol, 54% yield, 99% purity) as a white solid. ¹H NMR (400 MHz, DMSO-d₆) δ=10.87 (br s, 1H), 10.38 (br s, 1H), 7.85 (d, J=1.6 Hz, 1H), 7.77 (d, J=2.0 Hz, 1H), 6.23 (d, J=11.2 Hz, 2H), 5.36-5.28 (m, 1H), 4.28-4.19 (m, 2H), 4.05 (br dd, J=5.2, 12.4 Hz, 1H), 3.86 (br dd, J=3.2, 8.8 Hz, 2H), 2.84-2.74 (m, 1H), 2.54-2.53 (m, 1H), 2.46 (s, 3H), 2.16-2.02 (m, 1H), 2.00-1.91 (m, 1H). MS (ESI) m/z 489.2 [M+H]⁺.

### Example 55. Synthesis of Compound 54

To a solution of phenyl N-(3-chloro-4-methyl-phenyl)carbamate (43.9 mg, 168 μmol, 1.50 eq) and 3-(2-chloro-3-fluoro-4-(3-hydroxyazetidin-1-yl)phenyl)piperidine-2,6-dione LII (35.0 mg, 112 μmol, 1.00 eq) in dimethyl formamide (3 mL) was added sodium hydride (8.99 mg, 224 μmol, 60% purity, 2.01 eq) at 0° C. Then the mixture was stirred at 15° C. for 1 h. The reaction mixture was added to 1 M hydrochloric acid (20 mL) dropwise at 0° C., and then diluted with ethyl acetate (30 mL). The organic layer was separated and the aqueous phase was extracted with ethyl acetate (30 mL). The combined organic layers were washed with brine (50 mL), dried over sodium sulfate, filtered and concentrated under reduced pressure. The residue was purified by prep-HPLC (column: Phenomenex luna C18 150*25 mm*10 μm; mobile phase: [water (0.225% formic acid)-acetonitrile]; B %: 52%-82%, 10 min) and lyophilized to give 1-(3-chloro-4-(2,6-dioxopiperidin-3-yl)-2-fluorophenyl)azetidin-3-yl(3-chloro-4-methylphenyl)carbamate #54 (28.78 mg, 59.3 μmol, 53% yield) as an off-white solid. ¹H NMR (400 MHz, DMSO-d₆) δ=10.86 (s, 1H), 10.02 (s, 1H), 7.58 (s, 1H), 7.34-7.22 (m, 2H), 7.00 (d, J=7.6 Hz, 1H), 6.59 (t, J=8.8 Hz, 1H), 5.33-5.22 (m, 1H), 4.33 (t, J=7.2 Hz, 2H), 4.09 (dd, J=5.2, 12.4 Hz, 1H), 3.93 (dd, J=3.6, 8.8 Hz, 2H), 2.82-2.70 (m, 1H), 2.63 (br s, 1H), 2.30-2.19 (m, 4H), 2.00-1.88 (m, 1H). MS (ESI) m/z 480.1 [M+H]⁺.

### Example 56. Synthesis of Compound 55

To a solution of 3-(4-(3-hydroxyazetidin-1-yl)phenyl)piperidine-2,6-dione LIX (150 mg, 0.576 mmol, 1.00 eq) in dimethyl formamide (2 mL) were added phenyl (3-chloro-4-methylphenyl)carbamate (181 mg, 0.692 mmol, 1.20 eq) and sodium hydride (46.1 mg, 1.15 mmol, 60% purity, 2.00 eq). The mixture was stirred at 25° C. for 1 h. The pH of the mixture was adjusted to 7 with formic acid, the mixture was diluted with dimethyl formamide (1 mL). The residue was purified by Prep-HPLC (column: Phenomenex luna C18 150*25 mm*10 μm; mobile phase: [water (0.225% formic acid)-acetonitrile]; B %: 41%-71%, 10 min) and the desired eluent was lyophilized to afford 1-(4-(2,6-dioxopiperidin-3-yl)phenyl)azetidin-3-yl (3-chloro-4-methylphenyl)carbamate #55 (63.0 mg, 146 μmol, 25% yield, 99% purity) as a white solid. ¹H NMR (400 MHz, DMSO-d₆) δ=10.75 (br s, 1H), 9.99 (br s, 1H), 7.58 (s, 1H), 7.37-7.17 (m, 2H), 7.03 (d, J=8.4 Hz, 2H), 6.46 (d, J=8.4 Hz, 2H), 5.34-5.24 (m, 1H), 4.18 (t, J=7.6 Hz, 2H), 3.76 (dd, J=4.0, 8.8 Hz, 2H), 3.71 (dd, J=5.0, 11.1 Hz, 1H), 2.68-2.58 (m, 1H), 2.44 (br t, J=4.4 Hz, 1H), 2.25 (s, 3H), 2.19-2.05 (m, 1H), 2.04-1.95 (m, 1H). MS (ESI) m/z 428.0 [M+H]⁺.

### Example 57. Synthesis of Compound 56

To a solution of 3-(2-chloro-4-(3-hydroxyazetidin-1-yl)-5-methoxyphenyl)piperidine-2,6-dione LVI (100 mg, 308 μmol, 1.00 eq) in dimethyl formamide (0.500 mL) were added sodium hydride (27.1 mg, 677 umol, 60% purity, 2.20 eq) and phenyl (3-chloro-4-methylphenyl)carbamate (106 mg, 406 μmol, 1.32 eq). The mixture was stirred at 25° C. for 1 h. The pH of the mixture was adjusted to 7 with formic acid, then the mixture was diluted with dimethyl formamide (1 mL). The residue was purified by Prep-HPLC (column: Phenomenex luna C18 150*25 mm*10 μm; mobile phase: [water (0.225% formic acid)-acetonitrile]; B %: 47%-77%, 10 min) and the desired eluent was lyophilized to afford 1-(5-chloro-4-(2,6-dioxopiperidin-3-yl)-2-methoxyphenyl)azetidin-3-yl (3-chloro-4-methylphenyl)carbamate #56 (26.31 mg, 52.9 μmol, 17% yield, 99% purity) as a white solid. ¹H NMR (400 MHz, DMSO-d₆) δ=10.81 (s, 1H), 9.99 (br s, 1H), 7.58 (s, 1H), 7.33-7.20 (m, 2H), 6.80 (s, 1H), 6.44 (s, 1H), 5.24-5.15 (m, 1H), 4.27-4.18 (m, 2H), 4.03 (dd, J=5.1, 12.4 Hz, 1H), 3.81 (dd, J=3.9, 9.2 Hz, 2H), 3.71 (s, 3H), 2.82-2.66 (m, 1H), 2.57-2.52 (m, 1H), 2.38-2.30 (m, 1H), 2.25 (s, 3H), 1.96-1.87 (m, 1H). MS (ESI) m/z 492.1 [M+H]⁺.

### Example 58. Synthesis of Compound 57

To a mixture of 2-(2,6-dioxopiperidin-3-yl)-5-(3-hydroxyazetidin-1-yl)benzonitrile LXII (25.0 mg, 87.6 μmol, 1.00 eq) and phenyl (3-chloro-4-methylphenyl)carbamate (27.5 mg, 105 μmol, 1.20 eq) in dimethylformamide (1 mL) was added sodium hydride (5.26 mg, 131 μmol, 60% purity, 1.50 eq) at 0° C. The mixture was stirred at 20° C. for 2 h. The reaction mixture was added hydrochloric acid (1 M, 2.00 mL) and filtered to give a filter cake. The filter cake was purified by Prep-HPLC (column: Phenomenex Synergi C18 150*25 mm*10 μm; mobile phase: [water(0.225% formic acid)-acetonitrile]; B %: 45%-67%, 11 min) to afford 1-(3-cyano-4-(2,6-dioxopiperidin-3-yl)phenyl)azetidin-3-yl(3-chloro-4-methylphenyl)carbamate #57 (17.92 mg, 39.2 μmol, 45% yield, 99% purity) as a white solid. ¹H NMR (400 MHz, DMSO-d₆) δ=10.92 (s, 1H), 10.02 (br s, 1H), 7.58 (s, 1H), 7.32-7.28 (m, 2H), 7.26 (s, 1H), 6.92 (d, J=2.5 Hz, 1H), 6.78 (dd, J=2.6, 8.6 Hz, 1H), 5.36-5.26 (m, 1H), 4.26 (dd, J=6.5, 8.9 Hz, 2H), 4.00 (dd, J=4.9, 12.7 Hz, 1H), 3.88 (dd, J=3.8, 9.1 Hz, 2H), 2.88-2.76 (m, 1H), 2.58 (br d, J=3.0 Hz, 1H), 2.36-2.28 (m, 1H), 2.26 (s, 3H), 2.02 (ddd, J=2.6, 5.1, 7.8 Hz, 1H). MS (ESI) m/z 453.0 [M+H]⁺.

### Example 59. Synthesis of Compound 58

Step 1. To a solution of tert-butyl (4-(prop-1-en-2-yl)phenyl)carbamate (1.40 g, 6.00 mmol, 1.00 eq) in dichloromethane (10 mL) was added diethylzine (1 M, 12.0 mL, 2.00 eq) and diiodomethane (1.94 mL, 24.0 mmol, 4.00 eq) at 0° C. Then the mixture was stirred at 25° C. for 10 h. The reaction mixture was quenched by addition saturated ammonium chloride (5 mL), and then extracted with ethyl acetate (3×40 mL). The combined organic layers were washed with brine (10 mL), dried over sodium sulfate, filtered and concentrated under reduced pressure to give a residue. The residue was purified by column chromatography (SiO₂, petroleum ether/ethyl acetate=10/1) to afford tert-butyl (4-(1-methylcyclopropyl)phenyl)carbamate (0.150 g, 0.606 μmol, 10% yield) as yellow oil. ¹H NMR (400 MHz, CDCl₃) δ=7.17 (s, 2H), 7.13-7.08 (m, 2H), 6.31 (br s, 1H), 1.44 (s, 9H), 1.30 (s, 3H), 0.75-0.71 (m, 2H), 0.64-0.58 (m, 2H).

Step 2. To a solution of tert-butyl (4-(1-methylcyclopropyl)phenyl)carbamate (0.12 g, 485 μmol, 1.00 eq) in ethyl acetate (2 mL) was added hydrochloric acid/ethyl acetate (4 M, 2.00 mL, 16.5 eq). Then the mixture was stirred at 25° C. for 1 h. The reaction mixture was concentrated under reduced pressure to afford 4-(1-methylcyclopropyl)aniline (80.0 mg, 485 μmol, crude) as a yellow solid and was used for the next step without purification. MS (ESI) m/z 148.2 [M+H]⁺.

Step 3. To a solution of 4-(1-methylcyclopropyl)aniline (50.0 mg, 340 μmol, 1.00 eq) and potassium carbonate (93.9 mg, 679 μmol, 2.00 eq) in acetone (2 mL) was added phenyl carbonochloridate (51.1 μL, 408 μmol, 1.20 eq). Then the mixture was stirred at 25° C. for 10 h. The reaction mixture was concentrated under reduced pressure to give a residue. The residue was purified by column chromatography (SiO₂, petroleum ether/ethyl acetate=10/1) to afford phenyl (4-(1-methylcyclopropyl)phenyl)carbamate (80.0 mg, 299 μmol, 88% yield) as a yellow solid. ¹H NMR (400 MHz, CDCl₃) δ=7.35-7.23 (m, 4H), 7.17-7.10 (m, 5H), 1.32 (s, 3H), 0.80-0.73 (m, 2H), 0.68-0.60 (m, 2H).

Step 4. To a solution of phenyl (4-(1-methylcyclopropyl)phenyl)carbamate (40.0 mg, 150 μmol, 1.00 eq) and 3-(2,6-difluoro-4-(3-hydroxyazetidin-1-yl)phenyl)piperidine-2,6-dione XII (44.3 mg, 150 μmol, 1.00 eq) in N,N-dimethylformamide (1 mL) was added sodium hydride (12.0 mg, 299 μmol, 60% purity, 2.00 eq). Then the mixture was stirred at 25° C. for 10 h. The reaction mixture was quenched by addition acetic acid (0.500 mL). The filtrate was purified by Prep-HPLC (column: Phenomenex luna C18 150*25 mm*10 μm; mobile phase: [water (0.2% formic acid)-acetonitrile]; B %: 54%-74%, 10 min) and lyophilized to give a residue. The residue was further purified by Prep-HPLC (column: DAICEL CHIRALPAK AD (250 mm*30 mm*10 μm); mobile phase: [Neu-isopropanol]; B %: 60%-60%, 6.25; 30 min) to afford 1-(4-(2,6-dioxopiperidin-3-yl)-3,5-difluorophenyl)azetidin-3-yl (4-(1-methylcyclopropyl)phenyl)carbamate #58 (27.7 mg, 58.4 umol, 91% yield, 99% purity) as an off-white solid. ¹H NMR (400 MHz, DMSO-d₆) δ=10.87 (s, 1H), 9.79 (br s, 1H), 7.36 (br d, J=8.4 Hz, 2H), 7.14 (d, J=8.8 Hz, 2H), 6.23 (d, J=11.0 Hz, 2H), 5.34-5.21 (m, 1H), 4.28-4.18 (m, 2H), 4.05 (br dd, J=4.8, 12.2 Hz, 1H), 3.83 (br dd, J=3.6, 9.0 Hz, 2H), 2.88-2.73 (m, 1H), 2.46-2.36 (m, 1H), 2.15-2.03 (m, 1H), 2.01-1.91 (m, 1H), 1.35 (s, 3H), 0.81-0.74 (m, 2H), 0.74-0.68 (m, 2H). MS (ESI) m/z 470.1 [M+H]⁺.

### Example 60. Synthesis of Compound 59

To a stirred solution of phenyl (3-chloro-4-methylphenyl)carbamate (48.8 mg, 186.2 μmol, 1.10 eq) and 3-(2-chloro-4-(3-hydroxyazetidin-1-yl)-3-methoxyphenyl)piperidine-2,6-dione XVIII (73.3 mg, 169 μmol, 75% purity, 1.00 eq) in dimethylformamide (1 mL) at 0° C. was added sodium hydride (13.5 mg, 338 μmol, 60% purity, 2.00 eq), then the resulting mixture was stirred at 15° C. for 1.5 h. The reaction mixture was quenched by addition saturated ammonium chloride solution (5 mL), and then extracted with ethyl acetate (3×5 mL). The combined organic layers were washed with brine (3×10 mL), dried over sodium sulfate, filtered and concentrated under reduced pressure to give a residue. The residue was purified by prep-HPLC (formic acid condition, column: Phenomenex Gemini-NX C18 75*30 mm*3 μm; mobile phase: [water (0.225% formic acid)-acetonitrile]; B %: 45%-75%, 7 min). Repurification by prep-HPLC (Neutral condition, column: Waters Xbridge 150*25 mm*5 μm; mobile phase: [water (10 mM ammonium bicarbonate)-acetonitrile]; B %: 40%-70%, 10 min) and lyophilized to give 1-(3-chloro-4-(2,6-dioxopiperidin-3-yl)-2-methoxyphenyl)azetidin-3-yl (3-chloro-4-methylphenyl)carbamate #59 (3.75 mg, 7.63 μmol, 4.50% yield) as an off-white solid. ¹H NMR (400 MHz, DMSO-d₆) δ=10.84 (s, 1H), 10.03 (s, 1H), 7.60 (s, 1H), 7.31-7.25 (m, 2H), 6.93 (d, J=8.50 Hz, 1H), 6.51 (d, J=8.50 Hz, 1H), 5.29-5.24 (m, 1H), 4.29 (br t, J=7.69 Hz, 2H), 4.07 (dd, J=12.13, 5.0 Hz, 1H), 3.86 (dd, J=9.01, 4.0 Hz, 2H), 3.65 (s, 3H), 2.78-2.69 (m, 1H), 2.49-2.47 (m, 1H), 2.26 (s, 3H), 2.22-2.17 (m, 1H), 1.98-1.92 (m, 1H). MS (ESI) m/z 492.1 [M+H]⁺.

### Example 61. Synthesis of Compound 60

Step 1. To a solution of 4-(1-methylcyclopropyl)aniline (50.0 mg, 339 μmol, 1.00 eq) and potassium carbonate (93.9 mg, 679 μmol, 2.00 eq) in acetone (2 mL) was added phenyl carbonochloridate (51.1 μL, 408 μmol, 1.20 eq). Then the mixture was stirred at 25° C. for 10 h. The reaction mixture was quenched by addition water (8 mL), and then extracted with ethyl acetate (3×15 mL). The combined organic layers were washed with brine (10 mL), dried over sodium sulfate, filtered and concentrated under reduced pressure to afford phenyl (4-(1-methylcyclopropyl)phenyl)carbamate (95.0 mg, 355 μmol, crude) as a yellow solid. ¹H NMR (400 MHz, CDCl₃) δ=7.36-7.32 (m, 4H), 7.20 (d, J=2.4 Hz, 2H), 7.17-7.10 (m, 4H), 1.32 (s, 3H), 0.79-0.73 (m, 2H), 0.67-0.62 (m, 2H).

Step 2. To a solution of 3-(4-(3-aminoazetidin-1-yl)-2,6-difluorophenyl)piperidine-2,6-dione IX (44.2 mg, 150 umol, 1.00 eq) and triethylamine (41.7 μL, 299 μmol, 2.00 eq) in N,N-dimethylformamide (1 mL) was added phenyl (4-(1-methylcyclopropyl)phenyl)carbamate (40.0 mg, 150 μmol, 1.00 eq). Then the mixture was stirred at 25° C. for 1 h. The reaction mixture was filtered. The filtrate was purified by Prep-HPLC (column: Waters Xbridge 150*25 mm*5 μm; mobile phase: [water (10 mM ammonium bicarbonate)-acetonitrile]; B %: 42%-72%, 8 min) and lyophilized to give a residue. The residue was further purified by prep-HPLC (column: DAICEL CHIRALPAK AD (250 mm*30 mm*10 μm); mobile phase: [isopropanol-acetonitrile]; B %: 70%-70%, 6.7 min; 80 min) to afford 1-(1-(4-(2,6-dioxopiperidin-3-yl)-3,5-difluorophenyl)azetidin-3-yl)-3-(4-(1-methylcyclopropyl)phenyl)urea #60 (15.2 mg, 32.3 μmol, 30% yield, 99% purity) as a white solid. ¹H NMR (400 MHz, DMSO-d₆) δ=10.87 (br s, 1H), 8.43 (s, 1H), 7.29 (d, J=8.6 Hz, 2H), 7.08 (d, J=8.6 Hz, 2H), 6.77 (d, J=7.2 Hz, 1H), 6.18 (d, J=11.2 Hz, 2H), 4.62-4.51 (m, 1H), 4.13 (br t, J=7.6 Hz, 2H), 4.04 (br dd, J=4.8, 12.4 Hz, 1H), 3.64 (br t, J=6.2 Hz, 2H), 2.85-2.73 (m, 1H), 2.49-2.41 (m, 1H), 2.16-2.02 (m, 1H), 2.00-1.89 (m, 1H), 1.34 (s, 3H), 0.79-0.72 (m, 2H), 0.71-0.65 (m, 2H). MS (ESI) m/z 469.1 [M+H]⁺.

### Example 62. Synthesis of Compound 61

Step 1. To a solution of 3-phenylbicyclo[1.1.1]pentan-1-amine hydrochloride (80.0 mg, 408 μmol, 1.00 eq) and pyridine (164 μL, 2.04 mmol, 5.00 eq) in acetonitrile (2 mL) was added phenyl carbonochloridate (61.4 μL, 490 μmol, 1.20 eq). The reaction mixture was stirred at 20° C. for 1 h. The reaction mixture was poured into water (50 mL) and extracted with ethyl acetate (3×20 mL). The combined organic phase was separated, washed with brine (3×10 mL), dried over anhydrous sodium sulfate, filtered and concentrated to give phenyl (3-phenylbicyclo[1.1.1]pentan-1-yl)carbamate (100 mg, 358 μmol, 87% yield) as a white solid. ¹H NMR (400 MHz, DMSO-d₆) δ=8.54 (br s, 1H), 7.41-7.35 (m, 2H), 7.33-7.28 (m, 2H), 7.27-7.18 (m, 4H), 7.12 (br d, J=8.0 Hz, 2H), 2.25 (s, 6H). MS (ESI) m/z 280.0 [M+H]⁺.

Step 2. A mixture of 3-(2,6-difluoro-4-(3-hydroxyazetidin-1-yl)phenyl)piperidine-2,6-dione XII (80.0 mg, 270 μmol, 1.00 eq) in dimethyl formamide (1 mL) was added sodium hydride (16.2 mg, 405 μmol, 60% purity, 1.50 eq) and phenyl (3-phenylbicyclo[1.1.1]pentan-1-yl)carbamate (79.2 mg, 283 μmol, 1.05 eq) at 0° C. The reaction mixture was stirred at 20° C. for 1 h. The reaction mixture was added formic acid (2.00 mL) and filtered to give a filtrate, which was purified by Prep-HPLC (column: Phenomenex luna C18 150*25 mm*10 μm; mobile phase: [water (0.225% formic acid)-acetonitrile]; B %: 35%-65%, 10 min) and lyophilized to give 1-(4-(2,6-dioxopiperidin-3-yl)-3,5-difluorophenyl)azetidin-3-yl(3-phenylbicyclo[1.1.1] pentan-1-yl) carbamate #61 (55.71 mg, 114 μmol, 42% yield, 99% purity) as a white solid. ¹H NMR (400 MHz, DMSO-d₆) δ=10.87 (s, 1H), 8.22 (br s, 1H), 7.35-7.27 (m, 2H), 7.27-7.19 (m, 3H), 6.20 (d, J=11.2 Hz, 2H), 5.17 (br s, 1H), 4.17 (br t, J=7.2 Hz, 2H), 4.05 (dd, J=5.2, 12.4 Hz, 1H), 3.75 (br s, 2H), 2.85-2.73 (m, 1H), 2.49 (br s, 1H), 2.20 (s, 6H), 2.08 (dq, J=3.6, 13.2 Hz, 1H), 2.00-1.90 (m, 1H). MS (ESI) m/z 481.9 [M+H]⁺.

### Example 63. Synthesis of Compound 62

Step 1. To a solution of 3-fluorobicyclo[1.1.1]pentan-1-amine hydrochloride (180 mg, 1.31 mmol, 1.00 eq) and pyridine (527 μL 6.54 mmol, 5.00 eq) in acetonitrile (5 mL) was added phenyl carbonochloridate (196 μL, 1.57 mmol, 1.20 eq). The reaction mixture was stirred at 20° C. for 1 h. The reaction mixture was poured into water (50 mL) and extracted with ethyl acetate (3×20 mL). The combined organic phase was separated, washed with brine (3×10 mL), dried over anhydrous sodium sulfate, filtered and concentrated to give phenyl (3-fluorobicyclo[1.1.1]pentan-1-yl)carbamate (300 mg, crude) as a white solid. ¹H NMR (400 MHz, DMSO-d₆) δ=8.64 (br s, 1H), 7.40-7.35 (m, 2H), 7.26-7.18 (m, 1H), 7.11 (br d, J=7.6 Hz, 2H), 2.33 (d, J=1.6 Hz, 6H). MS (ESI) m/z 222.1 [M+H]⁺. Step 2. A mixture of 3-(2,6-difluoro-4-(3-hydroxyazetidin-1-yl)phenyl)piperidine-2,6-dione XII (80.0 mg, 270 μmol, 1.00 eq) in dimethyl formamide (1 mL) was added sodium hydride (16.2 mg, 405 μmol, 60% purity, 1.50 eq) and phenyl (3-fluorobicyclo[1.1.1]pentan-1-yl)carbamate (62.7 mg, 283 μmol, 1.05 eq) at 0° C. The reaction mixture was stirred at 20° C. for 1 h. The reaction mixture was added formic acid (2 mL) and filtered to give a filtrate, which was purified by Prep-HPLC (column: Phenomenex Luna C18 150*25 mm*10 μm; mobile phase: [water (0.225% formic acid)-acetonitrile]; B %: 37%-67%, 10 min) and lyophilized to give 1-(4-(2,6-dioxopiperidin-3-yl)-3,5-difluorophenyl)azetidin-3-yl(3-fluorobicyclo[1.1.1] pentan-1-yl)carbamate #62 (29.57 mg, 69.1 μmol, 25% yield, 99% purity) as a white solid. ¹H NMR (400 MHz, DMSO-d₆) δ=10.86 (br s, 1H), 8.30 (br s, 1H), 6.21 (s, 1H), 6.18 (s, 1H), 5.16 (br s, 1H), 4.15 (br t, J=7.6 Hz, 2H), 4.04 (br dd, J=5.2, 12.4 Hz, 1H), 3.79-3.66 (m, 2H), 2.84-2.73 (m, 1H), 2.49-2.46 (m, 1H), 2.27 (d, J=2.0 Hz, 6H), 2.15-2.02 (m, 1H), 2.00-1.89 (m, 1H). MS (ESI) m/z 423.9 [M+H]⁺.

### Example 64. Synthesis of Compound 63

Step 1. To a solution of bicyclo[1.1.1]pentan-1-amine hydrochloride (294 μL, 128 μmol, 1.00 eq) and pyridine (51.7 μL, 641 μmol, 5.00 eq) in acetonitrile (1 mL) was added phenyl carbonochloridate (48.2 μL, 384 μmol, 3.00 eq). The reaction mixture was stirred at 25° C. for 12 h. The reaction was concentrated to give a residue. The residue was purified by reversed-phase HPLC (column: spherical C8, 20-35 μm, 100 Å, SW 40 g, mobile phase: [water (0.1% formic acid)-acetonitrile]) to afford phenyl bicyclo[1.1.1]pentan-1-ylcarbamate (20.0 mg, 98.4 μmol, 77% yield) as a white solid. ¹H NMR (400 MHz, DMSO-d₆) δ=8.44 (br s, 1H), 7.40-7.36 (m, 2H), 7.24-7.18 (m, 1H), 7.08 (br d, J=8.0 Hz, 2H), 2.42 (s, 1H), 1.98 (s, 6H). MS (ESI) m/z 204.2 [M+H]⁺.

Step 2. To a mixture of phenyl bicyclo[1.1.1]pentan-1-ylcarbamate (20.0 mg, 98.4 μmol, 1.00 eq) in dimethyl formamide (1 mL) was added 3-(2,6-difluoro-4-(3-hydroxyazetidin-1-yl)phenyl)piperidine-2,6-dione XII (35.0 mg, 118 μmol, 1.20 eq) and sodium hydride (5.90 mg, 148 μmol, 60% purity, 1.50 eq) at 0° C. The reaction mixture was stirred at 20° C. for 1 h. The reaction mixture was added formic acid (0.500 mL) and filtered to give a filtrate, which was purified by Prep-HPLC (column: Phenomenex luna C18 150*25 mm*10 μm; mobile phase: [water (0.225% formic acid)-acetonitrile]; B %: 34%-64%, 10 min) and lyophilized to give 1-(4-(2,6-dioxopiperidin-3-yl)-3,5-difluorophenyl)azetidin-3-yl bicyclo[1.1.1]pentan-1-ylcarbamate #63 (21.47 mg, 52.4 μmol, 53% yield, 99% purity) as a white solid. ¹H NMR (400 MHz, DMSO-d₆) δ=10.86 (br s, 1H), 8.12 (br s, 1H), 6.18 (br d, J=11.2 Hz, 2H), 5.14 (br s, 1H), 4.16 (br s, 2H), 4.04 (br dd, J=4.4, 12.8 Hz, 1H), 3.72 (br s, 2H), 2.82-2.74 (m, 1H), 2.62-2.54 (m, 1H), 2.38 (s, 1H), 2.12-2.04 (m, 1H), 1.94 (s, 7H). MS (ESI) m/z 406.1 [M+H]⁺.

### Example 65. Synthesis of Compound 64

Step 1. To a mixture of 2,3-dimethlbenzoic acid (10.0 g, 66.6 mmol, 1.00 eq) in sulfuric acid (60 mL) was added potassium nitrate (7.41 g, 73.3 mmol, 1.10 eq) at 0° C. Then the mixture was stirred at 25° C. for 1 h. The reaction mixture was filtered and concentrated under reduced pressure to give a residue. The residue was purified by silica gel column chromatography (SiO₂, petroleum ether/ethyl acetate=1/0 to 0/1) to afford 2,3-dimethyl-5-nitrobenzoic acid (2.00 g, 10.3 mmol, 15% yield) as a yellow solid. ¹H NMR (400 MHz, CDCl₃) δ=8.60 (d, J=2.6 Hz, 1H), 8.14 (d, J=2.6 Hz, 1H), 2.57 (s, 3H), 2.40 (s, 3H).

Step 2. To a mixture of 2,3-dimethyl-5-nitrobenzoic acid (3.50 g, 17.9 mmol, 1.00 eq) in tetrahydrofuran (30 mL) was added borane dimethyl sulfide complex (10 M, 3.59 mL, 2.00 eq) at 0° C. Then the mixture was stirred at 25° C. for 12 h. The reaction mixture was partitioned between water (50 mL) and ethyl acetate (3×80 mL). The organic phases were separated, washed with brine (3×50 mL), dried over sodium sulfuric acid, filtered and concentrated under reduced pressure to give a residue. The residue was purified by Prep-HPLC (formic acid condition; column: Phenomenex Luna C8 250*50 mm*10 μm; mobile phase: [water (0.225% formic acid)-acetonitrile]; B %: 10%-50%, 20 min) to afford (2,3-dimethyl-5-nitrophenyl) methanol (1.50 g, 8.28 mmol, 46% yield) as a white solid. 1H NMR (400 MHz, CDCl₃) δ=8.15 (d, J=2.2 Hz, 1H), 7.99 (d, J=2.2 Hz, 1H), 4.81 (s, 2H), 2.41 (s, 3H), 2.33 (s, 3H).

Step 3. To a mixture of (2,3-dimethyl-5-nitrophenyl)methanol (800 mg, 4.42 mmol, 1.00 eq) in dichloromethane (10 mL) was added Dess-Martin (3.42 g, 8.07 mmol, 2.50 mL, 1.00 eq). Then the mixture was stirred at 25° C. for 12 h. The reaction mixture was concentrated under reduced pressure to give a residue. The residue was purified by column chromatography (SiO₂, petroleum ether/ethyl acetate=1/0 to 3/1) to afford 2,3-dimethyl-5-nitrobenzaldehyde (500 mg, 2.79 mmol) as a white solid. ¹H NMR (400 MHz, CDCl₃) δ=10.29 (s, 1H), 8.44 (d, J=2.6 Hz, 1H), 8.17 (d, J=2.4 Hz, 1H), 2.62 (s, 3H), 2.41 (s, 3H).

Step 4. To a mixture of 2,3-dimethyl-5-nitrobenzaldehyde (450 mg, 2.51 mmol, 1.00 eq) and 2-oxa-5-azabicyclo[2.2.1]heptane (409 mg, 3.01 mmol, 1.20 eq, hydrochloric acid) in dichloromethane (1 mL) was added sodium triacetoxyhydroborate (1.06 g, 5.02 mmol, 2.00 eq) and sodium borohydride acetate, (216 μL, 3.77 mmol, 1.50 eq). Then the mixture was stirred at 25° C. for 10 h. The reaction mixture was filtered and concentrated under reduced pressure to give a residue. The filtrate was purified by reversed-phase HPLC (C18, 40 g; condition: water/acetonitrile=1/0 to 0/1, 0.1% formic acid) to afford 5-(2,3-dimethyl-5-nitrobenzyl)-2-oxa-5-azabicyclo[2.2.1]heptane (250 mg, 953 μmol, 38% yield) as a white solid. ¹H NMR (400 MHz, CDCl₃) δ=8.08 (d, J=2.0 Hz, 1H), 7.96 (d, J=2.2 Hz, 1H), 4.47 (s, 1H), 4.15 (d, J=7.8 Hz, 1H), 3.86-3.75 (m, 2H), 3.71 (dd, J=1.6, 7.8 Hz, 1H), 3.49 (s, 1H), 2.94 (br d, J=9.8 Hz, 1H), 2.60 (d, J=10.0 Hz, 1H), 2.41 (s, 3H), 2.35 (s, 3H), 1.96 (br d, J=9.2 Hz, 1H), 1.80 (br d, J=9.6 Hz, 1H).

Step 5. To a mixture of 5-(2,3-dimethyl-5-nitrobenzyl)-2-oxa-5-azabicyclo[2.2.1]heptane (430 mg, 1.64 mmol, 1.00 eq), ferrous powder (458 mg, 8.20 mmol, 5.00 eq) and ammonium chloride (87.7 mg, 1.64 mmol, 1.00 eq) in ethanol (3 mL) and water (1.50 mL) was stirred at 60° C. for 10 h. The reaction mixture was filtered. The crude filtrate was purified by reversed phase column chromatography (C18, 80 g; condition: water/acetonitrile=1/0 to 0/1, 0.1% formic acid) to afford 3-(2-oxa-5-azabicyclo[2.2.1]heptan-5-ylmethyl)-4,5-dimethylaniline (300 mg, 1.29 mmol, 79% yield) as a white solid. ¹H NMR (400 MHz, CDCl₃) δ=6.63 (br s, 1H), 6.48 (d, J=2.4 Hz, 1H), 4.44 (s, 1H), 4.16-4.13 (m, 1H), 3.71-3.64 (m, 3H), 3.49 (br s, 1H), 2.93 (br d, J=10.0 Hz, 1H), 2.65 (br d, J=10.2 Hz, 1H), 2.23 (s, 3H), 2.15 (s, 3H), 1.93 (br d, J=8.8 Hz, 1H), 1.75 (br d, J=9.6 Hz, 1H).

Step 6. To a mixture of 3-(2-oxa-5-azabicyclo[2.2.1]heptan-5-ylmethyl)-4,5-dimethylaniline (300 mg, 1.29 mmol, 1.00 eq) and potassium carbonate (357 mg, 2.58 mmol, 2.00 eq) in acetone (3 mL) was added phenyl carbonochloridate (194 μL, 1.55 mmol, 1.20 eq) at 0° C. Then the mixture was stirred at 25° C. for 10 h. The reaction mixture was filtered. The filtrate was purified by reversed-phase HPLC (C18, 40 g; condition: water/acetonitrile=1/0 to 0/1, 0.1% formic acid) to afford phenyl (3-(2-oxa-5-azabicyclo[2.2.1]heptan-5-ylmethyl)-4,5-dimethylphenyl)carbamate (210 mg, 596 μmol, 46% yield) as a white solid. ¹H NMR (400 MHz, DMSO-d₆) δ=9.99 (br s, 1H), 7.46-7.39 (m, 2H), 7.35 (s, 1H), 7.26 (br t, J=7.4 Hz, 1H), 7.20 (br d, J=7.2 Hz, 3H), 4.37 (s, 1H), 3.93 (br d, J=7.6 Hz, 1H), 3.67 (br d, J=4.6 Hz, 2H), 3.54 (br d, J=7.6 Hz, 2H), 2.76 (br d, J=9.4 Hz, 1H), 2.47 (br s, 1H), 2.21 (s, 3H), 2.15 (s, 3H), 1.82 (br d, J=9.4 Hz, 1H), 1.60 (br d, J=9.4 Hz, 1H).

Step 7. To a mixture of phenyl (3-(2-oxa-5-azabicyclo[2.2.1]heptan-5-ylmethyl)-4,5-dimethylphenyl)carbamate (70.0 mg, 199 μmol, 1.00 eq) and 3-(2,6-difluoro-4-(3-hydroxyazetidin-1-yl)phenyl)piperidine-2,6-dione XII (53.0 mg, 179 μmol, 0.90 eq) in dimethylformamide (1 mL) was added sodium hydride (15.9 mg, 397 μmol, 60% purity, 2.00 eq) at 0° C. Then the mixture was stirred 25° C. for 1 h. The reaction mixture was filtered. The filtrate was purified by Prep-HPLC (neutral condition; column: Waters Xbridge 150*25 mm*5 μm; mobile phase: [water (10 mM sodium bicarbonate)-acetonitrile]; B %: 40%-70%, 10 min) to afford 1-(4-(2,6-dioxopiperidin-3-yl)-3,5-difluorophenyl)azetidin-3-yl(3-(2-oxa-5-azabicyclo[2.2.1]heptan-5-ylmethyl)-4,5-dimethylphenyl) carbamate #64 (13.5 mg, 24.0 μmol, 12% yield, 99% purity) as a white solid. ¹H NMR (400 MHz, DMSO-d₆) δ=10.87 (br s, 1H), 9.65 (br s, 1H), 7.28 (s, 1H), 7.15 (br s, 1H), 6.24 (s, 1H), 6.21 (s, 1H), 5.32-5.20 (m, 1H), 4.35 (s, 1H), 4.22 (br t, J=7.8 Hz, 2H), 4.05 (br dd, J=4.8, 12.5 Hz, 1H), 3.91 (d, J=7.2 Hz, 1H), 3.82 (br dd, J=3.8, 9.0 Hz, 2H), 3.62 (d, J=4.4 Hz, 2H), 3.53 (dd, J=1.6, 7.3 Hz, 1H), 3.44-3.39 (m, 2H), 2.84-2.74 (m, 1H), 2.73 (br d, J=10.0 Hz, 1H), 2.44 (br d, J=9.8 Hz, 1H), 2.19 (s, 3H), 2.13 (s, 3H), 2.11-2.04 (m, 1H), 1.99-1.91 (m, 1H), 1.79 (br d, J=8.2 Hz, 1H), 1.59 (br d, J=9.4 Hz, 1H). MS (ESI) m/z 555.2 [M+H]⁺.

### Example 66. Synthesis of Compound 65

Step 1. To a solution of 3-(piperidin-1-yl)bicyclo[1.1.1]pentan-1-amine hydrochloride (294 μL, 0.493 mmol, 1.00 eq) and phenyl carbonochloridate (185 μL, 1.48 mmol, 3.00 eq) in acetonitrile (1 mL) was added cesium carbonate (482 mg, 1.48 mmol, 3.00 eq). The reaction mixture was stirred at 25° C. for 2 h. The reaction was concentrated to give a residue. The residue was purified by reversed-phase HPLC (column: spherical C8, 20-35 μm, 100 Å, SW 40 g, mobile phase: [water (0.1% formic acid)-acetonitrile]) to afford phenyl (3-(piperidin-1-yl)bicyclo[1.1.1]pentan-1-yl)carbamate (100 mg, 0.349 μmol, 71% yield) as a white solid. MS (ESI) m/z 287.2 [M+H]⁺.

Step 2. To a mixture of phenyl (3-(piperidin-1-yl)bicyclo[1.1.1]pentan-1-yl)carbamate (20.0 mg, 69.8 μmol, 1.00 eq) in dimethyl formamide (1 mL) was added 3-(2,6-difluoro-4-(3-hydroxyazetidin-1-yl)phenyl) piperidine-2,6-dione XII (24.8 mg, 83.8 μmol, 1.20 eq) and sodium hydride (4.19 mg, 105 μmol, 60% purity, 1.50 eq) at 0° C. The reaction mixture was stirred at 20° C. for 1 h. The reaction mixture was added formic acid (0.500 mL) and filtered to give a filtrate, which was purified by Prep-HPLC (column: Phenomenex luna C18 150*25 mm*10 μm; mobile phase: [water (0.225% formic acid)-acetonitrile]; B %: 5%-35%, 10 min) and lyophilized to give 1-(4-(2,6-dioxopiperidin-3-yl)-3,5-difluorophenyl)azetidin-3-yl (3-(piperidin-1-yl)bicyclo[1.1.1]pentan-1-yl) carbamate #65 (20.87 mg, 42.3 μmol, 60% yield, 99% purity) as a white solid. ¹H NMR (400 MHz, DMSO-d₆) δ=10.86 (br d, J=0.8 Hz, 1H), 8.09 (br s, 1H), 6.19 (br d, J=11.2 Hz, 2H), 5.19-5.10 (m, 1H), 4.14 (br t, J=7.4 Hz, 2H), 4.04 (br dd, J=4.8, 12.6 Hz, 1H), 3.72 (br s, 2H), 2.84-2.73 (m, 1H), 2.48 (br s, 1H), 2.30 (br s, 4H), 2.13-2.03 (m, 1H), 1.98-1.91 (m, 1H), 1.86 (s, 6H), 1.51-1.43 (m, 4H), 1.35 (br d, J=4.9 Hz, 2H). MS (ESI) m/z 489.1 [M+H]⁺.

### Example 67. Synthesis of Compound 66

To a solution of 3-(4-(3-aminoazetidin-1-yl)phenyl)piperidine-2,6-dione XXII (108 mg, 417 μmol, 1.00 eq) in dimethyl formamide (1.10 mL) was added triethylamine (174 μL, 1.25 mmol, 3.00 eq) and phenyl (3-chloro-4-methylphenyl)carbamate (131 mg, 500 μmol, 1.20 eq). The mixture was stirred at 25° C. for 12 h. The pH of the mixture was adjusted to 7 with formic acid, then the mixture was diluted with dimethyl formamide (1 mL). The residue was purified by Prep-HPLC (column: Phenomenex luna C18 150*25 mm*10 μm; mobile phase: [water (0.225% formic acid)-acetonitrile]; B %: 31%-61%, 10 min) the desired eluent was lyophilized to afford 1-(3-chloro-4-methylphenyl)-3-(1-(4-(2,6-dioxopiperidin-3-yl)phenyl)azetidin-3-yl)urea #66 (15.4 mg, 33.9 μmol, 8% yield, 94% purity, formic acid) as a white solid. ¹H NMR (400 MHz, DMSO-d₆) δ=10.75 (s, 1H), 8.74 (s, 1H), 7.63 (d, J=1.6 Hz, 1H), 7.21-7.11 (m, 2H), 7.02 (br d, J=8.4 Hz, 3H), 6.43 (d, J=8.4 Hz, 2H), 4.56 (sxt, J=6.8 Hz, 1H), 4.10 (t, J=7.2 Hz, 2H), 3.70 (dd, J=4.8, 10.8 Hz, 1H), 3.57 (t, J=6.4 Hz, 2H), 2.63 (ddd, J=5.2, 11.4, 17.2 Hz, 1H), 2.49-2.41 (m, 1H), 2.23 (s, 3H), 2.17-2.06 (m, 1H), 2.04-1.95 (m, 1H). MS (ESI) m/z 427.0 [M+H]⁺.

### Example 68. Synthesis of Compound 67

To a solution of 3-(4-(3-aminoazetidin-1-yl)phenyl)piperidine-2,6-dione XXII (108 mg, 0.417 mmol, 1.00 eq) in dimethyl formamide (1.10 mL) was added triethylamine (126 mg, 1.25 mmol, 174 uL, 3.00 eq) and phenyl (3,5-dimethylphenyl)carbamate (121 mg, 0.400 mmol, 1.20 eq). The mixture was stirred at 25° C. for 12 h. The pH of the mixture was adjusted to 7 with formic acid, then the mixture was diluted with dimethyl formamide (1 mL). The residue was purified by Prep-HPLC (column: Phenomenex luna C18 150*25 mm*10 μm; mobile phase: [water (0.225% formic acid)-acetonitrile]; B %: 28%-58%, 10 min) followed by Prep-HPLC (column: Waters Xbridge 150*25 mm*5 μm; mobile phase: [Water-acetonitrile]; B %: 32%-62%, 10 min). The desired eluent was lyophilized to afford 1-(3,5-dimethylphenyl)-3-(1-(4-(2,6-dioxopiperidin-3-yl)phenyl)azetidin-3-yl)urea #67 (7.87 mg, 0.0188 mmol, 4% yield, 97% purity) as a white solid. ¹H NMR (400 MHz, DMSO-d₆) δ=10.75 (s, 1H), 8.32 (s, 1H), 7.04-6.98 (m, 4H), 6.77 (d, J=7.6 Hz, 1H), 6.55 (s, 1H), 6.43 (d, J=8.8 Hz, 2H), 4.59-4.49 (m, 1H), 4.10 (t, J=7.2 Hz, 2H), 3.71 (dd, J=4.8, 10.8 Hz, 1H), 3.55 (t, J=6.8 Hz, 2H), 2.65-2.57 (m, 1H), 2.48-2.41 (m, 1H), 2.19 (s, 6H), 2.15-2.06 (m, 1H), 2.04-1.95 (m, 1H). MS (ESI) m/z 407.1 [M+H]⁺.

### Example 69. Synthesis of Compound 68

To a solution of 3-(2,6-difluoro-4-(3-hydroxyazetidin-1-yl)phenyl)piperidine-2,6-dione XII (80.0 mg, 270 μmol, 1.00 eq) and phenyl (3-(fluoromethyl)bicyclo[1.1.1]pentan-1-yl)carbamate (76.2 mg, 324 μmol, 1.20 eq) in dimethylformamide (1 mL) was added sodium hydride (16.2 mg, 405 μmol, 60% purity, 1.50 eq) at 0° C. The mixture was stirred at 20° C. for 1 h. The mixture was quenched by formic acid (1 mL) and filtered. The filtrate was purified by prep-HPLC (column: Phenomenex Synergi C18 150*25 mm*10 μm; mobile phase: [water (0.225% formic acid)-acetonitrile]; B %: 31%-64%, 11 min) and lyophilized to afford 1-(4-(2,6-dioxopiperidin-3-yl)-3,5-difluorophenyl)azetidin-3-yl (3-(fluoromethyl)bicyclo[1.1.1] pentan-1-yl) carbamate #68 (32.73 mg, 74.1 μmol, 27% yield, 99% purity) as a white solid. 1H NMR (400 MHz, DMSO-d₆) δ=10.86 (s, 1H), 8.17 (br s, 1H), 6.19 (d, J=11.0 Hz, 2H), 5.25-5.02 (m, 1H), 4.53 (s, 1H), 4.42 (s, 1H), 4.14 (br t, J=7.5 Hz, 2H), 4.04 (dd, J=4.6, 12.7 Hz, 1H), 3.71 (br d, J=5.0 Hz, 2H), 2.82-2.72 (m, 1H), 2.53-2.52 (m, 1H), 2.13-2.03 (m, 1H), 1.99-1.93 (m, 1H), 1.91 (s, 6H). MS (ESI) m/z 438.2 [M+H]⁺.

### Example 70. Synthesis of Compound 69

To a solution of 3-(2,6-difluoro-4-(3-hydroxyazetidin-1-yl)phenyl)piperidine-2,6-dione XII (65.0 mg, 219 μmol, 1.00 eq) and phenyl (3-(difluoro(4-methoxyphenyl)methyl)bicyclo[1.1.1]pentan-1-yl)carbamate (86.7 mg, 241 μmol, 1.10 eq) in dimethylformamide (1 mL) was added sodium hydride (13.2 mg, 329 μmol, 60% purity, 1.50 eq) at 0° C. The mixture was stirred at 20° C. for 1 h. The mixture was quenched by formic acid (1 mL) and filtered. The filtrate was purified by prep-HPLC (column: Phenomenex Synergi C18 150*25 mm*10 μm; mobile phase: [water (0.225% formic acid)-acetonitrile]; B %: 50%-70%, 10 min) and lyophilized to afford 1-(4-(2,6-dioxopiperidin-3-yl)-3,5-difluorophenyl)azetidin-3-yl (3-(difluoro(4-methoxyphenyl)methyl)bicyclo[1.1.1]pentan-1-yl)carbamate #69 (54.98 mg, 96.9 μmol, 44% yield, 99% purity) as a white solid. ¹H NMR (400 MHz, DMSO-d₆) δ=10.85 (s, 1H), 8.25 (br s, 1H), 7.32 (d, J=8.9 Hz, 2H), 7.02 (d, J=8.7 Hz, 2H), 6.18 (d, J=11.0 Hz, 2H), 5.13 (br s, 1H), 4.13 (br t, J=7.5 Hz, 2H), 4.03 (br dd, J=4.6, 12.4 Hz, 1H), 3.79 (s, 3H), 3.69 (br d, J=5.3 Hz, 2H), 2.86-2.71 (m, 1H), 2.53-2.51 (m, 1H), 2.12-2.01 (m, 1H), 1.94 (s, 6H), 1.93-1.89 (m, 1H). MS (ESI) m/z 562.3 [M+H]⁺.

### Example 71. Synthesis of Compound 70

To a solution of 3-(2,6-difluoro-4-(3-hydroxyazetidin-1-yl)phenyl)piperidine-2,6-dione XII (45.0 mg, 152 μmol, 1.00 eq) and phenyl (3-(difluoromethyl)bicyclo[1.1.1]pentan-1-yl)carbamate (38.5 mg, 152 μmol, 1.00 eq) in dimethylformamide (1 mL) was added sodium hydride (6.07 mg, 152 μmol, 60% purity, 1.00 eq) at 0° C. The mixture was stirred at 20° C. for 1 h. The mixture was quenched by formic acid (1 mL) and filtered. The mixture was purified by prep-HPLC (column: Phenomenex luna C18 150*25 mm*10 μm; mobile phase: [water (0.225% formic acid)-acetonitrile]; B %: 33%-63%, 10 min) and lyophilized to afford 1-(4-(2,6-dioxopiperidin-3-yl)-3,5-difluorophenyl)azetidin-3-yl(3-(difluoromethyl)bicyclo[1.1.1] pentan-1-yl)carbamate #70 (28.38 mg, 61.7 μmol, 41% yield, 99% purity) as a white solid. 1H NMR (400 MHz, DMSO-d₆) δ=10.86 (s, 1H), 8.27 (br s, 1H), 6.20 (s, 1H), 6.17 (s, 1H), 6.15 (t, J=56.4 Hz, 1H), 5.15 (br s, 1H), 4.14 (br t, J=7.6 Hz, 2H), 4.04 (dd, J=4.8, 12.5 Hz, 1H), 3.72 (br s, 2H), 2.83-2.73 (m, 1H), 2.55-2.52 (m, 1H), 2.14-2.05 (m, 1H), 2.00 (s, 6H), 1.97-1.90 (m, 1H). MS (ESI) m/z 456.2 [M+H]⁺.

### Example 72. Synthesis of Compound 71

To a solution of 3-(2,6-difluoro-4-(3-hydroxyazetidin-1-yl)phenyl)piperidine-2,6-dione XII (75.0 mg, 253 μmol, 1.00 eq) and phenyl (3-morpholinobicyclo[1.1.1]pentan-1-yl)carbamate (80.3 mg, 278 μmol, 1.10 eq) in dimethylformamide (1 mL) was added sodium hydride (10.1 mg, 253 μmol, 60% purity, 1.00 eq) at 0° C. The mixture was stirred at 20° C. for 1 h. The mixture was quenched by formic acid (1 mL) and filtered. The filtrate was purified by prep-HPLC (column: Phenomenex luna C18 150*25 mm*10 μm; mobile phase: [water (0.225% formic acid)-acetonitrile]; B %: 7%-37%, 10 min) and lyophilized to afford 1-(4-(2,6-dioxopiperidin-3-yl)-3,5-difluorophenyl)azetidin-3-yl(3-morpholinobicyclo [1.1.1]pentan-1-yl) carbamate #71 (38.95 mg, 78.6 μmol, 31% yield, 99% purity) as a white solid. ¹H NMR (400 MHz, DMSO-d₆) δ=10.86 (s, 1H), 8.12 (br s, 1H), 6.20 (s, 1H), 6.17 (s, 1H), 5.13 (br s, 1H), 4.14 (br t, J=7.2 Hz, 2H), 4.03 (br dd, J=5.0, 12.5 Hz, 1H), 3.70 (br d, J=6.1 Hz, 2H), 3.60-3.53 (m, 4H), 2.83-2.73 (m, 1H), 2.56-2.52 (m, 1H), 2.35-2.29 (m, 4H), 2.13-2.02 (m, 1H), 1.98-1.91 (m, 1H), 1.87 (s, 6H). MS (ESI) m/z 491.3 [M+H]⁺.

### Example 73. Synthesis of Compound 72

To a solution of 3-(4-(3-aminoazetidin-1-yl)phenyl)piperidine-2,6-dione XXII (100 mg, 386 μmol, 1.00 eq) in dimethyl formamide (1.10 mL) was added phenyl (5-chloro-2-methoxy-4-methylphenyl)carbamate (124 mg, 424 μmol, 1.10 eq) and triethylamine (161 μL, 1.16 mmol, 3.00 eq). The mixture was stirred at 25° C. for 12 h. The pH was adjusted to around 7 by progressively adding formic acid, then the mixture was diluted with dimethyl formamide (1 mL). The residue was purified by prep-HPLC (column: Phenomenex luna C18 150*25 mm*10 μm; mobile phase: [water (formic acid)-acetonitrile]; B %: 34%-64%, 10 min) and prep-NPLC (column: Welch Ultimate XB-CN 250*50*10 μm; mobile phase: [Hexane-ethyl alcohol]; B %: 20%-60%, 15 min). Then the desired eluent was lyophilized to afford 1-(5-chloro-2-methoxy-4-methylphenyl)-3-(1-(4-(2,6-dioxopiperidin-3-yl)phenyl)azetidin-3-yl)urea #72 (25.62 mg, 53.8 μmol, 14% yield, 96% purity) as a white solid. ¹H NMR (400 MHz, DMSO-d₆) δ=10.75 (br s, 1H), 8.13 (s, 1H), 7.99 (s, 1H), 7.58 (br d, J=6.8 Hz, 1H), 7.02 (d, J=8.4 Hz, 2H), 6.96 (s, 1H), 6.43 (d, J=8.4 Hz, 2H), 4.57-4.49 (m, 1H), 4.11 (t, J=7.2 Hz, 2H), 3.82 (s, 3H), 3.71 (br dd, J=4.9, 11.2 Hz, 1H), 3.53-3.48 (m, 2H), 2.64-2.58 (m, 1H), 2.48-2.41 (m, 1H), 2.25 (s, 3H), 2.18-2.05 (m, 1H), 2.04-1.95 (m, 1H). MS (ESI) m/z 457.3 [M+H]⁺.

### Example 74. Synthesis of Compound 73

Step 1. To a solution of 3-phenylbicyclo[1.1.1]pentan-1-amine (80.0 mg, 409 μmol, 1.00 eq, hydrochloride) and pyridine (165 μL, 2.04 mmol, 5.00 eq) in acetonitrile (1 mL) was added phenyl carbonochloridate (154 μL, 1.23 mmol, 3.00 eq) at 0° C. The mixture was stirred at 20° C. for 1 h. The mixture was quenched by ice water (20 mL) and extracted with ethyl acetate (3×40 mL). The organic layers were washed with brine (30 mL), dried over anhydrous sodium sulfate, filtered and concentrated to afford a residue. The residue was purified by reversed phase (C18, 40 g; condition: water/acetonitrile=1/0 to 0/1, 0.1% formic acid) and lyophilized to afford phenyl (3-phenylbicyclo[1.1.1]pentan-1-yl)carbamate (110 mg, 394 μmol, 96% yield) as a white solid. ¹H NMR (400 MHz, DMSO-d₆) δ=8.54 (br s, 1H), 7.41-7.35 (m, 2H), 7.34-7.28 (m, 2H), 7.26-7.16 (m, 4H), 7.11 (br d, J=7.8 Hz, 2H), 2.24 (s, 6H).

Step 2. To a solution of 3-(4-(3-aminoazetidin-1-yl)-2,6-difluorophenyl)piperidine-2,6-dione IX (80.0 mg, 234 μmol, 1.00 eq, formate) and triethylamine (131 μL, 938 μmol, 4.00 eq) in dimethylformamide (1 mL) was added phenyl (3-phenylbicyclo[1.1.1]pentan-1-yl)carbamate (78.6 mg, 281 μmol, 1.20 eq) at 0° C. The mixture was stirred at 30° C. for 12 h. The mixture was filtered. The filtrate was purified by Prep-HPLC (column: Phenomenex Synergi C18 150*25 mm*10 μm; mobile phase: [water(formic acid)-acetonitrile]; B %: 37%-67%, 10 min) and lyophilized to afford 1-(1-(4-(2,6-dioxopiperidin-3-yl)-3,5-difluorophenyl)azetidin-3-yl)-3-(3-phenylbicyclo[1.1.1]pentan-1-yl)urea #73 (52.19 mg, 106 μmol, 45% yield, 98% purity) as a white solid. ¹H NMR (400 MHz, DMSO-d₆) δ=10.85 (br s, 1H), 7.32-7.26 (m, 2H), 7.25-7.16 (m, 3H), 6.73 (s, 1H), 6.43 (br d, J=7.6 Hz, 1H), 6.16 (s, 1H), 6.14 (s, 1H), 4.55-4.41 (m, 1H), 4.11-4.00 (m, 3H), 3.58 (br t, J=6.4 Hz, 2H), 2.83-2.73 (m, 1H), 2.53-2.52 (m, 1H), 2.18 (s, 6H), 2.10-2.02 (m, 1H), 1.98-1.90 (m, 1H). MS (ESI) m/z 481.3 [M+H]⁺.

### Example 75. Synthesis of Compound 74

To a solution of 3-(4-(3-aminoazetidin-1-yl)phenyl)piperidine-2,6-dione XXII (150 mg, 578 μmol, 1.00 eq) in dimethyl formamide (1.10 mL) was added triethylamine (242 μL, 1.74 mmol, 3.00 eq) and phenyl (4-(trifluoromethoxy)pyridin-2-yl)carbamate (190 mg, 636 μmol, 1.10 eq). The mixture was stirred at 25° C. for 12 h. The reaction mixture was diluted with water (20 mL) and extracted with ethyl acetate (3×8 mL). Then the organic phase was washed with brine (5 mL) and dried over anhydrous sodium sulfate, filtered and concentrated to give a residue. The residue was triturated with ethyl acetate at 25° C. for 5 min to afford 1-(1-(4-(2,6-dioxopiperidin-3-yl)phenyl)azetidin-3-yl)-3-(4-(trifluoromethoxy)pyridin-2-yl)urea #74 (123.62 mg, 256 μmol, 44% yield, 96% purity) as a white solid. ¹H NMR (400 MHz, DMSO-d₆) δ=10.77 (s, 1H), 9.47 (s, 1H), 8.31 (d, J=6.0 Hz, 1H), 8.09 (br d, J=7.2 Hz, 1H), 7.58 (s, 1H), 7.02 (d, J=8.4 Hz, 2H), 6.95 (br d, J=6.0 Hz, 1H), 6.44 (d, J=8.4 Hz, 2H), 4.66-4.56 (m, 1H), 4.13 (t, J=7.2 Hz, 2H), 3.71 (dd, J=4.6, 11.2 Hz, 1H), 3.63-3.56 (m, 2H), 2.64-2.59 (m, 1H), 2.48-2.41 (m, 1H), 2.18-2.06 (m, 1H), 2.04-1.94 (m, 1H). MS (ESI) m/z 464.2 [M+H]⁺.

### Example 76. Synthesis of Compound 75

Step 1. To a solution of methyl 3-aminobicyclo[1.1.1]pentane-1-carboxylate (80.0 mg, 450 μmol, 1.00 eq, hydrochloride) and pyridine (182 μL, 2.25 mmol, 5.00 eq) in acetonitrile (1 mL) was added phenyl carbonochloridate (169 μL, 1.35 mmol, 3.00 eq) at 0° C. The mixture was stirred at 20° C. for 1 h. The mixture was quenched by ice water (20 mL) and extracted with ethyl acetate (3×40 mL). The organic layers were washed with brine (30 mL), dried over anhydrous sodium sulfate, filtered and concentrated to afford a residue. The residue was purified by reversed phase column chromatography (C18, 40 g; condition: water/acetonitrile=1/0 to 0/1, 0.1% formic acid) and lyophilized to afford methyl 3-((phenoxycarbonyl)amino)bicyclo[1.1.1]pentane-1-carboxylate (90.0 mg, 344 μmol, 76% yield) as a white solid. ¹H NMR (400 MHz, DMSO-d₆) δ=8.60 (s, 1H), 7.41-7.34 (m, 2H), 7.25-7.18 (m, 1H), 7.15-7.05 (m, 2H), 3.61 (s, 3H), 2.23 (s, 6H).

Step 2. To a solution of 3-(2,6-difluoro-4-(3-hydroxyazetidin-1-yl)phenyl)piperidine-2,6-dione XII (80.0 mg, 270 μmol, 1.00 eq) and methyl 3-((phenoxycarbonyl)amino)bicyclo[1.1.1]pentane-1-carboxylate (77.6 mg, 297 μmol, 1.10 eq) in dimethylformamide (1 mL) was added sodium hydride (16.2 mg, 405 μmol, 60% purity, 1.50 eq) at 0° C. The mixture was stirred at 20° C. for 1 h. The mixture was quenched by formic acid (1 mL) at 0° C. and filtered. The filtrate was purified by Prep-HPLC (column: Phenomenex Synergi C18 150*25 mm*10 μm; mobile phase: [water (formic acid)-acetonitrile]; B %: 32%-52%, 10 min) and lyophilized to give a residue. The residue was further purified by Prep-HPLC (column: Phenomenex Synergi C18 150*25 mm*10 μm; mobile phase: [water (formic acid)-acetonitrile]; B %: 31%-61%, 9 min) and lyophilized to afford methyl 3-((((1-(4-(2,6-dioxopiperidin-3-yl)-3,5-difluorophenyl)azetidin-3-yl)oxy)carbonyl) amino)bicyclo[1.1.1]pentane-1-carboxylate #75 (29.04 mg, 61.4 μmol, 23% yield, 98% purity) as a white solid. ¹H NMR (400 MHz, DMSO-d₆) δ=10.85 (s, 1H), 8.26 (br s, 1H), 6.18 (br d, J=11.1 Hz, 2H), 5.14 (br d, J=1.2 Hz, 1H), 4.14 (br t, J=7.5 Hz, 2H), 4.03 (br dd, J=5.0, 12.5 Hz, 1H), 3.71 (br s, 2H), 3.60 (s, 3H), 2.85-2.73 (m, 1H), 2.55-2.52 (m, 1H), 2.18 (s, 6H), 2.13-2.05 (m, 1H), 1.98-1.91 (m, 1H). MS (ESI) m/z 464.1 [M+H]⁺.

### Example 77. Synthesis of Compound 76

Step 1. To a mixture of 4-fluoro-3-methylaniline (500 mg, 4.00 mmol, 1.00 eq) and pyridine (967 μL, 12.0 mmol, 3.00 eq) in acetonitrile (5 mL) was added phenyl carbonochloridate (750 μL, 5.99 mmol, 1.50 eq). The reaction mixture was stirred at 20° C. for 1 h. The reaction mixture was diluted with water (100 mL) and extracted with ethyl acetate (3×50 mL). The combined organic layers were washed with brine (50 mL), dried over anhydrous sodium sulfate filtered and concentrated under reduced pressure to give a residue. The residue was purified by flash silica gel chromatography (20.0 g, ethyl acetate/petroleum ether=0/1 to 1/4) to afford phenyl (4-fluoro-3-methylphenyl)carbamate (1.10 g, crude) as a white solid. ¹H NMR (400 MHz, DMSO-d₆) δ=10.19 (br s, 1H), 7.46-7.37 (m, 3H), 7.32 (td, J=3.8, 8.0 Hz, 1H), 7.28-7.23 (m, 1H), 7.23-7.19 (m, 2H), 7.10 (t, J=9.2 Hz, 1H), 2.21 (d, J=1.6 Hz, 3H).

Step 2. A mixture of 3-(2,6-difluoro-4-(3-hydroxyazetidin-1-yl) phenyl) piperidine-2,6-dione XII (106 mg, 359 μmol, 1.00 eq) in dimethyl formamide (1 mL) was added sodium hydride (28.0 mg, 717 umol, 60% purity, 2.00 eq) and phenyl (4-fluoro-3-methylphenyl)carbamate (96.8 mg, 394 μmol, 1.10 eq) at 0° C. The reaction mixture was stirred at 20° C. for 1 h. The filtrate was purified by Prep-HPLC (column: Phenomenex Luna C18 150*25 mm*10 μm; mobile phase: [water(formic acid)-acetonitrile]; B %: 41%-71%, 10 min) and lyophilized to afford 1-(4-(2,6-dioxopiperidin-3-yl)-3,5-difluorophenyl)azetidin-3-yl (4-fluoro-3-methylphenyl)carbamate #76 (56.55 mg, 125 μmol, 34% yield, 99% purity) as a white solid. ¹H NMR (400 MHz, DMSO-d₆) δ=10.86 (s, 1H), 9.85 (br s, 1H), 7.39-7.31 (m, 1H), 7.27 (br dd, J=4.2, 8.4 Hz, 1H), 7.06 (t, J=9.2 Hz, 1H), 6.22 (d, J=11.2 Hz, 2H), 5.30-5.23 (m, 1H), 4.21 (dd, J=7.2, 8.4 Hz, 2H), 4.04 (dd, J=5.1, 12.8 Hz, 1H), 3.82 (dd, J=3.6, 8.8 Hz, 2H), 2.84-2.72 (m, 1H), 2.52 (br d, J=2.0 Hz, 1H), 2.19 (d, J=1.6 Hz, 3H), 2.14-2.01 (m, 1H), 1.99-1.90 (m, 1H). MS (ESI) m/z 448.2 [M+H]⁺.

### Example 78. Synthesis of Compound 77

Step 1. A mixture of (1r,3r)-3-phenylcyclobutan-1-amine (50.0 mg, 340 μmol, 1.00 eq) and phenyl carbonochloridate (85.1 μL, 679 μmol, 2.00 eq) in pyridine (3 mL) was stirred at 25° C. for 12 h. The reaction was quenched by water (20 mL) and extracted with ethyl acetate (2×50 mL). The combined organic layers were washed with brine (20 mL), dried over anhydrous sodium sulfate, filtered and concentrated under reduced pressure to give a residue. The residue was purified by column chromatography (SiO₂, petroleum ether/ethyl acetate=20/1 and then 5/1) to afford phenyl ((1R,3R)-3-phenylcyclobutyl)carbamate (95.0 mg, crude) as a white solid. ¹H NMR (400 MHz, DMSO-d₆) δ=8.26 (d, J=7.2 Hz, 1H), 7.39-7.28 (m, 6H), 7.23-7.17 (m, 2H), 7.11 (d, J=7.6 Hz, 2H), 4.20-4.08 (m, 1H), 3.66-3.50 (m, 1H), 2.48-2.39 (m, 4H). MS (ESI) m/z 268.0 [M+H]⁺.

Step 2. A mixture of 3-(4-(3-aminoazetidin-1-yl)-2,6-difluorophenyl)piperidine-2,6-dione IX (36.5 mg, 123 μmol, 1.10 eq) in N,N-dimethyl formamide (3 mL) was added sodium hydride (8.98 mg, 225 μmol, 60% purity, 2.00 eq) at 0° C. The reaction was stirred at 0° C. for 30 min. Then a solution of phenyl ((1R,3R)-3-phenylcyclobutyl)carbamate (30.0 mg, 112 μmol, 1.00 eq) in N,N-dimethyl formamide (2 mL) was added dropwise to the mixture at 0° C. After stirring at 25° C. for 2 h, the reaction mixture was quenched by addition saturated ammonium chloride aqueous solution (30 mL) and extracted with ethyl acetate (2×30 mL). The combined organic layers were washed with brine (10 mL), dried over anhydrous sodium sulfate, filtered and concentrated under reduced pressure to give a residue. The residue was purified by Prep-HPLC (column: Phenomenex Synergi C18 150*25 mm*10 μm; mobile phase: [water(formic acid)-acetonitrile]; B %: 39%-59%, 10 min) and lyophilized to afford 1-(1-(4-(2,6-dioxopiperidin-3-yl)-3,5-difluorophenyl)azetidin-3-yl)-3-((1R,3R)-3-phenylcyclobutyl)urea #77 (12.0 mg, 25.6 μmol, 19% yield, 97% purity, formate) #77 as a white solid. ¹H NMR (400 MHz, DMSO-d₆) δ=10.86 (s, 1H), 8.48 (s, 1H), 7.37-7.24 (m, 4H), 7.23-7.16 (m, 1H), 6.59 (d, J=7.6 Hz, 1H), 6.53 (d, J=7.6 Hz, 1H), 6.15 (d, J=11.2 Hz, 2H), 4.54-4.45 (m, 1H), 4.21-4.14 (m, 1H), 4.10-4.00 (m, 3H), 3.58 (t, J=6.4 Hz, 2H), 3.50-3.46 (m, 1H), 2.89-2.72 (m, 1H), 2.40-2.25 (m, 5H), 2.14-2.01 (m, 1H), 1.99-1.89 (m, 1H). MS (ESI) m/z 468.9 [M+H]⁺.

### Example 79. Synthesis of Compound 78

Step 1. To a mixture of 3-fluoro-5-methylaniline (100 mg, 799 μmol, 1.00 eq), pyrimidine (129 μL, 1.60 mmol, 2.00 eq) in acetonitrile (5 mL) was added phenyl carbonochloridate (105 μL, 839 μmol, 1.05 eq). The reaction was stirred at 20° C. for 1 h. The reaction mixture was diluted with water (100 mL) and extracted with ethyl acetate (3×50 mL). The combined organic layers were washed with brine (50 mL), dried over anhydrous sodium sulfate, filtered and concentrated under reduced pressure to give a residue. The residue was purified by flash silica gel chromatography (20 g, ethyl acetate in petroleum ether was 0%-20%) to afford phenyl (3-fluoro-5-methylphenyl)carbamate (165 mg, 673 μmol, 84% yield) as a white solid. ¹H NMR (400 MHz, DMSO-d₆) δ=10.38 (br s, 1H), 7.47-7.40 (m, 2H), 7.30-7.24 (m, 1H), 7.24-7.23 (m, 1H), 7.22-7.21 (m, 1H), 7.10 (s, 1H), 6.74 (dd, J=1.8, 9.2 Hz, 1H), 2.28 (s, 3H).

Step 2. To a mixture of 3-(2,6-difluoro-4-(3-hydroxyazetidin-1-yl)phenyl)piperidine-2,6-dione XII (80.0 mg, 270 μmol, 1.00 eq) in dimethyl formamide (1 mL) were added phenyl (3-fluoro-5-methylphenyl)carbamate (72.9 mg, 297 umol, 1.10 eq) and sodium hydrogen (21.6 mg, 540 μmol, 60% purity, 2.00 eq). The reaction was stirred at 25° C. for 1 h. The reaction was purified by prep-HPLC (column: Phenomenex luna C18 150*25 mm*10 μm; mobile phase: [water (0.225% formic acid)-acetonitrile]; B %: 34%-64%, 10 min) to give 1-(4-(2,6-dioxopiperidin-3-yl)-3,5-difluorophenyl)azetidin-3-yl(3-fluoro-5-methylphenyl)carbamate #78 (100 mg, 223 μmol, 82% yield) as a white solid. ¹H NMR (400 MHz, DMSO-d₆) δ=10.88 (br s, 1H), 10.08 (s, 1H), 7.17 (br d, J=11.4 Hz, 1H), 7.05 (s, 1H), 6.68 (br d, J=9.2 Hz, 1H), 6.22 (d, J=10.6 Hz, 2H), 5.33-5.23 (m, 1H), 4.27-4.17 (m, 2H), 4.05 (br dd, J=4.9, 12.0 Hz, 1H), 3.83 (br dd, J=3.6, 9.0 Hz, 2H), 2.84-2.73 (m, 1H), 2.52 (br s, 1H), 2.29-2.23 (m, 3H), 2.13-2.02 (m, 1H), 1.99-1.89 (m, 1H). MS (ESI) m/z 448.2 [M+H]⁺.

### Example 80. Synthesis of Compound 79

Step 1. To a mixture of cyclohexanamine (230 μL, 2.02 mmol, 1.00 eq), pyridine (325 μL, 4.03 mmol, 2.00 eq) in acetonitrile (2.00 mL) was added phenyl carbonochloridate (265 μL, 2.12 mmol, 1.05 eq). The reaction was stirred at 0° C. for 1 h. The reaction mixture was diluted with water (100 mL) and extracted with ethyl acetate (3×50 mL). The combined organic layers were washed with brine (50 mL), dried over anhydrous sodium sulfate filtered and concentrated under reduced pressure to give a residue which purified by column chromatography (SiO₂, petroleum ether/ethyl acetate=10/1 to 5/1) to afford phenyl cyclohexylcarbamate (165 mg, 673 μmol, 84% yield) as a white solid.

Step 2. To a mixture of 3-(2,6-difluoro-4-(3-hydroxyazetidin-1-yl)phenyl)piperidine-2,6-dione XII (80.0 mg, 270 μmol, 1.00 eq) and sodium hydride (21.6 mg, 540 μmol, 60% purity, 2.00 eq) in dimethylformamide (1.mL) was added phenyl cyclohexylcarbamate (65.1 mg, 297 μmol, 1.10 eq). The reaction was stirred at 0° C. for 1 h. The pH of the mixture was adjusted to 7 with formic acid. The mixture was diluted with dimethyl formamide (1 mL). The reaction was purified by Prep-HPLC (column: Phenomenex luna C18 150*25 mm*10 μm; mobile phase: [water (formic acid)-acetonitrile]; B %: 37%-67%, 10 min) and Prep-HPLC (column: Phenomenex Luna C18 150*25 mm*10 μm; mobile phase: [water (formic acid)-acetonitrile]; B %: 30-66%, 10 min) to afford 1-(4-(2,6-dioxopiperidin-3-yl)-3,5-difluorophenyl)azetidin-3-yl cyclohexylcarbamate #79 (33.55 mg, 78.8 μmol, 29% yield, 99% purity) as a white solid. ¹H NMR (400 MHz, DMSO-d₆) δ=10.86 (br s, 1H), 7.34 (br d, J=7.9 Hz, 1H), 6.18 (d, J=11.0 Hz, 2H), 5.19-5.07 (m, 1H), 4.14 (br t, J=7.6 Hz, 2H), 4.03 (br dd, J=5.0, 12.6 Hz, 1H), 3.70 (br dd, J=3.2, 8.7 Hz, 2H), 3.27-3.20 (m, 1H), 2.87-2.73 (m, 1H), 2.47 (br s, 1H), 2.17-2.01 (m, 1H), 1.98-1.88 (m, 1H), 1.78-1.63 (m, 4H), 1.53 (br d, J=13.0 Hz, 1H), 1.28-1.05 (m, 5H). MS (ESI) m/z 422.1 [M+H]⁺.

### Example 81. Synthesis of Compound 80

Step 1. To a solution of phenyl carbonochloridate (103 uL, 822 μmol, 1.00 eq) and (1S,3S)-3-methylcyclobutan-1-amine (100 mg, 822 μmol, 1.00 eq, hydrochloride) in acetonitrile (1.00 mL) was added pyridine (331 μL, 4.11 mmol, 5.00 eq). The mixture was stirred at 25° C. for 1 h. The mixture was filtered to give filtrate. The filtrate was purified by reverse phase column chromatography (C18, 40 g; condition: water/acetonitrile=1/0 to 0/1, 0.1% formic acid) to afford phenyl ((1S,3S)-3-methylcyclobutyl)carbamate (90.0 mg, 438 μmol, 53% yield) as a white solid. MS (ESI) m/z 206.2 [M+H]⁺.

Step 2. To a solution of 3-(2,6-difluoro-4-(3-hydroxyazetidin-1-yl)phenyl)piperidine-2,6-dione XII (100 mg, 337 μmol, 1.00 eq) and phenyl ((1S,3S)-3-methylcyclobutyl)carbamate (69.2 mg, 337 μmol, 1.00 eq) in dimethylformamide (1 mL) was added sodium hydride (27.0 mg, 675 μmol, 60% purity, 2.00 eq) at 0° C. The mixture was stirred at 25° C. for 1 h. The mixture was quenched with hydrochloric acid (1 M, 0.5 mL) and filtered. The filtrate was purified by Prep-HPLC (column: Phenomenex Synergi C18 150*25 mm*10 μm; mobile phase: [water (formic acid)-acetonitrile]; B %: 37%-67%, 10 min) to afford 1-(4-(2,6-dioxopiperidin-3-yl)-3,5-difluorophenyl)azetidin-3-yl ((1S,3S)-3-methylcyclobutyl)carbamate #80 (58.3 mg, 143 μmol, 42% yield) as a white solid. ¹H NMR (400 MHz, DMSO-d₆) δ=10.87 (br s, 1H), 7.63 (d, J=8.0 Hz, 1H), 6.19 (d, J=11.2 Hz, 2H), 5.20-5.05 (m, 1H), 4.14 (br t, J=7.2 Hz, 2H), 4.04 (br dd, J=5.2, 12.4 Hz, 1H), 3.83-3.65 (m, 3H), 2.86-2.70 (m, 1H), 2.49-2.43 (m, 1H), 2.31 (dq, J=3.2, 7.6 Hz, 2H), 2.14-2.02 (m, 1H), 1.99-1.84 (m, 2H), 1.55-1.41 (m, 2H), 1.00 (d, J=6.8 Hz, 3H). MS (ESI) m/z 408.0 [M+H]⁺.

### Example 82. Synthesis of Compound 81

Step 1. To a solution of (S)-tetrahydrofuran-3-amine (500 mg, 5.74 mmol, 1.00 eq), pyridine (2.32 mL, 28.7 mmol, 5.00 eq) in acetonitrile (10 mL) was added phenyl carbonochloridate (2.16 mL, 17.2 mmol, 3.00 eq) at 0° C. The mixture was stirred at 20° C. for 1 h. The mixture was concentrated under reduced pressure. The residue was purified by column chromatography (SiO₂, petroleum ether/ethyl acetate=10/1 to 0/1) to afford (S)-phenyl (tetrahydrofuran-3-yl)carbamate (0.700 g, 3.38 mmol, 59% yield) as a white solid. ¹H NMR (400 MHz, DMSO-d₆) δ=8.05 (br d, J=6.6 Hz, 1H), 7.42-7.32 (m, 2H), 7.23-7.07 (m, 3H), 4.15-4.05 (m, 1H), 3.85-3.75 (m, 2H), 3.73-3.66 (m, 1H), 3.57-3.52 (m, 1H), 2.15-2.04 (m, 1H), 1.88-1.78 (m, 1H).

Step 2. To a solution of 3-(2,6-difluoro-4-(3-hydroxyazetidin-1-yl)phenyl)piperidine-2,6-dione XII (50.0 mg, 169 μmol, 1.00 eq) in dimethyl formamide (5 mL) was added sodium hydride (8.10 mg, 203 μmol, 60% purity, 1.20 eq) at 0° C. After that, (S)-phenyl (tetrahydrofuran-3-yl)carbamate (38.5 mg, 186 μmol, 1.10 eq) was added into the mixture. The mixture was stirred at 20° C. for 1 h. The mixture was quenched with acetic acid (0.100 mL) and filtered. The filtrate was purified by Prep-HPLC (column: Phenomenex Synergi C18 150*25 mm*10 μm; mobile phase: [water (formic acid)-acetonitrile]; B %: 23%-43%, 10 min), lyophilized to afford 1-(4-(2,6-dioxopiperidin-3-yl)-3,5-difluorophenyl)azetidin-3-yl ((S)-tetrahydrofuran-3-yl)carbamate #81 (10.02 mg, 24.2 μmol, 14% yield, 99% purity) as a white solid. ¹H NMR (400 MHz, DMSO-d₆) δ=10.85 (s, 1H), 7.69 (br d, J=6.5 Hz, 1H), 6.19 (d, J=11.0 Hz, 2H), 5.27-5.05 (m, 1H), 4.15 (br t, J=7.7 Hz, 2H), 4.03 (br dd, J=4.9, 12.3 Hz, 2H), 3.82-3.70 (m, 4H), 3.66 (dt, J=5.6, 8.1 Hz, 1H), 3.46 (dd, J=4.1, 8.9 Hz, 1H), 2.85-2.72 (m, 1H), 2.52 (br s, 1H), 2.14-2.00 (m, 2H), 1.98-1.89 (m, 1H), 1.82-1.69 (m, 1H). MS (ESI) m/z 410.2 [M+H]⁺.

### Example 83. Synthesis of Compound 82

Step 1. To a solution of cyclopropylmethanamine (500 mg, 7.03 mmol, 1.00 eq), pyridine (2.84 mL, 35.2 mmol, 5.00 eq) in acetonitrile (10 mL) was added phenyl carbonochloridate (2.64 mL, 21.1 mmol, 3.00 eq) at 0° C. The mixture was stirred at 20° C. for 1 h. The mixture was concentrated under reduced pressure. The residue was purified by column chromatography (petroleum ether/ethyl acetate=10/1 to 0/1) to afford phenyl (cyclopropylmethyl)carbamate (900 mg, 4.71 mmol, 67% yield) as a yellow solid. ¹H NMR (400 MHz, DMSO-d₆) δ=7.83 (br t, J=5.6 Hz, 1H), 7.40-7.34 (m, 2H), 7.22-7.16 (m, 1H), 7.11-7.06 (m, 2H), 2.95 (t, J=6.4 Hz, 2H), 2.72-2.70 (m, 1H), 0.90 (s, 1H), 0.49-0.36 (m, 2H), 0.25-0.13 (m, 2H).

The mixture of -(2,6-difluoro-4-(3-hydroxyazetidin-1-yl)phenyl)piperidine-2,6-dione XII (80.0 mg, 270 μmol, 1.00 eq) and phenyl (cyclopropylmethyl)carbamate (51.6 mg, 270 μmol, 1.00 eq) in dimethylformamide (1 mL) was added sodium hydride (21.6 mg, 540 μmol, 60% purity, 2.00 eq) at 0° C. for 1 h. The reaction quenched with 1 M hydrochloric acid (0.500 mL) and filtered to give filtrate. The crude product was purified by Prep-HPLC (column: Phenomenex Luna C18 150*25 mm*10 μm; mobile phase: [water (formic acid)-acetonitrile]; B %: 32%-62%, 10 min) to afford 1-(4-(2,6-dioxopiperidin-3-yl)-3,5-difluorophenyl)azetidin-3-yl (cyclopropylmethyl)carbamate #82 (33.4 mg, 84.2 μmol, 31% yield, 99% purity) as a white solid. ¹H NMR (400 MHz, DMSO-d₆) δ=10.86 (br s, 1H), 7.49 (t, J=5.6 Hz, 1H), 6.19 (d, J=10.8 Hz, 2H), 5.33-5.04 (m, 1H), 4.15 (br t, J=7.6 Hz, 2H), 4.04 (dd, J=5.2, 12.8 Hz, 1H), 3.72 (br dd, J=3.6, 8.8 Hz, 2H), 2.88 (t, J=6.4 Hz, 2H), 2.84-2.73 (m, 1H), 2.53 (br s, 1H), 2.16-2.01 (m, 1H), 2.00-1.89 (m, 1H), 0.95-0.83 (m, 1H), 0.44-0.35 (m, 2H), 0.20-0.10 (m, 2H). MS (ESI) m/z 394.1 [M+H]⁺.

### Example 84. Synthesis of Compound 83

Step 1. To a solution of (R)-tetrahydrofuran-3-amine (200 mg, 2.30 mmol, 1.00 eq) in acetonitrile (4 mL) was added pyridine (371 μL, 4.59 mmol, 2.00 eq) and phenyl carbonochloridate (575 μL, 4.59 mmol, 2.00 eq) at 0° C. The mixture was stirred at 20° C. for 1 h. The mixture was concentrated to give a residue. The residue was purified by flash silica gel chromatography (ISCO®; 120 g SepaFlash® Silica Flash Column, Eluent of 0-100% ethyl acetate/petroleum ether at 100 mL/min) to afford (R)-phenyl (tetrahydrofuran-3-yl)carbamate (200 mg, 965 μmol, 42% yield) as a white solid. ¹H NMR (400 MHz, DMSO-d₆) δ=8.03 (br d, J=6.4 Hz, 1H), 7.41-7.33 (m, 2H), 7.23-7.17 (m, 1H), 7.16-7.06 (m, 2H), 4.11 (ddd, J=4.0, 6.4, 10.0 Hz, 1H), 3.86-3.74 (m, 2H), 3.69 (dt, J=5.2, 8.0 Hz, 1H), 3.56 (dd, J=4.0, 8.8 Hz, 1H), 2.10 (qd, J=7.6, 12.8 Hz, 1H), 1.91-1.77 (m, 1H) MS (ESI) m/z 208.2 [M+H]⁺.

Step 2. To a solution of -(2,6-difluoro-4-(3-hydroxyazetidin-1-yl)phenyl)piperidine-2,6-dione XII (50.0 mg, 169 μmol, 1.00 eq) and sodium hydride (10.1 mg, 253 μmol, 60% purity, 1.50 eq) in dimethyl formamide (1 mL) was added (R)-phenyl (tetrahydrofuran-3-yl)carbamate (38.5 mg, 186 μmol, 1.10 eq) at 0° C., the mixture was stirred at 20° C. for 1 h. The mixture was quenched with hydrochloric acid (1 M, 0.500 mL) and filtered. The filtrate was purified by Prep-HPLC (column: Unisil 3-100 C18 Ultra 150*50 mm*3 μm; mobile phase: [water(formic acid)-acetonitrile]; B %: 16%-46%, 15 min) and lyophilized to afford 1-(4-(2,6-dioxopiperidin-3-yl)-3,5-difluorophenyl)azetidin-3-yl ((R)-tetrahydrofuran-3-yl)carbamate #83 (27.05 mg, 65.4 μmol, 39% yield, 99% purity) as a white solid. ¹H NMR (400 MHz, DMSO-d₆) δ=10.87 (s, 1H), 7.70 (d, J=6.8 Hz, 1H), 6.19 (d, J=11.2 Hz, 2H), 5.20-5.08 (m, 1H), 4.15 (br t, J=7.6 Hz, 2H), 4.04 (br dd, J=5.2, 12.4 Hz, 2H), 3.80-3.69 (m, 4H), 3.65 (dt, J=5.6, 8.2 Hz, 1H), 3.45 (dd, J=4.0, 9.2 Hz, 1H), 2.84-2.71 (m, 1H), 2.52 (br d, J=2.0 Hz, 1H), 2.14-1.99 (m, 2H), 1.98-1.89 (m, 1H), 1.80-1.69 (m, 1H). MS (ESI) m/z 410.2 [M+H]⁺.

### Example 85. Synthesis of Compound 84

Step 1. To a solution of (S)-tetrahydro-2H-pyran-3-amine (300 mg, 2.97 mmol, 1.00 eq), pyridine (1.20 mL, 14.8 mmol, 5.00 eq) in acetonitrile (6 mL) was added phenyl carbonochloridate (1.11 mL, 8.90 mmol, 3.00 eq) at 0° C. The mixture was stirred at 20° C. for 1 h. The mixture was concentrated under reduced pressure. The residue was purified by column chromatography (SiO₂, petroleum ether/ethyl acetate=10/1 to 0/1) to afford (S)-phenyl (tetrahydro-2H-pyran-3-yl)carbamate (580 mg, 2.62 mmol, 88% yield) as a white solid. ¹H NMR (400 MHz, DMSO-d₆) δ=7.77 (br d, J=7.8 Hz, 1H), 7.39-7.35 (m, 2H), 7.23-7.16 (m, 1H), 7.12-7.07 (m, 2H), 3.78 (dd, J=2.9, 10.9 Hz, 1H), 3.73-3.66 (m, 1H), 3.50-3.39 (m, 1H), 3.29-3.24 (m, 1H), 3.12 (dd, J=9.1, 10.8 Hz, 1H), 1.97-1.87 (m, 1H), 1.73-1.65 (m, 1H), 1.56-1.45 (m, 2H). MS (ESI) m/z 222.2 [M+H]⁺.

Step 2. To a solution of -(2,6-difluoro-4-(3-hydroxyazetidin-1-yl)phenyl)piperidine-2,6-dione XII (50.0 mg, 169 umol, 1.00 eq) in dimethyl formamide (5 mL) was added sodium hydride (8.10 mg, 203 μmol, 60% purity, 1.20 eq) at 0° C. (S)-phenyl (tetrahydro-2H-pyran-3-yl)carbamate (41.1 mg, 186 μmol, 1.10 eq) was then added at 0° C. The mixture was stirred at 20° C. for 1 h. The mixture was quenched with acetic acid (0.100 mL) and filtered. The filtrate was purified by Prep-HPLC (column: Waters Xbridge 150*25 mm*5 μm; mobile phase: [water (ammonium bicarbonate)-acetonitrile]; B %: 26%-56%, 9 min) and lyophilized to give 1-(4-(2,6-dioxopiperidin-3-yl)-3,5-difluorophenyl)azetidin-3-yl ((S)-tetrahydro-2H-pyran-3-yl)carbamate #84 (29.82 mg, 68.3 μmol, 40% yield, 97% purity) as a white solid. ¹H NMR (400 MHz, DMSO-d₆) δ=10.86 (br s, 1H), 7.42 (d, J=7.6 Hz, 1H), 6.19 (d, J=11.0 Hz, 2H), 5.38-4.98 (m, 1H), 4.14 (br t, J=7.6 Hz, 2H), 4.03 (br dd, J=5.1, 12.8 Hz, 1H), 3.80-3.61 (m, 4H), 3.38 (br s, 2H), 3.28-3.19 (m, 1H), 3.02 (br t, J=10.0 Hz, 1H), 2.86-2.71 (m, 1H), 2.07 (dq, J=3.9, 13.2 Hz, 1H), 1.98-1.89 (m, 1H), 1.88-1.77 (m, 1H), 1.72-1.58 (m, 1H), 1.52-1.25 (m, 2H). MS (ESI) m/z 424.2 [M+H]⁺.

### Example 86. Synthesis of Compound 85

Step 1. To a solution of (R)-tetrahydro-2H-pyran-3-amine (80.0 mg, 581 μmol, 1.00 eq, hydrochloride) in acetonitrile (2 mL) was added pyridine (93.9 μL, 92.0 mg, 1.16 mmol, 2.00 eq) and phenyl carbonochloridate (146 μL, 1.16 mmol, 2.00 eq) at 0° C. The mixture was stirred at 20° C. for 1 h. The mixture was concentrated to give a residue. The residue was purified by flash silica gel chromatography (ISCO®; 120 g SepaFlash® Silica Flash Column, Eluent of 0-100% ethyl acetate/petroleum ether at 100 mL/min) to afford (R)-phenyl (tetrahydro-2H-pyran-3-yl)carbamate (110 mg, 497 μmol, 86% yield) as a white solid. ¹H NMR (400 MHz, DMSO-d₆) δ=7.77 (br d, J=7.6 Hz, 1H), 7.43-7.34 (m, 2H), 7.25-7.17 (m, 1H), 7.09 (d, J=7.6 Hz, 2H), 3.82-3.67 (m, 2H), 3.44 (td, J=4.4, 8.4 Hz, 2H), 3.12 (dd, J=9.2, 10.8 Hz, 1H), 1.92 (br d, J=9.2 Hz, 1H), 1.76-1.65 (m, 1H), 1.57-1.43 (m, 2H). MS (ESI) m/z 222.0 [M+H]⁺.

Step 2. To a solution of -(2,6-difluoro-4-(3-hydroxyazetidin-1-yl)phenyl)piperidine-2,6-dione XII (50.0 mg, 169 μmol, 1.00 eq) in dimethyl formamide (1 mL) was added sodium hydride (10.1 mg, 253 μmol, 60% purity, 1.50 eq) and (R)-phenyl (tetrahydro-2H-pyran-3-yl)carbamate (41.1 mg, 186 μmol, 1.10 eq) at 0° C., the mixture was stirred at 20° C. for 1 h. The mixture was quenched with hydrochloric acid (1 M, 0.500 mL) and filtered. The filtrate was purified by Prep-HPLC (column: Unisil 3-100 C18 Ultra 150*50 mm*3 μm; mobile phase: [water (formic acid)-acetonitrile]; B %: 20%-50%, 15 min) and lyophilized to afford 1-(4-(2,6-dioxopiperidin-3-yl)-3,5-difluorophenyl)azetidin-3-yl((R)-tetrahydro-2H-pyran-3-yl)carbamate #85 (20.93 mg, 44.1 μmol, 26% yield, 99% purity, formate) as a white solid. ¹H NMR (400 MHz, DMSO-d₆) δ=10.87 (s, 1H), 7.43 (d, J=8.0 Hz, 1H), 6.19 (d, J=11.2 Hz, 2H), 5.25-5.03 (m, 1H), 4.14 (br t, J=7.6 Hz, 2H), 4.04 (br dd, J=5.2, 12.8 Hz, 1H), 3.80-3.64 (m, 4H), 3.47-3.36 (m, 2H), 3.28-3.16 (m, 1H), 3.02 (t, J=10.0 Hz, 1H), 2.84-2.71 (m, 1H), 2.07 (dq, J=3.6, 13.2 Hz, 1H), 1.99-1.89 (m, 1H), 1.84 (br dd, J=3.6, 12.0 Hz, 1H), 1.71-1.60 (m, 1H), 1.56-1.33 (m, 2H) MS (ESI) m/z 424.2 [M+H]⁺.

### Example 87. Synthesis of Compound 86

Step 1. To a solution of cyclopropanamine (607 μL, 8.76 mmol, 1.00 eq) in acetonitrile (5 mL) was added pyridine (1.41 mL, 17.5 mmol, 2.00 eq) and phenyl carbonochloridate (2.19 mL, 17.5 mmol, 2.00 eq) at 0° C. The mixture was stirred at 20° C. for 1 h. The mixture was concentrated to give a residue. The residue was purified by flash silica gel chromatography (ISCO®; 120 g SepaFlash® Silica Flash Column, Eluent of 0-100% ethyl acetate/petroleum ether at 100 mL/min) to afford phenyl cyclopropylcarbamate (1.40 g, 7.90 mmol, 90% yield) as a white solid. ¹H NMR (400 MHz, DMSO-d₆) δ=7.36 (br t, J=7.6 Hz, 2H), 7.25-7.18 (m, 1H), 7.14 (br d, J=7.6 Hz, 2H), 5.53-4.89 (m, 1H), 2.70 (br s, 1H), 0.87-0.76 (m, 2H), 0.69-0.53 (m, 2H).

Step 2. To a solution of -(2,6-difluoro-4-(3-hydroxyazetidin-1-yl)phenyl)piperidine-2,6-dione XII (50.0 mg, 169 μmol, 1.00 eq) in dimethyl formamide (1 mL) was added sodium hydride (10.1 mg, 253 μmol, 60% purity, 1.50 eq) and phenyl cyclopropylcarbamate (35.9 mg, 203 μmol, 1.20 eq) at 0° C., the mixture was stirred at 20° C. for 1 h. The mixture was quenched with hydrochloric acid (1 M, 0.500 mL) and filtered. The filtrate was purified by Prep-HPLC (column: Phenomenex luna C18 250*50 mm*15 μm; mobile phase: [water (formic acid)-acetonitrile]; B %: 30%-60%, 10 min) and lyophilized to afford 1-(4-(2,6-dioxopiperidin-3-yl)-3,5-difluorophenyl)azetidin-3-yl cyclopropylcarbamate #86 (28.85 mg, 67.1 μmol, 40% yield, 99% purity, formate) as a white solid. ¹H NMR (400 MHz, DMSO-d₆) δ=10.86 (br s, 1H), 7.57 (br s, 1H), 6.18 (br d, J=11.2 Hz, 2H), 5.13 (br s, 1H), 4.14 (br t, J=7.6 Hz, 2H), 4.03 (dd, J=5.2, 12.8 Hz, 1H), 3.70 (br d, J=6.0 Hz, 2H), 2.84-2.71 (m, 1H), 2.47 (br s, 2H), 2.16-2.00 (m, 1H), 1.99-1.87 (m, 1H), 0.66-0.53 (m, 2H), 0.45-0.33 (m, 2H). MS (ESI) m/z 380.1 [M+H]⁺.

### Example 88. Synthesis of Compound 87

Step 1. To a solution of 2,2,2-trifluoroethanamine (397 μL, 5.05 mmol, 1.00 eq) in acetonitrile (10 mL) was added pyridine (815 μL, 10.1 mmol, 2.00 eq) and phenyl carbonochloridate (1.26 mL, 10.1 mmol, 2.00 eq) at 0° C. The mixture was stirred at 20° C. for 1 h. The mixture was concentrated to give a residue. The residue was purified by flash silica gel chromatography (ISCO®; 120 g SepaFlash® Silica Flash Column, Eluent of 0-100% ethyl acetate/petroleum ether at 100 mL/min) to afford phenyl (2,2,2-trifluoroethyl)carbamate (360 mg, 1.64 mmol, 33% yield) as a white solid. ¹H NMR (400 MHz, DMSO-d₆) δ=8.55 (br t, J=6.2 Hz, 1H), 7.43-7.37 (m, 2H), 7.28-7.21 (m, 1H), 7.13 (br d, J=7.6 Hz, 2H), 3.97-3.83 (m, 2H). MS (ESI) m/z 220.1 [M+H]⁺.

Step 2. To a solution of -(2,6-difluoro-4-(3-hydroxyazetidin-1-yl)phenyl)piperidine-2,6-dione XII (50.0 mg, 169 μmol, 1.00 eq) in dimethyl formamide (1 mL) was added sodium hydride (10.1 mg, 253 μmol, 60% purity, 1.50 eq) and phenyl (2,2,2-trifluoroethyl)carbamate (44.4 mg, 203 μmol, 1.20 eq) at 0° C., the mixture was stirred at 20° C. for 1 h. The mixture was quenched with hydrochloric acid (1 M, 0.500 mL) and filtered. The filtrate was purified by Prep-HPLC (column: Phenomenex luna C18 250*50 mm*15 μm; mobile phase: [water (formic acid)-acetonitrile]; B %: 32%-62%, 10 min) and lyophilized to afford 1-(4-(2,6-dioxopiperidin-3-yl)-3,5-difluorophenyl)azetidin-3-yl (2,2,2-trifluoroethyl)carbamate #87 (26.75 mg, 62.9 μmol, 37% yield, 99% purity) as a white solid. ¹H NMR (400 MHz, DMSO-d₆) δ=10.86 (br s, 1H), 8.22 (t, J=6.4 Hz, 1H), 6.20 (d, J=11.2 Hz, 2H), 5.24-5.14 (m, 1H), 4.17 (br t, J=7.8 Hz, 2H), 4.04 (dd, J=5.2, 12.8 Hz, 1H), 3.88-3.69 (m, 4H), 2.84-2.71 (m, 1H), 2.48 (br s, 1H), 2.16-1.99 (m, 1H), 1.98-1.85 (m, 1H). MS (ESI) m/z 422.0 [M+H]⁺.

### Example 89. Synthesis of Compound 88

Step 1. To a solution of 3,3-difluorocyclobutanamine (500 mg, 4.67 mmol, 1.00 eq) in acetonitrile (10 mL) was added pyridine (754 μL, 9.34 mmol, 2.00 eq) and phenyl carbonochloridate (1.17 mL, 9.34 mmol, 2.00 eq). The mixture was stirred at 20° C. for 1 h. The mixture was concentrated to give a residue. The residue was purified by flash silica gel chromatography (ISCO®; 120 g SepaFlash® Silica Flash Column, eluent of 0-100% ethyl acetate/petroleum ether at 100 mL/min) to afford phenyl(3,3-difluorocyclobutyl) carbamate (680 mg, 2.99 mmol, 64% yield) as a white solid. ¹H NMR (400 MHz, DMSO-d₆) δ=8.28 (br d, J=6.4 Hz, 1H), 7.43-7.34 (m, 2H), 7.25-7.18 (m, 1H), 7.11 (d, J=7.6 Hz, 2H), 3.94 (td, J=7.2, 14.4 Hz, 1H), 3.04-2.86 (m, 2H), 2.72-2.61 (m, 2H).

Step 2. To a solution of -(2,6-difluoro-4-(3-hydroxyazetidin-1-yl)phenyl)piperidine-2,6-dione XII (80.0 mg, 270 μmol, 1.00 eq) and phenyl (3,3-difluorocyclobutyl)carbamate (61.4 mg, 270 μmol, 1.00 eq) in dimethylformamide (1 mL) was added sodium hydride (21.6 mg, 540 μmol, 60% purity, 2.00 eq) at 0° C. for 1 h. The reaction was quenched with 1 M hydrochloric acid (0.500 mL) and filtered to give a filtrate. The filtrate was purified by Prep-HPLC (column: Shim-pack C18 150*25 mm*10 μm; mobile phase: [water (formic acid)-acetonitrile]; B %: 28%-58%, 10 min) to afford 1-(4-(2,6-dioxopiperidin-3-yl)-3,5-difluorophenyl)azetidin-3-yl(3,3-difluorocyclobutyl)carbamate #88 (61.4 mg, 141 μmol, 52% yield, 99% purity) as a white solid. ¹H NMR (400 MHz, DMSO-d₆) δ=10.87 (s, 1H), 7.95 (br d, J=6.8 Hz, 1H), 6.19 (d, J=10.8 Hz, 2H), 5.26-5.04 (m, 1H), 4.16 (br t, J=7.6 Hz, 2H), 4.04 (dd, J=5.2, 12.8 Hz, 1H), 3.95-3.81 (m, 1H), 3.74 (br dd, J=3.2, 8.8 Hz, 2H), 2.95-2.83 (m, 2H), 2.82-2.70 (m, 1H), 2.66-2.54 (m, 2H), 2.48 (br s, 1H), 2.18-2.03 (m, 1H), 2.01-1.88 (m, 1H). MS (ESI) m/z 430.1 [M+H]⁺.

### Example 90. Synthesis of Compound 89

Step 1. To a solution of 2-methylpropan-2-amine (1.44 mL, 13.7 mmol, 1.00 eq) in acetonitrile (10 mL) was added pyridine (2.21 mL, 27.4 mmol, 2.00 eq) and phenyl carbonochloridate (3.43 mL, 27.4 mmol, 2.00 eq). The mixture was stirred at 20° C. for 1 h. The mixture was concentrated to give a residue. The residue was purified by flash silica gel chromatography (ISCO®; 120 g SepaFlash® Silica Flash Column, Eluent of 0-100% ethyl acetate/petroleum ether at 100 mL/min) to afford phenyl tert-butylcarbamate (1.70 g, 8.80 mmol, 64% yield) as a white solid. ¹H NMR (400 MHz, DMSO-d₆) δ=7.40-7.31 (m, 2H), 7.22-7.16 (m, 1H), 7.12 (d, J=8.0 Hz, 2H), 4.98 (br s, 1H), 1.56 (s, 2H), 1.40 (s, 9H).

Step 2. To a solution of phenyl tert-butylcarbamate (52.2 mg, 270 μmol, 1.00 eq) and -(2,6-difluoro-4-(3-hydroxyazetidin-1-yl)phenyl)piperidine-2,6-dione XII (80.0 mg, 270 μmol, 1.00 eq) in dimethylformamide (1 mL) was added sodium hydride (21.6 mg, 540 μmol, 60% purity, 2.00 eq) at 0° C. for 1 h. The reaction quenched with 1 M hydrochloric acid (0.500 mL) and filtered to give filtrate. The residue was purified by Prep-HPLC (column: Shim-pack C18 150*25*10 μm; mobile phase: [water (formic acid)-acetonitrile]; B %: 32%-62%, 10 min) to afford 1-(4-(2,6-dioxopiperidin-3-yl)-3,5-difluorophenyl)azetidin-3-yl tert-butylcarbamate #89 (51.4 mg, 128 μmol, 48% yield, 99% purity) as a white solid. ¹H NMR (400 MHz, DMSO-d₆) δ=10.87 (br s, 1H), 7.17 (br s, 1H), 6.20 (d, J=11.2 Hz, 2H), 5.12 (br s, 1H), 4.20-4.11 (m, 2H), 4.04 (dd, J=5.2, 12.4 Hz, 1H), 3.70 (br dd, J=3.6, 8.8 Hz, 2H), 2.84-2.72 (m, 1H), 2.48-2.44 (m, 1H), 2.14-2.02 (m, 1H), 2.00-1.89 (m, 1H), 1.23 (s, 9H) MS (ESI) m/z 396.1 [M+H]⁺.

### Example 91. Synthesis of Compound 90

Step 1. To a solution of cyclobutanamine (361 μL, 4.22 mmol, 1.00 eq) and pyridine (1.70 mL, 21.1 mmol, 5.00 eq) in acetonitrile (6 mL) was added phenyl carbonochloridate (1.59 mL, 12.7 mmol, 3.00 eq) at 0° C. The mixture was stirred at 20° C. for 1 h. The mixture was concentrated under reduced pressure to give a residue. The residue was purified by column chromatography (petroleum ether/ethyl acetate=10/1 to 0/1) to afford phenyl cyclobutylcarbamate (700 mg, 3.66 mmol, 87% yield) as a white solid. ¹H NMR (400 MHz, DMSO-d₆) δ=8.05 (br d, J=7.6 Hz, 1H), 7.40-7.33 (m, 2H), 7.22-7.16 (m, 1H), 7.08 (d, J=7.6 Hz, 2H), 4.08-3.91 (m, 1H), 2.22-2.13 (m, 2H), 2.01-1.93 (m, 2H), 1.67-1.55 (m, 2H) MS (ESI) m/z 192.0 [M+H]⁺.

Step 2. To a solution of 3-(2,6-difluoro-4-(3-hydroxyazetidin-1-yl)phenyl)piperidine-2,6-dione XII (80.0 mg, 270 μmol, 1.00 eq) and phenyl cyclobutylcarbamate (51.6 mg, 270 μmol, 1.00 eq) in dimethylformamide (1 mL) was added sodium hydride (21.6 mg, 540 μmol, 60% purity, 2.00 eq) at 0° C. and the reaction was stirred at 25° C. for 1 h. The reaction was quenched with 1 M hydrochloric acid (0.500 mL) and filtered to give a filtrate. The residue was purified by Prep-HPLC (column: Phenomenex luna C18 150*25 mm*10 μm; mobile phase: [water (formic acid)-acetonitrile]; B %: 32%-62%, 10 min) to afford 1-(4-(2,6-dioxopiperidin-3-yl)-3,5-difluorophenyl) azetidin-3-yl cyclobutylcarbamate #90 (13.5 mg, 33.9 μmol, 13% yield, 99% purity) as a white solid. ¹H NMR (400 MHz, DMSO-d₆) δ=10.87 (br s, 1H), 7.72 (d, J=8.0 Hz, 1H), 6.19 (d, J=11.2 Hz, 2H), 5.23-4.98 (m, 1H), 4.14 (br t, J=7.6 Hz, 2H), 4.04 (br dd, J=5.2, 12.8 Hz, 1H), 4.00-3.90 (m, 1H), 3.71 (br dd, J=3.6, 8.8 Hz, 2H), 2.84-2.72 (m, 1H), 2.48-2.45 (m, 1H), 2.20-2.05 (m, 3H), 1.98-1.82 (m, 3H), 1.64-1.50 (m, 2H). MS (ESI) m/z 394.1 [M+H]⁺.

### Example 92. Synthesis of Compound 91

Step 1. To a solution of cyclobutylmethanamine (300 mg, 3.52 mmol, 1.00 eq), pyridine (1.39 g, 17.6 mmol, 1.42 mL, 5.00 eq) in acetonitrile (6 mL) was added phenyl carbonochloridate (1.32 mL, 10.6 mmol, 3.00 eq) at 0° C. The mixture was stirred at 20° C. for 1 h. The mixture was concentrated under reduced pressure. The residue was purified by column chromatography (SiO₂, petroleum ether/ethyl acetate=10/1 to 0/1) to afford phenyl (cyclobutylmethyl)carbamate (700 mg, 3.41 mmol, 97% yield) as a white solid. ¹H NMR (400 MHz, DMSO-d₆) δ=7.75 (br t, J=5.6 Hz, 1H), 7.40-7.33 (m, 2H), 7.23-7.17 (m, 1H), 7.10-7.05 (m, 2H), 3.08 (dd, J=6.1, 6.8 Hz, 2H), 2.47-2.38 (m, 1H), 2.02-1.96 (m, 2H), 1.88-1.78 (m, 2H), 1.73-1.64 (m, 2H). MS (ESI) m/z 206.2 [M+H]⁺.

Step 2. To a solution of 3-(2,6-difluoro-4-(3-hydroxyazetidin-1-yl)phenyl)piperidine-2,6-dione XII (50.0 mg, 169 μmol, 1.00 eq) in dimethyl formamide (5 mL) was added sodium hydride (7.42 mg, 186 μmol, 60% purity, 1.10 eq) at 0° C. Phenyl (cyclobutylmethyl)carbamate (38.1 mg, 186 μmol, 1.10 eq) was then added into the reaction mixture at 0° C. The mixture was stirred at 20° C. for 1 h. The mixture was quenched with acetic acid (0.100 mL) and filtered. The filtrate was purified by Prep-HPLC (column: Waters Xbridge 150*25 mm*5 μm; mobile phase: [water(ammonium bicarbonate)-acetonitrile]; B %: 37%-67%, 9 min) and lyophilized to give 1-(4-(2,6-dioxopiperidin-3-yl)-3,5-difluorophenyl)azetidin-3-yl (cyclobutylmethyl)carbamate #91 (28.64 mg, 68.9 μmol, 40% yield, 98% purity) as a white solid. ¹H NMR (400 MHz, DMSO-d₆) δ=10.86 (br s, 1H), 7.41 (t, J=5.8 Hz, 1H), 6.18 (d, J=10.9 Hz, 2H), 5.23-5.00 (m, 1H), 4.14 (br t, J=7.7 Hz, 2H), 4.03 (dd, J=5.0, 12.6 Hz, 1H), 3.70 (br dd, J=3.8, 8.9 Hz, 2H), 3.00 (t, J=6.4 Hz, 2H), 2.84-2.71 (m, 1H), 2.47 (br s, 1H), 2.42-2.33 (m, 1H), 2.15-2.00 (m, 1H), 1.99-1.89 (m, 3H), 1.84-1.73 (m, 2H), 1.70-1.59 (m, 2H). MS (ESI) m/z 408.2 [M+H]⁺.

### Example 93. Synthesis of Compound 92

Step 1. To a mixture of cyclopentanamine (232 μL, 2.35 mmol, 1.00 eq), pyridine (379 μL, 4.70 mmol, 2.00 eq) in acetonitrile (2 mL) was added phenyl carbonochloridate (308 μL, 2.47 mmol, 1.05 eq). The reaction was stirred at 20° C. for 1 h. The reaction mixture was diluted with water (100 mL) and extracted with ethyl acetate (3×50 mL). The combined organic layers were washed with brine (50 mL), dried over anhydrous sodium sulfate, filtered and concentrated under reduced pressure to give a residue, which was purified by column chromatography (SiO₂, petroleum ether/ethyl acetate=10/1 to 5/1) to afford phenyl-cyclopentylcarbamate (300 mg, 1.46 mmol, 62% yield) as a white solid. MS (ESI) m/z 206.2 [M+H]⁺.

Step 2. To a mixture of 3-(2,6-difluoro-4-(3-hydroxyazetidin-1-yl)phenyl)piperidine-2,6-dione XII (80.0 mg, 270 μmol, 1.00 eq) and phenyl cyclopentylcarbamate (55.4 mg, 270 μmol, 1.00 eq) in dimethyl formamide (1 mL) was added sodium hydrogen (21.6 mg, 540 μmol, 60% purity, 2.00 eq). The reaction was stirred at 0° C. for 1 h. The pH of the mixture was adjusted to 7 with formic acid, then the mixture was diluted with dimethyl formamide (1.00 mL). The mixture was purified by Prep-HPLC (column: Phenomenex luna C18 150*25 mm*10 μm; mobile phase: [water (formic acid)-acetonitrile]; B %: 33%-63%, 10 min) to give 1-(4-(2,6-dioxopiperidin-3-yl)-3,5-difluorophenyl)azetidin-3-yl cyclopentylcarbamate #92 (68.0 mg, 165 umol, 61% yield, 99% purity) as a white solid. ¹H NMR (400 MHz, DMSO-d₆) δ=10.85 (br s, 1H), 7.39 (br d, J=7.2 Hz, 1H), 6.18 (d, J=11.2 Hz, 2H), 5.17-5.09 (m, 1H), 4.14 (br t, J=7.6 Hz, 2H), 4.03 (dd, J=5.2, 12.4 Hz, 1H), 3.82-3.75 (m, 1H), 3.71 (br dd, J=3.6, 8.8 Hz, 2H), 2.84-2.71 (m, 1H), 2.52 (br s, 1H), 2.11-2.01 (m, 1H), 1.98-1.89 (m, 1H), 1.80-1.71 (m, 2H), 1.64-1.55 (m, 2H), 1.52-1.43 (m, 2H), 1.43-1.32 (m, 2H). MS (ESI) m/z 408.3 [M+H]⁺.

### Example 94. Synthesis of Compound 93

Step 1. To a mixture of 4,4-dimethylcyclohexan-1-amine (300 mg, 2.36 mmol, 1.00 eq), pyridine (381 μL, 4.72 mmol, 2.00 eq) in acetonitrile (5 mL) were added phenyl carbonochloridate (310 μL, 2.48 mmol, 1.05 eq). The mixture was stirred at 20° C. for 1 h. The reaction mixture was diluted with water (100 mL) and extracted with ethyl acetate (3×50 mL). The combined organic layers were washed with brine (50 mL), dried over anhydrous sodium sulfate, filtered and concentrated under reduced pressure to give a residue, which was purified by column chromatography (SiO₂, petroleum ether/ethyl acetate=10/1 to 5/1) to afford phenyl (4,4-dimethylcyclohexyl)carbamate (200 mg, 0.808 mmol, 34% yield) as a white solid. MS (ESI) m/z 248.2[M+H]⁺.

Step 2. To a mixture of 3-(2,6-difluoro-4-(3-hydroxyazetidin-1-yl)phenyl)piperidine-2,6-dione XII (80.0 mg, 270 μmol, 1.00 eq) and sodium hydride (21.6 mg, 540 μmol, 60.0% purity, 2.00 eq) in dimethylformamide (1 mL) was added phenyl (4,4-dimethylcyclohexyl)carbamate (73.5 mg, 297 μmol, 1.10 eq). The reaction was stirred at 20° C. for 1 h. The pH of the mixture was adjusted to 7 with formic acid, then the mixture was diluted with dimethyl formamide (1 mL). The mixture was purified by Prep-HPLC (column: Phenomenex Luna C18 150*25 mm*10 μm; mobile phase: [water(formic acid)-acetonitrile]; B %: 45%-75%, 10 min) to give 1-(4-(2,6-dioxopiperidin-3-yl)-3,5-difluorophenyl)azetidin-3-yl (4,4-dimethylcyclohexyl) carbamate #93 (70.0 mg, 154 μmol, 57% yield, 99% purity) as a white solid. ¹H NMR (400 MHz, DMSO-d₆) δ=10.86 (br s, 1H), 7.30 (br d, J=8.0 Hz, 1H), 6.18 (d, J=11.2 Hz, 2H), 5.13 (dt, J=3.4, 6.4 Hz, 1H), 4.14 (br t, J=7.6 Hz, 2H), 4.03 (dd, J=5.2, 12.5 Hz, 1H), 3.71 (br dd, J=3.2, 8.6 Hz, 2H), 3.25-3.15 (m, 1H), 2.77 (ddd, J=5.6, 13.3, 17.2 Hz, 1H), 2.47 (br s, 1H), 2.13-2.00 (m, 1H), 1.98-1.88 (m, 1H), 1.58 (br dd, J=4.0, 8.7 Hz, 2H), 1.41-1.29 (m, 4H), 1.23-1.11 (m, 2H), 0.87 (d, J=4.4 Hz, 6H). MS (ESI) m/z 450.2[M+H]⁺.

### Example 95. Synthesis of Compound 94

Step 1. To a solution of tetrahydro-2H-pyran-4-amine (200 mg, 1.98 mmol, 1.00 eq), pyridine (319 μL, 3.95 mmol, 2.00 eq) and acetonitrile (2 mL) was added phenyl carbonochloridate (250 μL, 2.00 mmol, 1.01 eq). The mixture was stirred at 20° C. for 1 h. The reaction mixture was diluted with water (100 mL) and extracted with ethyl acetate (3×50 mL). The combined organic layers were washed with brine (50 mL), dried over anhydrous sodium sulfate, filtered and concentrated under reduced pressure to give a residue, which was purified by column chromatography (SiO₂, petroleum ether/ethyl acetate=10/1 to 5/1) to afford phenyl (tetrahydro-2H-pyran-4-yl)carbamate (400 mg, 1.81 mmol, 91% yield) as a white solid. MS (ESI) m/z 222.2[M+H]⁺.

Step 2. To a mixture of 3-(2,6-difluoro-4-(3-hydroxyazetidin-1-yl)phenyl)piperidine-2,6-dione XII (80.0 mg, 270 μmol, 1.00 eq) in dimethylformamide (1 mL) was added phenyl (tetrahydro-2H-pyran-4-yl)carbamate (65.7 mg, 297 μmol, 1.10 eq) and sodium hydride (21.6 mg, 540 μmol, 60% purity, 2.00 eq). The reaction was stirred at 20° C. for 1 h. The pH of the mixture was adjusted to 7 with formic acid, then the mixture was diluted with dimethyl formamide (1 mL). The reaction was purified by Prep-HPLC (column: Phenomenex luna C18 150*25 mm*10 μm; mobile phase: [water(formic acid)-acetonitrile]; B %: 24%-54%, 10 min.) to give 1-(4-(2,6-dioxopiperidin-3-yl)-3,5-difluorophenyl)azetidin-3-yl (tetrahydro-2H-pyran-4-yl)carbamate #94 (82.0 mg, 192 μmol, 71% yield, 99% purity) as a white solid. ¹H NMR (400 MHz, DMSO-d₆) δ=10.8 (br s, 1H), 7.47 (br d, J=7.2 Hz, 1H), 6.19 (d, J=11.2 Hz, 2H), 5.18-5.09 (m, 1H), 4.15 (br t, J=7.6 Hz, 2H), 4.03 (br dd, J=5.2, 12.2 Hz, 1H), 3.84-3.77 (m, 2H), 3.72 (br dd, J=3.2, 8.8 Hz, 2H), 3.55-3.44 (m, 2H), 3.28 (br s, 1H), 2.89-2.74 (m, 1H), 2.52 (br s, 1H), 2.14-2.00 (m, 1H), 1.98-1.89 (m, 1H), 1.69 (br d, J=10.8 Hz, 2H), 1.47-1.31 (m, 2H). MS (ESI) m/z 424.2[M+H]⁺.

### Example 96. Synthesis of Compound 95

Step 1. To a solution of oxetan-3-amine (100 mg, 1.37 mmol, 1.00 eq) and pyridine (552 μL, 6.84 mmol, 5.00 eq) in acetonitrile (2 mL) was added phenyl carbonochloridate (205 μL, 1.64 mmol, 1.20 eq) at 0° C. The mixture was stirred at 20° C. for 1 h. The reaction mixture was filtered to give filtrate. The residue was purified by reversed-phase HPLC (0.1% formic acid condition) to afford phenyl oxetan-3-ylcarbamate (150 mg, 0.776 mmol, 57% yield) as a white solid. MS (ESI) m/z 194.2 [M+H]⁺.

Step 2. To a solution of 3-(2,6-difluoro-4-(3-hydroxyazetidin-1-yl)phenyl)piperidine-2,6-dione XII (100 mg, 337 umol, 1.00 eq) and phenyl oxetan-3-ylcarbamate (65.2 mg, 337 μmol, 1.00 eq) in dimethylformamide (1.00 mL) was added sodium hydride (27.0 mg, 675 μmol, 60% purity, 2.00 eq) at 0° C. Then the mixture was stirred at 20° C. for 1 h. The reaction quenched with 1M hydrochloric acid (0.500 mL) and filtered to give filtrate. The filtrate was purified by Prep-HPLC (column: Phenomenex C18 75*30 mm*3 μm; mobile phase: [water (formic acid)-acetonitrile]; B %: 20%-50%, 7 min) to afford 1-(4-(2,6-dioxopiperidin-3-yl)-3,5-difluorophenyl)azetidin-3-yl oxetan-3-ylcarbamate #95 (58.8 mg, 147 μmol, 44% yield, 99% purity) as a white solid. ¹H NMR (400 MHz, DMSO-d₆) δ=10.87 (br s, 1H), 8.24 (br d, J=5.6 Hz, 1H), 6.19 (br d, J=11.2 Hz, 2H), 5.15 (br s, 1H), 4.74-4.60 (m, 3H), 4.45 (br s, 2H), 4.15 (br t, J=7.6 Hz, 2H), 4.04 (br dd, J=4.8, 12.4 Hz, 1H), 3.74 (br dd, J=3.2, 8.8 Hz, 2H), 2.85-2.71 (m, 1H), 2.61-2.53 (m, 1H), 2.14-2.01 (m, 1H), 2.00-1.90 (m, 1H). MS (ESI) m/z 395.9 [M+H]⁺.

### Example 97. Synthesis of Compound 96

To a solution of 2-(3,3-difluorocyclobutyl)acetic acid (30.0 mg, 199 μmol, 1.00 eq) and triethylamine (56.0 μL, 399 umol, 2.00 eq) in dioxane (1 mL) was added diphenylphosphoryl azide (69.3 uL, 319 μmol, 1.60 eq). The mixture was stirred at 25° C. for 1 h, then 3-(2,6-difluoro-4-(3-hydroxyazetidin-1-yl)phenyl)piperidine-2,6-dione XII (65.1 mg, 219 μmol, 1.10 eq) was added to the mixture and the mixture was stirred at 100° C. for 1 h. The mixture was purified by Prep-TLC (petroleum ether:ethyl acetate=1:1) and Prep-HPLC (column: Phenomenex luna C18 150*25 mm*10 μm; mobile phase: [water (formic acid)-acetonitrile]; B %: 39%-59%, 10 min) to afford 1-(4-(2,6-dioxopiperidin-3-yl)-3,5-difluorophenyl)azetidin-3-yl ((3,3-difluorocyclobutyl)methyl)carbamate #96 (15.31 mg, 34.1 μmol, 17% yield, 99% purity) as a white solid. ¹H NMR (400 MHz, DMSO-d₆) δ=10.87 (br s, 1H), 7.60 (t, J=5.6 Hz, 1H), 6.19 (d, J=11.2 Hz, 2H), 5.24-5.06 (m, 1H), 4.16 (br t, J=7.6 Hz, 2H), 4.04 (br dd, J=5.2, 12.8 Hz, 1H), 3.73 (br dd, J=3.6, 9.2 Hz, 2H), 3.11 (br t, J=5.6 Hz, 2H), 2.83-2.73 (m, 1H), 2.68-2.53 (m, 3H), 2.36-2.22 (m, 3H), 2.14-2.01 (m, 1H), 2.00-1.90 (m, 1H). MS (ESI) m/z 444.0 [M+H]⁺.

### Example 98. Synthesis of Compound 97

To a solution of 3,3-dimethylcyclobutane-1-carboxylic acid (48.0 mg, 374 μmol, 1.00 eq) and triethylamine (104 μL, 749 μmol, 2.00 eq) in dioxane (1 mL) was added diphenylphosphoryl azide (129 μL, 599 umol, 1.60 eq). The mixture was stirred at 25° C. for 1 h, then 3-(2,6-difluoro-4-(3-hydroxyazetidin-1-yl)phenyl)piperidine-2,6-dione XII (122 mg, 411 μmol, 1.10 eq) was added and the mixture was stirred at 100° C. for 1 h. The reaction mixture was diluted with water (10 mL) and extracted with ethyl acetate (3×10 mL). The combined organic phase was washed with brine (20 mL), dried over anhydrous sodium sulfate, filtered and concentrated under reduced pressure to give a residue. The residue was purified by Prep-TLC (petroleum ether:ethyl acetate=1:1) to give a crude product. The crude product was purified by Prep-HPLC (column: Phenomenex luna C18 150*25 mm*10 μm; mobile phase: [water(formic acid)-acetonitrile]; B %: 37%-67%, 10 min) to afford 1-(4-(2,6-dioxopiperidin-3-yl)-3,5-difluorophenyl)azetidin-3-yl (3,3-dimethylcyclobutyl)carbamate #97 (14.2 mg, 33.6 μmol, 9% yield) as a white solid. ¹H NMR (400 MHz, DMSO-d₆) δ=10.87 (br s, 1H), 7.64 (d, J=7.6 Hz, 1H), 6.19 (d, J=10.8 Hz, 2H), 5.12 (br t, J=3.6 Hz, 1H), 4.14 (br t, J=7.6 Hz, 2H), 4.04 (br dd, J=5.2, 12.4 Hz, 1H), 4.00-3.89 (m, 1H), 3.71 (br dd, J=3.2, 8.8 Hz, 2H), 2.85-2.72 (m, 1H), 2.47-2.41 (m, 1H), 2.15-2.05 (m, 1H), 2.04-1.92 (m, 3H), 1.78-1.67 (m, 2H), 1.08 (d, J=14.4 Hz, 6H). MS (ESI) m/z 422.1 [M+H]⁺.

### Example 99. Synthesis of Compound 98

Step 1. To a solution of bicyclo[2.2.2]octan-1-amine (220 mg, 1.36 mmol, 1.00 eq) and pyridine (330 μL, 4.08 mmol, 3.00 eq) in acetonitrile (5.00 mL) was added phenyl carbonochloridate (222 μL, 1.77 mmol, 1.30 eq) dropwise. The mixture was stirred at 25° C. for 2 h. The mixture was concentrated to give a residue. The residue was purified by reverse phase column chromatography (column: spherical C18, 20-45 μm, 100 Å, SW 40, mobile phase: [water (0.1% formic acid)-acetonitrile]) and lyophilized to give phenyl bicycle[2.2.2] octan-1-ylcarbamate (150 mg, 0.611 mmol, 45% yield) as a white solid. MS (ESI) m/z 246.0 [M+H]⁺.

Step 2. To a solution of 3-(2,6-difluoro-4-(3-hydroxyazetidin-1-yl)phenyl)piperidine-2,6-dione XII (40.0 mg, 135 μmol, 1.00 eq) XII and phenyl bicyclo[2.2.2]octan-1-ylcarbamate (36.4 mg, 148 μmol, 1.10 eq) in dimethyformamide (2.00 mL) was added sodium hydride (8.10 mg, 202 μmol, 60% purity, 1.50 eq) at 0° C. The mixture was stirred at 20° C. for 2 h. The pH of the mixture was adjusted to 5-6 by formic acid and filtered. The filtrate was purified by Prep-HPLC (column: Phenomenex Luna C18 150*25 mm*10 μm; mobile phase: [water (formic acid)-acetonitrile]; B %: 44%-74%, 9 min) and lyophilized to afford 1-(4-(2,6-dioxopiperidin-3-yl)-3,5-difluorophenyl)azetidin-3-yl bicyclo[2.2.2]octan-1-ylcarbamate #98 (36.0 mg, 80.5 μmol, 60% yield) as a white solid. 1H NMR (400 MHz, DMSO-d6) δ=10.85 (s, 1H), 6.99 (br s, 1H), 6.18 (d, J=11.0 Hz, 2H), 5.13-5.05 (m, 1H), 4.13 (br t, J=7.7 Hz, 2H), 4.03 (dd, J=5.3, 12.6 Hz, 1H), 3.68 (br dd, J=3.1, 8.8 Hz, 2H), 2.86-2.69 (m, 2H), 2.13-2.02 (m, 1H), 1.98-1.90 (m, 1H), 1.73-1.66 (m, 6H), 1.59-1.52 (m, 6H), 1.52-1.46 (m, 1H). MS (ESI) m/z 488.3 [M+H]+.

### Example 100. Synthesis of Compound 99

Step 1. To a mixture of (2R)-bicyclo[2.2.1]heptan-2-ol (50.0 g, 446 mmol, 1.00 eq), isoindoline-1,3-dione (78.7 g, 535 mmol, 1.20 eq) and diethyl azodicarboxylate (97.2 mL, 535 mmol, 1.20 eq) in tetrahydrofuran (500 mL) was added triphenylphosphine (140 g, 535 mmol, 1.2 eq) in portions at 0° C. The mixture was stirred at 25° C. for 12 h under nitrogen atmosphere. The mixture was diluted with water (1 L) and extracted with ethyl acetate (3×3 L). The combined organic layer was washed with brine (500 mL), dried over sodium sulfate, filtered, and concentrated under reduced pressure to give a residue. The residue was purified by column chromatography (SiO2, petroleum ether/ethyl acetate=10/1), reversed phase column chromatography (column: I.D.100 mm×H300 mm Welch Ultimate XB_C18 20-40 m; 120 Å, mobile phase: [water (0.1% formic acid)-acetonitrile). The desired fraction was collected and extracted with ethyl acetate (3×600 mL). The combined organic layer was washed with brine (100 mL), dried over sodium sulfate, filtered and concentrated under reduced pressure to afford 2-((2S)-bicyclo[2.2.1]heptan-2-yl)isoindoline-1,3-dione (6.50 g, 26.9 mmol, 6% yield) as a white solid. 1H NMR (400 MHz, DMSO-d6) δ=7.83-7.79 (m, 4H), 4.39-4.28 (m, 1H), 2.45-2.38 (m, 2H), 2.31 (br s, 1H), 1.78-1.69 (m, 1H), 1.67-1.59 (m, 1H), 1.53-1.40 (m, 3H), 1.37-1.27 (m, 2H).

Step 2. A mixture of 2-((2S)-bicyclo[2.2.1]heptan-2-yl)isoindoline-1,3-dione (3.00 g, 12.4 mmol, 1.00 eq) in hydrochloric acid (12 M, 50.0 mL, 48.3 eq) was stirred at 100° C. for 96 h. The mixture was concentrated under reduced pressure to give (2S)-bicyclo[2.2.1]heptan-2-amine (1.40 g, crude) as a yellow solid. To a mixture of (2S)-bicyclo[2.2.1]heptan-2-amine (700 mg, 6.30 mmol, 1.00 eq) and pyridine (32.0 mL, 396 mmol, 62.9 eq) in acetonitrile (30 mL) was added phenyl carbonochloridate (946 μL, 7.56 mmol, 1.20 eq) dropwise at 0° C. The mixture was stirred at 25° C. for 12 h. The mixture was concentrated under reduced pressure to give a residue. The residue was purified by reversed phase column chromatography (column: spherical C18, 20-45 μm, 100 Å, SW 120, mobile phase: [water (0.1% formic acid)-acetonitrile) and column chromatography (SiO2, petroleum ether/ethyl acetate=10/1) and concentrated under reduced pressure to afford phenyl (2S)-bicyclo[2.2.1]heptan-2-ylcarbamate (90.0 mg, 389 μmol, 6% yield) as a white solid. 1H NMR (400 MHz, DMSO-d6) δ=7.82 (br d, J=6.6 Hz, 1H), 7.37 (br t, J=7.8 Hz, 2H), 7.23-7.17 (m, 1H), 7.10 (br d, J=7.8 Hz, 2H), 3.78 (br dd, J=5.4, 10.3 Hz, 1H), 2.30 (br s, 1H), 2.15 (br s, 1H), 1.95-1.82 (m, 1H), 1.74-1.62 (m, 1H), 1.46-1.26 (m, 5H), 1.05-0.94 (m, 1H).

Step 3. To a mixture of phenyl (2S)-bicyclo[2.2.1]heptan-2-ylcarbamate (46.8 mg, 203 μmol, 1.20 eq) and 3-(2,6-difluoro-4-(3-hydroxyazetidin-1-yl)phenyl)piperidine-2,6-dione XII (50.0 mg, 169 μmol, 1.00 eq) in dimethyformamide (1 mL) was added sodium hydride (10.1 mg, 253 μmol, 60% purity, 1.50 eq) in portions at 0° C. The mixture was stirred at 25° C. for 0.5 h. The mixture was quenched with formic acid (0.2 mL) and filtered. The filtrate was purified by Prep-HPLC (column: Phenomenex Luna C18 150*25 mm*10 μm; mobile phase: [water (formic acid)-acetonitrile]; B %: 36%-66%, 10.5 min) and lyophilized to afford 1-(4-(2,6-dioxopiperidin-3-yl)-3,5-difluorophenyl)azetidin-3-yl(2S)-bicyclo[2.2.1] heptan-2-ylcarbamate #99 (26.8 mg, 61.2 μmol, 36% yield, 99% purity) as a white solid. 1H NMR (400 MHz, DMSO-d6) δ=10.85 (s, 1H), 7.45 (br d, J=6.8 Hz, 1H), 6.19 (d, J=11.0 Hz, 2H), 5.21-5.08 (m, 1H), 4.16 (br t, J=7.4 Hz, 2H), 4.04 (dd, J=5.0, 12.5 Hz, 1H), 3.79-3.66 (m, 3H), 2.83-2.73 (m, 1H), 2.53 (br d, J=1.8 Hz, 1H), 2.25 (br s, 1H), 2.17-2.01 (m, 2H), 1.99-1.91 (m, 1H), 1.88-1.75 (m, 1H), 1.65-1.52 (m, 1H), 1.47-1.21 (m, 5H), 0.98-0.82 (m, 1H). MS (ESI) m/z 434.1 [M+H]+.

### Example 101. Synthesis of Compound 100

Step 1. To a mixture of ((1R,4R)-4-aminocyclohexyl)methanol (500 mg, 3.87 mmol, 1.00 eq) and sodium bicarbonate (301 μL, 7.74 mmol, 2.00 eq) in water (2 mL) and tetrahydrofuran (2 mL) was added phenyl carbonochloridate (533 μL, 4.26 mmol, 1.10 eq) dropwise at 0° C. The mixture was stirred at 25° C. for 2 h. The reaction mixture was diluted with water (15 mL), extracted with ethyl acetate (3×15 mL). The combined organic layers were washed with brine (20 mL), dried over sodium sulfate, filtered and concentrated under reduced pressure to give a residue. It was purified by reversed phase column chromatography (column: spherical C18, 20-45 μm, 100 Å, SW 120, mobile phase: water (0.1% formic acid)-acetonitrile). The desired fraction was collected and extracted with ethyl acetate (3×15 mL). The combined organic layers were washed with brine (20 mL), dried over sodium sulfate, filtered and concentrated under reduced pressure to afford phenyl ((1R,4R)-4-(hydroxymethyl)cyclohexyl)carbamate (400 mg, 1.60 mmol, 41% yield) as a white solid. 1H NMR (400 MHz, DMSO-d6) δ=7.67 (br d, J=7.9 Hz, 1H), 7.40-7.33 (m, 2H), 7.21-7.15 (m, 1H), 7.08 (br d, J=7.8 Hz, 2H), 4.39 (t, J=5.3 Hz, 1H), 3.20 (t, J=5.8 Hz, 2H), 1.88 (br d, J=10.4 Hz, 2H), 1.75 (br d, J=11.9 Hz, 2H), 1.51-1.05 (m, 4H), 0.98-0.87 (m, 2H). MS (ESI) m/z 250.2 [M+H]+.

Step 2. To a mixture of 3-(2,6-difluoro-4-(3-hydroxyazetidin-1-yl)phenyl)piperidine-2,6-dione XII (100 mg, 337 μmol, 1.00 eq) and phenyl ((1R,4R)-4-(hydroxymethyl)cyclohexyl)carbamate (100 mg, 405 μmol, 1.20 eq) in dimethyformamide (1.50 mL) was added sodium hydride (20.2 mg, 506 μmol, 60% purity, 1.50 eq) at 0° C. in portions. The mixture was stirred at 25° C. for 1 h. The mixture was quenched by formic acid to adjust pH=5 and filtered. The filtrate was purified by Prep-HPLC (column: Phenomenex Luna C18 150*25 mm*10 μm; mobile phase: [water (formic acid)-acetonitrile]; B %: 24%-54%, 9 min) and lyophilized to afford 1-(4-(2,6-dioxopiperidin-3-yl)-3,5-difluorophenyl)azetidin-3-yl((1R,4R)-4-(hydroxymethyl)cyclohexyl) carbamate #100 (85.6 mg, 184 μmol, 54% yield, 97% purity) as a white solid. 1H NMR (400 MHz, DMSO-d6) δ=10.86 (br s, 1H), 7.31 (d, J=7.9 Hz, 1H), 6.18 (d, J=11.0 Hz, 2H), 5.16-5.08 (m, 1H), 4.37 (t, J=5.3 Hz, 1H), 4.14 (br t, J=7.6 Hz, 2H), 4.03 (dd, J=5.1, 12.6 Hz, 1H), 3.70 (m, 2H), 3.18 (m, 2H), 2.82-2.72 (m, 1H), 2.52 (br s, 1H), 2.12-2.01 (m, 1H), 1.98-1.90 (m, 1H), 1.84-1.69 (m, 4H), 1.26-1.08 (m, 3H), 0.95-0.84 (m, 2H). MS (ESI) m/z 452.0 [M+H]+.

### Example 102. Synthesis of Compound 101

Step 1. To a mixture of tert-butyl pyrrolidin-3-ylcarbamate (1.00 g, 5.37 mmol, 1.00 eq) in tetrahydrofuran (20 mL) was added sodium hydride (322 mg, 8.06 mmol, 60% purity, 1.50 eq) in portions at 0° C. The mixture was stirred at 0° C. for 0.5 h. Then the mixture was added iodoethane (429 μL, 5.37 mmol, 1.00 eq) dropwise. The mixture was stirred at 25° C. for 2 h. The mixture was quenched with water (150 mL) and extracted with ethyl acetate (3×80 mL). The combined organic layer was washed with brine (60 mL), dried over sodium sulfate, filtered and concentrated in vacuum to give tert-butyl (1-ethylpyrrolidin-3-yl)carbamate (1.00 g, 4.67 mmol, 87% yield) as a yellow solid. 1H NMR (400 MHz, DMSO-d6) δ=6.89 (br d, J=5.0 Hz, 1H), 3.86 (br s, 1H), 2.66 (br t, J=7.4 Hz, 1H), 2.45-2.32 (m, 4H), 2.23-2.14 (m, 1H), 2.06-1.92 (m, 1H), 1.59-1.47 (m, 1H), 1.36 (s, 9H), 1.03-0.93 (m, 3H).

Step 2. A mixture of tert-butyl (1-ethylpyrrolidin-3-yl)carbamate (1.00 g, 4.67 mmol, 1.00 eq) in hydrochloric acid/dioxane (4 M, 10.0 mL) was stirred at 25° C. for 1 h. The mixture was concentrated under reduced pressure to give 1-ethylpyrrolidin-3-amine (800 mg, crude) as yellow oil. To a mixture of 1-ethylpyrrolidin-3-amine (250 mg, 2.19 mmol, 1.00 eq), pyridine (884 μL, 10.9 mmol, 5.00 eq) in acetonitrile (5.00 mL) was added phenyl carbonochloridate (411 mg, 2.63 mmol, 329 uL, 1.20 eq) dropwise. The mixture was stirred at 25° C. for 2 h. The mixture was concentrated under reduced pressure. The residue was purified by reverse phase chromatography (column: spherical C18, 20-45 μm, 100 Å, SW 80, mobile phase: water (0.1% formic acid)-acetonitrile) and lyophilized to give phenyl (1-ethylpyrrolidin-3-yl) carbamate (200 mg, crude) as yellow oil. MS (ESI) m/z 235.2 [M+H]+.

Step 3. To a mixture of phenyl (1-ethylpyrrolidin-3-yl)carbamate (119 mg, 508 μmol, 1.50 eq), 3-(4-(3-aminoazetidin-1-yl)-2,6-difluorophenyl)piperidine-2,6-dione XII (100 mg, 339 μmol, 1.00 eq) in dimethylformamide (1 mL) was added triethylamine (141 μL, 1.02 mmol, 3.00 eq) dropwise. The mixture was stirred at 50° C. for 12 h. The mixture was adjusted pH<7 with formic acid (0.1 mL) and filtered. The filtrate was purified by Prep-HPLC (column: Phenomenex Luna C18 150*25 mm*10 μm; mobile phase: [water (formic acid)-acetonitrile]; B %: 1%-30%, 10 min), Prep-HPLC (column: Phenomenex Luna C18 150 mm*25 mm*10 μm; mobile phase: [water(formic acid)-acetonitrile]; B %: 0%-30%, 10 min) and lyophilized to give 1-(1-(4-(2,6-dioxopiperidin-3-yl)-3,5-difluorophenyl)azetidin-3-yl)-3-(1-ethylpyrrolidin-3-yl) #101 (5.03 mg, 9.92 μmol, 3% yield, 95% purity, formate) as a white solid. 1H NMR (400 MHz, DMSO-d6) δ=10.87 (br s, 1H), 8.26 (s, 1H), 6.64 (br d, J=7.6 Hz, 1H), 6.40-6.29 (m, 1H), 6.15 (d, J=11.0 Hz, 2H), 4.47 (td, J=6.7, 13.2 Hz, 1H), 4.12-3.98 (m, 4H), 3.55 (br t, J=6.6 Hz, 2H), 2.87-2.71 (m, 3H), 2.61-2.53 (m, 3H), 2.16-1.87 (m, 5H), 1.54 (dt, J=7.5, 12.8 Hz, 1H), 1.06 (t, J=7.2 Hz, 3H). MS (ESI) m/z 436.1 [M+H]+.

### Example 103. Synthesis of Compound 102

Step 1. To a solution of 4-methyltetrahydro-2H-pyran-4-amine (500 mg, 4.34 mmol, 1.00 eq) in acetonitrile (5 mL) was added pyridine (1.05 mL, 13.0 mmol, 3.00 eq) and phenyl carbonochloridate (0.653 mL, 5.21 mmol, 1.20 eq). The mixture was stirred at 20° C. for 1 h. The reaction mixture was quenched by hydrochloric acid (1 M, 20 mL), extracted with ethyl acetate (3×20 mL). The combined organic layers were dried over anhydrous sodium sulfate, filtered and concentrated under reduced pressure to give a residue. The residue was purified by column chromatography (SiO2, petroleum ether:ethyl acetate=20/1 to 5/1) and concentrated to afford phenyl (4-methyltetrahydro-2H-pyran-4-yl) carbamate (550 mg, 2.34 mmol, 54% yield, 96% purity) as a white solid. 1H NMR (400 MHz, CDCl3) δ=7.41-7.32 (m, 2H), 7.24-7.18 (m, 1H), 7.13 (d, J=7.5 Hz, 2H), 4.94 (s, 1H), 3.83-3.67 (m, 4H), 2.10-1.95 (m, 2H), 1.80-1.70 (m, 2H), 1.48 (s, 3H).

Step 2. To a solution of 3-(2,6-difluoro-4-(3-hydroxyazetidin-1-yl)phenyl)piperidine-2,6-dione XII (100 mg, 338 μmol, 1.00 eq) in N,N-dimethyl formamide (2 mL) was added sodium hydride (27.0 mg, 675 μmol, 60% purity, 2.00 eq) at 0° C. The mixture was stirred at 0° C. for 10 min. Then phenyl (4-methyltetrahydro-2H-pyran-4-yl)carbamate (95.3 mg, 405 μmol, 1.20 eq) was added. The mixture was stirred at 20° C. for 2 h. The pH of the mixture was adjusted to 5˜6 with formic acid and filtered. The filtrate was purified by Prep-HPLC (column: Phenomenex Luna C18 150*25 mm*10 μm; mobile phase: [water (formic acid)-acetonitrile]; B %: 24%-54%, 9 min) and lyophilized to afford 1-(4-(2,6-dioxopiperidin-3-yl)-3,5-difluorophenyl)azetidin-3-yl(4-methyltetrahydro-2H-pyran-4-yl)carbamate #102 (66.54 mg, 154 μmol, 46% yield, 99% purity) as a white solid. 1H NMR (400 MHz, DMSO-d6) δ=10.9 (s, 1H), 7.23 (s, 1H), 6.20 (d, J=11.0 Hz, 2H), 5.18-5.06 (m, 1H), 4.20-4.11 (m, 2H), 4.04 (dd, J=5.1, 12.7 Hz, 1H), 3.75-1.65 (m, 2H), 3.58-3.45 (m, 4H), 2.84-2.72 (m, 1H), 2.52 (br s, 1H), 2.12-2.02 (m, 1H), 1.95-1.85 (m, 3H), 1.50-1.40 (m, 2H), 1.25 (s, 3H). MS (ESI) m/z 438.2 [M+H]+.

### Example 104. Synthesis of Compound 103

Step 1. To a solution of (1S,4S)-4-methylcyclohexanamine hydrochloride (250 mg, 1.67 mmol, 1.00 eq) and phenyl carbonochloridate (313 μL, 2.51 mmol, 1.50 eq) in acetonitrile (10 mL) was added pyridine (134 μL, 1.67 mmol, 1.00 eq) at 25° C. The reaction mixture was stirred at 25° C. for 12 h. The reaction mixture was poured into water (30 mL). The mixture was extracted with ethyl acetate (3×30 mL) and the organic layers were collected. The combined organic layers were washed with brine (3×10 mL), dried over sodium sulfate, filtered and concentrated to give phenyl ((1S,4S)-4-methylcyclohexyl)carbamate (408 mg, crude) as a white solid and was used for next step directly.

Step 2. To a mixture of 3-(2,6-difluoro-4-(3-hydroxyazetidin-1-yl)phenyl)piperidine-2,6-dione XII (100 mg, 337 μmol, 1.00 eq) in dimethyl formamide (10 mL) was added sodium hydride (27.0 mg, 675 μmol, 60% purity, 2.00 eq) at 0° C. The mixture was stirred at 25° C. for 0.5 h. Then phenyl ((1S,4S)-4-methylcyclohexyl)carbamate (236 mg, 1.01 mmol, 3.00 eq) was added and the resulting reaction mixture was stirred at 25° C. for 1 h. The reaction mixture was quenched with formic acid (0.1 mL). The residue was purified by Prep-HPLC (column: Phenomenex Luna C18 150 mm*25 mm*10 μm; mobile phase: [water (formic acid)-acetonitrile]; B %: 42%-72%, 9 min). Then the residue was purified by Prep-TLC (SiO2, petroleum ether/ethyl acetate=3/1) to afford 1-(4-(2,6-dioxopiperidin-3-yl)-3,5-difluorophenyl) azetidin-3-yl((1S,4S)-4-methylcyclohexyl)carbamate #103 (18.0 mg, 40.1 μmol, 12% yield, 97% purity) as a white solid. 1H NMR (400 MHz, DMSO-d6) δ=10.86 (s, 1H), 7.33 (d, J=7.0 Hz, 1H), 6.19 (d, J=11.0 Hz, 2H), 5.13 (s, 1H), 4.21-4.09 (m, 2H), 4.03 (dd, J=5.4, 12.6 Hz, 1H), 3.72 (dd, J=3.6, 8.6 Hz, 2H), 3.53-3.45 (m, 1H), 2.83-2.72 (m, 1H), 2.14-2.02 (m, 1H), 2.00-1.89 (m, 1H), 1.60-1.34 (m, 8H), 1.32-1.24 (m, 2H), 0.87 (d, J=6.6 Hz, 3H). MS (ESI) m/z 436.2 [M+H]+.

### Example 105. Synthesis of Compound 104

Step 1. To a solution of (R)-2,3-dihydro-1H-inden-1-amine (705 mg, 4.50 mmol, 565 μL, 1.20 eq) in acetonitrile (10 mL) was added pyridine (1.52 mL, 18.8 mmol, 5.00 eq) and phenyl carbonochloridate (637 mg, 3.75 mmol, 1.00 eq). The mixture was stirred at 0° C. for 1 h. The reaction mixture was filtered and concentrated under reduced pressure to give a residue. It was purified by flash silica gel chromatography (ISCO; 20 g SepaFlash Silica Flash Column, Eluent of 0 to 30% ethyl acetate/petroleum ether gradient at 80 mL/min) to afford (R)-phenyl (2,3-dihydro-1H-inden-1-yl)carbamate (620 mg, 2.45 mmol, 65% yield) as a white solid. 1H NMR (400 MHz, DMSO-d6) δ=8.19 (br d, J=8.4 Hz, 1H), 7.43-7.37 (m, 2H), 7.34-7.28 (m, 1H), 7.28-7.20 (m, 4H), 7.16 (d, J=7.6 Hz, 2H), 5.09 (q, J=8.1 Hz, 1H), 3.01-2.89 (m, 1H), 2.80 (td, J=8.2, 16.0 Hz, 1H), 2.45 (dt, J=4.0, 8.2 Hz, 1H), 1.97-1.83 (m, 1H). MS (ESI) m/z 253.9 [M+H]+.

Step 2. To a solution of (R)-phenyl (2,3-dihydro-1H-inden-1-yl)carbamate (51.3 mg, 203 μmol, 1.20 eq) in dimethyl formamide (2 mL) were added 3-(2,6-difluoro-4-(3-hydroxyazetidin-1-yl)phenyl)piperidine-2,6-dione XII (50.0 mg, 169 μmol, 1.00 eq) and sodium hydride (13.5 mg, 338 μmol, 60% purity, 2.00 eq) at 0° C. The mixture was stirred at 0° C. for 1 h. The mixture was filtered and the filtrate was purified by Prep-HPLC (column: Phenomenex Synergi C18 150*25 mm*10 μm; mobile phase: [water (formic acid)-acetonitrile]; B %: 39%-69%, 10 min) to afford 1-(4-(2,6-dioxopiperidin-3-yl)-3,5-difluorophenyl)azetidin-3-yl((R)-2,3-dihydro-1H-inden-1-yl) carbamate #104 (13.31 mg, 28.9 μmol, 17% yield, 99% purity) as a white solid. 1H NMR (400 MHz, DMSO-d6) δ=10.86 (s, 1H), 7.83 (d, J=8.4 Hz, 1H), 7.25-7.14 (m, 4H), 6.20 (d, J=11.0 Hz, 2H), 5.26-5.17 (m, 1H), 5.02 (br d, J=8.2 Hz, 1H), 4.22-4.14 (m, 2H), 4.04 (dd, J=5.0, 12.5 Hz, 1H), 3.82-3.73 (m, 2H), 2.93-2.86 (m, 1H), 2.82-2.73 (m, 2H), 2.52 (br s, 1H), 2.44-2.34 (m, 1H), 2.14-2.01 (m, 1H), 2.00-1.89 (m, 1H), 1.81 (dd, J=8.5, 12.5 Hz, 1H). MS (ESI) m/z 456.1[M+H]+.

### Example 106. Synthesis of Compound 105

To a solution of 3-(2,6-difluoro-4-(3-hydroxyazetidin-1-yl)phenyl)piperidine-2,6-dione XII (100 mg, 337 μmol, 1.00 eq) in dimethylformamide (1 mL) was added di(1H-imidazol-1-yl)methanone (218 mg, 1.35 mmol, 4.00 eq). The mixture was stirred at 20° C. for 2 h. Then 2-oxa-6-azaspiro[3.3]heptane (33.4 mg, 0.337 mmol, 1.00 eq), triethylamine (46.9 μL, 0.337 mmol, 1.00 eq) and 3,4,5,7,8,9,10,10a-octahydropyrido a[1,2-α][1,4]diazepine (50.8 μL, 0.337 mmol, 1.00 eq) were added to the mixture and stirred at 20° C. for 2 h. The pH of the mixture was adjusted to 5-6 with formic acid and filtered. The filtrate was purified by Prep-HPLC (column: Phenomenex Luna C18 150*25 mm*10 μm; mobile phase: [water (formic acid)-acetonitrile]; B %: 20%-50%, 9 min) and lyophilized to afford 1-(4-(2,6-dioxopiperidin-3-yl)-3,5-difluorophenyl)azetidin-3-yl 2-oxa-6-azaspiro[3.3]heptane-6-carboxylate #105 (49.8 mg, 0.113 mmol, 34% yield, 96% purity) as an off white solid. 1H NMR (400 MHz, DMSO-d6) δ=10.86 (s, 1H), 6.19 (s, 1H), 6.17 (s, 1H), 5.15-5.08 (m, 1H), 4.64 (s, 4H), 4.17-4.01 (m, 7H), 3.73 (dd, J=3.8, 9.1 Hz, 2H), 2.83-2.71 (m, 1H), 2.52 (m, 1H), 2.14-2.00 (m, 1H), 1.98-1.89 (m, 1H). MS (ESI) m/z 422.0 [M+H]+.

### Example 107. Synthesis of Compound 106

To a solution of 3-(2,6-difluoro-4-(3-hydroxyazetidin-1-yl)phenyl)piperidine-2,6-dione XII (60.0 mg, 203 μmol, 1.00 eq) in dimethyl formamide (5 mL) was added 1,1′-carbonyldiimidazole (197 mg, 1.22 mmol, 6.00 eq). The reaction mixture was stirred at 25° C. for 2 h. Then (3R,5R,7R)-adamantan-1-ylmethanamine (66.9 mg, 405 μmol, 2.00 eq) was added to the mixture and the mixture was stirred at 25° C. for 12 h. The crude product was purified by reversed phase column chromatography (column: spherical C18, 20-45 μm, 100 Å, SW 40, mobile phase: [water (0.1% formic acid)-acetonitrile) and lyophilized to afford 1-(4-(2,6-dioxopiperidin-3-yl)-3,5-difluorophenyl)azetidin-3-yl((3R,5R,7R)-adamantan-1-ylmethyl) carbamate #106 (20.0 mg, 40.6 μmol, 20% yield, 99% purity) as a white solid. 1H NMR (400 MHz, DMSO-d6) δ=10.86 (s, 1H), 7.33 (t, J=6.4 Hz, 1H), 6.19 (d, J=11.2 Hz, 2H), 5.17-5.04 (m, 1H), 4.15 (t, J=7.8 Hz, 2H), 4.03 (dd, J=5.0, 12.8 Hz, 1H), 3.72 (dd, J=3.4, 8.6 Hz, 2H), 2.75 (dd, J=4.8, 13.1 Hz, 1H), 2.68 (d, J=6.5 Hz, 2H), 2.52 (s, 1H), 2.14-2.00 (m, 1H), 1.98-1.87 (m, 4H), 1.68-1.54 (m, 6H), 1.41 (s, 6H). MS (ESI) m/z 488.3 [M+H]+.

### Example 108. Synthesis of Compound 107

To a solution of 3-(2,6-difluoro-4-(3-hydroxyazetidin-1-yl)phenyl)piperidine-2,6-dione XII (100 mg, 0.338 mmol, 1.00 eq) in N,N-dimethyl formamide (2 mL) was added 1,1′-carbonyldiimidazole (328 mg, 2.03 mmol, 6.00 eq). The mixture was stirred at 25° C. for 2 h. Triethylamine (47.0 μL, 0.338 mmol, 1.00 eq) 2,3,4,6,7,8,9,10-octahydropyrimido[1,2-a]azepine (50.9 μL, 0.338 mmol, 1.00 eq) and (1R,3S,5R,7R)-adamantan-2-amine (51.1 mg, 0.338 mmol, 1.00 eq) were added to the mixture and stirred at 25° C. for another 14 h. The mixture was filtered and the filtrate was purified by Prep-HPLC (column: Phenomenex Luna C18 150*25 mm*10 μm; mobile phase: [water (formic acid)-acetonitrile]; B %: 45%-75%, 10.5 min) and further purified by Prep-HPLC (column: Phenomenex Luna C18 150*25 mm*10 μm; mobile phase: [water (formic acid)-acetonitrile]; B %: 49%-79%, 10 min) and lyophilized to afford 1-(4-(2,6-dioxopiperidin-3-yl)-3,5-difluorophenyl)azetidin-3-yl (1R,3S,5R,7R)-adamantan-2-ylcarbamate #107 (21.0 mg, 0.0434 mmol, 12% yield, 96% purity) as a white solid. 1H NMR (400 MHz, DMSO-d6) δ=10.85 (br s, 1H), 7.44 (br d, J=7.6 Hz, 1H), 6.19 (d, J=11.0 Hz, 2H), 5.20-5.10 (m, 1H), 4.15 (br t, J=7.6 Hz, 2H), 4.03 (dd, J=5.2, 12.7 Hz, 1H), 3.74 (br dd, J=3.3, 8.6 Hz, 2H), 3.60 (br d, J=7.0 Hz, 1H), 2.84-2.71 (m, 1H), 2.59-2.55 (m, 1H), 2.13-2.04 (m, 1H), 2.00 (br s, 1H), 1.97 (br s, 2H), 1.80 (br d, J=5.8 Hz, 4H), 1.75 (br d, J=13.6 Hz, 4H), 1.70-1.66 (m, 2H), 1.44 (br d, J=12.3 Hz, 2H). MS (ESI) m/z 474.3 [M+H]+.

### Example 109. Synthesis of Compound 108

Step 1. To a solution of 1,3-dimethoxypropan-2-amine (1.00 g, 8.39 mmol, 1.00 eq) and pyridine (1.35 mL, 16.8 mmol, 2.00 eq) in acetonitrile (15 mL) was added phenyl carbonochloridate (1.26 mL, 10.1 mmol, 1.20 eq) dropwise at 0° C. The mixture was stirred at 20° C. for 2 h. The mixture was diluted with hydrochloric acid (1 M, 50 mL) and extracted with acetonitrile (3×50 mL). The combined organic layers were dried over anhydrous sodium sulfate, filtered and concentrated under reduced pressure. The crude product was purified by column chromatography (SiO2, petroleum ether/ethyl acetate=50/1 to 10/1) to give phenyl(1,3-dimethoxypropan-2-yl)carbamate (1.30 g, 5.43 mmol, 65% yield) as a colorless oil. 1H NMR (400 MHz, DMSO-d6) δ=7.74 (br d, J=8.4 Hz, 1H), 7.41-7.34 (m, 2H), 7.25-7.16 (m, 1H), 7.09 (dd, J=0.9, 8.4 Hz, 2H), 3.89-3.75 (m, 1H), 3.39 (d, J=6.0 Hz, 4H), 3.27 (s, 6H). MS (ESI) m/z 240.2 [M+H]+.

Step 2. To a solution of phenyl(1,3-dimethoxypropan-2-yl)carbamate (96.9 mg, 405 μmol, 1.20 eq) and 3-(2,6-difluoro-4-(3-hydroxyazetidin-1-yl)phenyl)piperidine-2,6-dione XII (100 mg, 337 μmol, 1.00 eq) in dimethylformamide (2 mL) was added sodium hydride (20.3 mg, 506 μmol, 60% purity, 1.50 eq) at 0° C. The mixture was stirred at 20° C. for 2 h. The pH of the mixture was adjusted to 5-6 with formic acid and filtered. The filtrate was purified by Prep-HPLC (column: Phenomenex Luna C18 150*25 mm*10 μm; mobile phase: [water (formic acid)-acetonitrile]; B %: 23%-53%, 9 min) and lyophilized to afford 1-(4-(2,6-dioxopiperidin-3-yl)-3,5-difluorophenyl)azetidin-3-yl (1,3-dimethoxypropan-2-yl)carbamate #108 (63.3 mg, 143 μmol, 42% yield) as an off-white solid. 1H NMR (400 MHz, DMSO-d6) δ=10.85 (s, 1H), 7.37 (br d, J=8.5 Hz, 1H), 6.19 (d, J=11.0 Hz, 2H), 5.14 (tt, J=4.0, 6.3 Hz, 1H), 4.21-4.12 (m, 2H), 4.03 (br dd, J=5.0, 12.8 Hz, 1H), 3.79-3.69 (m, 3H), 3.40-3.35 (m, 1H), 3.30 (br s, 3H), 3.23 (s, 6H), 2.85-2.72 (m, 1H), 2.60-2.54 (m, 1H), 2.07 (dq, J=3.9, 12.9 Hz, 1H), 2.00-1.89 (m, 1H). MS (ESI) m/z 442.2 [M+H]+.

### Example 110. Synthesis of Compound 109

A mixture of 3-(2,6-difluoro-4-(3-hydroxyazetidin-1-yl)phenyl)piperidine-2,6-dione XII (100 mg, 337 μmol, 1.00 eq) and 1,1′-carbonyldiimidazole (109 mg, 675 μmol, 2.00 eq) in dimethyl formamide (1 mL) was stirred at 25° C. for 2 h. Then 3,3,3-trifluoropropan-1-amine (57.2 mg, 506 μmol, 1.50 eq), triethylamine (46.9 μL, 337 μmol, 1.00 eq) and 1,8-diazabicyclo[5.4.0]undec-7-ene (50.8 μL, 337 μmol, 1.00 eq) were added and the reaction mixture was stirred at 25° C. for another 12 h. The mixture was concentrated under reduced pressure to give a crude product. It was purified by reversed phase column chromatography (column: spherical C18, 20-45 μm, 100 Å, SW 80, mobile phase: [water (0.1% formic acid)-acetonitrile) to give 1-(4-(2,6-dioxopiperidin-3-yl)-3,5-difluorophenyl)azetidin-3-yl (3,3,3-trifluoropropyl)carbamate #109 (39.2 mg, 90.0 μmol, 26% yield) as an off-white solid. 1H NMR (400 MHz, DMSO-d6) δ=10.85 (s, 1H), 7.60 (br t, J=5.7 Hz, 1H), 6.19 (d, J=11.0 Hz, 2H), 5.20-5.13 (m, 1H), 4.16 (br t, J=7.5 Hz, 2H), 4.04 (br dd, J=5.2, 12.3 Hz, 1H), 3.72 (br dd, J=3.4, 8.8 Hz, 2H), 3.30-3.23 (m, 4H), 2.83-2.74 (m, 1H), 2.45-2.44 (m, 1H), 2.08 (br dd, J=3.9, 12.9 Hz, 1H), 1.97-1.92 (m, 1H). MS (ESI) m/z 436.0 [M+H]+.

### Example 111. Synthesis of Compound 110

To a solution of 3-(2,6-difluoro-4-(3-hydroxyazetidin-1-yl)phenyl)piperidine-2,6-dione XII (100 mg, 0.338 mmol, 1.00 eq) in N,N-dimethyl formamide (2 mL) was added di(1H-imidazol-1-yl)methanone (328 mg, 2.03 mmol, 6.00 eq). The mixture was stirred at 25° C. for 2 h. Then triethylamine (470 μL, 0.338 mmol, 1.00 eq), 2,3,4,6,7,8,9,10-octahydropyrimido[1,2-α]azepine (50.9 μL, 338 μmol, 1.00 eq) and (1R,2R,4S)-norbornan-2-amine (40.0 μL, 0.338 mmol, 1.00 eq) were added to the mixture and stirred at 25° C. for 14 h. The mixture was filtered and the filtrate was purified by Prep-HPLC (column: Phenomenex Luna C18 150*25 mm*10 μm; mobile phase: [water (formic acid)-acetonitrile]; B %: 37%-67%, 10.0 min) and further purified by Prep-HPLC (column: Phenomenex Luna C18 100*30 mm*5 um; mobile phase: [water (formic acid)-acetonitrile]; B %: 40%-70%, 8 min). The desired fraction was collected and lyophilized to afford 1-(4-(2,6-dioxopiperidin-3-yl)-3,5-difluorophenyl)azetidin-3-yl(1R,2R,4S)-bicyclo[2.2.1]heptan-2-ylcarbamate #110 (64.4 mg, 0.147 mmol, 43% yield, 99% purity) as a white solid. 1H NMR (400 MHz, DMSO-d6) δ=10.85 (s, 1H), 7.33 (br d, J=7.0 Hz, 1H), 6.18 (d, J=11.0 Hz, 2H), 5.20-5.06 (m, 1H), 4.14 (br t, J=7.3 Hz, 2H), 4.03 (br dd, J=4.8, 12.0 Hz, 1H), 3.71 (td, J=4.4, 8.4 Hz, 2H), 3.37 (br s, 1H), 2.84-2.71 (m, 1H), 2.56 (br d, J=3.5 Hz, 1H), 2.16 (br s, 1H), 2.06 (br s, 1H), 1.99-1.92 (m, 1H), 1.58-1.51 (m, 1H), 1.45-1.38 (m, 3H), 1.31-1.25 (m, 1H), 1.10-0.99 (m, 4H). MS (ESI) m/z 434.2 [M+H]+.

### Example 112. Synthesis of Compound 111

Step 1. To a mixture of (R)-3,3-difluorocyclopentanamine (250 mg, 2.06 mmol, 1.00 eq), pyridine (833 μL, 10.3 mmol, 5.00 eq) in acetonitrile (2 mL) was added phenyl carbonochloridate (310 μL, 2.48 mmol, 1.20 eq) dropwise at 0° C. The mixture was stirred at 25° C. for 1 h. The mixture was concentrated under reduced pressure. The residue was purified by reverse phase chromatography (column: spherical C18, 20-45 μm, 100 Å, SW 40, mobile phase: [water (0.1% formic acid)-acetonitrile) and lyophilized to give (R)-phenyl (3,3-difluorocyclopentyl)carbamate (300 mg, 1.24 mmol, 60% yield) as a white solid. 1H NMR (400 MHz, DMSO-d6) δ=8.06 (d, J=7.0 Hz, 1H), 7.41-7.33 (m, 2H), 7.24-7.16 (m, 1H), 7.11 (br d, J=7.8 Hz, 2H), 4.11-4.00 (m, 1H), 2.31-1.98 (m, 5H), 1.83-1.67 (m, 1H).

Step 2. To a mixture of (R)-phenyl(3,3-difluorocyclopentyl)carbamate (98.0 mg, 0.406 mmol, 1.20 eq) and 3-(4-(3-aminoazetidin-1-yl)-2,6-difluorophenyl)piperidine-2,6-dione XII (100 mg, 339 μmol, 1.00 eq) in dimethyformamide (1 mL) was added triethylamine (141 μL, 1.02 mmol, 3.00 eq) dropwise. The mixture was stirred at 50° C. for 12 h. The mixture was quenched with formic acid (0.2 mL) and filtered. The filtrate was purified by Prep-HPLC (column: Phenomenex Luna C18 150*25 mm*10 μm; mobile phase: [water (formic acid)-acetonitrile]; B %: 25%-55%, 10 min) and lyophilized immediately to give 1-((R)-3,3-difluorocyclopentyl)-3-(1-(4-(2,6-dioxopiperidin-3-yl)-3,5-difluorophenyl)azetidin-3-yl)urea #111 (27.62 mg, 0.0606 mmol, 17% yield, 97% purity) as a white solid. 1H NMR (400 MHz, DMSO-d6) δ=10.86 (br s, 1H), 6.58 (d, J=7.6 Hz, 1H), 6.27 (d, J=7.4 Hz, 1H), 6.15 (d, J=11.0 Hz, 2H), 4.55-4.42 (m, 1H), 4.11-4.00 (m, 4H), 3.57 (br t, J=6.7 Hz, 2H), 2.82-2.73 (m, 1H), 2.41-2.34 (m, 1H), 2.20-1.90 (m, 7H), 1.64-1.53 (m, 1H). MS (ESI) m/z 443.1 [M+H]+.

### Example 113. Synthesis of Compound 112

Step 1. To a solution of bicyclo[2.1.1]hexan-1-amine hydrochloride (80.0 mg, 0.599 mmol, 1.00 eq), pyridine (145 μL, 1.80 mmol, 3.00 eq) in acetonitrile (1 mL) was added phenyl carbonochloridate (90.0 μL, 0.718 mmol, 1.20 eq). The mixture was stirred at 20° C. for 2 h. The reaction mixture was quenched by hydrochloric acid (1 M, 10 mL) at 20° C. and extracted with ethyl acetate (3×20 mL). The combined organic layers were dried over anhydrous sodium sulfate, filtered and concentrated under reduced pressure to give a residue which was purified by Prep-TLC (SiO2, petroleum ether/ethyl acetate=5/1) and concentrated to afford phenyl bicyclo[2.1.1]hexan-1-ylcarbamate (80.0 mg, 0.313 mmol, 52% yield) as a white solid. 1H NMR (400 MHz, DMSO-d6) δ=8.28 (s, 1H), 7.40-7.34 (m, 2H), 7.22-7.16 (m, 1H), 7.11-7.06 (m, 2H), 2.27 (m, 1H), 1.84 (m, 2H), 1.74-1.68 (m, 2H), 1.64-1.56 (m, 2H), 1.29 (m, 2H).

Step 2. To a solution of 3-(2,6-difluoro-4-(3-hydroxyazetidin-1-yl)phenyl)piperidine-2,6-dione XII (80.0 mg, 270 μmol, 1.00 eq) in N,N-dimethyl formamide (1 mL) was added sodium hydride (21.6 mg, 540 μmol, 60% purity, 2.00 eq) at 0° C. The mixture was stirred at 0° C. for 10 min. Then phenyl bicyclo[2.1.1] hexan-1-ylcarbamate (80.0 mg, 367 μmol, 1.30 eq) was added and the mixture was stirred at 20° C. for 1 h. The pH of the mixture was adjusted to 5-6 with formic acid and filtered. The filtrate was purified by Prep-HPLC (column: Phenomenex Luna C18 150*25 mm*10 μm; mobile phase: [water (formic acid)-acetonitrile]; B %: 37%-67%, 9 min) and lyophilized to afford 1-(4-(2,6-dioxopiperidin-3-yl)-3,5-difluorophenyl)azetidin-3-ylbicyclo[2.1.1]hexan-1-ylcarbamate #112 (57.70 mg, 136 μmol, 51% yield, 97% purity) as a white solid. H NMR (400 MHz, DMSO-d6) δ=10.86 (s, 1H), 7.94 (br s, 1H), 6.19 (d, J=11.0 Hz, 2H), 5.14 (m, 1H), 4.14 (br t, J=7.6 Hz, 2H), 4.04 (m, 1H), 3.71 (m, 2H), 2.84-2.72 (m, 1H), 2.52 (br s, 1H), 2.24 (br s, 1H), 2.13-2.01 (m, 1H), 1.98-1.90 (m, 1H), 1.79 (m, 2H), 1.67-1.61 (m, 2H), 1.57 (m, 2H), 1.24 (br s, 2H). MS (ESI) m/z 420.1 [M+H]+.

### Example 114. Synthesis of Compound 113

To a solution of 3-(2,6-difluoro-4-(3-hydroxyazetidin-1-yl)phenyl)piperidine-2,6-dione XII (100 mg, 0.337 mmol, 1.00 eq) in dimethyl formamide (2 mL) was added 1,1′-carbonyldiimidazole (273 mg, 1.69 mmol, 5.00 eq) and triethylamine (140 μL, 1.01 mmol, 3.00 eq). The mixture was stirred at 25° C. for 0.5 h. Then the mixture was added 8-diazabicyclo[5,4,0] undec-7-ene (152 μL, 1.01 mmol, 3.00 eq) and 3,3-dimethylbutan-1-amine (54.5 μL, 0.405 mmol, 1.20 eq). The mixture was stirred at 25° C. for 2 h. The reaction mixture was diluted with water (30 mL) and exacted with ethyl acetate (3×30 mL). The organic phase was separated, washed with brine (2×10 mL), dried over anhydrous sodium sulfate, filtered and concentrated under reduced pressure to give a residue. The crude product was dissolved in dimethyl formamide (2 mL) and purified by Prep-HPLC (column: Phenomenex Synergi C18 150*25 mm*10 μm; mobile phase: [water (formic acid)-acetonitrile]; B %: 43%-73%, 10 min) and lyophilized to give 1-(4-(2,6-dioxopiperidin-3-yl)-3,5-difluorophenyl)azetidin-3-yl(3,3-dimethylbutyl)carbamate #113 (83.6 mg, 0.195 mmol, 58% yield, 98% purity, formate) as a white solid. 1H NMR (400 MHz, DMSO-d6) δ=10.86 (br s, 1H), 8.43 (s, 1H), 7.32 (t, J=5.6 Hz, 1H), 6.18 (d, J=11.0 Hz, 2H), 5.18-5.00 (m, 1H), 4.19-4.08 (m, 2H), 4.03 (br dd, J=12.6, 5.0 Hz, 1H), 3.70 (br dd, J=8.9, 3.6 Hz, 2H), 3.04-2.92 (m, 2H), 2.82-2.70 (m, 1H), 2.52 (br d, J=1.8 Hz, 1H), 2.07 (qd, J=13.0, 3.8 Hz, 1H), 1.98-1.89 (m, 1H), 1.40-1.26 (m, 2H), 0.87 (s, 9H). MS (ESI) m/z 424.3 [M+H]+.

### Example 115. Synthesis of Compound 114

To a solution of 3-(2,6-difluoro-4-(3-hydroxyazetidin-1-yl)phenyl)piperidine-2,6-dione XII (80.0 mg, 0.270 mmol, 1.00 eq) in dimethylformamide (0.500 mL) was added di(1H-imidazol-1-yl)methanone (175 mg, 1.08 mmol, 4.00 eq). The mixture was stirred at 20° C. for 2 h. Then 3,4,5,7,8,9,10,10a-octahydropyrido[1,2-a][1,4] diazepine (40.7 μL, 0.270 mmol, 1.00 eq), triethylamine (37.5 μL, 0.270 mmol, 1.00 eq), (1-methyl-4-piperidyl)methanamine (34.6 mg, 0.270 mmol, 1.00 eq) were added and the mixture was stirred at 20° C. for 12 h. The pH of the mixture was adjusted to 5-6 with formic acid and filtered. The filtrate was purified by Prep-HPLC (column: Phenomenex Luna C18 150*25 mm*10 μm; mobile phase: [water (formic acid)-acetonitrile]; B %: 0%-30%, 9 min) and lyophilized to afford 1-(4-(2,6-dioxopiperidin-3-yl)-3,5-difluorophenyl)azetidin-3-yl((1-methylpiperidin-4-yl)methyl)carbamate #114 (25.49 mg, 0.0547 μmol, 20% yield, 97% purity) as an off-white solid. 1H NMR (400 MHz, DMSO-d6) δ=10.86 (s, 1H), 8.25 (s, 1H), 7.46 (br t, J=5.8 Hz, 1H), 6.18 (d, J=11.0 Hz, 2H), 5.17-5.09 (m, 1H), 4.14 (br s, 2H), 4.03 (m, 1H), 3.71 (m, 2H), 2.94-2.86 (m, 4H), 2.83-2.73 (m, 1H), 2.52 (br s, 1H), 2.29 (s, 3H), 2.13-2.01 (m, 3H), 1.98-1.90 (m, 1H), 1.63 (m, 2H), 1.40 (m, 1H), 1.25-1.12 (m, 2H). MS (ESI) m/z 451.2 [M+H]+.

### Example 116. Synthesis of Compound 115

A mixture of 3-(2,6-difluoro-4-(3-hydroxyazetidin-1-yl)phenyl)piperidine-2,6-dione XII (100 mg, 0.338 mmol, 1.00 eq), 1,1′-carbonyldiimidazole (328 mg, 2.03 mmol, 6.00 eq) in dimethyl formamide (5 mL) was stirred at 25° C. for 2 h. Then (1S)-1-cyclohexylethanamine (100 μL, 0.675 mmol, 2.00 eq) was added and the mixture was stirred at 25° C. for 11 h. The pH of reaction mixture was adjusted to 6 with formic acid. Then the mixture was concentrated in vacuo. The residue was purified by prep-HPLC (column: Phenomenex Luna C18 150 mm*25 mm*10 μm; mobile phase: [water (formic acid)-acetonitrile]; B %: 48%-78%, 8 min). The desired fraction was collected and lyophilized to afford 1-(4-(2,6-dioxopiperidin-3-yl)-3,5-difluorophenyl)azetidin-3-yl((S)-1-cyclohexylethyl)carbamate #115 (65.0 mg, 0.140 mmol, 42% yield, 97% purity) as a white solid. 1H NMR (400 MHz, DMSO-d6) δ=10.86 (s, 1H), 7.23 (d, J=8.8 Hz, 1H), 6.19 (d, J=11.0 Hz, 2H), 5.18-5.07 (m, 1H), 4.19-4.10 (m, 2H), 4.03 (dd, J=4.8, 12.4 Hz, 1H), 3.75-3.65 (m, 2H), 3.29 (s, 1H), 2.85-2.70 (m, 1H), 2.48-2.45 (m, 1H), 2.14-1.99 (m, 1H), 1.98-1.89 (m, 1H), 1.73-1.63 (m, 4H), 1.59 (d, J=8.8 Hz, 1H), 1.30-1.04 (m, 4H), 0.99 (d, J=6.8 Hz, 3H), 0.94-0.82 (m, 2H). MS (ESI) m/z 450.2 [M+H]+.

### Example 117. Synthesis of Compound 116

To a solution of 3-(2,6-difluoro-4-(3-hydroxyazetidin-1-yl)phenyl)piperidine-2,6-dione XII (100 mg, 0.337 mmol, 1.00 eq) in dimethylformamide (1 mL) was added di(1H-imidazol-1-yl)methanone (218 mg, 1.35 mmol, 4.00 eq). The mixture was stirred at 20° C. for 2 h. Then tetrahydro-2H-thiopyran-4-amine (39.5 mg, 0.337 mmol, 1.00 eq), 3,4,5,7,8,9,10,10a-octahydropyrido[1,2-α][1,4]diazepine (50.8 μL, 0.337 mmol, 1.00 eq), triethylamine (46.9 μL, 0.337 mmol, 1.00 eq) were added. The mixture was stirred at 20° C. for 12 h. The pH of the mixture was adjusted to 5-6 with formic acid and filtered. The filtrate was purified by Prep-HPLC (column: Phenomenex Luna C18 150*25 mm*10 μm; mobile phase: [water (formic acid)-acetonitrile]; B %: 30%-60%, 9 min) and lyophilized to afford 1-(4-(2,6-dioxopiperidin-3-yl)-3,5-difluorophenyl)azetidin-3-yl(tetrahydro-2H-thiopyran-4-yl)carbamate #116 (21.49 mg, 0.0463 mmol, 13% yield, 97% purity) as a white solid. 1H NMR (400 MHz, DMSO-d6) δ=10.85 (s, 1H), 7.48 (br d, J=8.0 Hz, 1H), 6.18 (d, J=10.9 Hz, 2H), 5.21-5.09 (m, 1H), 4.14 (br t, J=7.6 Hz, 2H), 4.03 (dd, J=5.1, 12.6 Hz, 1H), 3.72 (m, 2H), 2.83-2.73 (m, 1H), 2.62 (m, 4H), 2.52 (d, J=1.9 Hz, 1H), 2.48 (br s, 1H), 2.11-1.92 (m, 4H), 1.56-1.44 (m, 2H). MS (ESI) m/z 440.0 [M+H]+.

### Example 118: Synthesis of Compound 117

A mixture of 3-(2,6-difluoro-4-(3-hydroxyazetidin-1-yl)phenyl)piperidine-2,6-dione XII (100 mg, 0.338 mmol, 1.00 eq), 1,1′-carbonyldiimidazole (328 mg, 2.03 mmol, 6.00 eq) in dimethyl formamide (5 mL) was stirred at 25° C. for 2 h. Then tetrahydropyran-4-ylmethanamine (77.8 mg, 0.675 mmol, 2.00 eq) was added and the mixture was stirred at 25° C. for 11 h. The pH of reaction mixture was adjusted to 6 with formic acid. The residue was purified by prep-HPLC (column: Phenomenex Luna C18 150 mm*25 mm*10 μm; mobile phase: [water (formic acid)-acetonitrile]; B %: 22%-52%, 10.5 min). The desired fraction was collected and lyophilized to afford 1-(4-(2,6-dioxopiperidin-3-yl)-3,5-difluorophenyl)azetidin-3-yl ((tetrahydro-2H-pyran-4-yl)methyl)carbamate #117 (30.0 mg, 0.0650 mmol, 19% yield, 95% purity) as a grey solid. 1H NMR (400 MHz, DMSO-d6) δ=10.85 (s, 1H), 7.46 (t, J=5.8 Hz, 1H), 6.19 (d, J=11.0 Hz, 2H), 5.23-5.07 (m, 1H), 4.15 (t, J=7.8 Hz, 2H), 4.04 (dd, J=5.0, 12.6 Hz, 1H), 3.83 (dd, J=2.8, 11.2 Hz, 2H), 3.72 (dd, J=3.4, 8.8 Hz, 2H), 3.29-3.18 (m, 2H), 2.89 (t, J=6.2 Hz, 2H), 2.84-2.71 (m, 1H), 2.60-2.53 (m, 1H), 2.15-2.01 (m, 1H), 2.00-1.87 (m, 1H), 1.61 (dt, J=3.6, 7.2 Hz, 1H), 1.54 (d, J=13.4 Hz, 2H), 1.12 (dq, J=4.4, 12.2 Hz, 2H). MS (ESI) m/z 438.0 [M+H]+.

### Example 119. Synthesis of Compound 118

A mixture of 3-(2,6-difluoro-4-(3-hydroxyazetidin-1-yl)phenyl)piperidine-2,6-dione XII (100 mg, 0.338 mmol, 1.00 eq), 1,1′-carbonyldiimidazole (328 mg, 2.03 mmol, 6.00 eq) in dimethyl formamide (5 mL) was stirred at 25° C. for 2 h. Then 2-methylsulfanylethanamine (61.6 mg, 0.675 mmol, 2.00 eq) was added and the mixture was stirred at 25° C. for 11 h. The pH of reaction mixture was adjusted to 6 with formic acid. Then the mixture was concentrated in vacuo. The residue was purified by prep-HPLC (column: Phenomenex Luna C18 150 mm*25 mm*10 μm; mobile phase: [water (formic acid)-acetonitrile]; B %: 27%-57%, 10.5 min). The desired fraction was collected and lyophilized to afford 1-(4-(2,6-dioxopiperidin-3-yl)-3,5-difluorophenyl)azetidin-3-yl(2-(methylthio)ethyl)carbamate #118 (45.0 mg, 0.108 mmol, 31% yield, 97% purity) as a white solid. 1H NMR (400 MHz, DMSO-d6) δ=10.85 (s, 1H), 7.51 (t, J=5.8 Hz, 1H), 6.19 (d, J=11.0 Hz, 2H), 5.23-5.08 (m, 1H), 4.15 (t, J=7.6 Hz, 2H), 4.03 (dd, J=5.2, 12.4 Hz, 1H), 3.72 (dd, J=3.2, 8.6 Hz, 2H), 3.17 (q, J=6.6 Hz, 2H), 2.83-2.72 (m, 1H), 2.54-2.52 (m, 2H), 2.48 (s, 1H), 2.14-2.00 (m, 4H), 1.98-1.89 (m, 1H). MS (ESI) m/z 414.0 [M+H]+.

### Example 120. Synthesis of Compound 119

Step 1. To a solution of (1S,4S)-4-methoxycyclohexanamine (234 mg, 1.81 mmol, 1.00 eq) and pyridine (730 μL, 9.06 mmol, 5.00 eq) in acetonitrile (4 mL) was added phenyl carbonochloridate (272 μL, 2.17 mmol, 1.20 eq) dropwise at 0° C. The reaction mixture was stirred at 20° C. for 1 h. The mixture was filtered. The filtrate was purified by reverse phase chromatography (column: spherical C18, 20-45 μm, 100 Å, SW 40, mobile phase: [water (0.1% formic acid)-acetonitrile)) and lyophilized to give phenyl ((1S,4S)-4-methoxycyclohexyl)carbamate (40.0 mg, 0.160 mmol, 9% yield) as a white solid.

Step 2. To a solution of 3-(2,6-difluoro-4-(3-hydroxyazetidin-1-yl)phenyl)piperidine-2,6-dione XII (65.0 mg, 219 μmol, 1.00 eq) in dimethyformamide (5 mL) was added sodium hydride (13.2 mg, 329 μmol, 60% purity, 1.50 eq) at 0° C. Then the mixture was added phenyl ((1S,4S)-4-methoxycyclohexyl)carbamate (60.2 mg, 241 μmol, 1.10 eq). The mixture was stirred at 20° C. for 2 h. The mixture was adjusted to pH=5-6 by adding formic acid (0.100 mL) and filtered. The filtrate was purified by Prep-HPLC (column: Phenomenex Luna C18 150*25 mm*10 um; mobile phase: [water (formic acid)-acetonitrile]; B %: 29%-59%, 9 min) and lyophilized to afford 1-(4-(2,6-dioxopiperidin-3-yl)-3,5-difluorophenyl) azetidin-3-yl ((1S,4S)-4-methoxycyclohexyl) carbamate #119 (23.6 mg, 51.3 μmol, 23% yield) as a white solid. 1H NMR (400 MHz, DMSO-d6) δ=10.85 (s, 1H), 7.34 (br d, J=7.6 Hz, 1H), 6.18 (d, J=11.0 Hz, 2H), 5.19-5.09 (m, 1H), 4.14 (br t, J=7.8 Hz, 2H), 4.03 (br dd, J=5.0, 12.5 Hz, 1H), 3.71 (br dd, J=3.1, 8.5 Hz, 2H), 3.28 (br d, J=2.5 Hz, 1H), 3.18 (s, 3H), 2.81-2.72 (m, 1H), 2.55-2.54 (m, 1H), 2.10-2.03 (m, 1H), 1.97-1.90 (m, 1H), 1.76 (br dd, J=3.0, 8.9 Hz, 2H), 1.52-1.37 (m, 7H). MS (ESI) m/z 450.1 [M+H]+.

### Example 121. Synthesis of Compound 120

Step 1. To a solution of tert-butyl pyrrolidin-3-ylcarbamate (3.00 g, 16.1 mmol, 1.00 eq) in methanol (30 mL) was added acetone (8.29 mL, 112 mmol, 7.00 eq), acetic acid (460 μL, 8.05 mmol, 0.500 eq) at 0° C. for 0.5 h. Then sodium cyanoborohydride (2.02 g, 32.2 mmol, 2.00 eq) was added, the mixture was stirred at 20° C. for 2 h. The mixture was diluted with water (100 mL), extracted with ethyl acetate (2×100 mL). The combined organic layers were dried over anhydrous sodium sulfate, filtered, concentrated under reduced pressure to give a residue. The residue was purified by reversed phase column chromatography (C18, 330 g; condition: water/acetonitrile=1/0 to 0/1, 0.1% ammonium hydroxide) and lyophilized to afford tert-butyl (1-isopropylpyrrolidin-3-yl)carbamate (2.00 g, 8.76 mmol, 54% yield) as a yellow oil. 1H NMR (400 MHz, CDCl3) δ=4.93 (br s, 1H), 4.15 (br s, 1H), 2.84 (br s, 1H), 2.70-2.56 (m, 2H), 2.34 (m, 2H), 2.24 (m, 1H), 1.62-1.55 (m, 1H), 1.43 (s, 9H), 1.07 (dd, J=4.1, 6.4 Hz, 6H).

Step 2. To a solution of tert-butyl (1-isopropylpyrrolidin-3-yl) carbamate (1.30 g, 5.69 mmol, 1.00 eq) in dioxane (10 mL) was added hydrochloric acid (4 M in dioxane, 3 mL). The mixture was stirred at 20° C. for 2 h. The mixture was concentrated under reduced pressure to afford 1-isopropylpyrrolidin-3-amine (0.900 g, 5.47 mmol, 95% yield, hydrochloride) as a white solid. 1H NMR (400 MHz, DMSO-d6) δ=8.87-8.41 (m, 3H), 4.06-3.85 (m, 1H), 3.79-3.63 (m, 1H), 3.57-3.45 (m, 3H), 3.25-3.09 (m, 1H), 2.42-1.89 (m, 2H), 1.29 (br d, J=6.5 Hz, 6H).

Step 3. To a solution of 1-isopropylpyrrolidin-3-amine (0.500 g, 3.04 mmol, 1.00 eq, hydrochloride), pyridine (735 μL, 9.11 mmol, 3.00 eq) in acetonitrile (5 mL) was added phenyl carbonochloridate (380 μL, 3.04 mmol, 1.00 eq). The mixture was stirred at 20° C. for 1 h. The mixture was concentrated under reduced pressure to give a residue. The residue was purified by reversed phase column chromatography (C18, 330 g; condition: water/acetonitrile=1/0 to 0/1, 0.1% formic acid) and lyophilized to afford phenyl (1-isopropylpyrrolidin-3-yl)carbamate (0.600 g, 2.42 mmol, 79% yield) as a yellow solid. 1H NMR (400 MHz, CDCl3) δ=7.55 (br s, 1H), 7.36-7.29 (m, 2H), 7.21-7.14 (m, 1H), 7.08 (br d, J=8.0 Hz, 2H), 4.60 (br s, 1H), 3.70 (br s, 1H), 3.55 (m, 1H), 3.31 (m, 2H), 3.05 (br d, J=1.6 Hz, 1H), 2.58-2.42 (m, 1H), 2.36-2.21 (m, 1H), 1.41 (br d, J=5.6 Hz, 6H).

Step 4. To a solution of 3-(4-(3-aminoazetidin-1-yl)-2,6-difluorophenyl)piperidine-2,6-dione XII (80.0 mg, 270 μmol, 1.00 eq), 3,4,5,7,8,9,10,10a-octahydropyrido[1,2-a][1,4]diazepine (122 μL, 812 μmol, 3.00 eq) in dimethylformamide (2 mL) was added phenyl (1-isopropylpyrrolidin-3-yl)carbamate (134 mg, 541 μmol, 2.00 eq). The mixture was stirred at 20° C. for 1 h. The pH of the mixture was adjusted to 5-6 with formic acid and filtered. The filtrate was purified by Prep-HPLC (column: Phenomenex Luna C18 150*25 mm*10 μm; mobile phase: [water (formic acid)-acetonitrile]; B %: 0%-29%, 9 min) and lyophilized to afford 1-(1-(4-(2,6-dioxopiperidin-3-yl)-3,5-difluorophenyl) azetidin-3-yl)-3-(1-isopropylpyrrolidin-3-yl) urea #120 (52.58 mg, 99.7 μmol, 36% yield, 94% purity) as an off white solid. 1H NMR (400 MHz, DMSO-d6) δ=10.85 (s, 1H), 8.16 (s, 1H), 6.61 (br d, J=7.5 Hz, 1H), 6.25 (br d, J=7.0 Hz, 1H), 6.15 (d, J=11.0 Hz, 2H), 4.55-4.42 (m, 1H), 4.15-4.00 (m, 4H), 3.56 (br t, J=6.6 Hz, 2H), 3.02-2.92 (m, 2H), 2.82-2.62 (m, 5H), 2.16-2.01 (m, 2H), 1.99-1.89 (m, 1H), 1.66-1.50 (m, 1H), 1.10 (dd, J=2.9, 6.3 Hz, 6H). MS (ESI) m/z 450.1 [M+H]+.

### Example 122. Synthesis of Compound 121

Step 1. To a solution of 1-methylpyrrolidin-3-amine (0.500 g, 4.99 mmol, 1.00 eq), pyridine (805 μL, 9.98 mmol, 2.00 eq) in acetonitrile (10 mL) was added phenyl carbonochloridate (859 mg, 5.49 mmol, 687 μL, 1.10 eq) at 0° C. The mixture was stirred at 20° C. for 2 h. The mixture was filtered. The filtrate was purified by reversed phase column chromatography (C18, 80 g; condition: water/acetonitrile=1/0 to 0/1, 0.1% formic acid) and lyophilized to afford phenyl (1-methylpyrrolidin-3-yl)carbamate (1.20 g, crude) as a yellow oil. MS (ESI) m/z 221.2 [M+H]+.

Step 2. To a solution of 3-(4-(3-aminoazetidin-1-yl)-2,6-difluorophenyl)piperidine-2,6-dione XII (100 mg, 338 μmol, 1.00 eq), 3,4,5,7,8,9,10,10a-octahydropyrido[1,2-α][1,4]diazepine (153 μL, 1.02 mmol, 3.00 eq) in dimethylformamide (2 mL) was added phenyl (1-methylpyrrolidin-3-yl)carbamate (111 mg, 507 μmol, 1.50 eq). The mixture was stirred at 20° C. for 1 h. The pH of the mixture was adjusted to 5-6 with formic acid and filtered. The filtrate was purified by Prep-HPLC (column: Phenomenex Luna C18 150*25 mm*10 μm; mobile phase: [water (formic acid)-acetonitrile]; B %: 0%-26%, 9 min) and lyophilized to afford 1-(1-(4-(2,6-dioxopiperidin-3-yl)-3,5-difluorophenyl)azetidin-3-yl)-3-(1-methylpyrrolidin-3-yl)urea #121 (34.22 mg, 79.6 μmol, 23% yield, 98% purity) as a yellow solid. 1H NMR (400 MHz, DMSO-d6) δ=10.85 (s, 1H), 8.20 (s, 1H), 6.62 (br d, J=7.5 Hz, 1H), 6.32 (br d, J=7.3 Hz, 1H), 6.14 (d, J=11.1 Hz, 2H), 4.50-4.44 (m, 1H), 4.12-4.02 (m, 4H), 3.55 (m, 2H), 2.91-2.73 (m, 3H), 2.61-2.55 (m, 2H), 2.48 (m, 1H), 2.42 (s, 3H), 2.19-2.03 (m, 2H), 1.98-1.90 (m, 1H), 1.63-1.53 (m, 1H). MS (ESI) m/z 422.2 [M+H]+.

### Example 123. Synthesis of Compound 122

To a solution of 3-(2,6-difluoro-4-(3-hydroxyazetidin-1-yl)phenyl)piperidine-2,6-dione XII (15.0 mg, 50.6 μmol, 1.00 eq) in dimethyl formamide (1 mL) was added triethylamine (10.6 μL, 75.9 μmol, 1.50 eq) and N,N′-carbonyl diimidazole (49.3 mg, 304 μmol, 6.00 eq) at 25° C. After addition, the mixture was stirred at 25° C. for 1 h. Then a solution of 2-methylbutan-1-amine (5.98 μL, 50.6 μmol, 1.00 eq) and 2,3,4,6,7,8,9,10-octahydropyrimido[1,2-a]azepine (11.6 mg, 75.9 μmol, 11.4 uL, 1.50 eq) in dimethyl formamide (0.500 mL) was added dropwise at 25° C. The resulting mixture was stirred at 25° C. for 2 h. The reaction mixture was filtered and the filtrate was purified by prep-HPLC (column: Phenomenex Synergi C18 150*25 mm*10 μm; mobile phase: [water (formic acid)-acetonitrile]; B %: 38%-68%, 10 min) to afford 1-(4-(2,6-dioxopiperidin-3-yl)-3,5-difluorophenyl) azetidin-3-yl (2-methylbutyl)carbamate #122 (14.86 mg, 36.3 μmol, 72% yield, 99% purity) as a gum yellow solid. 1H NMR (400 MHz, DMSO-d6) δ=10.85 (br s, 1H), 7.38 (br t, J=5.8 Hz, 1H), 6.18 (d, J=11.0 Hz, 2H), 5.17-5.09 (m, 1H), 4.15 (br t, J=7.6 Hz, 2H), 4.03 (dd, J=5.1, 12.5 Hz, 1H), 3.71 (br dd, J=3.1, 8.4 Hz, 2H), 2.99-2.87 (m, 1H), 2.85-2.71 (m, 2H), 2.07 (br dd, J=3.9, 12.9 Hz, 1H), 1.99-1.91 (m, 1H), 1.61-1.33 (m, 2H), 1.33-1.22 (m, 1H), 1.10-0.98 (m, 1H), 0.89-0.84 (m, 2H), 0.84-0.79 (m, 4H). MS (ESI) m/z 410.1 [M+H]⁺.

### Example 124. Synthesis of Compound 123

To a solution of 3-(2,6-difluoro-4-(3-hydroxyazetidin-1-yl)phenyl)piperidine-2,6-dione XII (70.0 mg, 236 μmol, 1.00 eq) in dimethylformamide (1 mL) was added di(1H-imidazol-1-yl)methanone (153 mg, 945 μmol, 4.00 eq). The mixture was stirred at 20° C. for 2 h. Then (R)-1-phenylethanamine (27.4 μL, 236 μmol, 1.00 eq), 3,4,5,7,8,9,10,10a-octahydropyrido[1,2-a][1,4]diazepine (35.6 μL, 236 μmol, 1.00 eq) and triethylamine (32.8 μL, 236 μmol, 1.00 eq) were added. The mixture was stirred at 20° C. for 12 h. The pH of the mixture was adjusted to 5-6 with drops of formic acid and filtered. The filtrate was purified by Prep-HPLC (column: Phenomenex Luna C18 150*25 mm*10 μm; mobile phase: [water (formic acid)-acetonitrile]; B %: 37%-67%, 9 min) and Prep-HPLC (column: Phenomenex Luna C18 150*25 mm*10 μm; mobile phase: [water (formic acid)-acetonitrile]; B %: 36%-66%, 9 min) and lyophilized to afford 1-(4-(2,6-dioxopiperidin-3-yl)-3,5-difluorophenyl)azetidin-3-yl((R)-1-phenylethyl)carbamate #123 (21.1 mg, 45.4 μmol, 19% yield, 96% purity) as a white solid. 1H NMR (400 MHz, DMSO-d6) δ=10.85 (s, 1H), 7.98 (br d, J=8.3 Hz, 1H), 7.37-7.27 (m, 4H), 7.25-7.18 (m, 1H), 6.18 (br d, J=11.1 Hz, 2H), 5.16-5.08 (m, 1H), 4.71-4.60 (m, 1H), 4.19-4.08 (m, 2H), 4.03 (dd, J=5.1, 12.4 Hz, 1H), 3.79-3.65 (m, 2H), 2.84-2.71 (m, 1H), 2.52 (br s, 1H), 2.13-2.01 (m, 1H), 1.98-1.89 (m, 1H), 1.34 (d, J=7.0 Hz, 3H). MS (ESI) m/z 444.1 [M+H]+.

### Example 125. Synthesis of Compound 124

Step 1. To a solution of (1R,4R)-4-methylcyclohexanamine hydrochloride (500 mg, 3.34 mmol, 1.00 eq) and pyridine (809 μL, 10.0 mmol, 3.00 eq) in acetonitrile (10 mL) was added phenyl carbonochloridate (502 μL, 4.01 mmol, 1.20 eq). The reaction mixture was stirred at 20° C. for 1 h. The reaction mixture was concentrated under reduced pressure to give a residue. The residue was purified by column chromatography (SiO2, petroleum ether/ethyl acetate=10/1) and concentrated under reduced pressure to afford phenyl ((1R,4R)-4-methylcyclohexyl)carbamate (280 mg, 1.20 mmol, 36% yield) as a white solid. 1H NMR (400 MHz, CDCl3) δ=7.36 (br t, J=7.7 Hz, 2H), 7.22-7.16 (m, 1H), 7.13 (br d, J=7.9 Hz, 2H), 4.98-4.65 (m, 1H), 3.64-3.28 (m, 1H), 2.08 (br d, J=11.3 Hz, 2H), 1.75 (br d, J=12.4 Hz, 2H), 1.43-1.30 (m, 1H), 1.27-1.16 (m, 2H), 1.12-1.00 (m, 2H), 0.91 (d, J=6.5 Hz, 3H). MS (ESI) m/z 436.1 [M+H]+.

Step 2. To a solution of 3-(2,6-difluoro-4-(3-hydroxyazetidin-1-yl)phenyl)piperidine-2,6-dione XII (70.0 mg, 236 μmol, 1.00 eq) in dimethylformamide (1 mL) was added sodium hydride (18.9 mg, 472 μmol, 60% purity, 2.00 eq) at 0° C. The mixture was stirred at 0° C. for 10 min. Then phenyl ((1R,4R)-4-methylcyclohexyl) carbamate (165 mg, 708 μmol, 3.00 eq) was added and the mixture was stirred at 20° C. for 2 h. The mixture was quenched with formic acid (pH=5-6) and the mixture was filtered. The filter cake was dissolved with dimethylformamide (2.00 mL) and purified by Prep-HPLC (column: Waters xbridge 150*25 mm 10 μm; mobile phase: [water (ammonium bicarbonate)-acetonitrile]; B %: 38%-68%, 11 min) and lyophilized to afford 1-(4-(2,6-dioxopiperidin-3-yl)-3,5-difluorophenyl)azetidin-3-yl((1R,4R)-4-methyl cyclohexyl) carbamate #124 (28.5 mg, 70.4 μmol, 29% yield, 99% purity) as a white solid. 1H NMR (400 MHz, DMSO-d6) δ=10.85 (s, 1H), 7.29 (d, J=7.9 Hz, 1H), 6.18 (d, J=11.0 Hz, 2H), 5.17-5.07 (m, 1H), 4.14 (m, 2H), 4.03 (dd, J=4.9, 12.5 Hz, 1H), 3.70 (m, 2H), 3.19 (m, 1H), 2.83-2.75 (m, 1H), 2.54 (m, 1H), 2.13-2.01 (m, 1H), 1.98-1.90 (m, 1H), 1.76 (m, 2H), 1.69-1.58 (m, 2H), 1.31-1.22 (m, 1H), 1.21-1.11 (m, 2H), 1.00-0.88 (m, 2H), 0.85 (d, J=6.5 Hz, 3H). MS (ESI) m/z 436.1 [M+H]+.

### Example 126. Synthesis of Compound 125

To a solution of 3-(2,6-difluoro-4-(3-hydroxyazetidin-1-yl)phenyl)piperidine-2,6-dione XII (70.0 mg, 236 μmol, 1.00 eq) in dimethylformamide (1 mL) was added di(1H-imidazol-1-yl)methanone (153 mg, 945 μmol, 4.00 eq). The mixture was stirred at 20° C. for 2 h. Then 3-methylbutan-1-amine (27.4 μL, 236 μmol, 1.00 eq), 3,4,5,7,8,9,10,10a-octahydropyrido[1,2-a][1,4]diazepine (35.6 μL, 236 μmol, 1.00 eq) and triethylamine (32.8 μL, 236 μmol, 1.00 eq) were added. The mixture was stirred at 20° C. for 12 h. The pH of the mixture was adjusted to 5-6 with drops of formic acid and filtered. The filtrate was purified by Prep-HPLC (column: Phenomenex Luna C18 150*25 mm*10 μm; mobile phase: [water (formic acid)-acetonitrile]; B %: 38%-68%, 9 min) and lyophilized to afford 1-(4-(2,6-dioxopiperidin-3-yl)-3,5-difluorophenyl)azetidin-3-yl isopentylcarbamate #125 (55.2 mg, 138 μmol, 58% yield, 99% purity) as a white solid. 1H NMR (400 MHz, DMSO-d6) δ=10.85 (s, 1H), 7.34 (t, J=5.6 Hz, 1H), 6.18 (d, J=11.0 Hz, 2H), 5.16-5.10 (m, 1H), 4.17-4.12 (m, 2H), 4.04 (m, 1H), 3.71 (m, 2H), 3.02-2.97 (m, 2H), 2.81-2.75 (m, 1H), 2.53 (br s, 1H), 2.13-2.03 (m, 1H), 1.99-1.90 (m, 1H), 1.56 (m, 1H), 1.29 (m, 2H), 0.86 (d, J=6.6 Hz, 6H). MS (ESI) m/z 410.0 [M+H]+.

### Example 127. Synthesis of Compound 126

Step 1. To a mixture of 1-(tert-butyl)azetidin-3-amine (500 mg, 3.90 mmol, 1.00 eq) and pyridine (1.57 mL, 19.5 mmol, 5.00 eq) in acetonitrile (10 mL) was added phenyl carbonochloridate (586 μL, 4.68 mmol, 1.20 eq) dropwise at 0° C. The mixture was stirred at 25° C. for 2 h. The crude product was purified by reverse phase chromatography (column: spherical C18, 20-45 μm, 100 Å, SW 120, mobile phase: water (0.1% formic acid)-acetonitrile) and lyophilized to give phenyl N-(1-tert-butylazetidin-3-yl)carbamate (1.00 g, crude) as a yellow solid. 1H NMR (400 MHz, DMSO-d6) δ=11.67 (br s, 1H), 7.43-7.35 (m, 2H), 7.29-7.20 (m, 1H), 7.12 (br d, J=7.6 Hz, 2H), 4.42-4.35 (m, 1H), 4.11-3.96 (m, 4H), 1.27-1.22 (m, 9H). MS (ESI) m/z 249.1 [M+H]+.

Step 2. To a mixture of phenyl N-(1-tert-butylazetidin-3-yl)carbamate (100 mg, 405 μmol, 1.20 eq) in dimethylformamide (1 mL) was added 3-(2,6-difluoro-4-(3-hydroxyazetidin-1-yl)phenyl)piperidine-2,6-dione XII (100 mg, 338 μmol, 1.00 eq) and sodium hydride (20.2 mg, 506 μmol, 60% purity, 1.50 eq) at 0° C. in portions. Then the mixture was stirred at 25° C. for 1 h. The mixture was adjusted pH to 5-6 by formic acid (0.100 mL) and filtered. The filtrate was purified by Prep-HPLC (column: Phenomenex Luna C18 150*25 mm*10 μm; mobile phase: [water (formic acid)-acetonitrile]; B %: 0%-30%, 9 min) and lyophilized to give 1-(4-(2,6-dioxopiperidin-3-yl)-3,5-difluorophenyl)azetidin-3-yl(1-(tert-butyl)azetidin-3-yl)carbamate #126 (33.73 mg, 71.8 μmol, 21% yield, 96% purity) as a yellow solid. 1H NMR (400 MHz, DMSO-d6) δ=10.85 (s, 1H), 7.92 (br d, J=7.3 Hz, 1H), 6.18 (d, J=11.0 Hz, 2H), 5.17-5.09 (m, 1H), 4.14 (br t, J=7.6 Hz, 2H), 4.07-4.00 (m, 2H), 3.72 (br dd, J=3.3, 8.6 Hz, 2H), 3.40 (br s, 2H), 3.16-3.10 (m, 2H), 2.82-2.73 (m, 1H), 2.52 (br s, 1H), 2.11-2.02 (m, 1H), 1.98-1.89 (m, 1H), 0.92 (s, 9H). MS (ESI) m/z 451.1 [M+H]+.

### Example 128. Synthesis of Compound 127

To a solution of 3-(2,6-difluoro-4-(3-hydroxyazetidin-1-yl)phenyl)piperidine-2,6-dione XII (80.0 mg, 270 μmol, 1.00 eq) in N,N-dimethyl formamide (0.500 mL) were added 1,1′-carbonyldiimidazole (262 mg, 1.62 mmol, 6.00 eq), triethylamine (37.6 μL, 270 μmol, 1.00 eq), 2,3,4,6,7,8,9,10-octahydropyrimido[1,2-a]azepine (40.7 μL, 270 μmol, 1.00 eq) and cyclohexylmethanamine (52.7 μL, 405 μmol, 1.50 eq). The reaction mixture was stirred at 25° C. for 12 h. To the reaction mixture was added formic acid (2 mL) and filtered. The filtrate was purified by Prep-HPLC (column: Phenomenex Luna C18 150*25 mm*10 μm; mobile phase: [water (formic acid)-acetonitrile]; B %: 37%-67%, 10.5 min) and lyophilized to afford 1-(4-(2,6-dioxopiperidin-3-yl)-3,5-difluorophenyl)azetidin-3-yl(cyclohexylmethyl) carbamate #127 (24.54 mg, 58.5 μmol, 22% yield, 97% purity) as an off-white solid. 1H NMR (400 MHz, DMSO-d6) δ=10.85 (s, 1H), 7.38 (br t, J=5.9 Hz, 1H), 6.18 (d, J=11.0 Hz, 2H), 5.19-5.04 (m, 1H), 4.14 (br t, J=7.6 Hz, 2H), 4.03 (br dd, J=5.0, 12.5 Hz, 1H), 3.71 (br dd, J=3.6, 8.8 Hz, 2H), 2.82 (br t, J=6.3 Hz, 2H), 2.79-2.71 (m, 1H), 2.52 (br d, J=1.9 Hz, 1H), 2.07 (dq, J=3.8, 13.0 Hz, 1H), 1.98-1.89 (m, 1H), 1.65 (br d, J=7.1 Hz, 5H), 1.36 (ddd, J=3.3, 7.3, 10.6 Hz, 1H), 1.21-1.07 (m, 3H), 0.93-0.72 (m, 2H). MS (ESI) m/z 436.1 [M+H]+.

### Example 129. Synthesis of Compound 128

To a mixture of 3-(2,6-difluoro-4-(3-hydroxyazetidin-1-yl)phenyl)piperidine-2,6-dione XII (50.0 mg, 168 μmol, 1.00 eq) in dimethylformamide (0.500 mL) was added 1,1′-carbonyldiimidazole (164 mg, 1.01 mmol, 6.00 eq). The reaction mixture was stirred at 25° C. for 1 h. Then 3-phenylcyclobutanamine (37.2 mg, 253 μmol, 1.50 eq) was added and the mixture was stirred at 50° C. for 11 h. Then triethylamine (35.2 μL, 253 μmol, 1.50 eq) and 1,8-diazabicyclo[5.4.0]undec-7-ene (38.1 μL, 253 μmol, 1.50 eq) was added and the mixture was stirred at 50° C. for 12 h. The reaction mixture was added formic acid (2 mL) and filtered. The filtrate was purified by Prep-HPLC (column: Phenomenex Luna C18 150*25 mm*10 μm; mobile phase: water (formic acid)-acetonitrile; B %: 42%-72%, 9 min) and lyophilized to afford 1-(4-(2,6-dioxopiperidin-3-yl)-3,5-difluorophenyl)azetidin-3-yl((1R,3R)-3 phenyl cyclobutyl) carbamate #128 (28 mg, 53.7 μmol, 31% yield, 99% purity) as a yellow solid. 1H NMR (400 MHz, DMSO-d6) δ=10.85 (br s, 1H), 7.91 (br d, J=7.2 Hz, 1H), 7.36-7.26 (m, 4H), 7.24-7.15 (m, 1H), 6.19 (d, J=11.2 Hz, 2H), 5.20-5.11 (m, 1H), 4.19-4.13 (m, 2H), 4.13-4.00 (m, 2H), 3.74 (dd, J=3.8, 8.8 Hz, 2H), 3.52 (td, J=7.2, 14.8 Hz, 1H), 2.78 (ddd, J=5.4, 13.0, 17.2 Hz, 1H), 2.53-2.53 (m, 1H), 2.42-2.35 (m, 4H), 2.14-2.03 (m, 1H), 2.00-1.91 (m, 1H). MS (ESI) m/z 470.2 [M+H]+.

### Example 130. Synthesis of Compound 129

To a solution of 3-(2,6-difluoro-4-(3-hydroxyazetidin-1-yl)phenyl)piperidine-2,6-dione XII (20.0 mg, 67.5 μmol, 1.00 eq) in N,N-dimethyl formamide (0.500 mL) was added 1,1′-carbonyldiimidazole (43.8 mg, 270 μmol, 4.00 eq), the mixture was stirred at 20° C. for 2 h. Then (R)-1-cyclohexylethanamine (8.59 μL, 67.5 μmol, 1.00 eq), triethylamine (9.40 μL, 67.5 μmol, 1.00 eq) and 2,3,4,6,7,8,9,10-octahydropyrimido[1,2-α]azepine (10.3 mg, 67.5 μmol, 10.2 μL, 1.00 eq) were added. The mixture was stirred at 20° C. for 12 h. The pH of the mixture was adjusted to 5-6 with formic acid and filtered. The filtrate was purified by Prep-HPLC (column: Phenomenex Luna C18 150*25 mm*10 μm; mobile phase: water (formic acid)-acetonitrile; B %: 45%-75%, 9 min) and lyophilized to afford 1-(4-(2,6-dioxopiperidin-3-yl)-3,5-difluorophenyl)azetidin-3-yl ((R)-1-cyclohexylethyl)carbamate #129 (8.09 mg, 17.6 μmol, 26% yield, 98% purity) as a white solid. 1H NMR (400 MHz, DMSO-d6) δ=10.86 (s, 1H), 7.23 (br d, J=8.8 Hz, 1H), 6.19 (d, J=11.0 Hz, 2H), 5.17-5.08 (m, 1H), 4.14 (m, 2H), 4.03 (m, 1H), 3.76-3.66 (m, 2H), 3.32-3.27 (m, 2H), 2.83-2.72 (m, 1H), 2.14-2.00 (m, 1H), 1.98-1.88 (m, 1H), 1.67 (m, 4H), 1.62-1.55 (m, 1H), 1.29-1.08 (m, 4H), 0.99 (d, J=6.8 Hz, 3H), 0.95-0.83 (m, 2H). MS (ESI) m/z 450.1 [M+H]+.

### Example 131. Synthesis of Compound 130

A mixture of 3-(2,6-difluoro-4-(3-hydroxyazetidin-1-yl)phenyl)piperidine-2,6-dione XII (0.100 g, 0.338 mmol, 1.00 eq) and 1,1′-carbonyldiimidazole (0.109 g, 0.675 mmol, 2.00 eq) in dimethyl formamide (1 mL) was stirred at 25° C. for 2 h. Then, (S)-1-phenylethanamine (61.4 mg, 506 μmol, 1.50 eq), triethylamine (43.4 μL, 0.338 mmol, 1.00 eq) and 2,3,4,6,7,8,9,10-octahydropyrimido[1,2-α]azepine (51.4 mg, 0.338 mmol, 1.00 eq) were added to the mixture and the resulting reaction mixture was stirred at 25° C. for another 12 h. The pH of reaction mixture was adjusted to 6 with formic acid. The resulting mixture was filtered and the filtrate was collected. The filtrate was purified by Prep-HPLC (column: Phenomenex Luna C18 150 mm*25 mm*10 μm; mobile phase: [water (formic acid)-acetonitrile]; B %: 36%-66%, 9 min). The desired fraction was collected and lyophilized to afford 1-(4-(2,6-dioxopiperidin-3-yl)-3,5-difluorophenyl)azetidin-3-yl((S)-1-phenylethyl) carbamate #130 (35.5 mg, 0.076 mmol, 23% yield, 95% purity) as a white solid. 1H NMR (400 MHz, DMSO-d6) δ=10.86 (s, 1H), 7.99 (d, J=8.4 Hz, 1H), 7.34-7.18 (m, 5H), 6.20 (s, 1H), 6.17 (s, 1H), 5.17-5.05 (m, 1H), 4.65 (quin, J=7.2 Hz, 1H), 4.18-4.08 (m, 2H), 4.03 (dd, J=5.0, 12.6 Hz, 1H), 3.78-3.63 (m, 2H), 2.83-2.71 (m, 1H), 2.53-2.52 (m, 1H), 2.15-1.99 (m, 1H), 1.98-1.88 (m, 1H), 1.34 (d, J=7.2 Hz, 3H). MS (ESI) m/z 444.1 [M+H]+.

### Example 132. Synthesis of Compound 131

To a solution of 3-(2,6-difluoro-4-(3-hydroxyazetidin-1-yl)phenyl)piperidine-2,6-dione XII (100 mg, 338 μmol, 1.00 eq) in N,N-dimethyl formamide (1 mL) was added 1,1′-carbonyldiimidazole (109 mg, 675 μmol, 2.00 eq), the mixture was stirred at 20° C. for 2 h. Then butan-1-amine (33.4 μL, 338 μmol, 1.00 eq), triethylamine (47.0 μL, 338 μmol, 1.00 eq), 2,3,4,6,7,8,9,10-octahydropyrimido[1,2-α]azepine (50.9 μL, 338 μmol, 1.00 eq) were added. The mixture was stirred at 20° C. for 12 h. The pH of the mixture was adjusted to 5-6 with formic acid and filtered. The filtrate was purified by Prep-HPLC (column: Phenomenex Luna C18 150*25 mm*10 μm; mobile phase: [water (formic acid)-acetonitrile]; B %: 33%-63%, 9 min) and lyophilized to afford 1-(4-(2,6-dioxopiperidin-3-yl)-3,5-difluorophenyl)azetidin-3-yl butylcarbamate #131 (28.3 mg, 67.6 μmol, 20% yield, 99% purity) as a white solid. 1H NMR (400 MHz, DMSO-d6) δ=10.97-10.74 (m, 1H), 7.36 (br t, J=5.8 Hz, 1H), 6.18 (d, J=11.0 Hz, 2H), 5.20-5.06 (m, 1H), 4.14 (br t, J=7.6 Hz, 2H), 4.03 (m, 1H), 3.71 (br dd, J=3.6, 8.9 Hz, 2H), 2.97 (q, J=6.6 Hz, 2H), 2.83-2.72 (m, 1H), 2.48-2.39 (m, 1H), 2.07 (dq, J=3.9, 13.0 Hz, 1H), 1.99-1.89 (m, 1H), 1.43-1.33 (m, 2H), 1.32-1.21 (m, 2H), 0.92-0.81 (m, 3H). MS (ESI) m/z 396.0 [M+H]+.

### Example 133. Synthesis of Compound 132

A mixture of 3-(2,6-difluoro-4-(3-hydroxyazetidin-1-yl)phenyl)piperidine-2,6-dione XII (80.0 mg, 0.270 mmol, 1.00 eq), di(1H-imidazol-1-yl)methanone (263 mg, 1.62 mmol, 6.00 eq) in dimethyl formamide (8 mL) was stirred at 25° C. for 2 h. Then, 2-methylpropan-1-amine (40.3 μL, 0.405 μmol, 1.50 eq) was added and the mixture was stirred at 25° C. for 11 h. The residue was purified by prep-HPLC (column: Phenomenex Luna C18 150 mm*25 mm*10 μm; mobile phase: [water (formic acid)-acetonitrile]; B %: 35%-65.0%, 8 min). The desired fraction was collected and lyophilized to give 1-(4-(2,6-dioxopiperidin-3-yl)-3,5-difluorophenyl)azetidin-3-yl isobutylcarbamate #132 (50.8 mg, 0.127 mmol, 47% yield, 99% purity) as a white solid. 1H NMR (400 MHz, DMSO-d6) δ=10.85 (s, 1H), 7.41 (t, J=5.8 Hz, 1H), 6.20 (s, 1H), 6.17 (s, 1H), 5.17-5.09 (m, 1H), 4.19-4.10 (m, 2H), 4.03 (dd, J=4.9, 12.6 Hz, 1H), 3.71 (dd, J=3.9, 8.9 Hz, 2H), 2.80 (t, J=6.4 Hz, 2H), 2.75 (dd, J=5.3, 13.1 Hz, 1H), 2.52 (br s, 1H), 2.15-1.99 (m, 1H), 1.98-1.88 (m, 1H), 1.72-1.60 (m, 1H), 0.84 (s, 3H), 0.82 (s, 3H). MS (ESI) m/z 396.1 [M+H]+.

### Example 134. Synthesis of Compound 133

Step 1. To a solution of (1R,4R)-4-methoxycyclohexanamine (420 mg, 3.25 mmol, 1.00 eq) in acetonitrile (5 mL) was added pyridine (262 μL, 3.25 mmol, 1.00 eq) and phenyl carbonochloridate (488 μL, 3.90 mmol, 1.20 eq). The mixture was stirred at 20° C. for 12 h. The crude product was purified by reversed-phase HPLC (formic acid condition) and concentrated to give phenyl ((1R, 4R)-4-methoxycyclohexyl)carbamate (160 mg, 641 μmol, 19% yield) as a white solid. 1H NMR (400 MHz, DMSO-d6) δ=7.73-7.66 (m, 1H), 7.39-7.33 (m, 2H), 7.23-7.16 (m, 1H), 7.12-7.06 (m, 2H), 3.23 (s, 3H), 3.13-3.06 (m, 1H), 2.02-1.96 (m, 2H), 1.92-1.84 (m, 2H), 1.48-0.99 (m, 5H).

Step 2. To a mixture of phenyl ((1R,4R)-4-methoxycyclohexyl)carbamate (100 mg, 405 μmol, 1.20 eq) and 3-(2,6-difluoro-4-(3-hydroxyazetidin-1-yl)phenyl)piperidine-2,6-dione XII (100 mg, 337 μmol, 1.00 eq) in dimethylformamide (0.500 mL) was added sodium hydride (20.2 mg, 506 μmol, 60% purity, 1.50 eq) in portions at 0° C. The mixture was stirred at 25° C. for 2 h. The mixture was quenched with formic acid (0.2 mL). The mixture was adjusted pH=5-6 with formic acid. The mixture was purified by prep-HPLC (column: Welch Ultimate C18 150*25 mm*5 μm; mobile phase: [water (formic acid)-acetonitrile]; B %: 29%-59%, 10.5 min) and lyophilized to give 1-(4-(2,6-dioxopiperidin-3-yl)-3,5-difluorophenyl)azetidin-3-yl ((1R,4R)-4-methoxycyclohexyl)carbamate #133 (63.9 mg, 132 μmol, 39% yield, 94% purity) as a white solid. 1H NMR (400 MHz, DMSO-d6) δ=10.85 (s, 1H), 7.33 (br d, J=7.4 Hz, 1H), 6.18 (br d, J=11.0 Hz, 2H), 5.13 (br s, 1H), 4.14 (br t, J=7.5 Hz, 2H), 4.03 (br dd, J=5.1, 12.7 Hz, 1H), 3.81-3.64 (m, 2H), 3.29 (br s, 1H), 3.21 (s, 3H), 3.10-3.00 (m, 1H), 2.84-2.71 (m, 1H), 2.12-2.02 (m, 1H), 1.98-1.89 (m, 3H), 1.79 (br d, J=10.4 Hz, 2H), 1.32-0.99 (m, 5H). MS (ESI) m/z 452.1 [M+H]+.

### Example 135. Synthesis of Compound 134

Step 1. To a solution of cycloheptanamine (562 μL, 4.42 mmol, 1.00 eq) in acetonitrile (5 mL) was added pyridine (713 μL, 8.83 mmol, 2.00 eq) and phenyl carbonochloridate (663 μL, 5.30 mmol, 1.20 eq) at 0° C. The mixture was stirred at 20° C. for 12 h. The mixture was concentrated under reduced pressure to give the crude product, which was purified by reversed-phase HPLC (column: spherical C18, 20-45 μm, 100 Å, SW 80, mobile phase: [water (0.1% formic acid)-acetonitrile]; B %: 5%-95%, 30 min). The desired fraction was collected and lyophilized to afford phenyl cycloheptylcarbamate (450 mg, 1.93 mmol, 43% yield) as a white solid. 1H NMR (400 MHz, DMSO-d6) δ=7.72 (br d, J=7.9 Hz, 1H), 7.39-7.32 (m, 2H), 7.23-7.13 (m, 1H), 7.08 (d, J=7.9 Hz, 2H), 3.52 (td, J=4.3, 8.5 Hz, 1H), 1.90-1.81 (m, 2H), 1.63-1.38 (m, I0H). MS (ESI) m/z 234.0 [M+H]+.

Step 2. To a solution of phenyl cycloheptylcarbamate (158 mg, 675 μmol, 2.00 eq) and 3-(2,6-difluoro-4-(3-hydroxyazetidin-1-yl)phenyl)piperidine-2,6-dione XII (100 mg, 338 μmol, 1.00 eq) in N,N-dimethyl formamide (2 mL) was added sodium hydride (27.0 mg, 675 μmol, 60% purity, 2.00 eq) at 0° C. The mixture was stirred at 0° C. for 1 h. The mixture was adjusted to pH=6 with formic acid, then the mixture was filtered and the filtrate was purified by Prep-HPLC (column: Phenomenex Luna C18 150*25 mm*10 μm; mobile phase: [water (formic acid)-acetonitrile]; B %: 41%-71%, 9 min). The desired fraction was collected and the aqueous solution was lyophilized to afford 1-(4-(2,6-dioxopiperidin-3-yl)-3,5-difluorophenyl)azetidin-3-yl cycloheptylcarbamate #134 (63.0 mg, 145 μmol, 42% yield) as a yellow solid. 1H NMR (400 MHz, DMSO-d6) δ=10.86 (s, 1H), 7.37 (br d, J=7.9 Hz, 1H), 6.18 (d, J=11.1 Hz, 2H), 5.17-5.05 (m, 1H), 4.14 (br t, J=7.6 Hz, 2H), 4.03 (br dd, J=5.0, 12.5 Hz, 1H), 3.70 (br dd, J=3.1, 8.7 Hz, 2H), 3.46 (td, J=4.3, 8.2 Hz, 1H), 2.85-2.70 (m, 1H), 2.52 (d, J=1.9 Hz, 1H), 2.18-2.00 (m, 1H), 1.99-1.86 (m, 1H), 1.83-1.69 (m, 2H), 1.63-1.28 (m, I0H). MS (ESI) m/z 436.0 [M+H]+.

### Example 136. Synthesis of Compound 135

Step 1. To a solution of 1-methylcyclohexanamine (190 mg, 1.68 mmol, 1.00 eq) in acetonitrile (5.00 mL) was added pyridine (406 μL, 5.04 mmol, 3.00 eq) and phenyl carbonochloridate (315 μL, 2.52 mmol, 1.50 eq). The mixture was stirred at 20° C. for 12 h. The reaction mixture was concentrated under reduced pressure to remove acetonitrile (5 mL). The residue was diluted with water (20 mL) and extracted with ethyl acetate (3×20 mL). The organic layers were washed with water (3×20 mL), dried over anhydrous sodium sulfate, filtered and concentrated under reduced pressure to give a residue. The residue was purified by column chromatography (SiO2, petroleum ether:ethyl acetate=15:1) to give phenyl (1-methylcyclohexyl)carbamate (132 mg, 515 μmol, 30% yield) as a yellow solid. 1H NMR (400 MHz, DMSO-d6) δ=7.40-7.31 (m, 3H), 7.21-7.16 (m, 1H), 7.09-7.05 (m, 2H), 1.56-1.38 (m, 6H), 1.37-1.21 (m, 7H).

Step 2. To a solution of 3-(2,6-difluoro-4-(3-hydroxyazetidin-1-yl)phenyl)piperidine-2,6-dione XII (93.1 mg, 314 μmol, 1.00 eq) in dimethyl formamide (5 mL) was added sodium hydride (25.1 mg, 629 μmol, 60% purity, 2.00 eq) at 0° C. The mixture was added phenyl N-(1-methylcyclohexyl) carbamate (110 mg, 471 μmol, 1.50 eq). The mixture was stirred at 20° C. for 2 h. The reaction mixture was quenched by addition formic acid (5 mL) at 0° C. and concentrated under reduced pressure to give a residue. The residue was purified by Prep-HPLC (column: Phenomenex Luna C18 100*30 mm*5 μm; mobile phase: [water (formic acid)-acetonitrile]; B %: 44%-74%, 8 min) and lyophilized to afford 1-(4-(2,6-dioxopiperidin-3-yl)-3,5-difluorophenyl)azetidin-3-yl (1-methylcyclohexyl)carbamate #135 (46.9 mg, 107 μmol, 34% yield, 99% purity) as a white solid. 1H NMR (400 MHz, DMSO-d6) δ=10.87 (s, 1H), 6.95 (br s, 1H), 6.20 (d, J=11.0 Hz, 2H), 5.11 (br d, J=3.50 Hz, 1H), 4.15 (br t, J=7.6 Hz, 2H), 4.04 (br dd, J=5.1, 12.6 Hz, 1H), 3.71 (br dd, J=3.60, 8.80 Hz, 2H), 2.84-2.72 (m, 1H), 2.53 (d, J=1.90 Hz, 1H), 2.08 (dq, J=3.3, 13.0 Hz, 1H), 1.99-1.86 (m, 3H), 1.49-1.34 (m, 5H), 1.32-1.22 (m, 3H), 1.20 (s, 3H). MS (ESI) m/z 435.9 [M+H]+.

### Example 137. Synthesis of Compound 136

Step 1. To a mixture of phenyl carbonochloridate (508 mg, 3.24 mmol, 1.10 eq) in acetonitrile (6 mL) was added pyridine (699 mg, 8.84 mmol, 3.00 eq) and 2,3-dihydro-1H-inden-2-amine hydrochloride (500 mg, 2.95 mmol, 1.00 eq) at 25° C. with stirring. The resulting mixture was stirred at 25° C. for 12 h. The mixture was filtered and the filtrate was concentrated under reduced pressure to give phenyl (2,3-dihydro-1H-inden-2-yl)carbamate (780 mg, crude) as a white solid. 1H NMR (400 MHz, DMSO-d6) δ=8.11 (d, J=6.6 Hz, 1H), 7.51-7.46 (m, 1H), 7.40-7.31 (m, 4H), 7.24-7.09 (m, 6H), 4.03 (q, J=7.2 Hz, 1H), 3.20 (dd, J=7.6, 15.8 Hz, 2H), 2.89 (dd, J=6.6, 15.9 Hz, 2H).

Step 2. To a mixture of phenyl (2,3-dihydro-1H-inden-2-yl)carbamate (205 mg, 0.810 mmol, 2.00 eq) in 2-methyltetrahydrofuran (5 mL) was added sodium hydride (48.6 mg, 1.22 mmol, 60% purity, 3.00 eq) at 0° C. with stirring. The mixture was stirred at 25° C. for 0.5 h. Then, 3-(2,6-difluoro-4-(3-hydroxyazetidin-1-yl)phenyl)piperidine-2,6-dione XII (120 mg, 0.405 mmol, 1.00 eq) was added and the resulting reaction mixture was stirred at 25° C. for 12 h. The residue was purified by prep-HPLC (column: Phenomenex Luna C18 150*25 mm*10 μm; mobile phase: [water (formic acid)-acetonitrile]; B %: 39%-69%, 9 min). The desired fraction was collected and lyophilized to afford 1-(4-(2,6-dioxopiperidin-3-yl)-3,5-difluorophenyl)azetidin-3-yl (2,3-dihydro-1H-inden-2-yl)carbamate #136 (11.4 mg, 0.0240 mmol, 11% yield, 99% purity) as a white solid. 1H NMR (400 MHz, DMSO-d6) δ=10.85 (s, 1H), 7.75 (d, J=6.8 Hz, 1H), 7.24-7.09 (m, 4H), 6.19 (d, J=11.2 Hz, 2H), 5.17 (s, 1H), 4.25 (dd, J=6.8, 13.9 Hz, 1H), 4.16 (t, J=7.4 Hz, 2H), 4.03 (dd, J=4.8, 12.2 Hz, 1H), 3.73 (d, J=6.6 Hz, 2H), 3.14 (dd, J=7.6, 15.9 Hz, 2H), 2.85-2.66 (m, 4H), 2.16-2.00 (m, 1H), 1.99-1.87 (m, 1H). MS (ESI) m/z 456.1[M+H]+.

### Example 138. Synthesis of Compound 137

Step 1. A mixture of phenylmethanamine (509 μL, 4.67 mmol, 1.00 eq), phenyl carbonochloridate (643 μL, 5.13 mmol, 1.10 eq) and pyridine (1.13 mL, 14.0 mmol, 3.00 eq) in acetonitrile (5 mL) was stirred at 25° C. for 12 h. The reaction mixture was poured into water (15.0 mL) and extracted with ethyl acetate (4×15 mL). The organic layers were collected. The combined organic layers were washed with brine (3×15 mL), dried over sodium sulfate, filtered and concentrated in vacuo to afford phenyl benzylcarbamate (1.28 g, crude) as a light yellow solid. 1H NMR (400 MHz, DMSO-d6) δ=8.59 (s, 1H), 7.42-7.30 (m, 8H), 7.30-7.24 (m, 1H), 7.23-7.17 (m, 1H), 4.28 (d, J=6.2 Hz, 2H).

Step 2. To a mixture of phenyl benzylcarbamate (184 mg, 810 μmol, 3.00 eq) in dimethyl formamide (5 mL) was added sodium hydride (21.6 mg, 540 μmol, 60% purity, 2.00 eq) at 0° C. with stirring. The mixture was stirred at 25° C. for 0.5 h. Then, 3-(2,6-difluoro-4-(3-hydroxyazetidin-1-yl)phenyl)piperidine-2,6-dione XII (80.0 mg, 270 μmol, 1.00 eq) was added and the resulting reaction mixture was stirred at 25° C. for 1 h. The reaction mixture was quenched with formic acid. The pH of reaction mixture was adjusted to 7 with formic acid. The resulting mixture was poured into water (50 mL) and the mixture was extracted with ethyl acetate (3×50 mL). The organic layers were collected. The combined organic layers were washed with brine (3×10 mL), dried over sodium sulfate, filtered and concentrated in vacuo. The residue was purified by prep-HPLC (column: Phenomenex Luna C18 150 mm×25 mm×10 μm; mobile phase: [water (formic acid)-acetonitrile]; B %: 37%-67%, 8 min) to give 1-(4-(2,6-dioxopiperidin-3-yl)-3,5-difluorophenyl)azetidin-3-yl benzylcarbamate #137 (55.3 mg, 124 μmol, 88% yield, 96% purity) as a white solid. 1H NMR (400 MHz, DMSO-d6) δ=10.85 (s, 1H), 7.96 (t, J=6.2 Hz, 1H), 7.40-7.17 (m, 5H), 6.20 (s, 1H), 6.18 (s, 1H), 5.21-5.12 (m, 1H), 4.24-4.11 (m, 4H), 4.04 (dd, J=5.0, 12.6 Hz, 1H), 3.74 (dd, J=3.4, 8.9 Hz, 2H), 2.84-2.71 (m, 1H), 2.52 (br s, 1H), 2.15-2.00 (m, 1H), 1.99-1.87 (m, 1H). MS (ESI) m/z 430.0 [M+H]+.

### Example 139. Synthesis of Compound 138

Step 1. To a solution of spiro[3.3]heptan-2-amine (200 mg, 1.80 mmol, 1.00 eq) in acetonitrile (2 mL) was added pyridine (290 μL, 3.60 mmol, 2.00 eq), then phenyl carbonochloridate (270 μL, 2.16 mmol, 1.20 eq) was added into the mixture at 0° C., the mixture was stirred at 0° C. for 1 h. The mixture was concentrated under reduce pressure to give a residue, which was purified by column chromatography (SiO2, petroleum ether/ethyl acetate=10:1) to afford phenyl spiro[3.3]heptan-2-ylcarbamate (350 mg, 1.51 mmol, 84% yield) as a white solid. 1H NMR (400 MHz, DMSO-d6) δ=7.96 (br d, J=7.9 Hz, 1H), 7.39-7.33 (m, 2H), 7.23-7.15 (m, 1H), 7.11-7.03 (m, 2H), 3.93-3.78 (m, 1H), 2.32-2.25 (m, 2H), 1.99 (t, J=7.2 Hz, 2H), 1.95-1.87 (m, 4H), 1.82-1.75 (m, 2H).

Step 2. To a solution of 3-(2,6-difluoro-4-(3-hydroxyazetidin-1-yl)phenyl)piperidine-2,6-dione XII (100 mg, 338 μmol, 1.00 eq) and phenyl spiro[3.3]heptan-2-ylcarbamate (94.0 mg, 406 μmol, 1.20 eq) in N,N-dimethyl formamide (1.5 mL) was added sodium hydride (27.0 mg, 675 μmol, 60% purity, 2.00 eq) at 0° C. The mixture was stirred at 25° C. for 1 h. The pH of the mixture was adjusted to around 9 by adding formic acid, then the mixture was filtered to give the filtrate, which was dissolved in N,N-dimethyl formamide (2.5 mL) and purified by Prep-HPLC (column: Phenomenex Luna C18 150*25 mm*10 μm; mobile phase: [water (formic acid)-acetonitrile]; B %: 41%-71%, 9 min) and lyophilized to afford 1-(4-(2,6-dioxopiperidin-3-yl)-3,5-difluorophenyl)azetidin-3-yl spiro[3.3]heptan-2-ylcarbamate #138 (48.5 mg, 111 μmol, 32% yield, 99% purity) as a white solid. 1H NMR (400 MHz, DMSO-d6) δ=10.85 (s, 1H), 7.61 (d, J=7.9 Hz, 1H), 6.19 (s, 1H), 6.16 (s, 1H), 5.14-5.08 (m, 1H), 4.13 (br t, J=7.6 Hz, 2H), 4.03 (br dd, J=4.9, 12.6 Hz, 1H), 3.83-3.76 (m, 1H), 3.70 (br dd, J=3.4, 8.8 Hz, 2H), 2.83-2.72 (m, 1H), 2.52 (br d, J=1.9 Hz, 1H), 2.23 (dt, J=2.8, 8.2 Hz, 2H), 2.07 (br dd, J=3.6, 13.1 Hz, 1H), 1.99-1.95 (m, 2H), 1.95-1.89 (m, 1H), 1.88-1.82 (m, 4H), 1.80-1.73 (m, 2H). MS (ESI) m/z 434.4 [M+H]+.

### Example 140. Synthesis of Compound 139

Step 1. To a solution of cyclopentylmethanamine hydrochloride (500 mg, 3.69 mmol, 1.00 eq) and pyridine (893 μL, 11.1 mmol, 3.00 eq) in acetonitrile (10 mL) was added phenyl carbonochloridate (554 μL, 4.42 mmol, 1.20 eq). The reaction mixture was stirred at 20° C. for 1 h. The reaction mixture was diluted with water (20 mL), extracted with ethyl acetate (3×20 mL). The combined organic layers were dried over anhydrous sodium sulfate, filtered and concentrated under reduced pressure to give a residue. The residue was purified by column chromatography (SiO2, petroleum ether/ethyl acetate=20/1 to 10/1) and concentrated under reduced pressure to afford phenyl (cyclopentylmethyl)carbamate (450 mg, 2.05 mmol, 55% yield) as a white solid. 1H NMR (400 MHz, CDCl3) δ=7.37-7.17 (m, 3H), 7.13-7.04 (m, 2H), 4.97 (br s, 1H), 3.26-2.96 (m, 2H), 2.03 (td, J=7.5, 15.0 Hz, 1H), 1.72 (br d, J=6.3 Hz, 2H), 1.63-1.39 (m, 4H), 1.23-1.10 (m, 2H).

Step 2. To a solution of 3-(2,6-difluoro-4-(3-hydroxyazetidin-1-yl)phenyl)piperidine-2,6-dione XII (100 mg, 337 μmol, 1.00 eq) in dimethylformamide (1 mL) was added sodium hydride (27.0 mg, 675 μmol, 60% purity, 2.00 eq) at 0° C. The mixture was stirred at 0° C. for 10 min. Then phenyl (cyclopentylmethyl)carbamate (222 mg, 1.01 mmol, 3.00 eq) was added and the mixture was stirred at 20° C. for 12 h. The mixture was quenched with formic acid (pH=5-6) and filtered. The filtrate was purified by Prep-HPLC (column: Phenomenex Luna C18 150*25 mm*10 μm; mobile phase: [water (formic acid)-acetonitrile]; B %: 38%-68%, 9 min) and lyophilized to afford 1-(4-(2,6-dioxopiperidin-3-yl)-3,5-difluorophenyl)azetidin-3-yl (cyclopentylmethyl)carbamate #139 (47.28 mg, 109 μmol, 32% yield, 98% purity) as a white solid. 1H NMR (400 MHz, DMSO-d6) δ=10.85 (s, 1H), 7.42 (t, J=5.8 Hz, 1H), 6.18 (d, J=11.0 Hz, 2H), 5.17-5.08 (m, 1H), 4.14 (br t, J=7.6 Hz, 2H), 4.03 (dd, J=4.9, 12.6 Hz, 1H), 3.71 (br dd, J=3.7, 8.7 Hz, 2H), 2.90 (t, J=6.5 Hz, 2H), 2.83-2.71 (m, 1H), 2.52 (br s, 1H), 2.13-2.01 (m, 1H), 2.00-1.89 (m, 2H), 1.63 (dt, J=6.8, 11.5 Hz, 2H), 1.57-1.43 (m, 4H), 1.20-1.11 (m, 2H). MS (ESI) m/z 422.1 [M+H]+.

### Example 141. Synthesis of Compound 140

Step 1. To a mixture of (2R)-bicyclo[2.2.1]heptan-2-amine (500 mg, 4.50 mmol, 1.00 eq) and pyridine (1.81 mL, 22.4 mmol, 5.00 eq) in acetonitrile (5 mL) was added phenyl carbonochloridate (675 μL, 5.40 mmol, 1.20 eq) dropwise at 0° C. The mixture was stirred at 25° C. for 2 h. The mixture was added water (30 mL) and extracted with ethyl acetate (3×30 mL). The combined organic phase was washed with brine (30 mL), dried over anhydrous sodium sulfate, filtered and concentrated in vacuum. The residue was purified by column chromatography (SiO2, petroleum ether/ethyl acetate=1/0 to 3/1) to give phenyl (2R)-bicyclo[2.2.1]heptan-2-ylcarbamate (280 mg, 1.21 mmol, 26% yield) as a white solid. 1H NMR (400 MHz, DMSO-d6) δ=7.69 (br d, J=6.8 Hz, 1H), 7.41-7.32 (m, 2H), 7.24-7.15 (m, 1H), 7.09 (br d, J=7.8 Hz, 2H), 3.39-3.35 (m, 1H), 2.20 (br s, 1H), 2.15 (br s, 1H), 1.66-1.56 (m, 1H), 1.52-1.34 (m, 4H), 1.08 (br d, J=7.3 Hz, 3H).

Step 2. To a solution of 3-(2,6-difluoro-4-(3-hydroxyazetidin-1-yl)phenyl)piperidine-2,6-dione XII (100 mg, 337 μmol, 1.00 eq), phenyl (2R)-bicyclo[2.2.1]heptan-2-ylcarbamate (93.7 mg, 405 μmol, 1.20 eq) in dimethylformamide (1 mL) was added sodium hydride (20.2 mg, 506 μmol, 60% purity, 1.50 eq) at 0° C. The mixture was stirred at 25° C. for 1 h. The mixture was quenched with formic acid (0.2 mL) and filtered. The filtrate was purified by prep-HPLC (column: Phenomenex Luna C18 150*25 mm*10 μm; mobile phase: [water (formic acid)-acetonitrile]; B %: 39%-69%, 9 min) and lyophilized to give 1-(4-(2,6-dioxopiperidin-3-yl)-3,5-difluorophenyl)azetidin-3-yl(2R)-bicyclo[2.2.1]heptan-2-ylcarbamate #140 (52.79 mg, 115 μmol, 34% yield, 95% purity) as a white solid. 1H NMR (400 MHz, DMSO-d6) δ=10.85 (br s, 1H), 7.33 (br d, J=7.0 Hz, 1H), 6.18 (d, J=11.0 Hz, 2H), 5.21-5.04 (m, 1H), 4.14 (br t, J=7.3 Hz, 2H), 4.03 (br dd, J=5.0, 12.4 Hz, 1H), 3.72 (br dd, J=4.3, 8.5 Hz, 2H), 3.30-3.25 (m, 1H), 2.84-2.71 (m, 1H), 2.52 (br d, J=1.9 Hz, 1H), 2.18-2.01 (m, 3H), 1.98-1.90 (m, 1H), 1.61-1.52 (m, 1H), 1.46-1.25 (m, 4H), 1.14-0.97 (m, 3H). MS (ESI) m/z 434.1 [M+H]+.

### Example 142. Synthesis of Compound 141

Step 1. To a solution of oxetan-3-ylmethanamine (100 mg, 1.15 mmol, 1.00 eq) in acetonitrile (2 mL) was added pyridine (463 μL, 5.74 mmol, 5.00 eq), then the mixture was added phenyl carbonochloridate (143 μL, 1.15 mmol, 1.00 eq) at 0° C. The mixture was stirred at 20° C. for 1 h. The reaction mixture was filtered to give filtrate, which was purified by reversed-phase HPLC (C18, 40 g; condition: water/acetonitrile=1/0 to 0/1, 0.1% formic acid) to afford phenyl (oxetan-3-ylmethyl)carbamate (120 mg, 0.579 mmol, 50% yield) as a white solid.

Step 2. To a solution of 3-(2,6-difluoro-4-(3-hydroxyazetidin-1-yl)phenyl)piperidine-2,6-dione XII (171 mg, 0.579 mmol, 1.00 eq) and phenyl (oxetan-3-ylmethyl)carbamate (120 mg, 0.579 mmol, 1.00 eq) in dimethylformamide (2 mL) was added sodium hydride (46.3 mg, 1.16 mmol, 60% purity, 2.00 eq) at 0° C. The mixture was stirred at 0° C. for 1 h. The reaction mixture was quenched with 1M hydrochloric acid (0.5 mL) and filtered. The filtrate was purified by Prep-HPLC (column: Waters Xbridge 150×25 mm×5 μm; mobile phase: [water-acetonitrile]; B %: 19%-49%, 9 min) and lyophilized to give 1-(4-(2,6-dioxopiperidin-3-yl)-3,5-difluorophenyl) azetidin-3-yl (oxetan-3-ylmethyl)carbamate #141 (54.8 mg, 0.133 mmol, 23% yield, 99% purity) as a white solid. 1H NMR (400 MHz, DMSO-d6) δ=10.87 (br s, 1H), 7.60 (t, J=5.6 Hz, 1H), 6.19 (d, J=11.2 Hz, 2H), 5.24-5.06 (m, 1H), 4.59 (dd, J=6.0, 7.6 Hz, 2H), 4.26 (t, J=6.0 Hz, 2H), 4.15 (br t, J=7.6 Hz, 2H), 4.04 (br dd, J=5.2, 12.8 Hz, 1H), 3.73 (br dd, J=3.6, 8.8 Hz, 2H), 3.27 (br t, J=6.4 Hz, 2H), 3.09-2.98 (m, 1H), 2.84-2.75 (m, 1H), 2.47-2.39 (m, 1H), 2.08 (br dd, J=3.6, 13.2 Hz, 1H), 2.00-1.89 (m, 1H). MS (ESI) m/z 410.0 [M+H]+.

### Example 143. Synthesis of Compound 142

Step 1. To a solution of 4,4-difluoropiperidine (500 mg, 4.13 mmol, 1.00 eq) and pyridine (666 μL, 8.26 mmol, 2.00 eq) in acetonitrile (5 mL) was added phenyl carbonochloridate (620 μL, 4.95 mmol, 1.20 eq) in portions at 0° C. The mixture was stirred at 0° C. for 1 h. The reaction mixture was concentrated under reduced pressure to give a residue. The residue was purified by reversed phase column chromatography (C18, 80 g; condition: water/acetonitrile=1/0 to 0/1, 0.1% formic acid) and lyophilized to afford phenyl 4,4-difluoropiperidine-1-carboxylate (600 mg, 2.49 mmol, 60% yield) as a white solid. 1H NMR (400 MHz, DMSO-d6) δ=7.42-7.36 (m, 2H), 7.26-7.20 (m, 1H), 7.15 (d, J=7.7 Hz, 2H), 3.73-3.53 (m, 4H), 2.07 (br s, 4H).

Step 2. To a solution of 3-(2,6-difluoro-4-(3-hydroxyazetidin-1-yl)phenyl)piperidine-2,6-dione XII (60.0 mg, 203 μmol, 1.00 eq) and phenyl 4,4-difluoropiperidine-1-carboxylate (53.7 mg, 223 μmol, 1.10 eq) in dimethyl formamide (1 mL) was added sodium hydride (12.2 mg, 304 μmol, 60% purity, 1.50 eq) at 0° C. The mixture was stirred at 25° C. for 1 h. The mixture was quenched by formic acid (1 mL) at 0° C. and filtered. The filtrate was purified by Prep-HPLC (column: Phenomenex Synergi C18 150*25 mm*10 μm; mobile phase: [water (formic acid)-acetonitrile]; B %: 42%-62%, 10 min) and lyophilized to afford 1-(4-(2,6-dioxopiperidin-3-yl)-3,5-difluorophenyl)azetidin-3-yl 4,4-difluoropiperidine-1-carboxylate #142 (24.94 mg, 56.0 μmol, 28% yield, 99% purity) as an off-white solid. 1H NMR (400 MHz, DMSO-d6) δ=10.86 (s, 1H), 6.19 (d, J=11.1 Hz, 2H), 5.22-5.12 (m, 1H), 4.17 (br t, J=7.8 Hz, 2H), 4.04 (dd, J=5.1, 12.5 Hz, 1H), 3.80 (br dd, J=3.6, 9.0 Hz, 2H), 3.52 (br d, J=2.6 Hz, 4H), 2.87-2.71 (m, 1H), 2.54-2.52 (m, 1H), 2.14-2.04 (m, 1H), 2.03-1.91 (m, 5H). MS (ESI) m/z 444.1 [M+H]⁺.

### Example 144. Synthesis of Compound 143

Step 1. To a solution of piperidine (0.673 mL, 6.81 mmol, 1.00 eq) and pyridine (1.13 mL, 13.6 mmol, 2.00 eq) in acetonitrile (5 mL) was added phenyl carbonochloridate (1.02 mL, 8.17 mmol, 1.20 eq) in portions at 0° C. The mixture was stirred at 0° C. for 1 h. The reaction mixture was concentrated under reduced pressure to give a residue. The residue was purified by reversed phase column chromatography (C18, 120 g; condition: water/acetonitrile=1/0 to 0/1, 0.1% formic acid) and lyophilized to afford phenyl piperidine-1-carboxylate (900 mg, 4.38 mmol, 64% yield) as a white solid. 1H NMR (400 MHz, DMSO-d6) δ=7.41-7.35 (m, 2H), 7.24-7.18 (m, 1H), 7.13-7.07 (m, 2H), 3.54 (br s, 2H), 3.40 (br s, 2H), 1.63-1.51 (m, 6H).

Step 2. To a solution of 3-(2,6-difluoro-4-(3-hydroxyazetidin-1-yl)phenyl)piperidine-2,6-dione XII (60.0 mg, 203 μmol, 1.00 eq) and phenyl piperidine-1-carboxylate (45.7 mg, 223 μmol, 1.10 eq) in dimethylformamide (1 mL) was added sodium hydride (12.2 mg, 304 μmol, 60% purity, 1.50 eq) at 0° C. The mixture was stirred at 25° C. for 1 h. The mixture was quenched by formic acid (1 mL) at 0° C. and filtered. The filtrate was purified by Prep-HPLC (column: Phenomenex Synergi C18 150*25 mm*10 μm; mobile phase: [water (formic acid)-acetonitrile]; B %: 44%-64%, 10 min) and lyophilized to afford 1-(4-(2,6-dioxopiperidin-3-yl)-3,5-difluorophenyl)azetidin-3-yl piperidine-1-carboxylate #143 (31.31 mg, 76.1 μmol, 38% yield, 99% purity) as an off-white solid. 1H NMR (400 MHz, DMSO-d6) δ=10.86 (s, 1H), 6.18 (d, J=11.0 Hz, 2H), 5.17-5.11 (m, 1H), 4.16 (br t, J=7.7 Hz, 2H), 4.04 (br dd, J=4.8, 12.5 Hz, 1H), 3.75 (br dd, J=3.4, 8.8 Hz, 2H), 3.44-3.35 (m, 4H), 2.83-2.73 (m, 1H), 2.58-2.54 (m, 1H), 2.13-2.02 (m, 1H), 1.99-1.88 (m, 1H), 1.54 (br d, J=4.5 Hz, 2H), 1.49-1.42 (m, 4H). MS (ESI) m/z 408.2 [M+H]+.

### Example 145. Synthesis of Compound 144

Step 1. To a solution of pyrrolidine (0.587 mL 7.03 mmol, 1.00 eq) and pyridine (1.13 mL, 14.1 mmol, 2.00 eq) in acetonitrile (10 mL) was added phenyl carbonochloridate (1.06 mL, 8.44 mmol, 1.20 eq) in portions at 0° C. The mixture was stirred at 0° C. for 1 h. The reaction mixture was concentrated under reduced pressure to give a residue. The residue was purified by reversed phase column chromatography (C18, 80 g; condition: water/acetonitrile=1/0 to 0/1, 0.1% formic acid) and lyophilized to afford phenyl pyrrolidine-1-carboxylate (1.00 g, 5.23 mmol, 74% yield) as a white solid. 1H NMR (400 MHz, DMSO-d6) δ=7.41-7.34 (m, 2H), 7.23-7.17 (m, 1H), 7.12 (d, J=8.0 Hz, 2H), 3.49 (t, J=6.5 Hz, 2H), 3.33-3.31 (m, 2H), 1.94-1.80 (m, 4H).

Step 2. To a solution of 3-(2,6-difluoro-4-(3-hydroxyazetidin-1-yl)phenyl)piperidine-2,6-dione XII (60.0 mg, 203 μmol, 1.00 eq) and phenyl pyrrolidine-1-carboxylate (42.6 mg, 223 μmol, 1.10 eq) in dimethylformamide (1 mL) was added sodium hydride (12.2 mg, 304 μmol, 60% purity, 1.50 eq) at 0° C. The mixture was stirred at 25° C. for 1 h. The mixture was quenched by formic acid (1 mL) at 0° C. and filtered. The filtrate was purified by Prep-HPLC (column: Phenomenex Synergi C18 150*25 mm*10 μm; mobile phase: [water (formic acid)-acetonitrile]; B %: 38%-58%, 10 min) and lyophilized to afford 1-(4-(2,6-dioxopiperidin-3-yl)-3,5-difluorophenyl)azetidin-3-yl pyrrolidine-1-carboxylate #144 (31.23 mg, 77.8 μmol, 38% yield, 98% purity) as an off-white solid. 1H NMR (400 MHz, DMSO-d6) δ=10.86 (s, 1H), 6.19 (d, J=11.0 Hz, 2H), 5.19-5.12 (m, 1H), 4.16 (br t, J=7.7 Hz, 2H), 4.04 (dd, J=5.0, 12.5 Hz, 1H), 3.75 (br dd, J=3.6, 8.7 Hz, 2H), 3.31-3.29 (m, 2H), 3.25 (br t, J=6.4 Hz, 2H), 2.83-2.72 (m, 1H), 2.53-2.52 (m, 1H), 2.14-2.01 (m, 1H), 1.99-1.90 (m, 1H), 1.80 (td, J=6.7, 13.2 Hz, 4H). MS (ESI) m/z 394.2 [M+H]+.

### Example 146: Synthesis of Compound 145

Step 1. A mixture of 3-chloro-4-methylaniline (3.00 g, 21.2 mmol, 1.00 eq) and dimethyl sulfate (2.01 mL, 21.2 mmol, 1.00 eq) in water (30 mL) was stirred at 0° C. for 1 h. Then 30% sodium hydroxide solution (50 mL) was added. The mixture was extracted with ethyl acetate (80 mL) and washed with water (30 mL). The combined organic phase was dried over anhydrous sodium sulfate (20 mg), filtered and concentrated reduced pressure to give the crude product, which was purified by column chromatography (SiO2, petroleum ether/ethyl acetate=50/1 to 1/1) to afford 3-chloro-N,4-dimethylaniline (520 mg, 3.34 mmol, 15% yield) as a yellow oil. 1H NMR (400 MHz, CDCl3) δ=7.01 (d, J=8.3 Hz, 1H), 6.63 (d, J=2.4 Hz, 1H), 6.44 (dd, J=2.4, 8.3 Hz, 1H), 2.81 (s, 3H), 2.27 (s, 3H).

Step 2. To a solution of 3-chloro-N,4-dimethylaniline (200 mg, 1.29 mmol, 1.00 eq) in acetonitrile (3 mL) was added pyridine (207 μL, 2.57 mmol, 2.00 eq) and phenyl carbonochloridate (193 μL, 1.54 mmol, 1.20 eq) dropwise at 0° C. The mixture was stirred at 0° C. for 1 h. The mixture was concentrated under reduced pressure to give the crude product, which was purified by column chromatography (SiO2, petroleum ether/ethyl acetate=1/0 to 5/1) to afford phenyl (3-chloro-4-methylphenyl)(methyl)carbamate (320 mg, 1.16 mmol, 90% yield) as colorless oil. 1H NMR (400 MHz, CDCl3) δ=7.45-7.29 (m, 3H), 7.26-7.09 (m, 5H), 3.41 (br s, 3H), 2.38 (s, 3H). MS (ESI) m/z 275.8 [M+H]+.

Step 3. To a solution of 3-(2,6-difluoro-4-(3-hydroxyazetidin-1-yl)phenyl)piperidine-2,6-dione XII (100 mg, 338 μmol, 1.00 eq) and phenyl (3-chloro-4-methylphenyl)(methyl)carbamate (102 mg, 371 μmol, 1.10 eq) in N,N-dimethyl formamide (2 mL) was added sodium hydride (30.0 mg, 750 μmol, 60% purity, 2.22 eq) at 0° C. The mixture was stirred at 25° C. for 1 h. The mixture was diluted with water (50 mL) and extracted with ethyl acetate (3×30 mL). The combined organic layers were washed with water (10 mL) and dried over anhydrous sodium sulfate. After filtration, the filtrate was concentrated under reduced pressure to give the crude product, which was purified by column chromatography (SiO2, petroleum ether/ethyl acetate=20/1 to 1/1) and further purified by Prep-HPLC (column: Phenomenex Luna C18 150*25 mm*10 μm; mobile phase: [water (formic acid)-acetonitrile]; B %: 46%-76%, 9 min) and lyophilized to afford 1-(4-(2,6-dioxopiperidin-3-yl)-3,5-difluorophenyl)azetidin-3-yl(3-chloro-4-methylphenyl)(methyl) carbamate #145 (32.02 mg, 66 μmol, 19% yield, 98% purity) as a white solid. 1H NMR (400 MHz, DMSO-d6) δ=10.86 (s, 1H), 7.46 (d, J=2.3 Hz, 1H), 7.35 (d, J=8.3 Hz, 1H), 7.24 (dd, J=2.3, 8.1 Hz, 1H), 6.19 (s, 1H), 6.17 (s, 1H), 5.23-5.16 (m, 1H), 4.16 (br t, J=7.6 Hz, 2H), 4.03 (dd, J=5.1, 12.6 Hz, 1H), 3.74 (br d, J=3.4 Hz, 2H), 3.23 (s, 3H), 2.82-2.72 (m, 1H), 2.52 (d, J=1.9 Hz, 1H), 2.31 (s, 3H), 2.10-2.01 (m, 1H), 1.97-1.89 (m, 1H). MS (ESI) m/z 478.0 [M+H]+.

### Example 146: Compound Binding to CRBN by HTRF Assay

Compound activity was monitored in a Homogenous Time-Resolved Fluorescence (HTRF) assay using 1-[5-({2-[2-(2-{[2-(2,6-dioxopiperidin-3-yl)-1,3-dioxo-2,3-dihydro-1H-isoindol-4-yl]oxy}acetamido)ethoxy]ethyl}carbamoyl)pentyl]-3,3-dimethyl-2-[(1E,3E)-5-[(2E)-1,3,3-trimethyl-5-sulfo-2,3-dihydro-1H-indol-2-ylidene]penta-1,3-dien-1-yl]-3H-indol-1-ium-5-sulfonate as a fluorescent probe. Biochemical assays were conducted in Greiner white 384 well HiBase plates (Cat. No 784075-25) in 10 μL total volume. A one pot detection solution of CRBN-DDB1 (2.5 nM), Anti-His Terbium Cryptate Gold (1X, PerkinElmer Cat. #: 61HI2TLB), and Cy5-Thalidomide (100 nM, Tenova Cat.: T52461) was prepared in 20 mM HEPES, 20 mM NaCl, 0.2 mM TCEP, 0.2 mM EDTA, and 0.005% Tween20 was dispensed to each assay plate. Compounds were stored in dry, ambient temperatures at 10 mM. A 10-point, 1:3 dilution series was prepared from 10 mM stock concentrations in Echo-compatible LDV plates. 10 nL of each compound dilution series was dispensed into assays wells using an Echo 650 (Labcyte inc. USA). 10 nL of 10 mM Lenalidomide was transferred into the active-control wells for the assay and 10 nL of DMSO was transferred into the neutral-control wells. The assay was then allowed to incubate for 30 min at ambient temperature after transferring compound. Plate measurements were taken on a Pherastar FSX (BMG Labtech, Germany) using the HTRF Red filter (Ex. 337 nm, em1: 620 nm, em2: 665 nm) (Flashes: 50, Integration time: 60-400 us, Z-height: 10 mm, Ratio-multiplier: 10,000). The HTRF signal was then subsequently normalized to the neutral and active controls. Analysis and IC50 values were derived using KNIME analytics (KNIME Zurich) transformation and fitting within Collaborative Drug Discovery (Collaborative Drug Discovery USA). Ki was derived from the geometric mean of the IC50 values using the Cheng-Prustoff transformation.

### Example 147: Compound Activity by Immunofluorescence Assay

The representative compounds were tested in an immunofluorescence assay for their activity to degrade GSPT1. CAL-51 cells were purchased from DSMZ (cat. Number ACC302), sub-cultured in 90% Dulbecco's MEM (4.5 g/L glucose, Gibco 11965)+10% heat inactivated FBS (BioConcept, 2-01F136I) and incubated at 37° C., 5% CO₂. For the assay imaging microtiter plate Cell Carrier 96 Ultra (Perkin Elmer 6055302) were pre-coated with Fibronectin (Sigma F085, 30 μL at 0.2 μg/mL) in PBS (100 μL, Gibco 14190) for 45 min at room temperature, rinsed with PBS and CAL-51 cells (30K cells/well) were plated and let to adhere overnight. Cells were treated with compounds typically using a serial dilution ranging from 30 μM to 0.1 nM for 6 h. Compounds were stored at 10 mM DMSO stocks. Vehicle (DMSO), positive (CC-885, 10 μM) and rescue controls (positive control plus 0.2 μM bortezomib) were also included at this stage. Cells were subsequently rinsed with PBS and fixed in 10% Formalin solution (50 μL, Sigma HT5011)) for 20 mins at room temperature. Following three consecutive PBS washes (100 μL), cells were permeabilized in 0.1% Triton X-100 in PBS (Sigma 93443, 50 μL) for 15 min. at room temperature. Following three further PBS washes, 50 μL blocking buffer (1% BSA, Sigma A4503, in PBS) was added for 45 min for signal-to noise reduction. Primary antibody (human GSPT1, Sigma HPA052488) was diluted in blocking buffer (dil.1/300, 35 μL/well) and incubated with the cells overnight at 4° C. After three PBS washes, Alexa-fluor 488 coupled secondary antibodies (Invitrogen, A32731, dil. 1/1000), Alexa-fluor 647-Phalloidin (Invitrogen, A22287, dil. 1/200) and DAPI (Thermo, #62248, dil. 1/1000) were diluted in blocking buffer and incubated with the samples for 2 h at room temperature. After three final PBS washes, samples were conserved in 100 μL PBS in the dark, until measurement. Image acquisition was performed on the Operetta High-Content Imager (Perkin-Elmer). Fluorescence intensity of Alexa-F 5 luor 488 (GSPT1), Alexa-Fluor 647 (Actin) and DAPI (Nucleus) were measured. For the determination of GSPT1 DC50 values, a custom algorithm implemented in the PerkinElmer image analysis software Harmony-Acapella® was developed. After user-defined setting of adjustment parameters, the analysis was run identically without human intervention for all image fields. DAPI staining of the nuclei was used to determine the location of cells using standard nuclei detection modules. Segmentation artifacts were removed by threshold-based filters for area, roundness and intensity. The outline of the cells was determined analogously from the sum of the normalized, smoothed DAPI and Actin channel, starting from each nucleus. The Alexa-Fluor 488 (GSPT1) signal intensity in each cell was finally measured, in order to obtain a Mean intensity per cell. GSPT1 degradation (DC50) was calculated after normalization to controls and data import in CDD vault Database, using non-linear regression.

In Table 2, each compound is assigned a class (HTRF class) indicating the ability for Cereblon binding by means of their HTRF IC₅₀ values according to Example 3: A, B, C or D. According to the code, A represents an IC₅₀ value of ≤100 nM, B represents an IC₅₀ value>100 nM and ≤1000 nM, C represents an IC₅₀ value>1000 nM. IF Class assigns each compound a code indicating the ability for GSPT1 degradation: A, B or C. According to the code, A represents a DC₅₀ value of ≤30 nM, B represents a DC₅₀ value>30 nM and ≤300 nM and C represents a DC₅₀ value of >300 nM.

TABLE 2
HTRF Binding of compounds to CRBN
and Activity for GSPT1 degradation.

| Compound | HTRF Class | IF Class |
|---|---|---|
| 1 | B | A |
| 2 | B | A |
| 3 | B | A |
| 4 | B | B |
| 5 | A | B |
| 6 | B | B |
| 7 | B | B |
| 8 | B | A |
| 9 | A | A |
| 10 | A | A |
| 11 | B | A |
| 12 | A | A |
| 13 | A | A |
| 14 | B | A |
| 15 | A | B |
| 16 | B | B |
| 17 | B | B |
| 18 | B | B |
| 19 | B | A |
| 20 | C | B |
| 21 | B | A |
| 22 | B | B |
| 23 | A | A |
| 24 | A | A |
| 25 | A | C |
| 26 | A | A |
| 27 | A | B |
| 28 | B | B |
| 29 | C | B |
| 30 | A | A |
| 31 | B | C |
| 32 | B | A |
| 33 | B | A |
| 34 | B | B |
| 35 | B | C |
| 36 | C | B |
| 37 | B | A |
| 38 | B | B |
| 39 | B | A |
| 40 | B | A |
| 41 | B | C |
| 42 | B | B |
| 43 | B | A |
| 44 | B | B |
| 45 | B | A |
| 46 | B | B |
| 47 | B | B |
| 48 | B | A |
| 49 | B | B |
| 50 | B | B |
| 51 | B | A |
| 52 | B | A |
| 53 | B | B |
| 54 | C | B |
| 55 | C | B |
| 56 | C | B |
| 57 | C | B |
| 58 | B | B |
| 59 | C | C |
| 60 | B | A |
| 61 | B | A |
| 62 | B | C |
| 63 | B | C |
| 64 | A | A |
| 65 | A | A |
| 66 | B | B |
| 67 | B | B |
| 68 | A | B |
| 69 | B | A |
| 70 | B | A |
| 71 | B | C |
| 72 | C | B |
| 73 | B | A |
| 74 | B | C |
| 75 | B | B |
| 76 | A | A |
| 77 | B | A |
| 78 | B | A |
| 79 | B | B |
| 80 | A | A |
| 81 | B | C |
| 82 | B | C |
| 83 | B | C |
| 84 | B | C |
| 85 | B | C |
| 86 | B | C |
| 87 | B | C |
| 88 | B | C |
| 89 | B | C |
| 90 | B | C |
| 91 | B | B |
| 92 | B | C |
| 93 | A | A |
| 94 | B | C |
| 95 | B | C |
| 96 | B | B |
| 97 | A | A |
| 98 | — | A |
| 105 | — | C |
| 106 | — | A |
| 107 | — | B |
| 108 | — | C |
| 109 | — | C |
| 111 | — | C |
| 112 | — | B |
| 113 | — | A |
| 114 | — | C |
| 115 | — | A |
| 116 | — | A |
| 117 | — | C |
| 118 | — | C |
| 119 | — | C |
| 120 | — | C |
| 121 | — | C |
| 122 | — | B |
| 123 | A | A |
| 124 | A | A |
| 125 | B | A |
| 126 | A | C |
| 127 | B | A |
| 128 | B | A |
| 129 | A | B |
| 130 | B | C |
| 131 | B | B |
| 132 | B | C |
| 133 | B | B |
| 134 | A | A |
| 135 | A | A |
| 136 | A | A |
| 137 | A | B |
| 138 | A | A |
| 139 | A | A |
| 140 | A | B |
| 141 | A | C |
| 142 | B | C |
| 143 | B | C |
| 144 | B | C |
| 145 | A | B |

### EQUIVALENTS

While specific embodiments have been discussed, the above specification is illustrative and not restrictive. Many variations of the embodiments will become apparent to those skilled in the art upon review of this specification. The full scope of what is disclosed should be determined by reference to the claims, along with their full scope of equivalents, and the specification, along with such variations.

What is claimed is:

1. A compound of Formula (X): 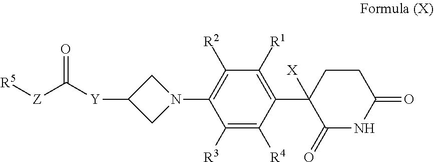
or a pharmaceutically acceptable salt thereof,
wherein:
X is H or deuterium;
Y is O or NH;
Z is NR^{A};
each of R¹, R², R³, and R⁴ is independently H, halogen, cyano, or C₁₋₆ alkoxy;
R⁵ is C₁₋₆ alkyl, C₂₋₆ alkenyl, C₂₋₆ alkynyl, C₃₋₁₀ cycloalkyl, 3 to 10 membered heterocyclyl, aryl, or heteroaryl, wherein each of C₁₋₆ alkyl, C₂₋₆ alkenyl, C₂₋₆ alkynyl, C³⁻¹⁰ cycloalkyl, C₆₋₁₀ aryl, and heteroaryl is optionally substituted with one or more substituents each independently selected from R⁶;
or R^{A} and R⁵ are joined together to form a 3 to 10 membered nitrogen-containing heterocyclyl optionally substituted with one or more substituents each independently selected from R⁶;
each occurrence of R⁶ is independently halogen, cyano, —S(CH₃), C₁₋₆ alkyl, C₁₋₆ alkoxy, C₁₋₆ thioalkoxy, C₃₋₁₀ cycloalkyl, 3 to 10 membered heterocyclyl, aryl, heteroaryl, —C(O)OR^{B}, or —OC(O)R^{C}, wherein each of C₁₋₆ alkyl, C₁₋₆ alkoxy, C₃₋₁₀ cycloalkyl, 3 to 10 membered heterocyclyl, aryl, and heteroaryl is optionally substituted with one or more substituents each independently selected from R⁷,
or two R⁶ are joined to together to form a 3 to 10 membered ring optionally substituted with one or more substituents each independently selected from R⁷;
each occurrence of R⁷ is independently halogen, —OH, C₁₋₆ alkyl, 3 to 10 membered heterocyclyl, or aryl, wherein each of C₁₋₆ alkyl, 3 to 10 membered heterocyclyl, and aryl is optionally substituted with R⁸;
each occurrence of R⁸ is C₁₋₆ alkoxy;
R^{A} is H or C₁₋₃ alkyl, or R^{A} and R⁵ are joined together to form a 3 to 10 membered nitrogen-containing heterocyclyl optionally substituted with one or more substituents each independently selected from R⁶; and
each of R^{B} and R^{C} is independently H or C₁₋₆ alkyl.

2. The compound of claim 1, wherein the compound is a compound of Formula (I): 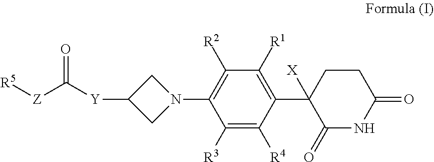
or a pharmaceutically acceptable salt thereof,
wherein:
X is H or deuterium;
Y is O or NH;
Z is NR^{A};
each of R¹, R², R³, and R⁴ is independently H, halogen, cyano, or C₁₋₆ alkoxy;
R⁵ is C₁₋₆ alkyl, C₂₋₆ alkenyl, C₂₋₆ alkynyl, C₃₋₁₀ cycloalkyl, 3 to 10 membered heterocyclyl, aryl, or heteroaryl, wherein each of C₁₋₆ alkyl, C₂₋₆ alkenyl, C₂₋₆ alkynyl, C³⁻¹⁰ cycloalkyl, C₆₋₁₀ aryl, and heteroaryl is optionally substituted with one or more occurrences of R⁶;
or R^{A} and R⁵ are joined together to form a 3 to 10 membered nitrogen-containing heterocyclyl optionally substituted with one or more occurrences of R⁶;
each occurrence of R⁶ is independently halogen, cyano, C₁₋₆ alkyl, C₁₋₆ alkoxy, C₁₋₆ thioalkoxy, C₃₋₁₀ cycloalkyl, 3 to 10 membered heterocyclyl, aryl, heteroaryl, —C(O)OR^{B}, or —OC(O)R^{C}, wherein each of C₁₋₆ alkyl, C₁₋₆ alkoxy, C₃₋₁₀ cycloalkyl, 3 to 10 membered heterocyclyl, aryl, and heteroaryl is optionally substituted with one or more occurrences of R⁷,
or two R⁶ are joined to together to form a 3 to 10 membered ring optionally substituted with one or more occurrences of R⁷;
each occurrence of R⁷ is independently halogen, C₁₋₆ alkyl, 3 to 10 membered heterocyclyl, or aryl, wherein each of C₁₋₆ alkyl, 3 to 10 membered heterocyclyl, and aryl is optionally substituted with R⁸;
each occurrence of R⁸ is C₁₋₆ alkoxy;
R^{A} is H, or R^{A} and R⁵ are joined together to form a 3 to 10 membered nitrogen-containing heterocyclyl optionally substituted with one or more occurrences of R⁶; and
each of R^{B} and R^{C} is independently H or C₁₋₆ alkyl.

3. The compound of claim 1, wherein X is H, Y is O or NH, and Z is O or NH.

4. The compound of claim 1, wherein R⁶ is halogen, cyano, C₁₋₆ alkyl, or C₁₋₆ alkoxy, wherein each of C₁₋₆ alkyl or C₁₋₆ alkoxy is optionally substituted with one or more occurrences of halogen, and R⁷ is halogen or C₁₋₆ alkyl.

5. The compound of claim 1, wherein m is 1, 2, or 3, n is independently 1, 2, or 3, and p is 1.

6. A compound of Formula (II): 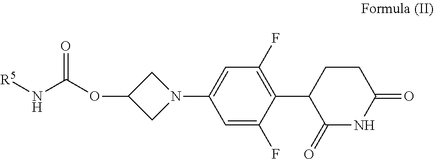
or a pharmaceutically acceptable salt thereof,
wherein:
R⁵ is heteroaryl, aryl, or C₃₋₁₀ cycloalkyl, wherein each of heteroaryl, aryl, and C₃₋₁₀ cycloalkyl is optionally substituted with one or more occurrences of R⁶;
each occurrence of R⁶ is independently halogen, cyano, C₁₋₆ alkyl, C₁₋₆ alkoxy, C³⁻¹⁰ cycloalkyl, C₃₋₁₀ heterocyclyl, phenyl, or 5-membered heteroaryl, or 6-membered heteroaryl, wherein each of C₁₋₆ alkyl, C₁₋₆ alkoxy, C₃₋₁₀ cycloalkyl, 3 to 10 membered heterocyclyl, phenyl, 5-membered heteroaryl, and 6-membered heteroaryl is optionally substituted with one or more occurrences of R⁷; and
each occurrence of R⁷ is independently halogen or C₁₋₆ alkyl.

7. The compound of claim 6, wherein R⁵ is C₃₋₁₀ cycloalkyl, C⁶⁻¹⁰ aryl, 5-membered heteroaryl, or 6-membered heteroaryl.

8. The compound of claim 6, wherein R⁶ is halogen, cyano, C₁₋₆ alkyl, or C₁₋₆ alkoxy, wherein each of C₁₋₆ alkyl or C₁₋₆ alkoxy is optionally substituted with one or more occurrences of halogen, and R⁷ is halogen or C₁₋₆ alkyl.

9. The compound of claim 6, wherein m is 1, 2, or 3, each occurrence of n is independently 1, 2, or 3, and p is 1.

10. A compound of Formula (III): 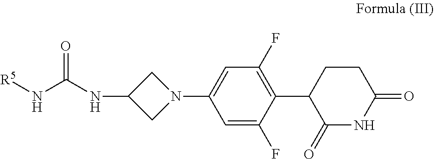
or a pharmaceutically acceptable salt thereof,
wherein:
R⁵ is heteroaryl, aryl, or C₃₋₁₀ cycloalkyl, wherein each of heteroaryl, aryl, and C₃₋₁₀ cycloalkyl is optionally substituted with one or more substituents each independently selected from R⁶;
each occurrence of R⁶ is independently halogen, cyano, C₁₋₆ alkyl, C₁₋₆ alkoxy, C³⁻¹⁰ cycloalkyl, 3 to 10 membered heterocyclyl, phenyl, or 5-membered heteroaryl, or 6-membered heteroaryl, wherein each of C₁₋₆ alkyl, C₁₋₆ alkoxy, C₃₋₁₀ cycloalkyl, 3 to 10 membered heterocyclyl, phenyl, 5-membered heteroaryl, and 6-membered heteroaryl is optionally substituted with one or more substituents each independently selected from R⁷; and
each occurrence of R⁷ is independently halogen or C₁₋₆ alkyl.

11. The compound of claim 10, wherein R⁵ is C₃₋₁₀ cycloalkyl, C₆₋₁₀ aryl, 5-membered heteroaryl, or 6-membered heteroaryl.

12. The compound of claim 10, wherein R⁶ is halogen, cyano, C₁₋₆ alkyl, or C₁₋₆ alkoxy, wherein each of C₁₋₆ alkyl or C₁₋₆ alkoxy is optionally substituted with one or more occurrences of halogen, and R⁷ is halogen or C₁₋₆ alkyl.

13. The compound of claim 10, wherein m is 1, 2, or 3, each occurrence of n is independently 1, 2, or 3, and p is 1.

14. A compound of Formula (XI): 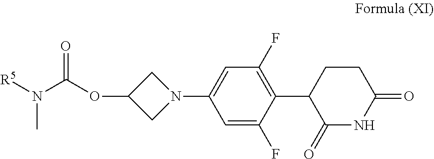
or a pharmaceutically acceptable salt thereof,
wherein:
R⁵ is C₁₋₆ alkyl, C₂₋₆ alkenyl, C₂₋₆ alkynyl, C₃₋₁₀ cycloalkyl, 3 to 10 membered heterocyclyl, aryl, or heteroaryl, wherein each of C₁₋₆ alkyl, C₂₋₆ alkenyl, C₂₋₆ alkynyl, C³⁻¹⁰ cycloalkyl, C₆₋₁₀ aryl, and heteroaryl is optionally substituted with one or more substituents each independently selected from R⁶;
each occurrence of R⁶ is independently halogen, cyano, C₁₋₆ alkyl, C₁₋₆ alkoxy, C³⁻¹⁰ cycloalkyl, 3 to 10 membered heterocyclyl, phenyl, or 5-membered heteroaryl, or 6-membered heteroaryl, wherein each of C₁₋₆ alkyl, C₁₋₆ alkoxy, C₃₋₁₀ cycloalkyl, 3 to 10 membered heterocyclyl, phenyl, 5-membered heteroaryl, and 6-membered heteroaryl is optionally substituted with one or more substituents each independently selected from R⁷; and
each occurrence of R⁷ is independently halogen or C₁₋₆ alkyl.

15. The compound of claim 14, wherein R⁶ is halogen, cyano, C₁₋₆ alkyl, or C₁₋₆ alkoxy, wherein each of C₁₋₆ alkyl or C₁₋₆ alkoxy is optionally substituted with one or more occurrences of halogen, and R⁷ is halogen or C₁₋₆ alkyl.

16. The compound of claim 14, wherein m is 1, 2, or 3, each occurrence of n is independently 1, 2, or 3, and p is 1.

17. A compound selected from the group consisting of: 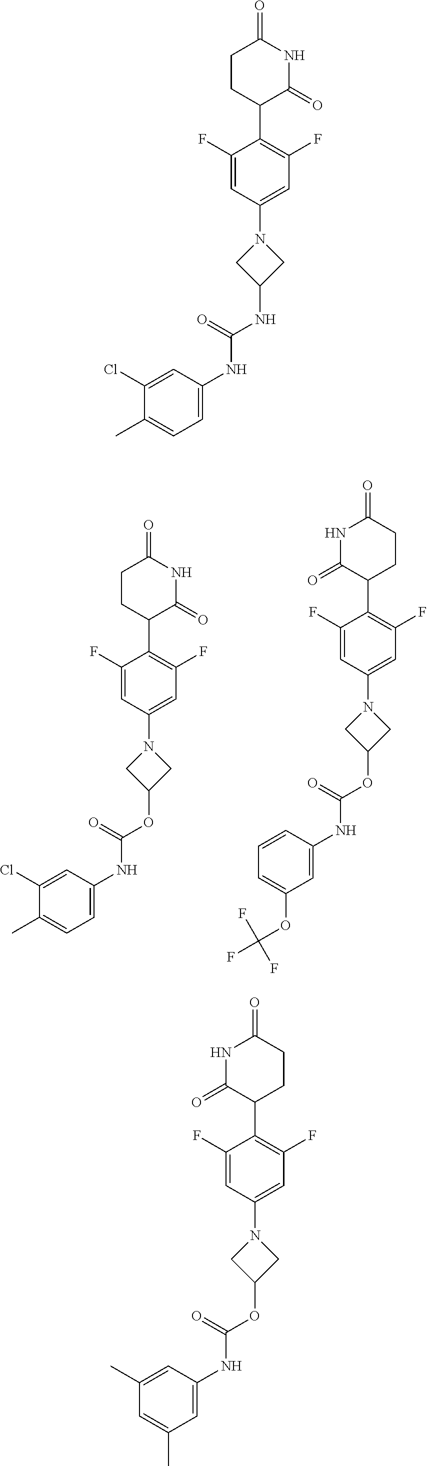 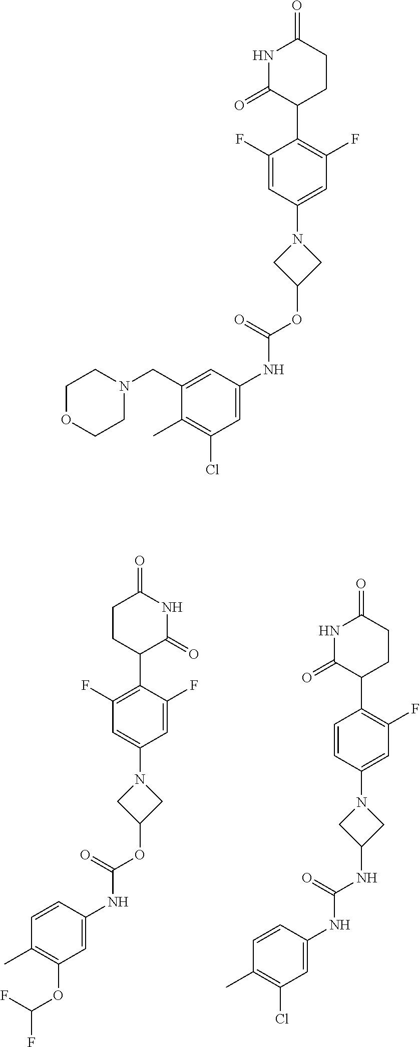 [SMILES: Cc1c(Cl)cc(NC(=O)OC2CN(c3cc(F)c(C4CCC(=O)NC4=O)c(F)c3)C2)cc1CN1CCOCC1 ; Cc1ccc(NC(=O)OC2CN(c3cc(F)c(C4CCC(=O)NC4=O)c(F)c3)C2)cc1OC(F)F ; Cc1ccc(NC(=O)NC2CN(c3ccc(C4CCC(=O)NC4=O)c(F)c3)C2)cc1Cl] 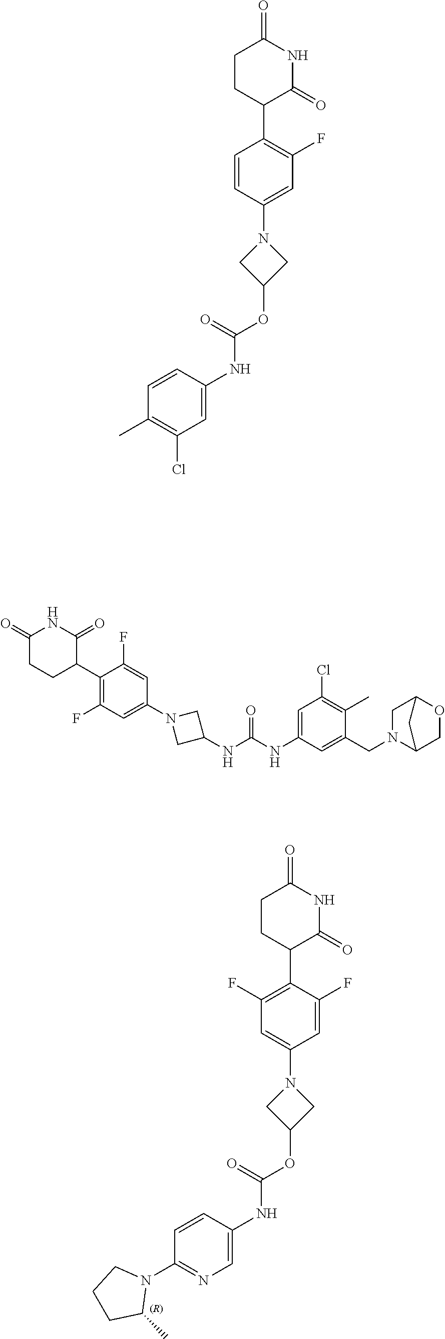 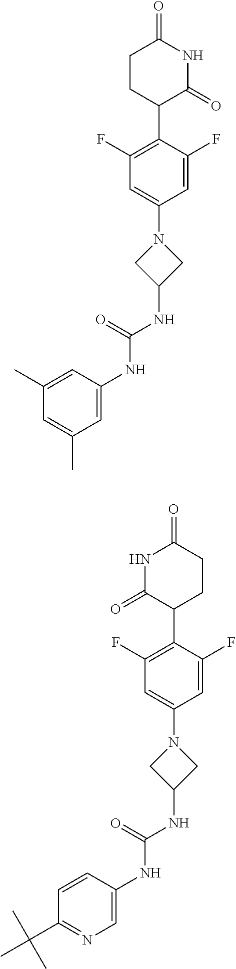 [SMILES: Cc1cc(C)cc(NC(=O)NC2CN(c3cc(F)c(C4CCC(=O)NC4=O)c(F)c3)C2)c1 ; CC(C)(C)c1ccc(NC(=O)NC2CN(c3cc(F)c(C4CCC(=O)NC4=O)c(F)c3)C2)cn1] 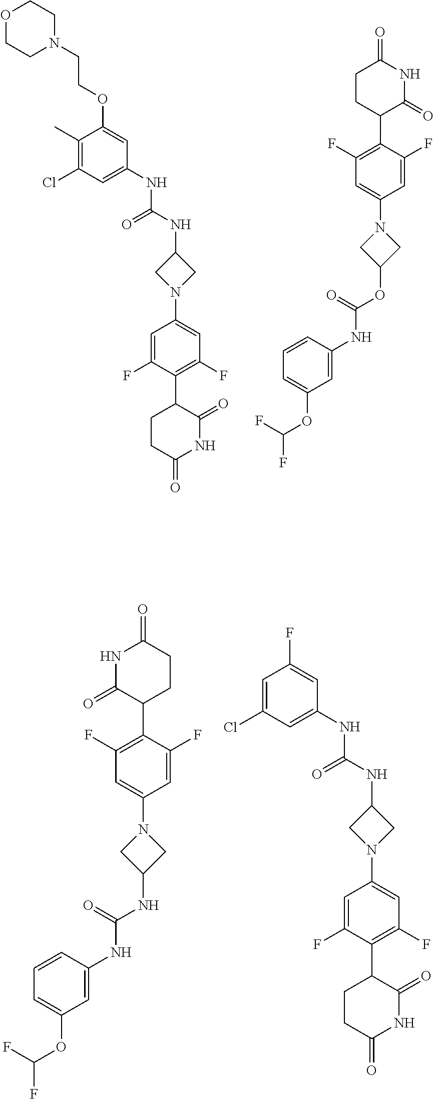 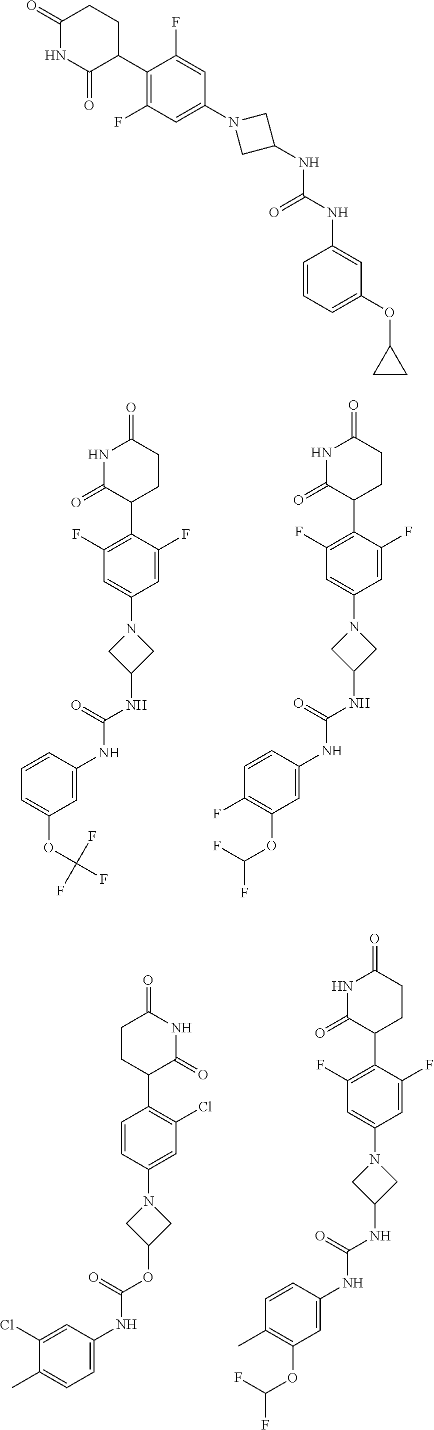 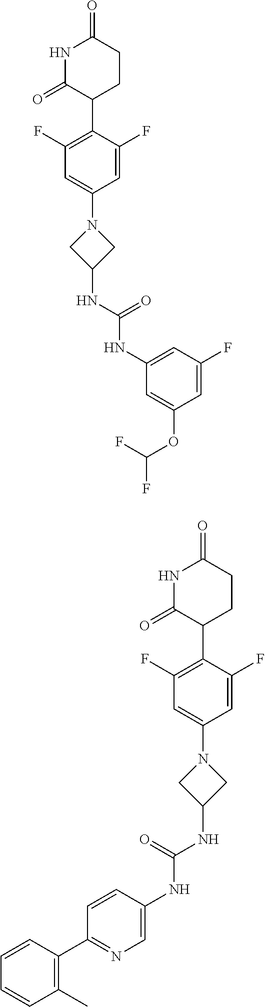 [SMILES: O=C1CCC(c2c(F)cc(N3CC(NC(=O)Nc4cc(F)cc(OC(F)F)c4)C3)cc2F)C(=O)N1 ; Cc1ccccc1-c1ccc(NC(=O)NC2CN(c3cc(F)c(C4CCC(=O)NC4=O)c(F)c3)C2)cn1] 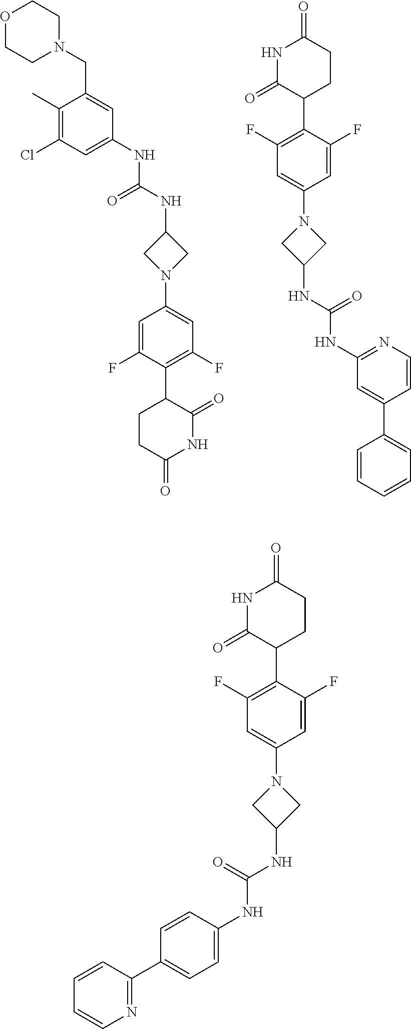 [SMILES: Cc1c(Cl)cc(NC(=O)NC2CN(c3cc(F)c(C4CCC(=O)NC4=O)c(F)c3)C2)cc1CN1CCOCC1 ; O=C1CCC(c2c(F)cc(N3CC(NC(=O)Nc4cc(-c5ccccc5)ccn4)C3)cc2F)C(=O)N1 ; O=C1CCC(c2c(F)cc(N3CC(NC(=O)Nc4ccc(-c5ccccn5)cc4)C3)cc2F)C(=O)N1] 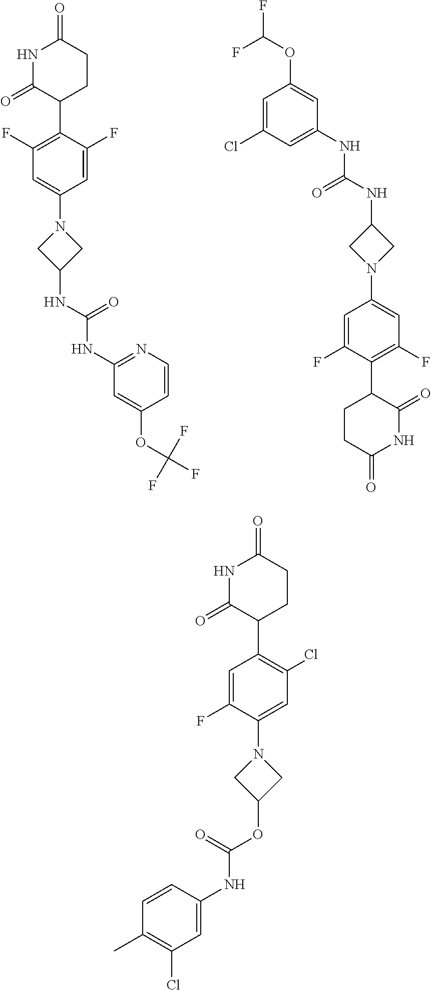 [SMILES: O=C1CCC(c2c(F)cc(N3CC(NC(=O)Nc4cc(OC(F)(F)F)ccn4)C3)cc2F)C(=O)N1 ; O=C1CCC(c2c(F)cc(N3CC(NC(=O)Nc4cc(Cl)cc(OC(F)F)c4)C3)cc2F)C(=O)N1 ; Cc1ccc(NC(=O)OC2CN(c3cc(Cl)c(C4CCC(=O)NC4=O)cc3F)C2)cc1Cl] 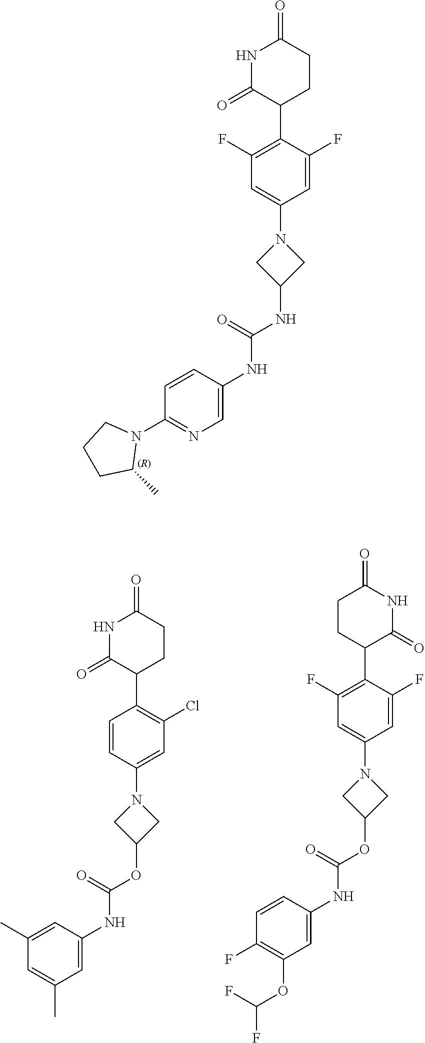 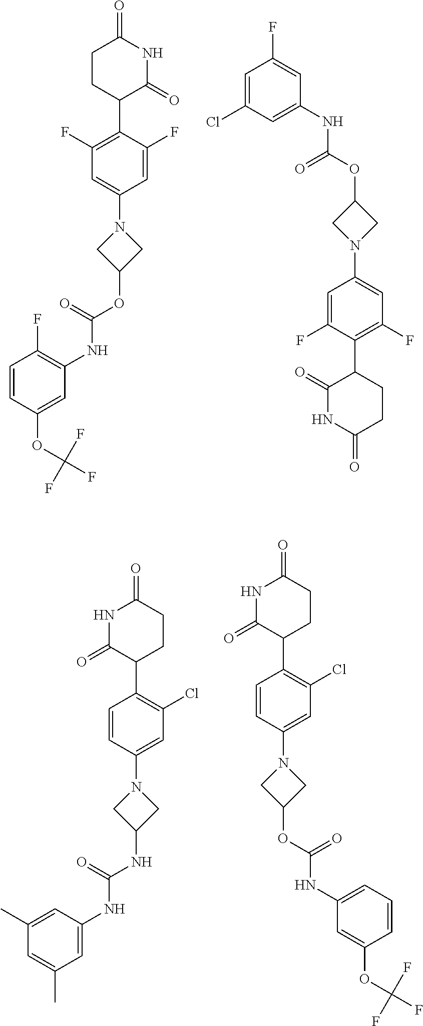 [SMILES: O=C1CCC(c2c(F)cc(N3CC(OC(=O)Nc4cc(OC(F)(F)F)ccc4F)C3)cc2F)C(=O)N1 ; O=C1CCC(c2c(F)cc(N3CC(OC(=O)Nc4cc(F)cc(Cl)c4)C3)cc2F)C(=O)N1 ; Cc1cc(C)cc(NC(=O)NC2CN(c3ccc(C4CCC(=O)NC4=O)c(Cl)c3)C2)c1 ; O=C1CCC(c2ccc(N3CC(OC(=O)Nc4cccc(OC(F)(F)F)c4)C3)cc2Cl)C(=O)N1] 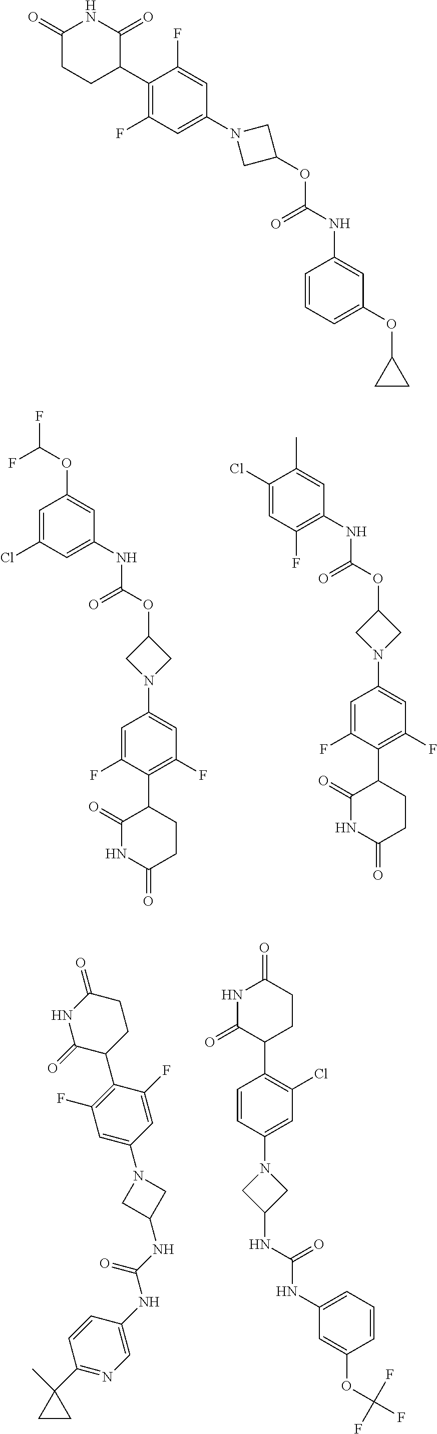 [SMILES: O=C1CCC(c2c(F)cc(N3CC(OC(=O)Nc4cccc(OC5CC5)c4)C3)cc2F)C(=O)N1 ; O=C1CCC(c2c(F)cc(N3CC(OC(=O)Nc4cc(Cl)cc(OC(F)F)c4)C3)cc2F)C(=O)N1 ; Cc1cc(NC(=O)OC2CN(c3cc(F)c(C4CCC(=O)NC4=O)c(F)c3)C2)c(F)cc1Cl ; CC1(c2ccc(NC(=O)NC3CN(c4cc(F)c(C5CCC(=O)NC5=O)c(F)c4)C3)cn2)CC1 ; O=C1CCC(c2ccc(N3CC(NC(=O)Nc4cccc(OC(F)(F)F)c4)C3)cc2Cl)C(=O)N1] 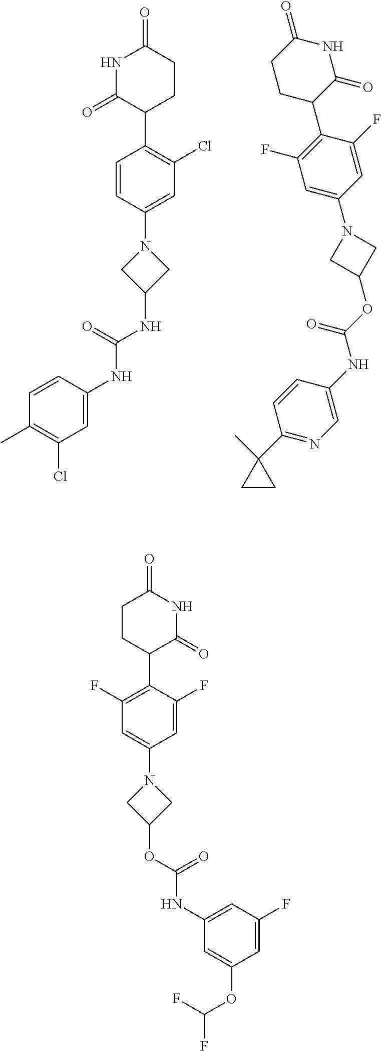 [SMILES: Cc1ccc(NC(=O)NC2CN(c3ccc(C4CCC(=O)NC4=O)c(Cl)c3)C2)cc1Cl ; CC1(c2ccc(NC(=O)OC3CN(c4cc(F)c(C5CCC(=O)NC5=O)c(F)c4)C3)cn2)CC1 ; O=C1CCC(c2c(F)cc(N3CC(OC(=O)Nc4cc(F)cc(OC(F)F)c4)C3)cc2F)C(=O)N1] 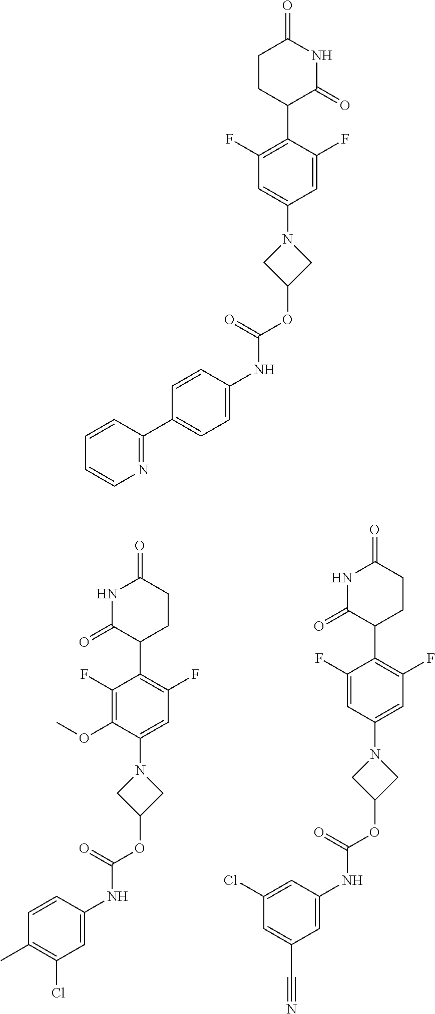 [SMILES: O=C1CCC(c2c(F)cc(N3CC(OC(=O)Nc4ccc(-c5ccccn5)cc4)C3)cc2F)C(=O)N1 ; COc1c(N2CC(OC(=O)Nc3ccc(C)c(Cl)c3)C2)cc(F)c(C2CCC(=O)NC2=O)c1F ; N#Cc1cc(Cl)cc(NC(=O)OC2CN(c3cc(F)c(C4CCC(=O)NC4=O)c(F)c3)C2)c1] 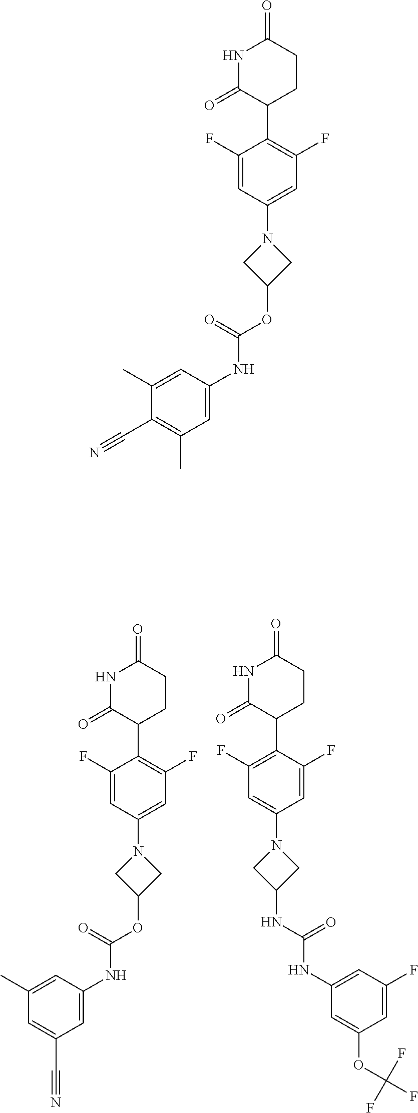 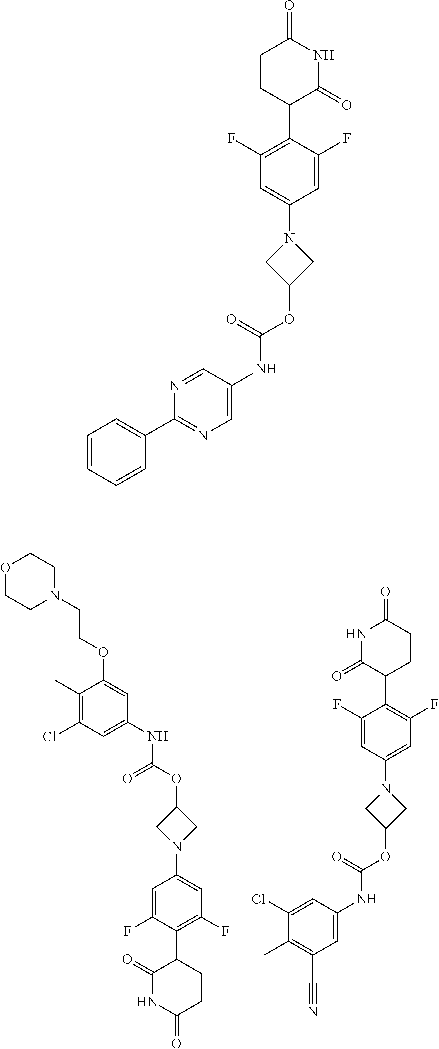 [SMILES: O=C1CCC(c2c(F)cc(N3CC(OC(=O)Nc4cnc(-c5ccccc5)nc4)C3)cc2F)C(=O)N1 ; Cc1c(Cl)cc(NC(=O)OC2CN(c3cc(F)c(C4CCC(=O)NC4=O)c(F)c3)C2)cc1OCCN1CCOCC1 ; Cc1c(Cl)cc(NC(=O)OC2CN(c3cc(F)c(C4CCC(=O)NC4=O)c(F)c3)C2)cc1C#N] 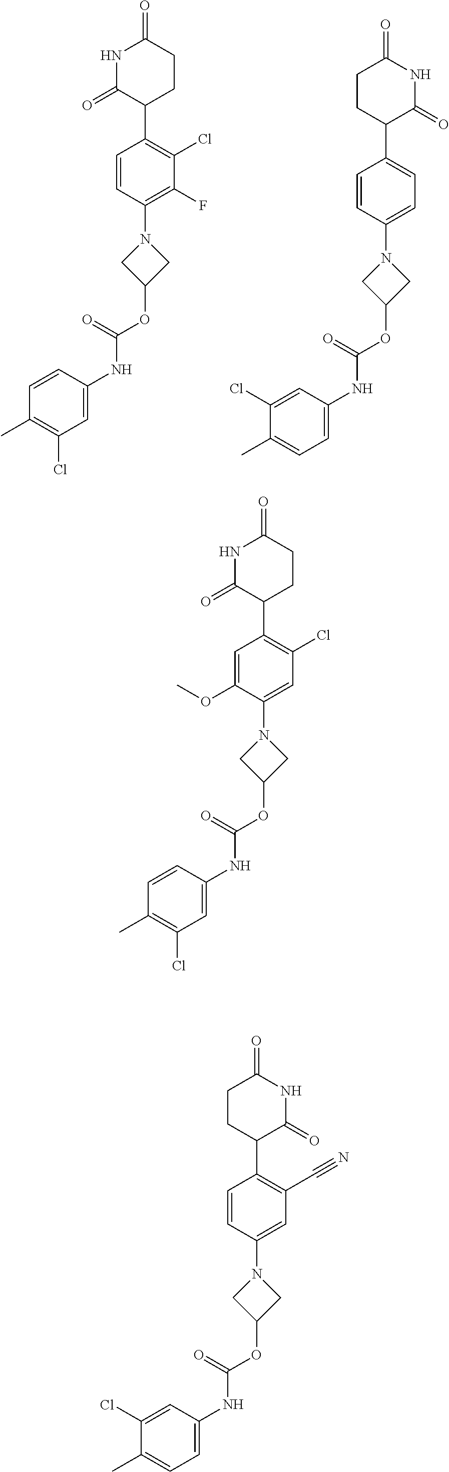 [SMILES: Cc1ccc(NC(=O)OC2CN(c3ccc(C4CCC(=O)NC4=O)c(Cl)c3F)C2)cc1Cl ; Cc1ccc(NC(=O)OC2CN(c3ccc(C4CCC(=O)NC4=O)cc3)C2)cc1Cl ; COc1cc(C2CCC(=O)NC2=O)c(Cl)cc1N1CC(OC(=O)Nc2ccc(C)c(Cl)c2)C1 ; Cc1ccc(NC(=O)OC2CN(c3ccc(C4CCC(=O)NC4=O)c(C#N)c3)C2)cc1Cl] 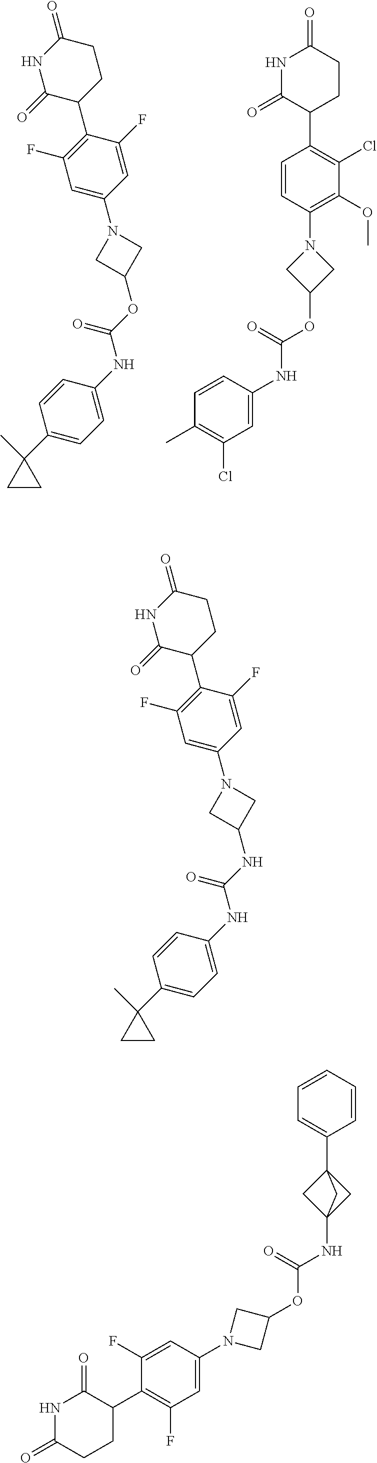 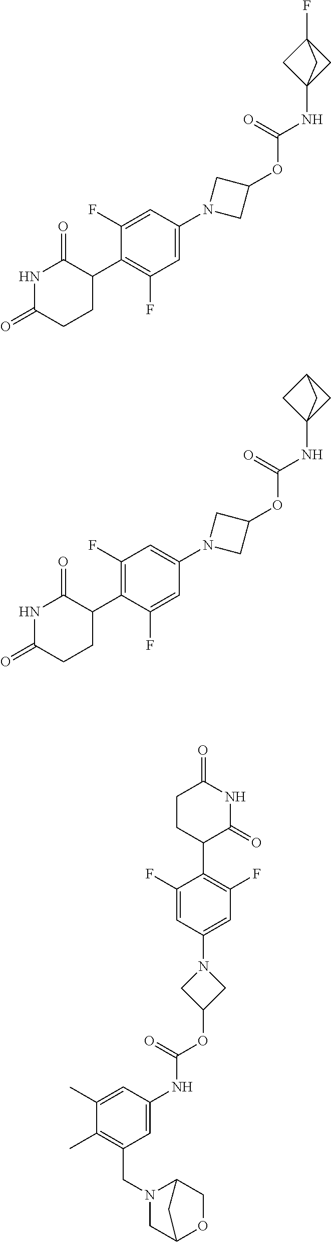 [SMILES: O=C1CCC(c2c(F)cc(N3CC(OC(=O)NC45CC(F)(C4)C5)C3)cc2F)C(=O)N1 ; O=C1CCC(c2c(F)cc(N3CC(OC(=O)NC45CC(C4)C5)C3)cc2F)C(=O)N1 ; Cc1cc(NC(=O)OC2CN(c3cc(F)c(C4CCC(=O)NC4=O)c(F)c3)C2)cc(CN2CC3CC2CO3)c1C] 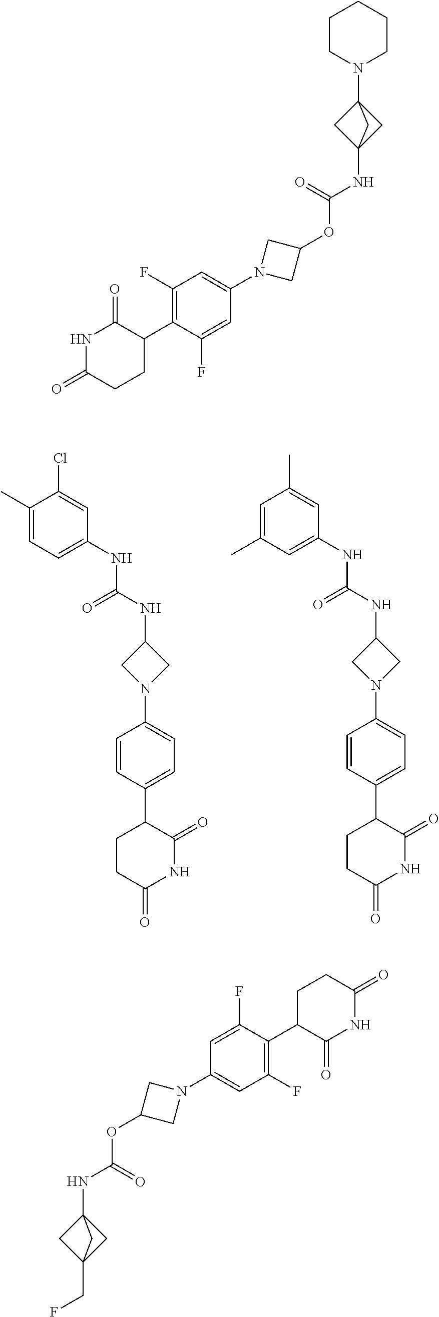 [SMILES: O=C1CCC(c2c(F)cc(N3CC(OC(=O)NC45CC(N6CCCCC6)(C4)C5)C3)cc2F)C(=O)N1 ; Cc1ccc(NC(=O)NC2CN(c3ccc(C4CCC(=O)NC4=O)cc3)C2)cc1Cl ; Cc1cc(C)cc(NC(=O)NC2CN(c3ccc(C4CCC(=O)NC4=O)cc3)C2)c1 ; O=C1CCC(c2c(F)cc(N3CC(OC(=O)NC45CC(CF)(C4)C5)C3)cc2F)C(=O)N1] 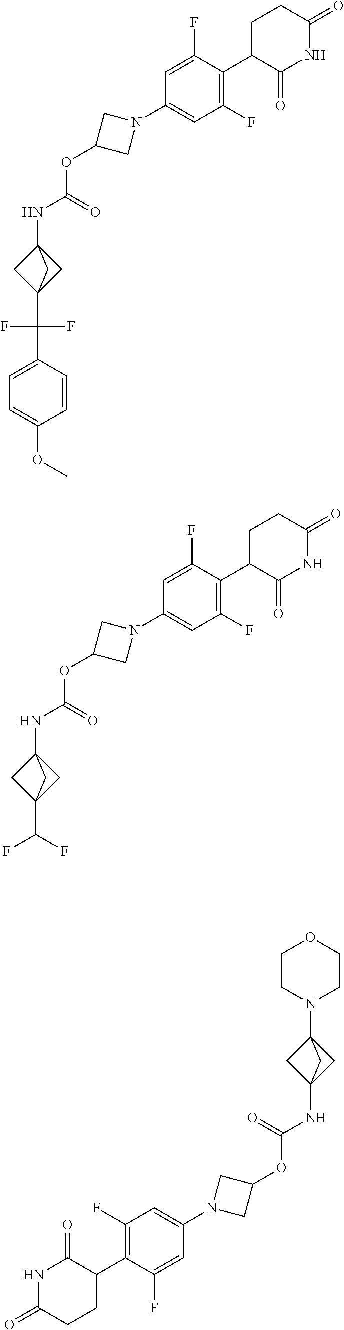 [SMILES: COc1ccc(C(F)(F)C23CC(NC(=O)OC4CN(c5cc(F)c(C6CCC(=O)NC6=O)c(F)c5)C4)(C2)C3)cc1 ; O=C1CCC(c2c(F)cc(N3CC(OC(=O)NC45CC(C(F)F)(C4)C5)C3)cc2F)C(=O)N1 ; O=C1CCC(c2c(F)cc(N3CC(OC(=O)NC45CC(N6CCOCC6)(C4)C5)C3)cc2F)C(=O)N1] 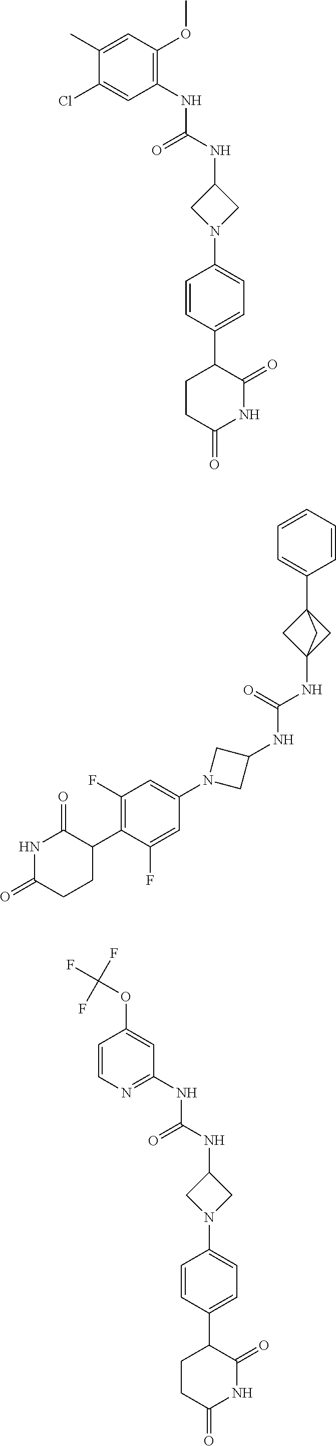 [SMILES: COc1cc(C)c(Cl)cc1NC(=O)NC1CN(c2ccc(C3CCC(=O)NC3=O)cc2)C1 ; O=C1CCC(c2c(F)cc(N3CC(NC(=O)NC45CC(c6ccccc6)(C4)C5)C3)cc2F)C(=O)N1 ; O=C1CCC(c2ccc(N3CC(NC(=O)Nc4cc(OC(F)(F)F)ccn4)C3)cc2)C(=O)N1] 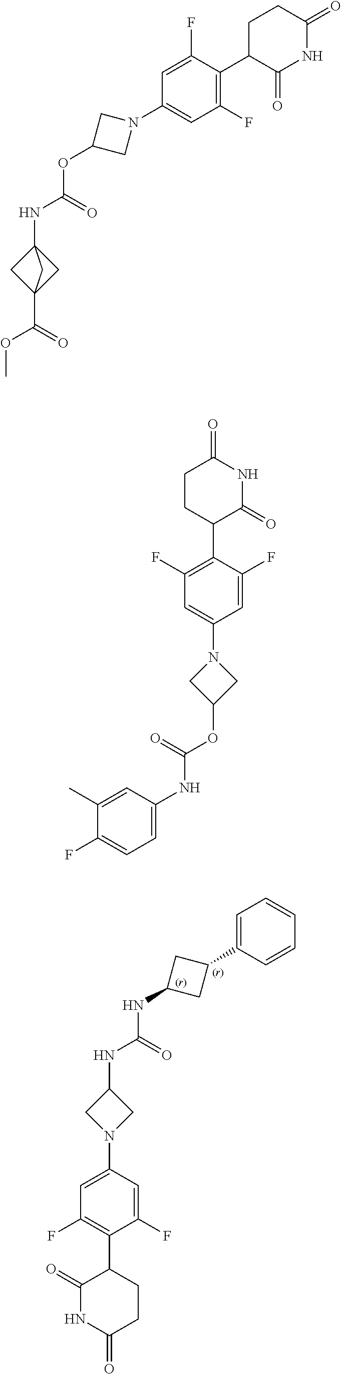 [SMILES: COC(=O)C12CC(NC(=O)OC3CN(c4cc(F)c(C5CCC(=O)NC5=O)c(F)c4)C3)(C1)C2 ; Cc1cc(NC(=O)OC2CN(c3cc(F)c(C4CCC(=O)NC4=O)c(F)c3)C2)ccc1F ; O=C1CCC(c2c(F)cc(N3CC(NC(=O)N[C@H]4C[C@H](c5ccccc5)C4)C3)cc2F)C(=O)N1] 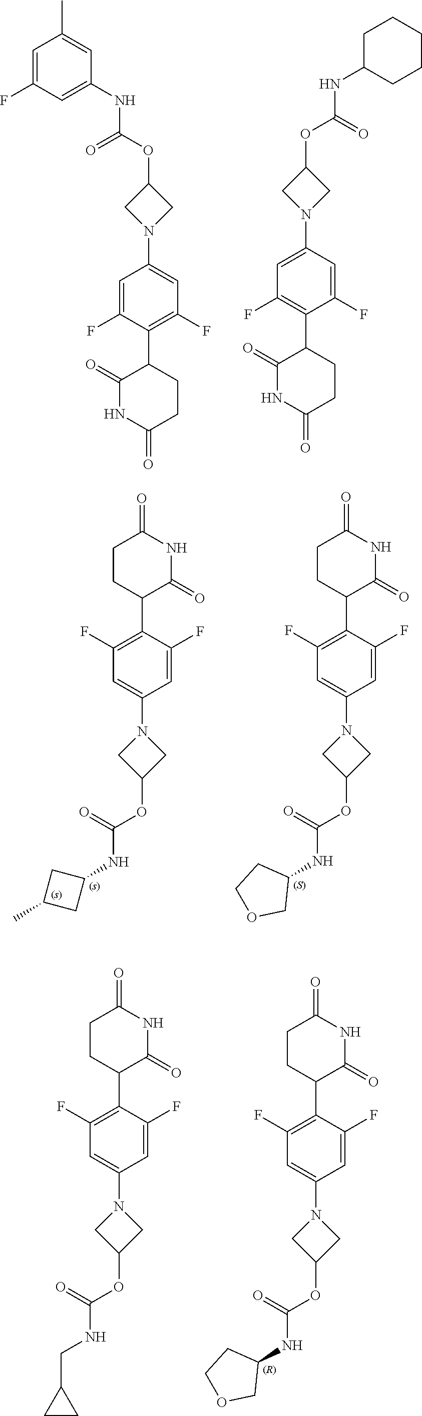 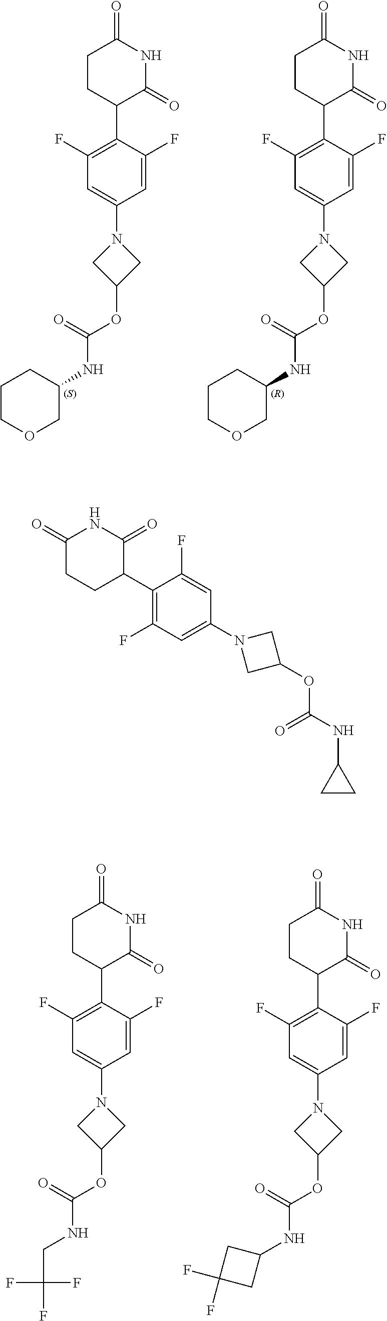 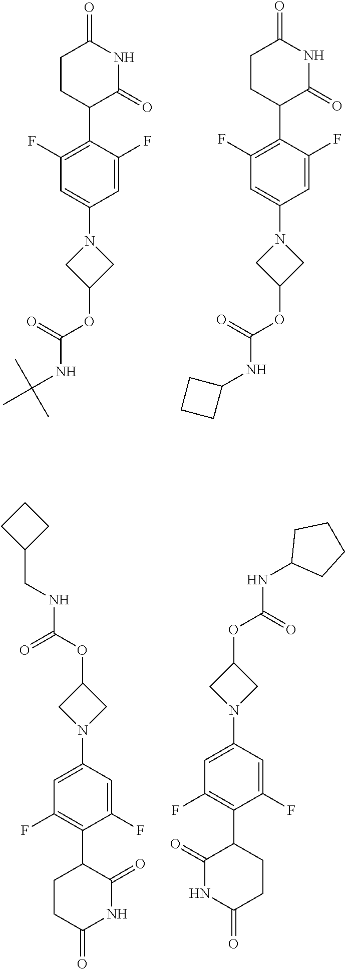 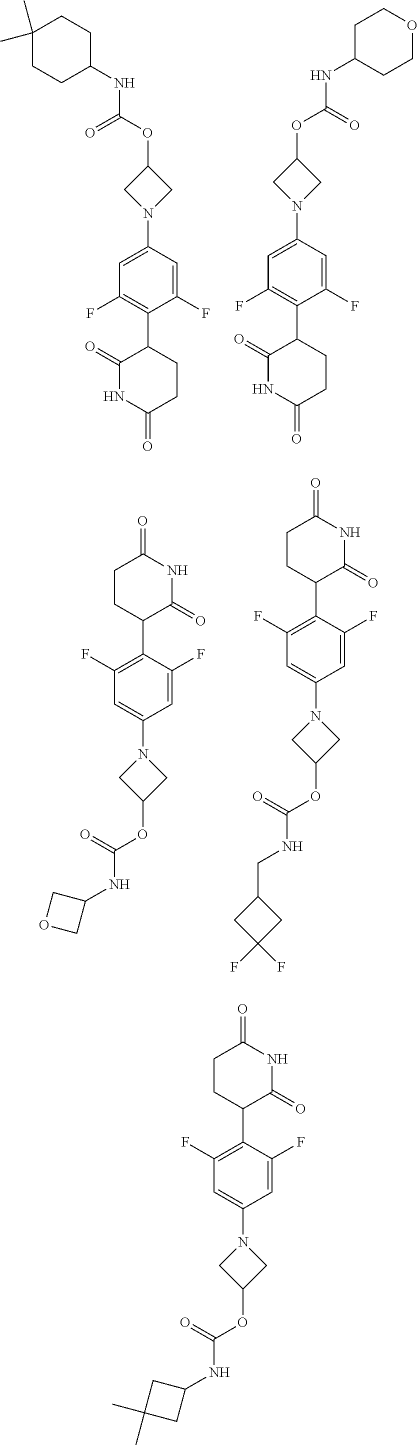 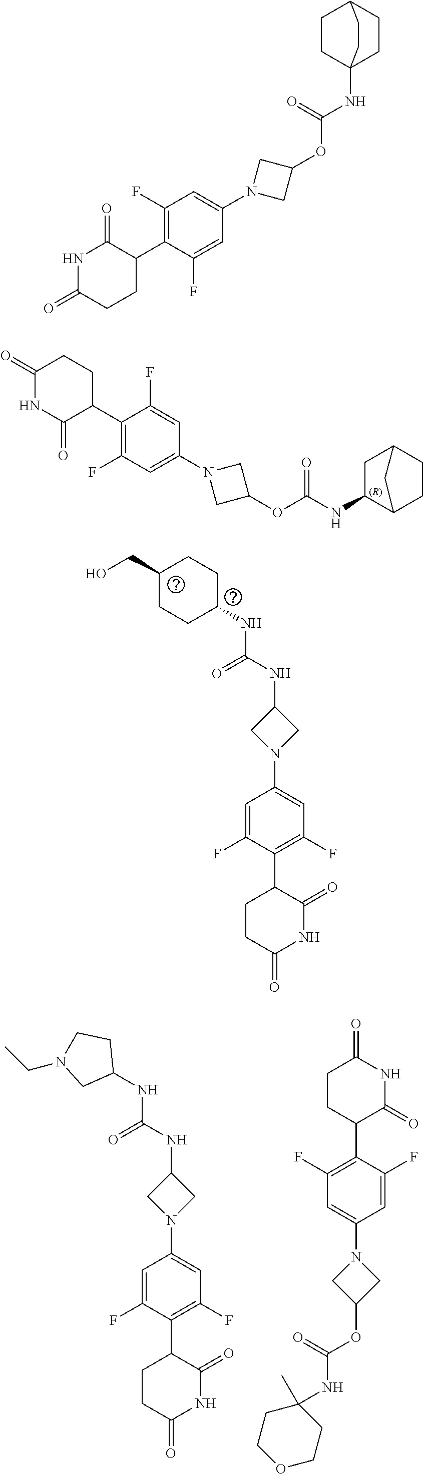 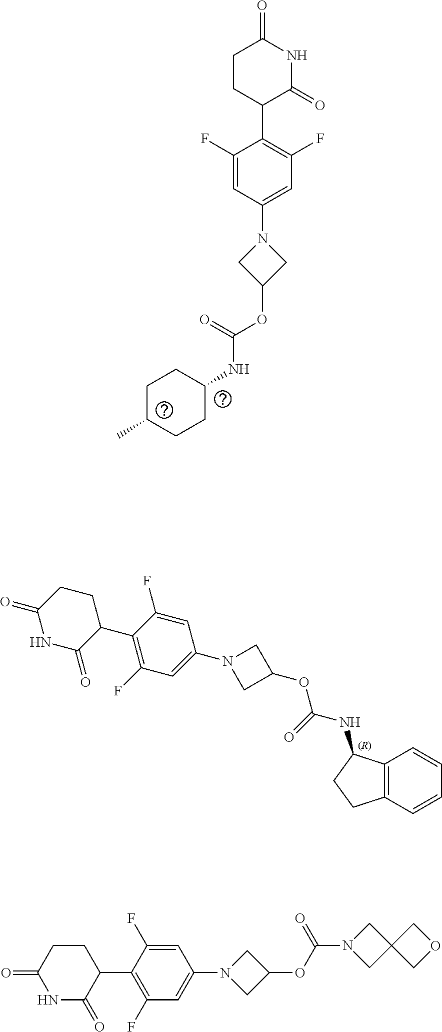 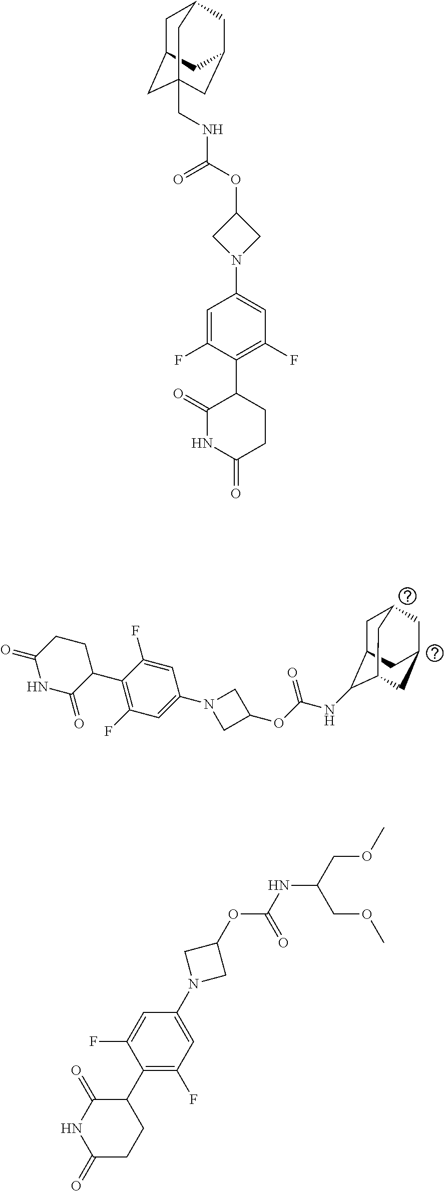 [SMILES: O=C1CCC(c2c(F)cc(N3CC(OC(=O)NCC45C[C@H]6C[C@@H](C4)C[C@@H](C5)C6)C3)cc2F)C(=O)N1 ; O=C1CCC(c2c(F)cc(N3CC(OC(=O)NC4[C@H]5C[C@@H]6C[C@@H](C[C@H]4C6)C5)C3)cc2F)C(=O)N1 ; COCC(COC)NC(=O)OC1CN(c2cc(F)c(C3CCC(=O)NC3=O)c(F)c2)C1] 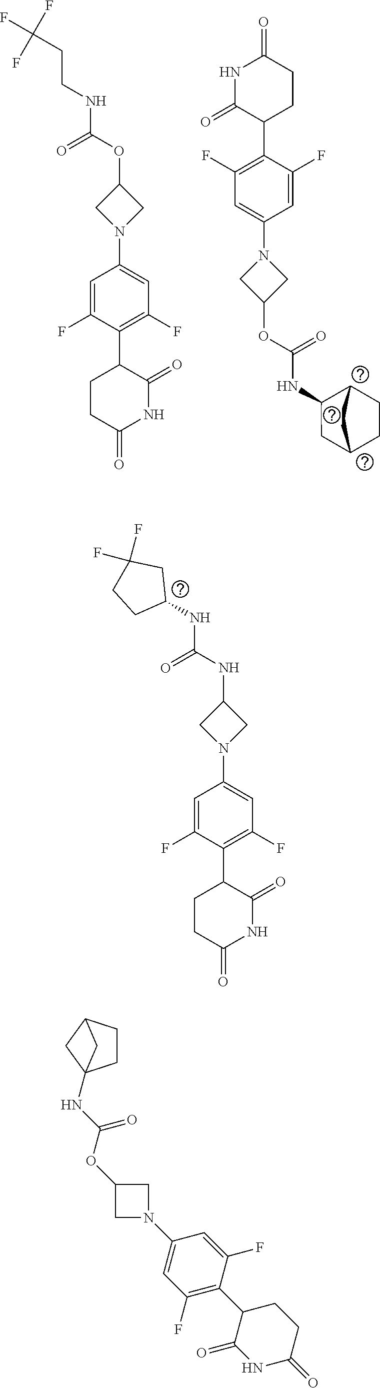 [SMILES: O=C1CCC(c2c(F)cc(N3CC(OC(=O)NCCC(F)(F)F)C3)cc2F)C(=O)N1 ; O=C1CCC(c2c(F)cc(N3CC(OC(=O)N[C@@H]4C[C@H]5CC[C@@H]4C5)C3)cc2F)C(=O)N1 ; O=C1CCC(c2c(F)cc(N3CC(NC(=O)N[C@@H]4CCC(F)(F)C4)C3)cc2F)C(=O)N1 ; O=C1CCC(c2c(F)cc(N3CC(OC(=O)NC45CCC(C4)C5)C3)cc2F)C(=O)N1] 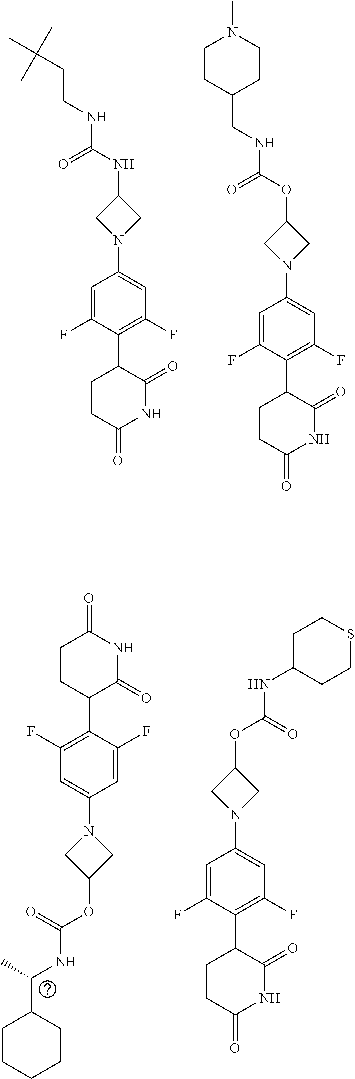 [SMILES: CC(C)(C)CCNC(=O)NC1CN(c2cc(F)c(C3CCC(=O)NC3=O)c(F)c2)C1 ; CN1CCC(CNC(=O)OC2CN(c3cc(F)c(C4CCC(=O)NC4=O)c(F)c3)C2)CC1 ; C[C@H](NC(=O)OC1CN(c2cc(F)c(C3CCC(=O)NC3=O)c(F)c2)C1)C1CCCCC1 ; O=C1CCC(c2c(F)cc(N3CC(OC(=O)NC4CCSCC4)C3)cc2F)C(=O)N1] 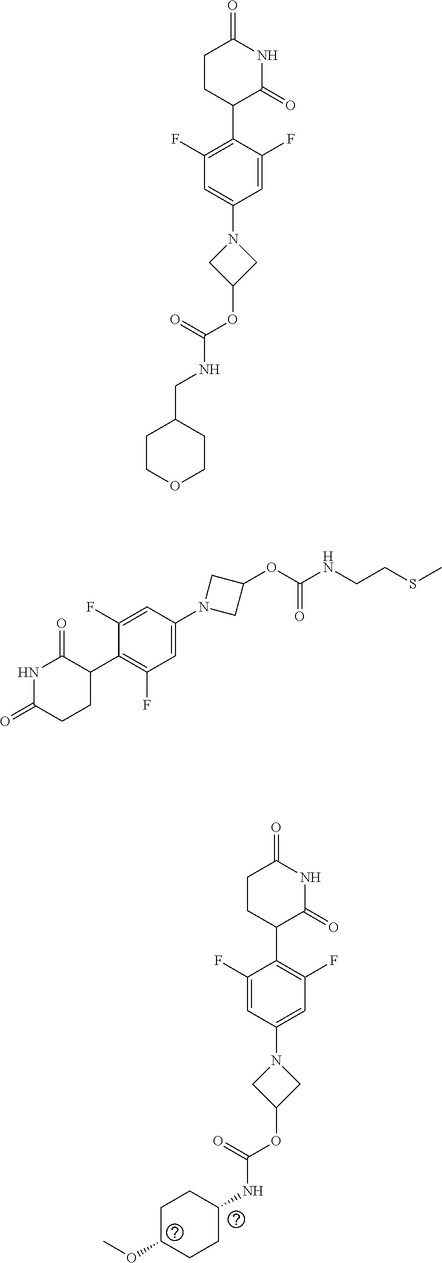 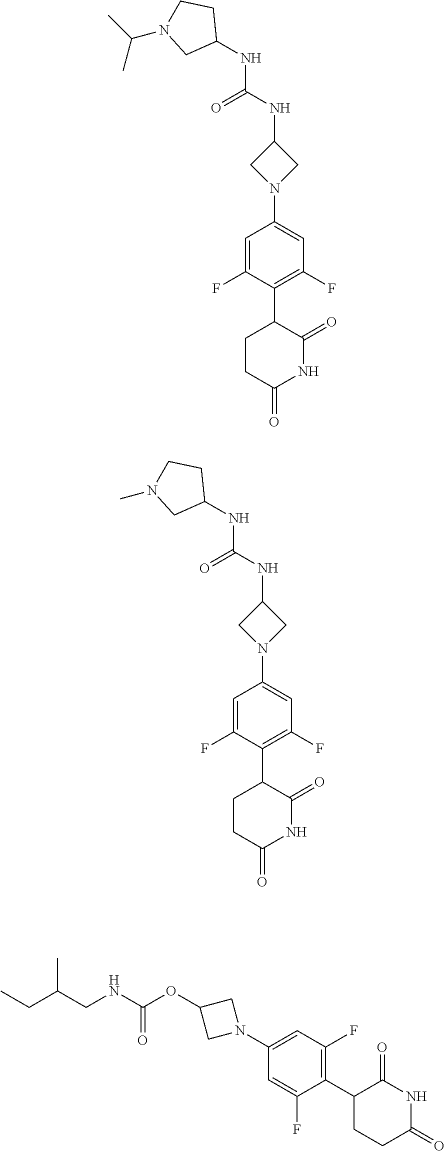 [SMILES: CC(C)N1CCC(NC(=O)NC2CN(c3cc(F)c(C4CCC(=O)NC4=O)c(F)c3)C2)C1 ; CN1CCC(NC(=O)NC2CN(c3cc(F)c(C4CCC(=O)NC4=O)c(F)c3)C2)C1 ; CCC(C)CNC(=O)OC1CN(c2cc(F)c(C3CCC(=O)NC3=O)c(F)c2)C1] 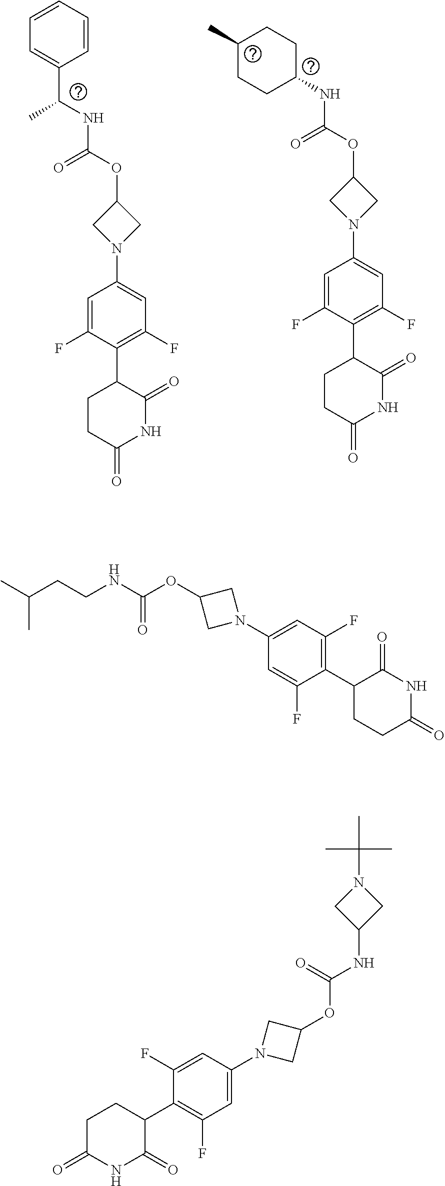 [SMILES: C[C@@H](NC(=O)OC1CN(c2cc(F)c(C3CCC(=O)NC3=O)c(F)c2)C1)c1ccccc1 ; C[C@H]1CC[C@H](NC(=O)OC2CN(c3cc(F)c(C4CCC(=O)NC4=O)c(F)c3)C2)CC1 ; CC(C)CCNC(=O)OC1CN(c2cc(F)c(C3CCC(=O)NC3=O)c(F)c2)C1 ; CC(C)(C)N1CC(NC(=O)OC2CN(c3cc(F)c(C4CCC(=O)NC4=O)c(F)c3)C2)C1] 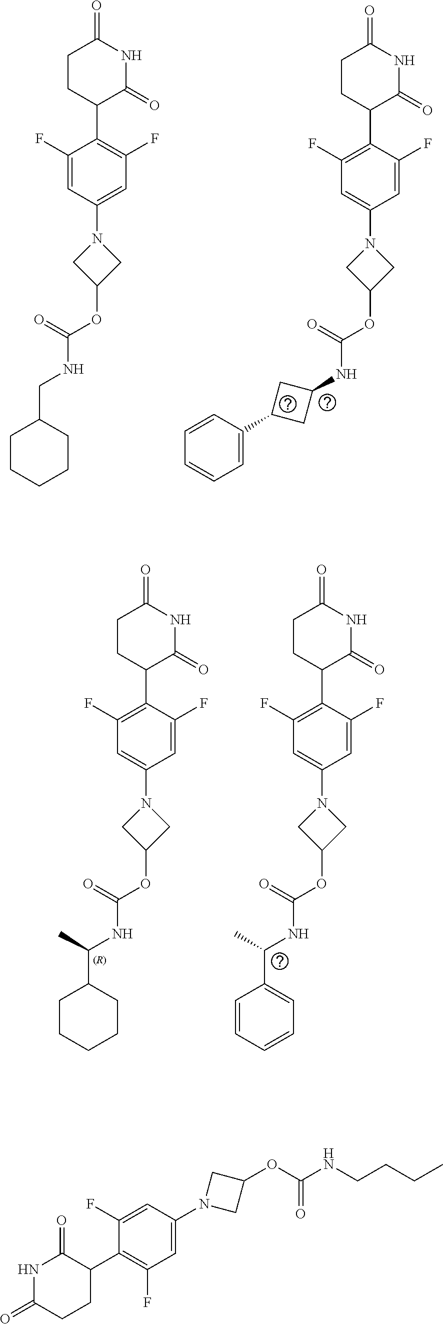 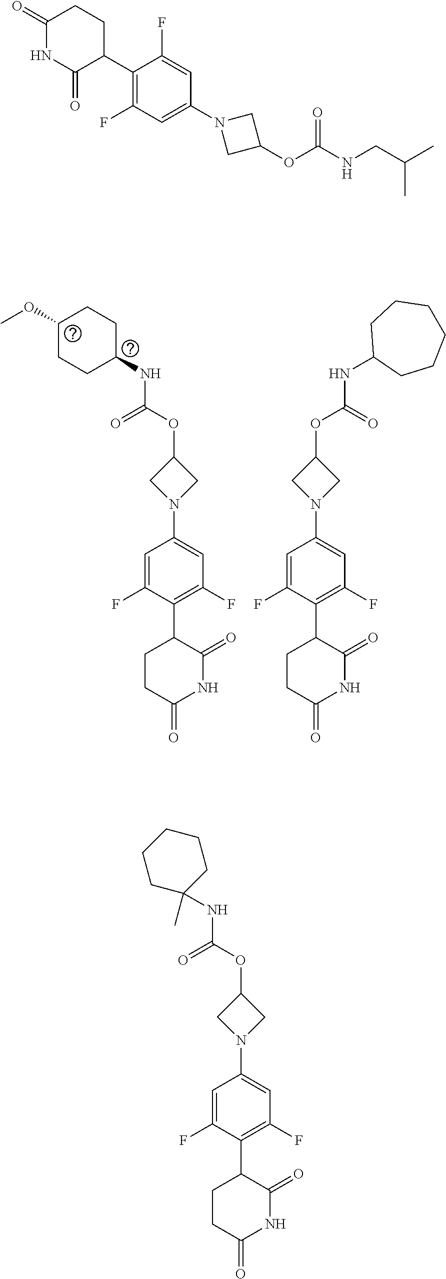 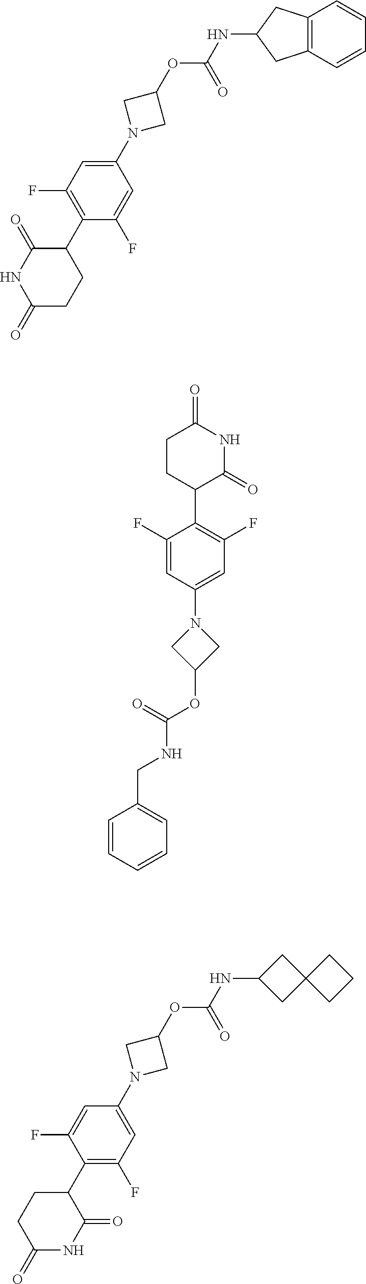 [SMILES: O=C1CCC(c2c(F)cc(N3CC(OC(=O)NC4Cc5ccccc5C4)C3)cc2F)C(=O)N1 ; O=C1CCC(c2c(F)cc(N3CC(OC(=O)NCc4ccccc4)C3)cc2F)C(=O)N1 ; O=C1CCC(c2c(F)cc(N3CC(OC(=O)NC4CC5(CCC5)C4)C3)cc2F)C(=O)N1] 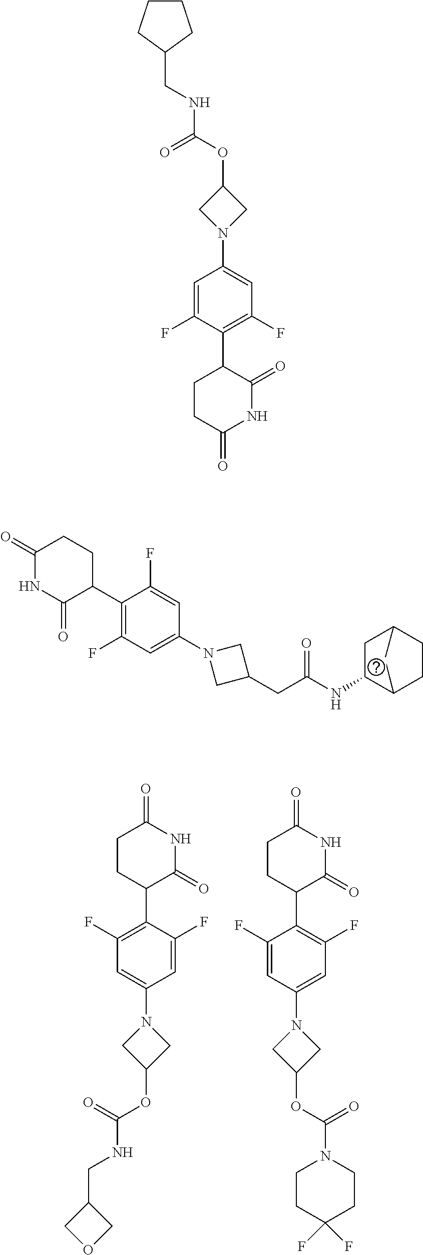 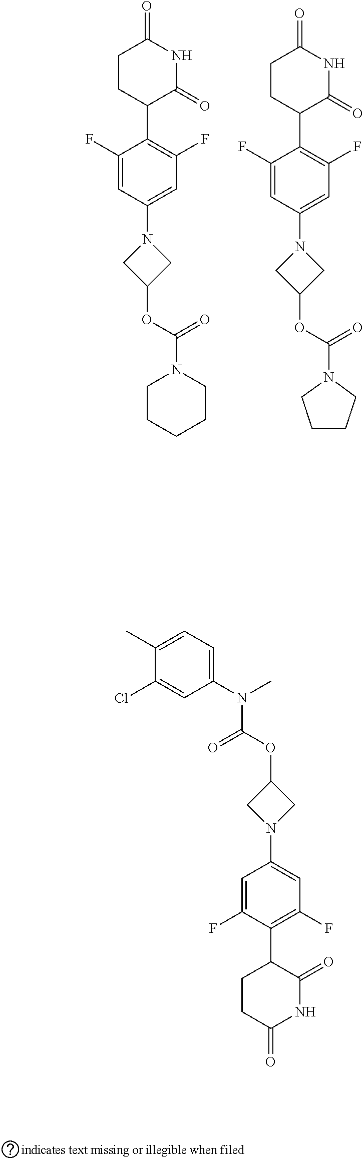
and pharmaceutically acceptable salts thereof.

18. A pharmaceutical composition comprising the compound of claim 1, or pharmaceutically acceptable salt thereof, and a pharmaceutically acceptable excipient.

19. A method of degrading GSPT1 in a subject suffering from cancer, comprising administering to the subject an effective amount of the compound of claim 1.

20. A method of treating cancer in a subject in need thereof, comprising administering to the subject a therapeutically effective amount of the compound of claim 1.
